(19) United States
(12) Patent Application Publication
(10) Pub. No.: US 2012/0058984 A1
(43) Pub. Date: Mar. 8, 2012

(54) PYRIMIDINE DERIVATIVES USED AS ITK INHIBITORS

(76) Inventors: Catherine Mary Alder, Herfordshire (GB); Ian Robert Baldwin, Herfordshire (GB); Nicholas Paul Barton, Herfordshire (GB); Amanda Jennifer Campbell, Herfordshire (GB); Aurelie Cecile Champigny, Herfordshire (GB); John David Harling, Herfordshire (GB); Aoife Caitriona Maxwell, Herfordshire (GB); Juliet Kay Simpson, Herfordshire (GB); Ian Edward David Smith, Herfordshire (GB); Christopher John Tame, Herfordshire (GB); Caroline Wilson, Herfordshire (GB); James Michael Woolven, Herfordshire (GB)

(21) Appl. No.: 13/257,098

(22) PCT Filed: Mar. 15, 2010
(86) PCT No.: PCT/EP10/53289

§ 371 Date: Nov. 15, 2011

Related U.S. Application Data
(60) Provisional application No. 61/160,898, filed on Mar. 17, 2009.

(51) Int. Cl. A61K 31/506; C07D 417/14; A61K 31/5377; A61K 31/541; A61P 11/00; A61P 37/08; A61P 37/00; A61P 37/06; A61P 29/00; A61P 31/18; A61P 7/06; A61P 25/00; A61P 11/06; A61P 11/08; A61P 17/00; A61P 17/06; A61P 3/10; A61P 5/16; A61P 27/02; A61P 1/00; C07D 417/12

(52) U.S. Cl. 514/210.02; 544/324; 514/275; 544/122; 514/234.2; 514/252.18; 544/80; 514/232.5; 544/58.2; 514/228.5; 544/238; 514/252.02

(57) ABSTRACT

The invention is directed to certain novel compounds. Specifically, the invention is directed to compounds of formula (I):

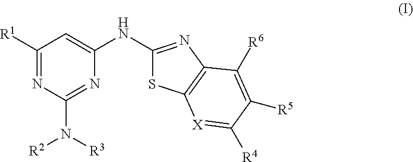

and salts thereof. The compounds of the invention are inhibitors of kinase activity, in particular Itk activity.

### FIELD OF THE INVENTION

The present invention is directed to certain novel compounds which are inhibitors of kinase activity, processes for their preparation, pharmaceutical compositions comprising the compounds, and the use of the compounds or the compositions in the treatment of various disorders. More specifically, the compounds of the invention are inhibitors of the activity or function of Itk (interleukin-2 inducible tyrosine kinase). Compounds which are inhibitors of the activity or function of Itk may be useful in the treatment of disorders such as respiratory diseases including asthma, chronic obstructive pulmonary disease (COPD) and bronchitis; allergic diseases including allergic rhinitis and atopic dermatitis; autoimmune diseases including rheumatoid arthritis, multiple sclerosis, psoriasis, type I diabetes, T cell mediated hypersensitivities, Guillain-Barre Syndrome and Hashimoto's thyroiditis; transplant rejection; graft versus host disease; inflammatory disorders including conjunctivitis, contact dermatitis, inflammatory bowel disease and chronic inflammation; HIV; aplastic anemia; and pain including inflammatory pain.

### BACKGROUND OF THE INVENTION

Interleukin-2 inducible tyrosine kinase (Itk) is a non-receptor tyrosine kinase of the Tec family, which is also known as Tsk or Emt. Other members of the Tec family include: Tec, Btk, Txk and Bmx. The Tec family kinases are predominantly expressed in haematopoietic cells, however Bmx and Tec have a wider expression profile. The Tec family kinases share a common domain structure: an amino-terminal pleckstrin homology (PH) domain (absent in Txk), a tec homology domain (containing one or two proline rich regions), followed by Src homology SH3 and SH2 domains, and a carboxy-terminal kinase domain. The PH domain binds to PtdIn(3,4,5)P3, and is responsible for locating the Tec kinase to the plasma membrane, whilst the PRR, SH3 and SH2 domains are involved in protein-interactions important in formation of the signalling complex.

Itk expression is restricted to T cells, NK and mast cells. Itk is the predominant Tec family kinase in naïve T cells, which also express Txk and Tec. Upon activation via the T cell receptor or interleukin-2 (IL-2), the expression of Itk increases. There is some evidence that Itk is preferentially expressed in Th2 over Th1 cells, in contrast to Txk which is present at higher levels in Th1 cells (1).

Itk plays a key role in T cell receptor signalling. Itk is recruited to the plasma membrane through interaction with PtdIns(3,4,5)P3, which is generated by PI3kinase. Itk forms a complex with several signalling and scaffold proteins including SLP76 and LAT. Itk is transphorphorylated by Lck. Activated Itk phosphorylates PLCγ, leading to the generation of Ins(1,4,5)P3 (required for calcium flux within the cells) and diacylglycerol (activates members of the protein kinase C family and RAS guanyl-releasing protein. This results in the activation of mitogen-activated protein kinases (including JNK and ERK) and other effectors that regulate gene transcription, leading to the secretion of cytokines (reviewed in ref 2). In addition to the role of Itk in PLCγ activation and Ca²⁺ mobilisation, Itk may also contribute to TCR-induced actin reorganisation, and formation of the immune synapse. However, regulation of the actin cytoskeleton may not require kinase activity (3), suggesting the importance of Itk as a scaffold protein. In addition to the T cell receptor, Itk may also be activated via the chemokine receptor CXCR4 (4) in T cells, and via the FcεRI in mast cells (5).

There is considerable evidence suggesting that T cells play a key role in the pathogenesis of asthma. The inhibition of T cell cytokines will dampen down the inflammatory cascade involved in the asthmatic response. Cyclosporin A (CsA), which is thought to exert its major effect via inhibition of T cell cytokine release, has shown significant improvement in lung function in two trials with severe asthmatics (6,7). There is also evidence that CsA is steroid sparing and may lead to fewer exacerbations (7). A further trial reported some benefit of CsA but was non-significant (8). CsA does have actions on other cell types (e.g. mast cells) in addition to T cells. However, following allergen challenge in allergic asthmatics, CsA inhibited the late phase but not the early phase response (9), suggesting that effects on mast cells are unlikely to play a key role in the beneficial effect seen of CsA. Furthermore, daclizumab, an antibody against the anti-IL-2Rα chain (CD25) of activated lymphocytes improved pulmonary function and asthma control in patients with moderate to severe chronic asthma (10), supporting anti-T cell therapy for asthma.

Inhibition of Itk represents a potential novel therapy for asthma, by inhibiting T cell cytokine release. The key role for Itk in T cell receptor signalling has been demonstrated using Itk−/− mice and siRNA. In vitro activation of CD4+ cells from Itk knockout mice show reduced levels of Th2 (11) or both Th1 and Th2 (12) cytokines compared to wild type. Naïve T cells from Itk knockout mice can differentiate normally into either Th1 or Th2 cells if cultured in vitro under appropriate cytokine conditions, suggesting that Itk is not required for Th2 cell differentiation (12). Studies differ in the reported effect of Itk knockout on cytokine release upon re-stimulation, showing either a selective reduction in Th2 or reduction in both Th1 and Th2 cytokines (12, 13). Itk siRNA inhibits cytokine release (Th1 and Th2) from human peripheral blood T cells following activation either with anti-CD3/CD28 or in response to recall antigen in vitro.

Itk−/− mice show reduced lung Th2 cytokine production, cell influx, mast cell degranulation and airway hyperreactivity to methacholine in murine Ova challenge models (14,15,16). In addition to these knockout studies there is also evidence that an Itk inhibitor is effective at reducing cellular influx in an ova murine model of allergic asthma (17). These studies, together with the in vitro profile of Itk inhibitors in human T cells, suggests that Itk is a potential novel target for asthma therapy.

Inhibition of Itk may be beneficial in a variety of T-cell mediated diseases. In addition to asthma, Itk may play a role in other allergic diseases such as allergic rhinitis and atopic dermatitis. Single nucleotide polymorphisms in Itk have been associated with atopy (18) and seasonal allergic rhinitis (19). Itk mRNA levels in the peripheral blood T cells of atopic dermatitis patient is elevated in T cells from affected patients, compared to healthy controls (20).

### REFERENCES

1. Miller et al. Immunity (2004) 21, 67-80
2. Schwartzberg et al. Nature Reviews Immunol (2005) 5, 284-295
3. Grasis et al. J Immunol (2003) 170, 3971-3976
4. Fischer et al. J Biol Chem (2004) 279, (28), 29816-29820
5. Kawakami et al. J Immunol (1995) 155, 3556-3562
6. Alexander et al. The Lancet (1992) 339, 324-328
7. Lock et al. Am J Respir Crit Care Med (1996) 153, 509-514
8. Nizankowska et al. Eur Respir J (1995) 8, 1091-1099
9. Sihra et al. Thorax (1997) 52, 447-452
10. Busse et al. Am J Respir Crit Care med (2008) 178, 1002-1008
11. Fowell et al. Immunity (1999) 11, 399-409
12. Schaeffer et al. Nature Immunol (2001) 2, (12) 1183-1188
13. Au-Yeung et al. J Immunol (2006) 176, 3895-3899
14. Ferrara et al. J Allergy Clin Immunol (2006) 117, 780-786
15. Forssell et al. Am J Respir Cell Mol Biol (2005) 32, 511-520
16. Mueller and August J Immunol (2003) 170, 5056-5063
17. Lin et al., Biochemistry (2004) 43, (34) 11056-11062
18. Graves et al. J Allergy Clin Immunol (2005) 116, 650-656
19. Benson et al. Allergy (2009) DOI:10.111/j.1398-9995.2009.01991.x
20. Matsumoto et al. Int Arch Allergy Immunol (2002) 129, 327-340

Attempts have been made to prepare compounds which inhibit Itk activity and a number of such compounds have been disclosed in the art. However, in view of the number of pathological responses which are mediated by Itk, there remains a continuing need for inhibitors of Itk which can be used in the treatment of a variety of conditions.

The present inventors have discovered novel compounds which are inhibitors of kinase activity, in particular Itk activity. Compounds which are Itk inhibitors may be useful in the treatment of disorders associated with inappropriate kinase activity, in particular inappropriate Itk activity, for example in the treatment and prevention of disorders mediated by Itk mechanisms. Such disorders include respiratory diseases including asthma, chronic obstructive pulmonary disease (COPD) and bronchitis; allergic diseases including allergic rhinitis and atopic dermatitis; autoimmune diseases including rheumatoid arthritis, multiple sclerosis, psoriasis, type I diabetes, T cell mediated hypersensitivities, Guillain-Barre Syndrome and Hashimoto's thyroiditis; transplant rejection; graft versus host disease; inflammatory disorders including conjunctivitis, contact dermatitis, inflammatory bowel disease and chronic inflammation; HIV; aplastic anemia; and pain including inflammatory pain.

In one embodiment, compounds of the invention may show selectivity for Itk over other kinases.

### SUMMARY OF THE INVENTION

The invention is directed to certain novel compounds. Specifically, the invention is directed to compounds of formula (I)

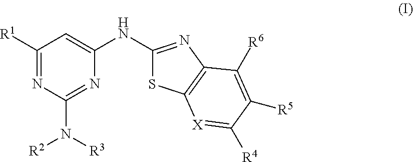

wherein R¹ to R⁶ and X are as defined below, and salts thereof.

The compounds are inhibitors of kinase activity, in particular Itk activity. Compounds which are Itk inhibitors may be useful in the treatment of disorders associated with inappropriate Itk activity, such as asthma. Accordingly, the invention is further directed to pharmaceutical compositions comprising a compound of formula (I) or a pharmaceutically acceptable salt thereof. The invention is still further directed to methods of inhibiting Itk activity and treatment of disorders associated therewith using a compound of formula (I) or a pharmaceutically acceptable salt thereof, or a pharmaceutical composition comprising a compound of formula (I) or a pharmaceutically acceptable salt thereof. The invention is yet further directed towards processes for the preparation of the compounds of the invention.

### DETAILED DESCRIPTION OF THE INVENTION

In one embodiment, the invention is directed to compounds of formula (I)

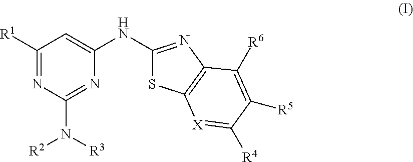

wherein

R¹ is hydrogen, —CR⁷R⁸R⁹, —CH₂OR²⁴, —CH₂NR²⁵R²⁶ or —CH₂-6-membered heteroaryl wherein the 6-membered heteroaryl contains one or two nitrogen atoms and is optionally substituted by one or two substituents independently selected from C₁₋₆alkyl and —OH;

R² is hydrogen or methyl;

R³ is C₁₋₆alkyl substituted by —OH or —NH₂,

C₃₋₆cycloalkyl substituted by C₁₋₆alkyl, —OH, —NR²⁷R²⁸, —CO₂H or —CONH₂,

—(CH₂)ₘ6-membered heterocyclyl wherein the 6-membered heterocyclyl contains one or two heteroatoms independently selected from nitrogen and oxygen and is optionally substituted by —SO₂CH₃ or C₁₋₆alkyl optionally substituted by —CO₂H, naphthyl substituted by —CO₂H, or

—(CH₂)ₙphenyl wherein the phenyl is substituted by one or two substituents independently selected from −OR¹⁰, —SR¹¹, halo, —CO₂H, —SO₂NHR¹², C₁₋₆alkyl optionally substituted by —OH, —CO₂H or —CONR¹³R¹⁴, C₂₋₆alkenyl optionally substituted by —CO₂H and C₃₋₆cycloalkyl optionally substituted by —CO₂H;

R⁴ is hydrogen, —OR¹⁵, halo, —CF₃, —CN, —NO₂, —NR¹⁶R¹⁷, —CO₂R¹⁸, —SO₂CH₃, —NHSO₂CH₃, C₁₋₆alkyl optionally substituted by —OH, —CN, —CO₂R¹⁹ or —CONH₂, pyridinyl optionally substituted by —OR²⁹, —CH₂NR³⁰R³¹ or —CN, or 5-membered heteroaryl wherein the 5-membered heteroaryl contains one or two heteroatoms independently selected from oxygen and nitrogen and is optionally substituted by C₁₋₆alkyl;

R⁵ and R⁶ are each independently hydrogen or fluoro;

R⁷ and R⁸ are both hydrogen, or R⁷ and R⁸ are both fluoro;

R⁹ is hydrogen, C₁₋₆alkyl, or phenyl optionally substituted by fluoro;

R¹⁰ is hydrogen or C₁₋₆alkyl optionally substituted by —CO₂R²⁰;

R¹¹ is C₁₋₆alkyl optionally substituted by —CO₂H;

R¹² is hydrogen or —COC₁₋₆alkyl;

R¹³ and R¹⁴ are each independently hydrogen or C₁₋₆alkyl optionally substituted by —OH, or R¹³ and R¹⁴, together with the nitrogen atom to which they are attached, are linked to form a 6-membered heterocyclyl optionally containing an oxygen atom;

R¹⁵, R²¹ and R²² are each independently C₁₋₆alkyl;

R¹⁶ and R¹⁷ are each independently hydrogen, —COR²¹, —CO₂R²², or C₁₋₆alkyl optionally substituted by one or two —OH groups, or R¹⁶ and R¹⁷, together with the nitrogen atom to which they are attached, are linked to form a 4-, 5- or 6-membered heterocyclyl wherein the 4-membered heterocyclyl is optionally substituted by oxo and the 5- or 6-membered heterocyclyl optionally contains an oxygen atom, a sulphur atom or a further nitrogen atom and is optionally substituted by one or two substituents independently selected from oxo, —OH, —NH₂ and C₁₋₆alkyl optionally substituted by —OH or —NH₂;

R¹⁸, R¹⁹, R²⁰, R²⁴, R³², R³³, R³⁶, R³⁷, R³⁸, R³⁹, R⁴⁰, R⁴¹, R⁴² and R⁴³ are each independently hydrogen or C₁₋₆alkyl;

R²³ is hydrogen or halo;

R²⁵ is hydrogen or C₁₋₆alkyl optionally substituted by —OR³² and R²⁶ is C₁₋₈alkyl optionally substituted by —OR³³, —NR³⁴R³⁵ or —CF₃, or 5- or 6-membered heterocyclyl wherein the 5- or 6-membered heterocyclyl contains a heteroatom selected from oxygen, sulphur and nitrogen and is optionally substituted by one or two oxo substituents, or R²⁵ and R²⁶, together with the nitrogen atom to which they are attached, are linked to form a 4-, 5- or 6-membered heterocyclyl wherein the 4-membered heterocyclyl is optionally substituted by one or two substituents independently selected from halo and the 5- or 6-membered heterocyclyl optionally contains an oxygen atom, a sulphur atom or a further nitrogen atom and is optionally substituted by one or two substituents independently selected from oxo, C₁₋₆alkyl optionally substituted by —OR³⁶, halo, —OR³⁷ and —CO₂R³⁸;

R²⁷ and R²⁸ are each hydrogen, or R²⁷ and R²⁸, together with the nitrogen atom to which they are attached, are linked to form a 6-membered heterocyclyl wherein the 6-membered heterocyclyl optionally contains an oxygen atom;

R²⁹ is tetrahydropyran, or C₁₋₆alkyl optionally substituted by —OR³⁹ or —NR⁴⁰R⁴¹;

R³⁰ is hydrogen and R³¹ is C₁₋₆alkyl optionally substituted by —OR⁴², or R³⁰ and R³¹, together with the nitrogen atom to which they are attached, are linked to form a 6-membered heterocyclyl wherein the 6-membered heterocyclyl optionally contains an oxygen atom or a further nitrogen atom and is optionally substituted by one or two substituents independently selected from C₁₋₆alkyl;

R³⁴ is hydrogen or C₁₋₆alkyl and R³⁵ is —CO₂R⁴³, or R³⁴ and R³⁵, together with the nitrogen atom to which they are attached, are linked to form a 5- or 6-membered heterocyclyl wherein the 5- or 6-membered heterocyclyl optionally contains an oxygen atom or a sulphur atom and is optionally substituted by one or two oxo substituents;

X is —N— or —CR²³—;

and

m and n are each independently 0, 1, 2 or 3;

and salts thereof (hereinafter “compounds of the invention”).

In a further embodiment, the invention is directed to compounds of formula (IA)

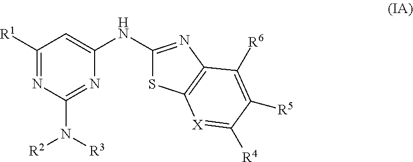

wherein

R¹ is hydrogen or —CR⁷R⁸R⁹;

R² is hydrogen or methyl;

R³ is C₁₋₆alkyl substituted by —OH or —NH₂,

C₃₋₆cycloalkyl substituted by C₁₋₆alkyl, —OH, —NH₂, —CO₂H or —CONH₂,

—(CH₂)ₘ6-membered heterocyclyl wherein the 6-membered heterocyclyl contains one or two heteroatoms independently selected from nitrogen and oxygen and is optionally substituted by —SO₂CH₃ or C₁₋₆alkyl optionally substituted by —CO₂H, naphthyl substituted by —CO₂H, or

—(CH₂)ₙphenyl wherein the phenyl is substituted by one or two substituents independently selected from −OR¹⁰, —SR¹¹, halo, —CO₂H, —SO₂NHR¹², C₁₋₆alkyl optionally substituted by —OH, —CO₂H or —CONR¹³R¹⁴, C₂₋₆alkenyl optionally substituted by —CO₂H and C₃₋₆cycloalkyl optionally substituted by —CO₂H;

R⁴ is hydrogen, —OR¹⁵, halo, —CF₃, —CN, —NO₂, —NR¹⁶R¹⁷, —CO₂R¹⁸, —SO₂CH₃, —NHSO₂CH₃, or C₁₋₆alkyl optionally substituted by —OH, —CN, —CO₂R¹⁹ or —CONH₂;

R⁵ and R⁶ are each independently hydrogen or fluoro;

R⁷ and R⁸ are both hydrogen, or R⁷ and R⁸ are both fluoro;

R⁹ is C₁₋₆alkyl or phenyl optionally substituted by fluoro;

R¹⁰ is hydrogen or C₁₋₆alkyl optionally substituted by —CO₂R²⁰;

R¹¹ is C₁₋₆alkyl optionally substituted by —CO₂H;

R¹² is hydrogen or —COC₁₋₆alkyl;

R¹³ and R¹⁴ are each independently hydrogen or C₁₋₆alkyl optionally substituted by —OH, or R¹³ and R¹⁴, together with the nitrogen atom to which they are attached, are linked to form a 6-membered heterocyclyl optionally containing an oxygen atom;

R¹⁵, R²¹ and R²² are each independently C₁₋₆alkyl;

R¹⁶ and R¹⁷ are each independently hydrogen, —COR²¹, —CO₂R²², or C₁₋₆alkyl optionally substituted by one or two —OH groups, or R¹⁶ and R¹⁷, together with the nitrogen atom to which they are attached, are linked to form a 4-, 5- or 6-membered heterocyclyl wherein the 4-membered heterocyclyl is optionally substituted by oxo and the 5- or 6-membered heterocyclyl optionally contains an oxygen atom or a further nitrogen atom and is optionally substituted by one or two substituents independently selected from oxo, —OH, —NH₂ and C₁₋₆alkyl optionally substituted by —OH or —NH₂;

R¹⁸, R¹⁹ and R²⁰ are each independently hydrogen or C₁₋₆alkyl;

R²³ is hydrogen or halo;

X is —N— or —CR²³—;

and

m and n are each independently 0, 1, 2 or 3;

and salts thereof.

In one embodiment, R¹ is hydrogen or —CR⁷R⁸R⁹. In another embodiment, R¹ is —CR⁷R⁸R⁹ or —CH₂NR²⁵R²⁶. In another embodiment, R¹ is —CR⁷R⁸R⁹. In a further embodiment, R¹ is —CH₂NR²⁵R²⁶.

The skilled artisan will appreciate that when R¹ is —CH₂-6-membered heteroaryl wherein the 6-membered heteroaryl contains one or two heteroatoms and is optionally substituted by —OH, the R¹ group may be drawn as the corresponding keto tautomer. For example, 3(2H)-pyridazinone may be drawn as follows:

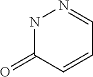

All such tautomeric forms are included whether such tautomers exist in equilibrium or predominantly in one form.

In one embodiment, R² is hydrogen.

In one embodiment, R³ is C₁₋₆alkyl substituted by —OH or —NH₂,

C₃₋₆cycloalkyl substituted by C₁₋₆alkyl, —OH, —NH₂, —CO₂H or —CONH₂, —(CH₂)ₘ6-membered heterocyclyl wherein the 6-membered heterocyclyl contains one or two heteroatoms independently selected from nitrogen and oxygen and is optionally substituted by —SO₂CH₃ or C₁₋₆alkyl optionally substituted by —CO₂H, naphthyl substituted by —CO₂H, or

—(CH₂)ₙphenyl wherein the phenyl is substituted by one or two substituents independently selected from −OR¹⁰, —SR¹¹, halo, —CO₂H, —SO₂NHR¹², C₁₋₆alkyl optionally substituted by —OH, —CO₂H or —CONR¹³R¹⁴, C₂₋₆alkenyl optionally substituted by —CO₂H and C₃₋₆cycloalkyl optionally substituted by —CO₂H. In another embodiment, R³ is C₁₋₆alkyl substituted by —OH or —NH₂,

C₃₋₆cycloalkyl substituted by C₁₋₆alkyl, —OH, —NH₂, —CO₂H or —CONH₂,

—(CH₂)ₘ6-membered heterocyclyl wherein the 6-membered heterocyclyl contains one or two heteroatoms independently selected from nitrogen and oxygen and is substituted by —SO₂CH₃ or C₁₋₆alkyl optionally substituted by —CO₂H,

naphthyl substituted by —CO₂H, or

—(CH₂)ₙphenyl wherein the phenyl is substituted by one or two substituents independently selected from —OR¹⁰, —SR¹¹, halo, —CO₂H, —SO₂NHR¹², C₁₋₆alkyl optionally substituted by —OH, —CO₂H or —CONR¹³R¹⁴, C₂₋₆alkenyl optionally substituted by —CO₂H and C₃₋₆cycloalkyl optionally substituted by —CO₂H. In another embodiment, R³ is C₁₋₆alkyl substituted by —OH, C₃₋₆cycloalkyl substituted by —OH or —CO₂H, or —(CH₂)ₙphenyl wherein the phenyl is substituted by one or two substituents independently selected from −OR¹⁰, halo, —SO₂NHR¹² and C₁₋₆alkyl optionally substituted by —CO₂H. In another embodiment, R³ is C₁₋₆alkyl substituted by —OH, C₃₋₆cycloalkyl substituted by —OH, or —(CH₂)ₙphenyl wherein the phenyl is substituted by C₁₋₆alkyl optionally substituted by —CO₂H. In a further embodiment, R³ is C₃₋₆cycloalkyl substituted by —OH.

In one embodiment, R⁴ is hydrogen, —OR¹⁵, halo, —CF₃, —CN, —NO₂, —NR¹⁶R¹⁷, —CO₂R¹⁸, —SO₂CH₃, —NHSO₂CH₃, or C₁₋₆alkyl optionally substituted by —OH, —CN, —CO₂R¹⁹ or —CONH₂. In another embodiment, R⁴ is hydrogen, —OR¹⁵, halo, —CN, —NO₂, —NR¹⁶R¹⁷, —SO₂CH₃, or C₁₋₆alkyl optionally substituted by —OH, —CN, —CO₂R¹⁹ or —CONH₂. In another embodiment, R⁴ is hydrogen, —OR¹⁵, —CN, —NO₂, —NR¹⁶R¹⁷, or C₁₋₆alkyl optionally substituted by —OH, —CN or —CONH₂. In another embodiment, R⁴ is —NR¹⁶R¹⁷, -pyridinyl optionally substituted by —OR²⁹, —CH₂NR³⁰R³¹ or —CN, or 5-membered heteroaryl wherein the 5-membered heteroaryl contains one or two heteroatoms independently selected from oxygen and nitrogen and is optionally substituted by C₁₋₆alkyl. In a further embodiment, R⁴ is —NR¹⁶R¹⁷, -pyridinyl optionally substituted by —CN, or 5-membered heteroaryl wherein the 5-membered heteroaryl contains one or two heteroatoms independently selected from oxygen and nitrogen.

In one embodiment, R⁵ is hydrogen.

In one embodiment, R⁶ is hydrogen.

In one embodiment, R⁷ and R⁸ are both hydrogen.

In one embodiment, R⁹ is C₁₋₆alkyl or phenyl optionally substituted by fluoro. In another embodiment, R⁹ is C₁₋₆alkyl. In another embodiment, R⁹ is C₁₋₄alkyl. In another embodiment, R⁹ is phenyl optionally substituted by fluoro. In a further embodiment, R⁹ is phenyl.

In one embodiment, R¹⁰ is C₁₋₆alkyl optionally substituted by —CO₂R²⁰. In a further embodiment, R¹⁰ is C₁₋₄alkyl optionally substituted by —CO₂R²⁰.

In one embodiment, R¹¹ is C₁₋₄alkyl optionally substituted by —CO₂H.

In one embodiment, R¹² is hydrogen.

In one embodiment, R¹³ and R¹⁴ are each independently hydrogen or C₁₋₆alkyl optionally substituted by —OH. In a further embodiment, R¹³ and R¹⁴, together with the nitrogen atom to which they are attached, are linked to form a 6-membered heterocyclyl optionally containing an oxygen atom.

In one embodiment, R¹⁵ is C₁₋₄alkyl. In a further embodiment, R¹⁵ is methyl.

In one embodiment, R¹⁶ and R¹⁷ are each independently hydrogen, —COR²¹, —CO₂R²², or C₁₋₆alkyl optionally substituted by one or two —OH groups, or R¹⁶ and R¹⁷, together with the nitrogen atom to which they are attached, are linked to form a 4-, 5- or 6-membered heterocyclyl wherein the 4-membered heterocyclyl is optionally substituted by oxo and the 5- or 6-membered heterocyclyl optionally contains an oxygen atom or a further nitrogen atom and is optionally substituted by one or two substituents independently selected from oxo, —OH, —NH₂ and C₁₋₆alkyl optionally substituted by —OH or —NH₂. In another embodiment, R¹⁶ and R¹⁷ are each independently hydrogen or C₁₋₆alkyl optionally substituted by one or two —OH groups, or R¹⁶ and R¹⁷, together with the nitrogen atom to which they are attached, are linked to form a 4-, 5- or 6-membered heterocyclyl wherein the 4-membered heterocyclyl is optionally substituted by oxo and the 5- or 6-membered heterocyclyl optionally contains an oxygen atom or a further nitrogen atom and is optionally substituted by one or two substituents independently selected from oxo, —OH and —NH₂. In another embodiment, R¹⁶ and R¹⁷ are each independently C₁₋₆alkyl optionally substituted by one or two —OH groups. In another embodiment, R¹⁶ and R¹⁷, together with the nitrogen atom to which they are attached, are linked to form a 4- or 5-membered heterocyclyl wherein the 4-membered heterocyclyl is optionally substituted by oxo and the 5-membered heterocyclyl optionally contains an oxygen atom or a further nitrogen atom and is optionally substituted by one or two oxo groups. In a further embodiment, R¹⁶ and R¹⁷, together with the nitrogen atom to which they are attached, are linked to form a 5-membered heterocyclyl wherein the 5-membered heterocyclyl optionally contains an oxygen atom and is optionally substituted by one or two oxo groups.

In one embodiment, R¹⁸ is hydrogen or C₁₋₄alkyl.

In one embodiment, R¹⁹ is hydrogen or C₁₋₄alkyl.

In one embodiment, R²⁰ is hydrogen or C₁₋₄alkyl. In a further embodiment, R²⁰ is hydrogen.

In one embodiment, R²¹ is C₁₋₄alkyl, for example methyl.

In one embodiment, R²² is C₁₋₄alkyl, for example methyl.

In one embodiment, R²³ is hydrogen or bromo. In a further embodiment, R²³ is hydrogen.

In one embodiment, R²⁴ is hydrogen or C₁₋₄alkyl.

In one embodiment, R²⁵ and R²⁶, together with the nitrogen atom to which they are attached, are linked to form a 5- or 6-membered heterocyclyl wherein the 5- or 6-membered heterocyclyl optionally contains an oxygen atom, a sulphur atom or a further nitrogen atom and is optionally substituted by one or two substituents independently selected from oxo, C₁₋₆alkyl optionally substituted by —OR³⁶, halo, —OR³⁷ and —CO₂R³⁸. In a further embodiment, R²⁵ and R²⁶, together with the nitrogen atom to which they are attached, are linked to form a 6-membered heterocyclyl wherein the 6-membered heterocyclyl optionally contains an oxygen atom and is optionally substituted by one or two substituents independently selected from C₁₋₆alkyl and halo.

In one embodiment, R²⁷ and R²⁸ are each hydrogen. In a further embodiment, R²⁷ and R²⁸, together with the nitrogen atom to which they are attached, are linked to form a 6-membered heterocyclyl wherein the 6-membered heterocyclyl optionally contains an oxygen atom.

In one embodiment, R²⁹ is C₁₋₆alkyl optionally substituted by —OR³⁹ or —NR⁴⁰R⁴¹.

In one embodiment, R³⁰ is hydrogen and R³¹ is C₁₋₆alkyl optionally substituted by —OR⁴². In a further embodiment, R³⁰ and R³¹, together with the nitrogen atom to which they are attached, are linked to form a 6-membered heterocyclyl wherein the 6-membered heterocyclyl contains an oxygen atom or a further nitrogen atom and is optionally substituted by one or two substituents independently selected from C₁₋₆alkyl.

In one embodiment, R³² is C₁₋₄alkyl, for example methyl.

In one embodiment, R³³ is hydrogen or C₁₋₄alkyl.

In one embodiment, R³⁴ is hydrogen and R³⁵ is —CO₂R⁴³. In a further embodiment, R³⁴ and R³⁵, together with the nitrogen atom to which they are attached, are linked to form a 5- or 6-membered heterocyclyl wherein the 5- or 6-membered heterocyclyl optionally contains an oxygen atom or a sulphur atom and is optionally substituted by one or two oxo substituents.

In one embodiment, R³⁶ is C₁₋₄alkyl, for example methyl.

In one embodiment, R³⁷ is C₁₋₄alkyl, for example methyl.

In one embodiment, R³⁸ is hydrogen or C₁₋₄alkyl.

In one embodiment, R³⁹ is hydrogen.

In one embodiment, R⁴⁰ is C₁₋₄alkyl, for example methyl.

In one embodiment, R⁴¹ is C₁₋₄alkyl, for example methyl.

In one embodiment, R⁴² is hydrogen or C₁₋₄alkyl.

In one embodiment, R⁴³ is C₁₋₄alkyl.

In one embodiment, X is —N—. In a further embodiment, X is —CR²³—.

In one embodiment, m is 0 or 1.

In one embodiment, n is 0, 2 or 3. In a further embodiment, n is 0.

It is to be understood that the present invention covers all combinations of substituent groups described hereinabove.

Compounds of the invention include the compounds of Examples 1 to 260 and salts thereof.

In one embodiment, the compound of the invention is:
trans-4-{[4-[(6-methyl-1,3-benzothiazol-2-yl)amino]-6-(phenylmethyl)-2-pyrimidinyl]amino}cyclohexanol;
trans-4-{[4-[(6-chloro-1,3-benzothiazol-2-yl)amino]-6-(phenylmethyl)-2-pyrimidinyl]amino}cyclohexanol;
trans-4-[(4-(phenylmethyl)-6-{[6-(trifluoromethyl)-1,3-benzothiazol-2-yl]amino}-2-pyrimidinyl)amino]cyclohexanol;
trans-4-{[4-[(6-bromo-1,3-benzothiazol-2-yl)amino]-6-(phenylmethyl)-2-pyrimidinyl]amino}cyclohexanol;
trans-4-{[4-[(6-fluoro-1,3-benzothiazol-2-yl)amino]-6-(phenylmethyl)-2-pyrimidinyl]amino}cyclohexanol;
trans-4-{[4-[(6-ethyl-1,3-benzothiazol-2-yl)amino]-6-(phenylmethyl)-2-pyrimidinyl]amino}cyclohexanol;
trans-4-{[4-[(5-fluoro-1,3-benzothiazol-2-yl)amino]-6-(phenylmethyl)-2-pyrimidinyl]amino}cyclohexanol;
trans-4-{[4-{[5-(methyloxy)[1,3]thiazolo[5,4-b]pyridin-2-yl]amino}-6-(phenylmethyl)-2-pyrimidinyl]amino}cyclohexanol;
trans-4-{[4-[(5-chloro[1,3]thiazolo[5,4-b]pyridin-2-yl)amino]-6-(phenylmethyl)-2-pyrimidinyl]amino}cyclohexanol;
(4-{[4-[(6-ethyl-1,3-benzothiazol-2-yl)amino]-6-(phenylmethyl)-2-pyrimidinyl]amino}-1-piperidinyl)acetic acid;
trans-4-{[4-(phenylmethyl)-6-([1,3]thiazolo[5,4-b]pyridin-2-ylamino)-2-pyrimidinyl]amino}cyclohexanol;
trans-4-{[4-[(6-nitro-1,3-benzothiazol-2-yl)amino]-6-(phenylmethyl)-2-pyrimidinyl]amino}cyclohexanol;
(2-{[2-[(trans-4-hydroxycyclohexyl)amino]-6-(phenylmethyl)-4-pyrimidinyl]amino}-1,3-benzothiazol-6-yl)acetonitrile;
5-{[4-[(6-methyl-1,3-benzothiazol-2-yl)amino]-6-(phenylmethyl)-2-pyrimidinyl]amino}-1-pentanol;
2-hydroxy-5-(2-{[4-[(6-methyl-1,3-benzothiazol-2-yl)amino]-6-(phenylmethyl)-2-pyrimidinyl]amino}ethyl)benzenesulfonamide;
4-{[4-[(6-methyl-1,3-benzothiazol-2-yl)amino]-6-(phenylmethyl)-2-pyrimidinyl]amino}-1-butanol;
5-(2-{[4-[(6-ethyl-1,3-benzothiazol-2-yl)amino]-6-(phenylmethyl)-2-pyrimidinyl]amino}ethyl)-2-hydroxybenzenesulfonamide;
4-(2-{[4-[(6-methyl-1,3-benzothiazol-2-yl)amino]-6-(phenylmethyl)-2-pyrimidinyl]amino}ethyl)benzenesulfonamide;
4-{[4-[(6-ethyl-1,3-benzothiazol-2-yl)amino]-6-(phenylmethyl)-2-pyrimidinyl]amino}-1-butanol;
5-{[4-[(6-ethyl-1,3-benzothiazol-2-yl)amino]-6-(phenylmethyl)-2-pyrimidinyl]amino}-1-pentanol;
4-{[4-{[5-(methyloxy)[1,3]thiazolo[5,4-b]pyridin-2-yl]amino}-6-(phenylmethyl)-2-pyrimidinyl]amino}-1-butanol;
5-{[4-{[5-(methyloxy)[1,3]thiazolo[5,4-b]pyridin-2-yl]amino}-6-(phenylmethyl)-2-pyrimidinyl]amino}-1-pentanol;
trans-4-{[4-[(5-ethyl[1,3]thiazolo[5,4-b]pyridin-2-yl)amino]-6-(phenylmethyl)-2-pyrimidinyl]amino}cyclohexanol;
2-{[2-[(trans-4-hydroxycyclohexyl)amino]-6-(phenylmethyl)-4-pyrimidinyl]amino}-1,3-benzothiazole-6-carbonitrile;
ethyl 2-{[2-[(trans-4-hydroxycyclohexyl)amino]-6-(phenylmethyl)-4-pyrimidinyl]amino}-1,3-benzothiazole-6-carboxylate;
ethyl (2-{[2-[(trans-4-hydroxycyclohexyl)amino]-6-(phenylmethyl)-4-pyrimidinyl]amino}-1,3-benzothiazol-6-yl)acetate;
ethyl 3-(2-{[2-[(trans-4-hydroxycyclohexyl)amino]-6-(phenylmethyl)-4-pyrimidinyl]amino}-1,3-benzothiazol-6-yl)propanoate;
trans-4-{[4-{[6-(1-methylethyl)-1,3-benzothiazol-2-yl]amino}-6-(phenylmethyl)-2-pyrimidinyl]amino}cyclohexanol;
4-(2-{[4-[(6-ethyl-1,3-benzothiazol-2-yl)amino]-6-(2-methylpropyl)-2-pyrimidinyl]amino}ethyl)benzenesulfonamide;
4-(2-{[4-[(6-ethyl-1,3-benzothiazol-2-yl)amino]-6-(phenylmethyl)-2-pyrimidinyl]amino}ethyl)benzenesulfonamide;
trans-4-{[4-[(6-iodo-1,3-benzothiazol-2-yl)amino]-6-(phenylmethyl)-2-pyrimidinyl]amino}cyclohexanol;
5-(2-{[4-[(6-ethyl-1,3-benzothiazol-2-yl)amino]-6-(2-methylpropyl)-2-pyrimidinyl]amino}ethyl)-2-hydroxybenzenesulfonamide;
(1R,3S)-3-{[4-[(6-ethyl-1,3-benzothiazol-2-yl)amino]-6-(phenylmethyl)-2-pyrimidinyl]amino}cyclopentanecarboxylic acid;
trans-4-({4-(phenylmethyl)-6-[(6-propyl-1,3-benzothiazol-2-yl)amino]-2-pyrimidinyl}amino)cyclohexanol;
(1R,2S)-2-{[4-[(6-ethyl-1,3-benzothiazol-2-yl)amino]-6-(phenylmethyl)-2-pyrimidinyl]amino}cyclohexanecarboxylic acid;
5-{[4-[(6-ethyl-1,3-benzothiazol-2-yl)amino]-6-(2-methylpropyl)-2-pyrimidinyl]amino}-1-pentanol;
4-(2-{[4-{[6-(1-methylethyl)-1,3-benzothiazol-2-yl]amino}-6-(phenylmethyl)-2-pyrimidinyl]amino}ethyl)benzenesulfonamide;
4-{[4-[(6-ethyl-1,3-benzothiazol-2-yl)amino]-6-(2-methylpropyl)-2-pyrimidinyl]amino}-1-butanol;
trans-4-{[4-{[6-(1,1-dimethylethyl)-1,3-benzothiazol-2-yl]amino}-6-(phenylmethyl)-2-pyrimidinyl]amino}cyclohexanol;
3-{[4-[(6-ethyl-1,3-benzothiazol-2-yl)amino]-6-(phenylmethyl)-2-pyrimidinyl]amino}cyclohexanecarboxylic acid;
trans-4-({4-[(6-methyl-1,3-benzothiazol-2-yl)amino]-2-pyrimidinyl}amino)cyclohexanol;
N4-(6-ethyl-1,3-benzothiazol-2-yl)-N2-(1-methyl-4-piperidinyl)-6-(phenylmethyl)-2,4-pyrimidinediamine;
N4-(6-ethyl-1,3-benzothiazol-2-yl)-N2-{[4-(methylsulfonyl)-2-morpholinyl]methyl}-6-(phenylmethyl)-2,4-pyrimidinediamine;
4-(2-{[4-{[6-(1,1-dimethylethyl)-1,3-benzothiazol-2-yl]amino}-6-(phenylmethyl)-2-pyrimidinyl]amino}ethyl)benzenesulfonamide;
4-[[4-[(6-ethyl-1,3-benzothiazol-2-yl)amino]-6-(phenylmethyl)-2-pyrimidinyl](methyl)amino]-1-butanol trifluoroacetate;
(1R,2R)-2-{[4-[(6-ethyl-1,3-benzothiazol-2-yl)amino]-6-(phenylmethyl)-2-pyrimidinyl]amino}cyclohexanol;
(1R,2R)-2-{[4-[(6-ethyl-1,3-benzothiazol-2-yl)amino]-6-(phenylmethyl)-2-pyrimidinyl]amino}cyclohexanecarboxylic acid;
trans-4-{[4-[(4-fluoro-1,3-benzothiazol-2-yl)amino]-6-(phenylmethyl)-2-pyrimidinyl]amino}cyclohexanol;
(1S,2R)-2-{[4-[(6-ethyl-1,3-benzothiazol-2-yl)amino]-6-(phenylmethyl)-2-pyrimidinyl]amino}cyclohexanecarboxamide;
N4-(6-ethyl-1,3-benzothiazol-2-yl)-N2-(trans-4-methylcyclohexyl)-6-(phenylmethyl)-2,4-pyrimidinediamine;
4-(3-{[4-[(6-ethyl-1,3-benzothiazol-2-yl)amino]-6-(phenylmethyl)-2-pyrimidinyl]amino}propyl)benzenesulfonamide;
3-{[4-{[6-(methyloxy)-1,3-benzothiazol-2-yl]amino}-6-(phenylmethyl)-2-pyrimidinyl]amino}-1-propanol;
trans-4-{[4-{[6-(methylsulfonyl)-1,3-benzothiazol-2-yl]amino}-6-(phenylmethyl)-2-pyrimidinyl]amino}cyclohexanol;
trans-4-{[4-[(7-bromo-1,3-benzothiazol-2-yl)amino]-6-(phenylmethyl)-2-pyrimidinyl]amino}cyclohexanol;
3-(4-{[4-[(6-ethyl-1,3-benzothiazol-2-yl)amino]-6-(phenylmethyl)-2-pyrimidinyl]amino}phenyl)propanoic acid;
3-(4-{[4-{[5-(methyloxy)[1,3]thiazolo[5,4-b]pyridin-2-yl]amino}-6-(phenylmethyl)-2-pyrimidinyl]amino}phenyl)propanoic acid;
3-(4-{[4-[(6-ethyl-1,3-benzothiazol-2-yl)amino]-6-(phenylmethyl)-2-pyrimidinyl]amino}phenyl)propanoic acid;
(2E)-3-(4-{[4-[(6-ethyl-1,3-benzothiazol-2-yl)amino]-6-(phenylmethyl)-2-pyrimidinyl]amino}phenyl)-2-propenoic acid;
(4-{[4-[(6-ethyl-1,3-benzothiazol-2-yl)amino]-6-(phenylmethyl)-2-pyrimidinyl]amino}phenyl)acetic acid;
3-(4-{[4-[(5-ethyl[1,3]thiazolo[5,4-b]pyridin-2-yl)amino]-6-(phenylmethyl)-2-pyrimidinyl]amino}phenyl)propanoic acid;
(4-{[4-[(6-ethyl-1,3-benzothiazol-2-yl)amino]-6-(phenylmethyl)-2-pyrimidinyl]amino}-3-fluorophenyl)acetic acid;
3-[(4-{[4-[(6-methyl-1,3-benzothiazol-2-yl)amino]-6-(phenylmethyl)-2-pyrimidinyl]amino}phenyl)oxy]propanoic acid;
2-(4-{[4-[(6-ethyl-1,3-benzothiazol-2-yl)amino]-6-(phenylmethyl)-2-pyrimidinyl]amino}phenyl)cyclopropanecarboxylic acid;
2-(4-{[4-[(6-ethyl-1,3-benzothiazol-2-yl)amino]-6-(2-methylpropyl)-2-pyrimidinyl]amino}-3-fluorophenyl)propanoic acid;
[(4-{[4-[(6-methyl-1,3-benzothiazol-2-yl)amino]-6-(phenylmethyl)-2-pyrimidinyl]amino}phenyl)thio]acetic acid;
2-(4-{[4-[(6-ethyl-1,3-benzothiazol-2-yl)amino]-6-(phenylmethyl)-2-pyrimidinyl]amino}phenyl)cyclopropanecarboxylic acid;
[(4-{[4-[(6-ethyl-1,3-benzothiazol-2-yl)amino]-6-(phenylmethyl)-2-pyrimidinyl]amino}phenyl)oxy]acetic acid;
2-[(4-{[4-[(6-ethyl-1,3-benzothiazol-2-yl)amino]-6-(phenylmethyl)-2-pyrimidinyl]amino}phenyl)oxy]-2-methylpropanoic acid;
3-[(4-{[4-[(6-ethyl-1,3-benzothiazol-2-yl)amino]-6-(2-methylpropyl)-2-pyrimidinyl]amino}phenyl)oxy]propanoic acid;
3-(4-{[4-({6-[(ethyloxy)carbonyl]-1,3-benzothiazol-2-yl}amino)-6-(phenylmethyl)-2-pyrimidinyl]amino}phenyl)propanoic acid;
3-methyl-4-{[4-{[5-(methyloxy)[1,3]thiazolo[5,4-b]pyridin-2-yl]amino}-6-(phenylmethyl)-2-pyrimidinyl]amino}benzenesulfonamide;
3-methyl-4-{[4-[(6-methyl-1,3-benzothiazol-2-yl)amino]-6-(phenylmethyl)-2-pyrimidinyl]amino}benzenesulfonamide;
[(4-{[4-[(6-ethyl-1,3-benzothiazol-2-yl)amino]-6-(2-methylpropyl)-2-pyrimidinyl]amino}phenyl)thio]acetic acid;
4-{[4-{[5-(methyloxy)[1,3]thiazolo[5,4-b]pyridin-2-yl]amino}-6-(phenylmethyl)-2-pyrimidinyl]amino}benzenesulfonamide;
2-(4-{[4-[(6-ethyl-1,3-benzothiazol-2-yl)amino]-6-(phenylmethyl)-2-pyrimidinyl]amino}phenyl)acetamide;
4-{[4-[(6-ethyl-1,3-benzothiazol-2-yl)amino]-6-(2-methylpropyl)-2-pyrimidinyl]amino}benzenesulfonamide;
4-{[4-[(6-methyl-1,3-benzothiazol-2-yl)amino]-6-(2-methylpropyl)-2-pyrimidinyl]amino}benzenesulfonamide;
4-{[4-[(6-ethyl-1,3-benzothiazol-2-yl)amino]-6-(2-methylpropyl)-2-pyrimidinyl]amino}-3-methylbenzenesulfonamide;
4-{[4-[(6-ethyl-1,3-benzothiazol-2-yl)amino]-6-(phenylmethyl)-2-pyrimidinyl]amino}-3-methylbenzenesulfonamide;
4-{[4-[(6-ethyl-1,3-benzothiazol-2-yl)amino]-6-(phenylmethyl)-2-pyrimidinyl]amino}benzenesulfonamide;
4-{[4-[(6-ethyl-1,3-benzothiazol-2-yl)amino]-6-(phenylmethyl)-2-pyrimidinyl]amino}benzoic acid;
(4-{[4-{[6-(1-methylethyl)-1,3-benzothiazol-2-yl]amino}-6-(phenylmethyl)-2-pyrimidinyl]amino}phenyl)acetic acid;
(4-{[4-{[6-(1,1-dimethylethyl)-1,3-benzothiazol-2-yl]amino}-6-(phenylmethyl)-2-pyrimidinyl]amino}phenyl)acetic acid;
methyl[(4-{[4-[(6-ethyl-1,3-benzothiazol-2-yl)amino]-6-(phenylmethyl)-2-pyrimidinyl]amino}phenyl)oxy]acetate;
(4-{[4-{[6-(cyanomethyl)-1,3-benzothiazol-2-yl]amino}-6-(phenylmethyl)-2-pyrimidinyl]amino}phenyl)acetic acid;
(3-fluoro-4-{[4-[(6-methyl-1,3-benzothiazol-2-yl)amino]-6-(phenylmethyl)-2-pyrimidinyl]amino}phenyl)acetic acid;
3-(4-{[4-[(5-chloro[1,3]thiazolo[5,4-b]pyridin-2-yl)amino]-6-(phenylmethyl)-2-pyrimidinyl]amino}phenyl)propanoic acid;
(4-{[4-{[6-(methyloxy)-1,3-benzothiazol-2-yl]amino}-6-(phenylmethyl)-2-pyrimidinyl]amino}phenyl)acetic acid;
2-(4-{[4-{[6-(methyloxy)-1,3-benzothiazol-2-yl]amino}-6-(phenylmethyl)-2-pyrimidinyl]amino}phenyl)ethanol;
4-{[4-{[6-(methyloxy)-1,3-benzothiazol-2-yl]amino}-6-(phenylmethyl)-2-pyrimidinyl]amino}phenol;
6-{[4-[(6-ethyl-1,3-benzothiazol-2-yl)amino]-6-(phenylmethyl)-2-pyrimidinyl]amino}-2-naphthalenecarboxylic acid;
(3-fluoro-4-{[4-{[5-(methyloxy)[1,3]thiazolo[5,4-b]pyridin-2-yl]amino}-6-(phenylmethyl)-2-pyrimidinyl]amino}phenyl)acetic acid;
2-(4-{[4-{[5-(methyloxy)[1,3]thiazolo[5,4-b]pyridin-2-yl]amino}-6-(phenylmethyl)-2-pyrimidinyl]amino}phenyl)cyclopropanecarboxylic acid;
trans-4-{[4-(1,3-benzothiazol-2-ylamino)-6-(phenylmethyl)-2-pyrimidinyl]amino}cyclohexanol;
trans-4-{[4-{[6-(methyloxy)-1,3-benzothiazol-2-yl]amino}-6-(phenylmethyl)-2-pyrimidinyl]amino}cyclohexanol;
4-(2-{[4-{[6-(methyloxy)-1,3-benzothiazol-2-yl]amino}-6-(phenylmethyl)-2-pyrimidinyl]amino}ethyl)benzenesulfonamide;
3-(4-{[4-{[6-(methyloxy)-1,3-benzothiazol-2-yl]amino}-6-(phenylmethyl)-2-pyrimidinyl]amino}phenyl)propanoic acid;
2-{[2-[(trans-4-hydroxycyclohexyl)amino]-6-(phenylmethyl)-4-pyrimidinyl]amino}-1,3-benzothiazole-6-carboxylic acid;
(2-{[2-[(trans-4-hydroxycyclohexyl)amino]-6-(phenylmethyl)-4-pyrimidinyl]amino}-1,3-benzothiazol-6-yl)acetic acid;
3-(2-{[2-[(trans-4-hydroxycyclohexyl)amino]-6-(phenylmethyl)-4-pyrimidinyl]amino}-1,3-benzothiazol-6-yl)propanoic acid;
2-(2-{[2-[(trans-4-hydroxycyclohexyl)amino]-6-(phenylmethyl)-4-pyrimidinyl]amino}-1,3-benzothiazol-6-yl)acetamide;
3-(2-{[2-[(trans-4-hydroxycyclohexyl)amino]-6-(phenylmethyl)-4-pyrimidinyl]amino}-1,3-benzothiazol-6-yl)propanamide;
trans-4-{[4-{[6-(2-hydroxyethyl)-1,3-benzothiazol-2-yl]amino}-6-(phenylmethyl)-2-pyrimidinyl]amino}cyclohexanol;
trans-4-{[4-{[6-(hydroxymethyl)-1,3-benzothiazol-2-yl]amino}-6-(phenylmethyl)-2-pyrimidinyl]amino}cyclohexanol;
trans-4-{[4-{[6-(3-hydroxypropyl)-1,3-benzothiazol-2-yl]amino}-6-(phenylmethyl)-2-pyrimidinyl]amino}cyclohexanol;
trans-4-{[4-[(6-amino-1,3-benzothiazol-2-yl)amino]-6-(phenylmethyl)-2-pyrimidinyl]amino}cyclohexanol;
N-(2-{[2-[(trans-4-hydroxycyclohexyl)amino]-6-(phenylmethyl)-4-pyrimidinyl]amino}-1,3-benzothiazol-6-yl)acetamide;
methyl (2-{[2-[(trans-4-hydroxycyclohexyl)amino]-6-(phenylmethyl)-4-pyrimidinyl]amino}-1,3-benzothiazol-6-yl)carbamate;
N-(2-{[2-[(trans-4-hydroxycyclohexyl)amino]-6-(phenylmethyl)-4-pyrimidinyl]amino}-1,3-benzothiazol-6-yl)methanesulfonamide;
1-(2-{[2-[(trans-4-hydroxycyclohexyl)amino]-6-(phenylmethyl)-4-pyrimidinyl]amino}-1,3-benzothiazol-6-yl)-2-azetidinone;
1-(2-{[2-[(trans-4-hydroxycyclohexyl)amino]-6-(phenylmethyl)-4-pyrimidinyl]amino}-1,3-benzothiazol-6-yl)-2-pyrrolidinone;
1-(2-{[2-[(trans-4-hydroxycyclohexyl)amino]-6-(phenylmethyl)-4-pyrimidinyl]amino}-1,3-benzothiazol-6-yl)-2-piperidinone;
1-(2-{[2-[(trans-4-hydroxycyclohexyl)amino]-6-(phenylmethyl)-4-pyrimidinyl]amino}-1,3-benzothiazol-6-yl)-2,5-pyrrolidinedione;
1-(2-{[2-[(trans-4-hydroxycyclohexyl)amino]-6-(phenylmethyl)-4-pyrimidinyl]amino}-1,3-benzothiazol-6-yl)-2-imidazolidinone;
3-(2-{[2-[(trans-4-hydroxycyclohexyl)amino]-6-(phenylmethyl)-4-pyrimidinyl]amino}-1,3-benzothiazol-6-yl)-2,4-imidazolidinedione;
3-(2-{[2-[(trans-4-hydroxycyclohexyl)amino]-6-(phenylmethyl)-4-pyrimidinyl]amino}-1,3-benzothiazol-6-yl)-1,3-oxazolidin-2-one;
trans-4-{[4-{[5-(dimethylamino)[1,3]thiazolo[5,4-b]pyridin-2-yl]amino}-6-(phenylmethyl)-2-pyrimidinyl]amino}cyclohexanol;
3-(4-{[4-{[5-(dimethylamino)[1,3]thiazolo[5,4-b]pyridin-2-yl]amino}-6-(phenylmethyl)-2-pyrimidinyl]amino}phenyl)propanoic acid;
3-(4-{[4-{[5-(dimethylamino)[1,3]thiazolo[5,4-b]pyridin-2-yl]amino}-6-(phenylmethyl)-2-pyrimidinyl]amino}phenyl)-N,N-bis(2-hydroxyethyl)propanamide;
trans-4-{[4-{[5-(4-morpholinyl)[1,3]thiazolo[5,4-b]pyridin-2-yl]amino}-6-(phenylmethyl)-2-pyrimidinyl]amino}cyclohexanol;
trans-4-[(4-(phenylmethyl)-6-{[5-(1-pyrrolidinyl)[1,3]thiazolo[5,4-b]pyridin-2-yl]amino}-2-pyrimidinyl)amino]cyclohexanol;
trans-4-[(4-(phenylmethyl)-6-{[5-(1-piperidinyl)[1,3]thiazolo[5,4-b]pyridin-2-yl]amino}-2-pyrimidinyl)amino]cyclohexanol;
trans-4-[(4-(phenylmethyl)-6-{[5-(1-piperazinyl)[1,3]thiazolo[5,4-b]pyridin-2-yl]amino}-2-pyrimidinyl)amino]cyclohexanol;
trans-4-{[4-({5-[(2-hydroxyethyl)(methyl)amino][1,3]thiazolo[5,4-b]pyridin-2-yl}amino)-6-(phenylmethyl)-2-pyrimidinyl]amino}cyclohexanol;
trans-4-{[4-({5-[(3-hydroxypropyl)(methyl)amino][1,3]thiazolo[5,4-b]pyridin-2-yl}amino)-6-(phenylmethyl)-2-pyrimidinyl]amino}cyclohexanol;
3-[(2-{[2-[(trans-4-hydroxycyclohexyl)amino]-6-(phenylmethyl)-4-pyrimidinyl]amino}[1,3]thiazolo[5,4-b]pyridin-5-yl)(methyl)amino]-1,2-propanediol;
1-(2-{[2-[(trans-4-hydroxycyclohexyl)amino]-6-(phenylmethyl)-4-pyrimidinyl]amino}[1,3]thiazolo[5,4-b]pyridin-5-yl)-4-piperidinol;
trans-4-{[4-{[5-(4-amino-1-piperidinyl)[1,3]thiazolo[5,4-b]pyridin-2-yl]amino}-6-(phenylmethyl)-2-pyrimidinyl]amino}cyclohexanol;
trans-4-{[4-({5-[(3R)-3-amino-1-pyrrolidinyl][1,3]thiazolo[5,4-b]pyridin-2-yl}amino)-6-(phenylmethyl)-2-pyrimidinyl]amino}cyclohexanol;
trans-4-{[4-({5-[(3S)-3-amino-1-pyrrolidinyl][1,3]thiazolo[5,4-b]pyridin-2-yl}amino)-6-(phenylmethyl)-2-pyrimidinyl]amino}cyclohexanol;
trans-4-{[4-({5-[4-(2-hydroxyethyl)-1-piperazinyl][1,3]thiazolo[5,4-b]pyridin-2-yl}amino)-6-(phenylmethyl)-2-pyrimidinyl]amino}cyclohexanol;
trans-4-{[4-({5-[4-(2-aminoethyl)-1-piperazinyl][1,3]thiazolo[5,4-b]pyridin-2-yl}amino)-6-(phenylmethyl)-2-pyrimidinyl]amino}cyclohexanol;
3-(4-{[4-{[5-(4-morpholinyl)[1,3]thiazolo[5,4-b]pyridin-2-yl]amino}-6-(phenylmethyl)-2-pyrimidinyl]amino}phenyl)propanoic acid;
N²-{4-[3-(4-morpholinyl)-3-oxopropyl]phenyl}-N⁴-[5-(4-morpholinyl)[1,3]thiazolo[5,4-b]pyridin-2-yl]-6-(phenylmethyl)-2,4-pyrimidinediamine;
3-{4-[(4-(phenylmethyl)-6-{[5-(1-piperazinyl)[1,3]thiazolo[5,4-b]pyridin-2-yl]amino}-2-pyrimidinyl)amino]phenyl}propanoic acid;
trans-4-[(4-[difluoro(4-fluorophenyl)methyl]-6-{[5-(methyloxy)[1,3]thiazolo[5,4-b]pyridin-2-yl]amino}-2-pyrimidinyl)amino]cyclohexanol;
trans-4-[(4-(1,1-difluoroethyl)-6-{[6-(methyloxy)-1,3-benzothiazol-2-yl]amino}-2-pyrimidinyl)amino]cyclohexanol;
N2-(trans-4-aminocyclohexyl)-6-[difluoro(4-fluorophenyl)methyl]-N4-[6-(methyloxy)-1,3-benzothiazol-2-yl]-2,4-pyrimidinediamine;
trans-4-[(4-[difluoro(4-fluorophenyl)methyl]-6-{[6-(methyloxy)-1,3-benzothiazol-2-yl]amino}-2-pyrimidinyl)amino]cyclohexanol;
trans-4-({4-[difluoro(4-fluorophenyl)methyl]-6-[(6-methyl-1,3-benzothiazol-2-yl)amino]-2-pyrimidinyl}amino)cyclohexanol;
trans-4-({4-[(6-bromo-1,3-benzothiazol-2-yl)amino]-6-[difluoro(4-fluorophenyl)methyl]-2-pyrimidinyl}amino)cyclohexanol;
trans-4-[(4-(1,1-difluoroethyl)-6-{[6-(trifluoromethyl)-1,3-benzothiazol-2-yl]amino}-2-pyrimidinyl)amino]cyclohexanol;
trans-4-({4-(1,3-benzothiazol-2-ylamino)-6-[difluoro(4-fluorophenyl)methyl]-2-pyrimidinyl}amino)cyclohexanol;
trans-4-{[4-(1,3-benzothiazol-2-ylamino)-6-(1,1-difluoroethyl)-2-pyrimidinyl]amino}cyclohexanol;
4-[(4-[difluoro(4-fluorophenyl)methyl]-6-{[6-(methyloxy)-1,3-benzothiazol-2-yl]amino}-2-pyrimidinyl)amino]-1-butanol;
N2-(4-aminobutyl)-6-[difluoro(4-fluorophenyl)methyl]-N4-[6-(methyloxy)-1,3-benzothiazol-2-yl]-2,4-pyrimidinediamine;
6-[difluoro(4-fluorophenyl)methyl]-N4-[6-(methyloxy)-1,3-benzothiazol-2-yl]-N2-{[4-(methylsulfonyl)-2-morpholinyl]methyl}-2,4-pyrimidinediamine;
trans-4-[(4-[difluoro(4-fluorophenyl)methyl]-6-{[6-(ethyloxy)-1,3-benzothiazol-2-yl]amino}-2-pyrimidinyl)amino]cyclohexanol;
3-[(4-[difluoro(4-fluorophenyl)methyl]-6-{[6-(methyloxy)-1,3-benzothiazol-2-yl]amino}-2-pyrimidinyl)amino]-1-propanol;
trans-4-[(4-[difluoro(4-fluorophenyl)methyl]-6-{[6-(trifluoromethyl)-1,3-benzothiazol-2-yl]amino}-2-pyrimidinyl)amino]cyclohexanol;
6-[difluoro(4-fluorophenyl)methyl]-N4-[6-(methyloxy)-1,3-benzothiazol-2-yl]-N2-{[1-(methylsulfonyl)-3-piperidinyl]methyl}-2,4-pyrimidinediamine;
{4-[(4-[difluoro(4-fluorophenyl)methyl]-6-{[5-(methyloxy)[1,3]thiazolo[5,4-b]pyridin-2-yl]amino}-2-pyrimidinyl)amino]phenyl}acetic acid;
4-[(4-[difluoro(4-fluorophenyl)methyl]-6-{[6-(methyloxy)-1,3-benzothiazol-2-yl]amino}-2-pyrimidinyl)amino]benzenesulfonamide;
2-{4-[(4-[difluoro(4-fluorophenyl)methyl]-6-{[6-(methyloxy)-1,3-benzothiazol-2-yl]amino}-2-pyrimidinyl)amino]phenyl}ethanol;
N-({4-[(4-[difluoro(4-fluorophenyl)methyl]-6-{[6-(ethyloxy)-1,3-benzothiazol-2-yl]amino}-2-pyrimidinyl)amino]phenyl}sulfonyl)acetamide;
trans-4-({4-[(6-bromo-1,3-benzothiazol-2-yl)amino]-2-pyrimidinyl}amino)cyclohexanol;
trans-4-({4-[(6-{5-[(1-methylethyl)oxy]-3-pyridinyl}-1,3-benzothiazol-2-yl)amino]-2-pyrimidinyl}amino)cyclohexanol;
trans-4-({4-[(6-{5-[(2-hydroxyethyl)oxy]-3-pyridinyl}-1,3-benzothiazol-2-yl)amino]-2-pyrimidinyl}amino)cyclohexanol;
trans-4-({4-[(6-{5-[(4-methyl-1-piperazinyl)methyl]-3-pyridinyl}-1,3-benzothiazol-2-yl)amino]-2-pyrimidinyl}amino)cyclohexanol;
trans-4-{[4-({6-[5-(4-morpholinylmethyl)-3-pyridinyl]-1,3-benzothiazol-2-yl}amino)-2-pyrimidinyl]amino}cyclohexanol;
trans-4-[(4-{[6-(5-{[2-(dimethylamino)ethyl]oxy}-3-pyridinyl)-1,3-benzothiazol-2-yl]amino}-2-pyrimidinyl)amino]cyclohexanol;
trans-4-{[4-({6-[5-(tetrahydro-2H-pyran-4-yloxy)-3-pyridinyl]-1,3-benzothiazol-2-yl}amino)-2-pyrimidinyl]amino}cyclohexanol;
trans-4-{[4-({6-[5-({[2-(methyloxy)ethyl]amino}methyl)-3-pyridinyl]-1,3-benzothiazol-2-yl}amino)-2-pyrimidinyl]amino}cyclohexanol;
trans-4-[(4-{[6-(5-{[(2-hydroxyethyl)amino]methyl}-3-pyridinyl)-1,3-benzothiazol-2-yl]amino}-2-pyrimidinyl)amino]cyclohexanol;
trans-4-[(4-{[6-(5-{[(2R,6S)-2,6-dimethyl-4-morpholinyl]methyl}-3-pyridinyl)-1,3-benzothiazol-2-yl]amino}-2-pyrimidinyl)amino]cyclohexanol;
trans-4-({4-[(5-chloro[1,3]thiazolo[5,4-b]pyridin-2-yl)amino]-6-methyl-2-pyrimidinyl}amino)cyclohexanol;
trans-4-[(4-(phenylmethyl)-6-{[5-(1H-pyrazol-4-yl)[1,3]thiazolo[5,4-b]pyridin-2-yl]amino}-2-pyrimidinyl)amino]cyclohexanol;
trans-4-[(4-(phenylmethyl)-6-{[6-(1H-pyrazol-4-yl)-1,3-benzothiazol-2-yl]amino}-2-pyrimidinyl)amino]cyclohexanol;
N²-(trans-4-aminocyclohexyl)-6-(phenylmethyl)-N⁴-[5-(1H-pyrazol-4-yl)[1,3]thiazolo[5,4-b]pyridin-2-yl]-2,4-pyrimidinediamine;
trans-4-{[4-{[6-(3-methyl-1H-pyrazol-4-yl)-1,3-benzothiazol-2-yl]amino}-6-(phenylmethyl)-2-pyrimidinyl]amino}cyclohexanol;
trans-4-[(4-(phenylmethyl)-6-{[6-(4-pyridinyl)-1,3-benzothiazol-2-yl]amino}-2-pyrimidinyl)amino]cyclohexanol;
trans-4-{[4-({6-[6-(methyloxy)-3-pyridinyl]-1,3-benzothiazol-2-yl}amino)-6-(phenylmethyl)-2-pyrimidinyl]amino}cyclohexanol;
5-(2-{[2-[(trans-4-hydroxycyclohexyl)amino]-6-(phenylmethyl)-4-pyrimidinyl]amino}-1,3-benzothiazol-6-yl)-3-pyridinecarbonitrile;
trans-4-{[4-({6-[5-(methyloxy)-3-pyridinyl]-1,3-benzothiazol-2-yl}amino)-6-(phenylmethyl)-2-pyrimidinyl]amino}cyclohexanol;
5-(2-{[2-[(trans-4-hydroxycyclohexyl)amino]-6-(phenylmethyl)-4-pyrimidinyl]amino}-1,3-benzothiazol-6-yl)-2-pyridinecarbonitrile;
trans-4-{[4-{[6-(4-isoxazolyl)-1,3-benzothiazol-2-yl]amino}-6-(phenylmethyl)-2-pyrimidinyl]amino}cyclohexanol;
3-(2-{[2-[(trans-4-hydroxycyclohexyl)amino]-6-(phenylmethyl)-4-pyrimidinyl]amino}[1,3]thiazolo[5,4-b]pyridin-5-yl)-1,3-oxazolidin-2-one;
trans-4-{[4-{[6-(1,1-dioxido-2-isothiazolidinyl)-1,3-benzothiazol-2-yl]amino}-6-(phenylmethyl)-2-pyrimidinyl]amino}cyclohexanol;
N²-(trans-4-aminocyclohexyl)-N⁴-(5-chloro[1,3]thiazolo[5,4-b]pyridin-2-yl)-6-(phenylmethyl)-2,4-pyrimidinediamine;
trans-4-([4-[(6-bromo-1,3-benzothiazol-2-yl)amino]-6-[(methyloxy)methyl]-2-pyrimidinyl]amino)cyclohexanol;
trans-4-{[4-[(6-bromo-1,3-benzothiazol-2-yl)amino]-6-(hydroxymethyl)-2-pyrimidinyl]amino}cyclohexanol;
trans-4-({4-(4-morpholinylmethyl)-6-[(6-nitro-1,3-benzothiazol-2-yl)amino]-2-pyrimidinyl}amino)cyclohexanol;
trans-4-({4-[(6-bromo-1,3-benzothiazol-2-yl)amino]-6-[cis-(3,5-dimethyl-4-morpholinyl)methyl]-2-pyrimidinyl}amino)cyclohexanol;
trans-4-({4-[(6-bromo-1,3-benzothiazol-2-yl)amino]-6-[trans-(3,5-dimethyl-4-morpholinyl)methyl]-2-pyrimidinyl}amino)cyclohexanol;
trans-4-{[4-[(6-bromo-1,3-benzothiazol-2-yl)amino]-6-(1-piperidinylmethyl)-2-pyrimidinyl]amino}cyclohexanol;
trans-4-({4-[(6-bromo-1,3-benzothiazol-2-yl)amino]-6-[(diethylamino)methyl]-2-pyrimidinyl}amino)cyclohexanol;
trans-4-{[4-[(6-bromo-1,3-benzothiazol-2-yl)amino]-6-(1-pyrrolidinylmethyl)-2-pyrimidinyl]amino}cyclohexanol;
trans-4-[(4-[(6-bromo-1,3-benzothiazol-2-yl)amino]-6-{[(1,1,3,3-tetramethylbutyl)amino]methyl}-2-pyrimidinyl)amino]cyclohexanol;
trans-4-{[4-[(6-bromo-1,3-benzothiazol-2-yl)amino]-6-(4-morpholinylmethyl)-2-pyrimidinyl]amino}cyclohexanol;
trans-4-[(4-[(6-bromo-1,3-benzothiazol-2-yl)amino]-6-{[(2R,6S)-2,6-dimethyl-4-morpholinyl]methyl}-2-pyrimidinyl)amino]cyclohexanol;
trans-4-({4-[(6-bromo-1,3-benzothiazol-2-yl)amino]-6-[(3,3-difluoro-1-piperidinyl)methyl]-2-pyrimidinyl}amino)cyclohexanol;
5-(2-{[2-[(trans-4-hydroxycyclohexyl)amino]-6-(1-piperidinylmethyl)-4-pyrimidinyl]amino}-1,3-benzothiazol-6-yl)-3-pyridinecarbonitrile;
5-(2-{[2-[(trans-4-hydroxycyclohexyl)amino]-6-(4-morpholinylmethyl)-4-pyrimidinyl]amino}-1,3-benzothiazol-6-yl)-3-pyridinecarbonitrile;
5-[2-({6-{[(2R,6S)-2,6-dimethyl-4-morpholinyl]methyl}-2-[(trans-4-hydroxycyclohexyl)amino]-4-pyrimidinyl}amino)-1,3-benzothiazol-6-yl]-3-pyridinecarbonitrile;
5-[2-({6-[(3,3-difluoro-1-piperidinyl)methyl]-2-[(trans-4-hydroxycyclohexyl)amino]-4-pyrimidinyl}amino)-1,3-benzothiazol-6-yl]-3-pyridinecarbonitrile;
trans-4-{[4-[(6-{5-[(1-methylethyl)oxy]-3-pyridinyl}-1,3-benzothiazol-2-yl)amino]-6-(4-morpholinylmethyl)-2-pyrimidinyl]amino}cyclohexanol;
trans-4-{[4-[(6-{5-[(1-methylethyl)oxy]-3-pyridinyl}-1,3-benzothiazol-2-yl)amino]-6-(1-piperidinylmethyl)-2-pyrimidinyl]amino}cyclohexanol;
trans-4-{[4-({6-[5-({[2-(methyloxy)ethyl]amino}methyl)-3-pyridinyl]-1,3-benzothiazol-2-yl}amino)-6-(4-morpholinylmethyl)-2-pyrimidinyl]amino}cyclohexanol;
1-(2-{[2-[(trans-4-hydroxycyclohexyl)amino]-6-(1-piperidinylmethyl)-4-pyrimidinyl]amino}-1,3-benzothiazol-6-yl)-2,5-pyrrolidinedione;
1-(2-{[2-[(trans-4-hydroxycyclohexyl)amino]-6-(4-morpholinylmethyl)-4-pyrimidinyl]amino}-1,3-benzothiazol-6-yl)-2,5-pyrrolidinedione;
1-(2-{[2-[(trans-4-hydroxycyclohexyl)amino]-6-(1-piperidinylmethyl)-4-pyrimidinyl]amino}-1,3-benzothiazol-6-yl)-2-pyrrolidinone;
3-(2-{[2-[(trans-4-hydroxycyclohexyl)amino]-6-(1-piperidinylmethyl)-4-pyrimidinyl]amino}-1,3-benzothiazol-6-yl)-1,3-oxazolidin-2-one;
3-(2-{[2-[(trans-4-hydroxycyclohexyl)amino]-6-(4-morpholinylmethyl)-4-pyrimidinyl]amino}-1,3-benzothiazol-6-yl)-1,3-oxazolidin-2-one;
3-[2-({6-{[(2R,6S)-2,6-dimethyl-4-morpholinyl]methyl}-2-[(trans-4-hydroxycyclohexyl)amino]-4-pyrimidinyl}amino)-1,3-benzothiazol-6-yl]-1,3-oxazolidin-2-one;
3-[2-({6-[(3,3-difluoro-1-piperidinyl)methyl]-2-[(trans-4-hydroxycyclohexyl)amino]-4-pyrimidinyl}amino)-1,3-benzothiazol-6-yl]-1,3-oxazolidin-2-one;
trans-4-[(4-(1-piperidinylmethyl)-6-{[6-(1H-pyrazol-4-yl)-1,3-benzothiazol-2-yl]amino}-2-pyrimidinyl)amino]cyclohexanol;
trans-4-[(4-(4-morpholinylmethyl)-6-{[6-(1H-pyrazol-4-yl)-1,3-benzothiazol-2-yl]amino}-2-pyrimidinyl)amino]cyclohexanol;
trans-4-[(4-{[(2R,6S)-2,6-dimethyl-4-morpholinyl]methyl}-6-{[6-(1H-pyrazol-4-yl)-1,3-benzothiazol-2-yl]amino}-2-pyrimidinyl)amino]cyclohexanol;
trans-4-[(4-[(3,3-difluoro-1-piperidinyl)methyl]-6-{[6-(1H-pyrazol-4-yl)-1,3-benzothiazol-2-yl]amino}-2-pyrimidinyl)amino]cyclohexanol;
3-(4-{[4-(1-piperidinylmethyl)-6-([1,3]thiazolo[5,4-b]pyridin-2-ylamino)-2-pyrimidinyl]amino}phenyl)propanoic acid;
N²-[trans-4-(4-morpholinyl)cyclohexyl]-6-(1-piperidinylmethyl)-N⁴-[1,3]thiazolo[5,4-b]pyridin-2-yl-2,4-pyrimidinediamine;
1-{[2-[(trans-4-hydroxycyclohexyl)amino]-6-([1,3]thiazolo[5,4-b]pyridin-2-ylamino)-4-pyrimidinyl]methyl}-2-piperazinone;
1-{[2-[(trans-4-hydroxycyclohexyl)amino]-6-([1,3]thiazolo[5,4-b]pyridin-2-ylamino)-4-pyrimidinyl]methyl}-L-proline;
trans-4-{[4-{[(1,1-dimethylbutyl)amino]methyl}-6-([1,3]thiazolo[5,4-b]pyridin-2-ylamino)-2-pyrimidinyl]amino}cyclohexanol;
trans-4-{[4-{[(1,2-dimethylpropyl)amino]methyl}-6-([1,3]thiazolo[5,4-b]pyridin-2-ylamino)-2-pyrimidinyl]amino}cyclohexanol;
trans-4-[(4-([1,3]thiazolo[5,4-b]pyridin-2-ylamino)-6-{[(1,2,2-trimethylpropyl)amino]methyl}-2-pyrimidinyl)amino]cyclohexanol;
trans-4-{[4-[(2,2-dimethyl-1-pyrrolidinyl)methyl]-6-([1,3]thiazolo[5,4-b]pyridin-2-ylamino)-2-pyrimidinyl]amino}cyclohexanol;
trans-4-{[4-(1-pyrrolidinylmethyl)-6-([1,3]thiazolo[5,4-b]pyridin-2-ylamino)-2-pyrimidinyl]amino}cyclohexanol;
trans-4-{[4-[(diethylamino)methyl]-6-([1,3]thiazolo[5,4-b]pyridin-2-ylamino)-2-pyrimidinyl]amino}cyclohexanol;
trans-4-{[4-{[(1-ethylpropyl)amino]methyl}-6-([1,3]thiazolo[5,4-b]pyridin-2-ylamino)-2-pyrimidinyl]amino}cyclohexanol;
trans-4-{[4-{[(1-methylethyl)amino]methyl}-6-([1,3]thiazolo[5,4-b]pyridin-2-ylamino)-2-pyrimidinyl]amino}cyclohexanol;
trans-4-{[4-{[(1,1-dimethylethyl)amino]methyl}-6-([1,3]thiazolo[5,4-b]pyridin-2-ylamino)-2-pyrimidinyl]amino}cyclohexanol;
trans-4-{[4-({[1,1-dimethyl-2-(4-morpholinyl)ethyl]amino}methyl)-6-([1,3]thiazolo[5,4-b]pyridin-2-ylamino)-2-pyrimidinyl]amino}cyclohexanol;
trans-4-{[4-{[(2-hydroxy-1,1-dimethylethyl)amino]methyl}-6-([1,3]thiazolo[5,4-b]pyridin-2-ylamino)-2-pyrimidinyl]amino}cyclohexanol;
trans-4-{[4-[(2-methyl-1-pyrrolidinyl)methyl]-6-([1,3]thiazolo[5,4-b]pyridin-2-ylamino)-2-pyrimidinyl]amino}cyclohexanol;
trans-4-{[4-[(2,5-dimethyl-1-pyrrolidinyl)methyl]-6-([1,3]thiazolo[5,4-b]pyridin-2-ylamino)-2-pyrimidinyl]amino}cyclohexanol;
trans-4-{[4-{[methyl(2-methylpropyl)amino]methyl}-6-([1,3]thiazolo[5,4-b]pyridin-2-ylamino)-2-pyrimidinyl]amino}cyclohexanol;
trans-4-{[4-{[(2-methylpropyl)amino]methyl}-6-([1,3]thiazolo[5,4-b]pyridin-2-ylamino)-2-pyrimidinyl]amino}cyclohexanol;
trans-4-{[4-(1-piperidinylmethyl)-6-([1,3]thiazolo[5,4-b]pyridin-2-ylamino)-2-pyrimidinyl]amino}cyclohexanol;
trans-4-{[4-[(4-methyl-1-piperazinyl)methyl]-6-([1,3]thiazolo[5,4-b]pyridin-2-ylamino)-2-pyrimidinyl]amino}cyclohexanol;
trans-4-{[4-{[4-(1,1-dimethylethyl)-1-piperazinyl]methyl}-6-([1,3]thiazolo[5,4-b]pyridin-2-ylamino)-2-pyrimidinyl]amino}cyclohexanol;
trans-4-{[4-({4-[2-(methyloxy)ethyl]-1-piperazinyl}methyl)-6-([1,3]thiazolo[5,4-b]pyridin-2-ylamino)-2-pyrimidinyl]amino}cyclohexanol;
trans-4-{[4-[(4,4-difluoro-1-piperidinyl)methyl]-6-([1,3]thiazolo[5,4-b]pyridin-2-ylamino)-2-pyrimidinyl]amino}cyclohexanol;
trans-4-{[4-(4-morpholinylmethyl)-6-([1,3]thiazolo[5,4-b]pyridin-2-ylamino)-2-pyrimidinyl]amino}cyclohexanol;
trans-4-{[4-{[ethyl(1-methylethyl)amino]methyl}-6-([1,3]thiazolo[5,4-b]pyridin-2-ylamino)-2-pyrimidinyl]amino}cyclohexanol;
4-{[2-[(trans-4-hydroxycyclohexyl)amino]-6-([1,3]thiazolo[5,4-b]pyridin-2-ylamino)-4-pyrimidinyl]methyl}-2-piperazinone;
trans-4-{[4-[(1,1-dioxido-4-thiomorpholinyl)methyl]-6-([1,3]thiazolo[5,4-b]pyridin-2-ylamino)-2-pyrimidinyl]amino}cyclohexanol;
1,1-dimethylethyl[2-({[2-[(trans-4-hydroxycyclohexyl)amino]-6-([1,3]thiazolo[5,4-b]pyridin-2-ylamino)-4-pyrimidinyl]methyl}amino)ethyl]carbamate;
1-[2-({[2-[(trans-4-hydroxycyclohexyl)amino]-6-([1,3]thiazolo[5,4-b]pyridin-2-ylamino)-4-pyrimidinyl]methyl}amino)ethyl]-2-pyrrolidinone;
1,1-dimethylethyl 1-{[2-[(trans-4-hydroxycyclohexyl)amino]-6-([1,3]thiazolo[5,4-b]pyridin-2-ylamino)-4-pyrimidinyl]methyl}-L-prolinate;
methyl 1-{[2-[(trans-4-hydroxycyclohexyl)amino]-6-([1,3]thiazolo[5,4-b]pyridin-2-ylamino)-4-pyrimidinyl]methyl}-L-prolinate;
trans-4-{[4-[(3,3-difluoro-1-pyrrolidinyl)methyl]-6-([1,3]thiazolo[5,4-b]pyridin-2-ylamino)-2-pyrimidinyl]amino}cyclohexanol;
trans-4-{[4-[(3,3-difluoro-1-piperidinyl)methyl]-6-([1,3]thiazolo[5,4-b]pyridin-2-ylamino)-2-pyrimidinyl]amino}cyclohexanol;
trans-4-{[4-[(2,2-dimethyl-4-morpholinyl)methyl]-6-([1,3]thiazolo[5,4-b]pyridin-2-ylamino)-2-pyrimidinyl]amino}cyclohexanol;
trans-4-{[4-{[(2R,6S)-2,6-dimethyl-4-morpholinyl]methyl}-6-([1,3]thiazolo[5,4-b]pyridin-2-ylamino)-2-pyrimidinyl]amino}cyclohexanol;
trans-4-[(4-([1,3]thiazolo[5,4-b]pyridin-2-ylamino)-6-{[(2,2,2-trifluoroethyl)amino]methyl}-2-pyrimidinyl)amino]cyclohexanol;
trans-4-{[4-({bis[2-(methyloxy)ethyl]amino}methyl)-6-([1,3]thiazolo[5,4-b]pyridin-2-ylamino)-2-pyrimidinyl]amino}cyclohexanol;
trans-4-{[4-({methyl[2-(methyloxy)ethyl]amino}methyl)-6-([1,3]thiazolo[5,4-b]pyridin-2-ylamino)-2-pyrimidinyl]amino}cyclohexanol;
trans-4-{[4-({2-[(methyloxy)methyl]-1-pyrrolidinyl}methyl)-6-([1,3]thiazolo[5,4-b]pyridin-2-ylamino)-2-pyrimidinyl]amino}cyclohexanol;
trans-4-{[4-{[4-(methyloxy)-1-piperidinyl]methyl}-6-([1,3]thiazolo[5,4-b]pyridin-2-ylamino)-2-pyrimidinyl]amino}cyclohexanol;
trans-4-{[4-[(tetrahydro-3-furanylamino)methyl]-6-([1,3]thiazolo[5,4-b]pyridin-2-ylamino)-2-pyrimidinyl]amino}cyclohexanol;
trans-4-{[4-[(3,3-difluoro-1-azetidinyl)methyl]-6-([1,3]thiazolo[5,4-b]pyridin-2-ylamino)-2-pyrimidinyl]amino}cyclohexanol;
trans-4-{[4-{[2-(2-methylpropyl)-4-morpholinyl]methyl}-6-([1,3]thiazolo[5,4-b]pyridin-2-ylamino)-2-pyrimidinyl]amino}cyclohexanol;
trans-4-{[4-{[(1,1-dioxidotetrahydro-3-thienyl)amino]methyl}-6-([1,3]thiazolo[5,4-b]pyridin-2-ylamino)-2-pyrimidinyl]amino}cyclohexanol;
trans-4-{[4-{[(1,1-dioxidotetrahydro-3-thienyl)(methyl)amino]methyl}-6-([1,3]thiazolo[5,4-b]pyridin-2-ylamino)-2-pyrimidinyl]amino}cyclohexanol;
trans-4-{[4-{[(3R)-3-(1-methylethyl)-4-morpholinyl]methyl}-6-([1,3]thiazolo[5,4-b]pyridin-2-ylamino)-2-pyrimidinyl]amino}cyclohexanol;
trans-4-{[4-{[(1,1-dioxidotetrahydro-2H-thiopyran-4-yl)amino]methyl}-6-([1,3]thiazolo[5,4-b]pyridin-2-ylamino)-2-pyrimidinyl]amino}cyclohexanol;
trans-4-{[4-[(3,3-dimethyl-1-piperidinyl)methyl]-6-([1,3]thiazolo[5,4-b]pyridin-2-ylamino)-2-pyrimidinyl]amino}cyclohexanol;
trans-4-{[4-(hydroxymethyl)-6-([1,3]thiazolo[5,4-b]pyridin-2-ylamino)-2-pyrimidinyl]amino}cyclohexanol;
4-{[2-[(trans-4-hydroxycyclohexyl)amino]-6-([1,3]thiazolo[5,4-b]pyridin-2-ylamino)-4-pyrimidinyl]methyl}-6-methyl-3(2H)-pyridazinone; or
   a salt thereof.

In another embodiment, the compound of the invention is:
trans-4-{[4-[(6-methyl-1,3-benzothiazol-2-yl)amino]-6-(phenylmethyl)-2-pyrimidinyl]amino}cyclohexanol;
trans-4-{[4-[(6-chloro-1,3-benzothiazol-2-yl)amino]-6-(phenylmethyl)-2-pyrimidinyl]amino}cyclohexanol;
trans-4-[(4-(phenylmethyl)-6-{[6-(trifluoromethyl)-1,3-benzothiazol-2-yl]amino}-2-pyrimidinyl)amino]cyclohexanol;
trans-4-{[4-[(6-bromo-1,3-benzothiazol-2-yl)amino]-6-(phenylmethyl)-2-pyrimidinyl]amino}cyclohexanol;
trans-4-{[4-[(6-fluoro-1,3-benzothiazol-2-yl)amino]-6-(phenylmethyl)-2-pyrimidinyl]amino}cyclohexanol;
trans-4-{[4-[(6-ethyl-1,3-benzothiazol-2-yl)amino]-6-(phenylmethyl)-2-pyrimidinyl]amino}cyclohexanol;
trans-4-{[4-[(5-fluoro-1,3-benzothiazol-2-yl)amino]-6-(phenylmethyl)-2-pyrimidinyl]amino}cyclohexanol;
trans-4-{[4-{[5-(methyloxy)[1,3]thiazolo[5,4-b]pyridin-2-yl]amino}-6-(phenylmethyl)-2-pyrimidinyl]amino}cyclohexanol;
trans-4-{[4-[(5-chloro[1,3]thiazolo[5,4-b]pyridin-2-yl)amino]-6-(phenylmethyl)-2-pyrimidinyl]amino}cyclohexanol;
(4-{[4-[(6-ethyl-1,3-benzothiazol-2-yl)amino]-6-(phenylmethyl)-2-pyrimidinyl]amino}-1-piperidinyl)acetic acid;
trans-4-{[4-(phenylmethyl)-6-([1,3]thiazolo[5,4-b]pyridin-2-ylamino)-2-pyrimidinyl]amino}cyclohexanol;
trans-4-{[4-[(6-nitro-1,3-benzothiazol-2-yl)amino]-6-(phenylmethyl)-2-pyrimidinyl]amino}cyclohexanol;
(2-{[2-[(trans-4-hydroxycyclohexyl)amino]-6-(phenylmethyl)-4-pyrimidinyl]amino}-1,3-benzothiazol-6-yl)acetonitrile;
5-{[4-[(6-methyl-1,3-benzothiazol-2-yl)amino]-6-(phenylmethyl)-2-pyrimidinyl]amino}-1-pentanol;
2-hydroxy-5-(2-{[4-[(6-methyl-1,3-benzothiazol-2-yl)amino]-6-(phenylmethyl)-2-pyrimidinyl]amino}ethyl)benzenesulfonamide;
4-{[4-[(6-methyl-1,3-benzothiazol-2-yl)amino]-6-(phenylmethyl)-2-pyrimidinyl]amino}-1-butanol;
5-(2-{[4-[(6-ethyl-1,3-benzothiazol-2-yl)amino]-6-(phenylmethyl)-2-pyrimidinyl]amino}ethyl)-2-hydroxybenzenesulfonamide;
4-(2-{[4-[(6-methyl-1,3-benzothiazol-2-yl)amino]-6-(phenylmethyl)-2-pyrimidinyl]amino}ethyl)benzenesulfonamide;
4-{[4-[(6-ethyl-1,3-benzothiazol-2-yl)amino]-6-(phenylmethyl)-2-pyrimidinyl]amino}-1-butanol;
5-{[4-[(6-ethyl-1,3-benzothiazol-2-yl)amino]-6-(phenylmethyl)-2-pyrimidinyl]amino}-1-pentanol;
4-{[4-{[5-(methyloxy)[1,3]thiazolo[5,4-b]pyridin-2-yl]amino}-6-(phenylmethyl)-2-pyrimidinyl]amino}-1-butanol;
5-{[4-{[5-(methyloxy)[1,3]thiazolo[5,4-b]pyridin-2-yl]amino}-6-(phenylmethyl)-2-pyrimidinyl]amino}-1-pentanol;
trans-4-{[4-[(5-ethyl[1,3]thiazolo[5,4-b]pyridin-2-yl)amino]-6-(phenylmethyl)-2-pyrimidinyl]amino}cyclohexanol;
2-{[2-[(trans-4-hydroxycyclohexyl)amino]-6-(phenylmethyl)-4-pyrimidinyl]amino}-1,3-benzothiazole-6-carbonitrile;
ethyl 2-{[2-[(trans-4-hydroxycyclohexyl)amino]-6-(phenylmethyl)-4-pyrimidinyl]amino}-1,3-benzothiazole-6-carboxylate;
ethyl(2-{[2-[(trans-4-hydroxycyclohexyl)amino]-6-(phenylmethyl)-4-pyrimidinyl]amino}-1,3-benzothiazol-6-yl)acetate;
ethyl 3-(2-{[2-[(trans-4-hydroxycyclohexyl)amino]-6-(phenylmethyl)-4-pyrimidinyl]amino}-1,3-benzothiazol-6-yl)propanoate;
trans-4-{[4-{[6-(1-methylethyl)-1,3-benzothiazol-2-yl]amino}-6-(phenylmethyl)-2-pyrimidinyl]amino}cyclohexanol;
4-(2-{[4-[(6-ethyl-1,3-benzothiazol-2-yl)amino]-6-(2-methylpropyl)-2-pyrimidinyl]amino}ethyl)benzenesulfonamide;
4-(2-{[4-[(6-ethyl-1,3-benzothiazol-2-yl)amino]-6-(phenylmethyl)-2-pyrimidinyl]amino}ethyl)benzenesulfonamide;
trans-4-{[4-[(6-iodo-1,3-benzothiazol-2-yl)amino]-6-(phenylmethyl)-2-pyrimidinyl]amino}cyclohexanol;
5-(2-{[4-[(6-ethyl-1,3-benzothiazol-2-yl)amino]-6-(2-methylpropyl)-2-pyrimidinyl]amino}ethyl)-2-hydroxybenzenesulfonamide;
(1R,3S)-3-{[4-[(6-ethyl-1,3-benzothiazol-2-yl)amino]-6-(phenylmethyl)-2-pyrimidinyl]amino}cyclopentanecarboxylic acid;
trans-4-({4-(phenylmethyl)-6-[(6-propyl-1,3-benzothiazol-2-yl)amino]-2-pyrimidinyl}amino)cyclohexanol;
(1R,2S)-2-{[4-[(6-ethyl-1,3-benzothiazol-2-yl)amino]-6-(phenylmethyl)-2-pyrimidinyl]amino}cyclohexanecarboxylic acid;
5-{[4-[(6-ethyl-1,3-benzothiazol-2-yl)amino]-6-(2-methylpropyl)-2-pyrimidinyl]amino}-1-pentanol;
4-(2-{[4-{[6-(1-m ethylethyl)-1,3-benzothiazol-2-yl]amino}-6-(phenylmethyl)-2-pyrimidinyl]amino}ethyl)benzenesulfonamide;
4-{[4-[(6-ethyl-1,3-benzothiazol-2-yl)amino]-6-(2-methylpropyl)-2-pyrimidinyl]amino}-1-butanol;
trans-4-{[4-{[6-(1,1-dimethylethyl)-1,3-benzothiazol-2-yl]amino}-6-(phenylmethyl)-2-pyrimidinyl]amino}cyclohexanol;
3-{[4-[(6-ethyl-1,3-benzothiazol-2-yl)amino]-6-(phenylmethyl)-2-pyrimidinyl]amino}cyclohexanecarboxylic acid;
trans-4-({4-[(6-methyl-1,3-benzothiazol-2-yl)amino]-2-pyrimidinyl}amino)cyclohexanol;
N4-(6-ethyl-1,3-benzothiazol-2-yl)-N2-(1-methyl-4-piperidinyl)-6-(phenylmethyl)-2,4-pyrimidinediamine;
N4-(6-ethyl-1,3-benzothiazol-2-yl)-N2-{[4-(methylsulfonyl)-2-morpholinyl]methyl}-6-(phenylmethyl)-2,4-pyrimidinediamine;
4-(2-{[4-{[6-(1,1-dimethylethyl)-1,3-benzothiazol-2-yl]amino}-6-(phenylmethyl)-2-pyrimidinyl]amino}ethyl)benzenesulfonamide;
4-[[4-[(6-ethyl-1,3-benzothiazol-211)amino]-6-(phenylmethyl)-2-pyrimidinyl](methyl)amino]-1-butanol trifluoroacetate;
(1R,2R)-2-{[4-[(6-ethyl-1,3-benzothiazol-2-yl)amino]-6-(phenylmethyl)-2-pyrimidinyl]amino}cyclohexanol;
(1R,2R)-2-{[4-[(6-ethyl-1,3-benzothiazol-2-yl)amino]-6-(phenylmethyl)-2-pyrimidinyl]amino}cyclohexanecarboxylic acid;
trans-4-{[4-[(4-fluoro-1,3-benzothiazol-2-yl)amino]-6-(phenylmethyl)-2-pyrimidinyl]amino}cyclohexanol;
(1S,2R)-2-{[4-[(6-ethyl-1,3-benzothiazol-2-yl)amino]-6-(phenylmethyl)-2-pyrimidinyl]amino}cyclohexanecarboxamide;
N4-(6-ethyl-1,3-benzothiazol-2-yl)-N2-(trans-4-methylcyclohexyl)-6-(phenylmethyl)-2,4-pyrimidinediamine;
4-(3-{[4-[(6-ethyl-1,3-benzothiazol-2-yl)amino]-6-(phenylmethyl)-2-pyrimidinyl]amino}propyl)benzenesulfonamide;
3-{[4-{[6-(methyloxy)-1,3-benzothiazol-2-yl]amino}-6-(phenylmethyl)-2-pyrimidinyl]amino}-1-propanol;
trans-4-{[4-{[6-(methylsulfonyl)-1,3-benzothiazol-2-yl]amino}-6-(phenylmethyl)-2-pyrimidinyl]amino}cyclohexanol;
trans-4-{[4-[(7-bromo-1,3-benzothiazol-2-yl)amino]-6-(phenylmethyl)-2-pyrimidinyl]amino}cyclohexanol;
3-(4-{[4-[(6-ethyl-1,3-benzothiazol-2-yl)amino]-6-(phenylmethyl)-2-pyrimidinyl]amino}phenyl)propanoic acid;
3-(4-{[4-{[5-(methyloxy)[1,3]thiazolo[5,4-b]pyridin-2-yl]amino}-6-(phenylmethyl)-2-pyrimidinyl]amino}phenyl)propanoic acid;
3-(4-{[4-[(6-ethyl-1,3-benzothiazol-2-yl)amino]-6-(phenylmethyl)-2-pyrimidinyl]amino}phenyl)propanoic acid;
(2E)-3-(4-{[4-[(6-ethyl-1,3-benzothiazol-2-yl)amino]-6-(phenylmethyl)-2-pyrimidinyl]amino}phenyl)-2-propenoic acid;
(4-{[4-[(6-ethyl-1,3-benzothiazol-2-yl)amino]-6-(phenylmethyl)-2-pyrimidinyl]amino}phenyl)acetic acid;
3-(4-{[4-[(5-ethyl[1,3]thiazolo[5,4-b]pyridin-2-yl)amino]-6-(phenylmethyl)-2-pyrimidinyl]amino}phenyl)propanoic acid;
(4-{[4-[(6-ethyl-1,3-benzothiazol-2-yl)amino]-6-(phenylmethyl)-2-pyrimidinyl]amino}-3-fluorophenyl)acetic acid;
3-[(4-{[4-[(6-methyl-1,3-benzothiazol-2-yl)amino]-6-(phenylmethyl)-2-pyrimidinyl]amino}phenyl)oxy]propanoic acid;
2-(4-{[4-[(6-ethyl-1,3-benzothiazol-2-yl)amino]-6-(phenylmethyl)-2-pyrimidinyl]amino}phenyl)cyclopropanecarboxylic acid;
2-(4-{[4-[(6-ethyl-1,3-benzothiazol-2-yl)amino]-6-(2-methylpropyl)-2-pyrimidinyl]amino}-3-fluorophenyl)propanoic acid;
[(4-{[4-[(6-methyl-1,3-benzothiazol-2-yl)amino]-6-(phenylmethyl)-2-pyrimidinyl]amino}phenyl)thio]acetic acid;
2-(4-{[4-[(6-ethyl-1,3-benzothiazol-2-yl)amino]-6-(phenylmethyl)-2-pyrimidinyl]amino}phenyl)cyclopropanecarboxylic acid;
[(4-{[4-[(6-ethyl-1,3-benzothiazol-2-yl)amino]-6-(phenylmethyl)-2-pyrimidinyl]amino}phenyl)oxy]acetic acid;
2-[(4-{[4-[(6-ethyl-1,3-benzothiazol-2-yl)amino]-6-(phenylmethyl)-2-pyrimidinyl]amino}phenyl)oxy]-2-methylpropanoic acid;
3-[(4-{[4-[(6-ethyl-1,3-benzothiazol-2-yl)amino]-6-(2-methylpropyl)-2-pyrimidinyl]amino}phenyl)oxy]propanoic acid;
3-(4-{[4-({6-[(ethyloxy)carbonyl]-1,3-benzothiazol-2-yl}amino)-6-(phenylmethyl)-2-pyrimidinyl]amino}phenyl)propanoic acid;
3-methyl-4-{[4-{[5-(methyloxy)[1,3]thiazolo[5,4-b]pyridin-2-yl]amino}-6-(phenylmethyl)-2-pyrimidinyl]amino}benzenesulfonamide;
3-methyl-4-{[4-[(6-methyl-1,3-benzothiazol-2-yl)amino]-6-(phenylmethyl)-2-pyrimidinyl]amino}benzenesulfonamide;
[(4-{[4-[(6-ethyl-1,3-benzothiazol-2-yl)amino]-6-(2-methylpropyl)-2-pyrimidinyl]amino}phenyl)thio]acetic acid;
4-{[4-{[5-(methyloxy)[1,3]thiazolo[5,4-b]pyridin-2-yl]amino}-6-(phenylmethyl)-2-pyrimidinyl]amino}benzenesulfonamide;
2-(4-{[4-[(6-ethyl-1,3-benzothiazol-2-yl)amino]-6-(phenylmethyl)-2-pyrimidinyl]amino}phenyl)acetamide;
4-{[4-[(6-ethyl-1,3-benzothiazol-2-yl)amino]-6-(2-methylpropyl)-2-pyrimidinyl]amino}benzenesulfonamide;
4-{[4-[(6-methyl-1,3-benzothiazol-2-yl)amino]-6-(2-methylpropyl)-2-pyrimidinyl]amino}benzenesulfonamide;
4-{[4-[(6-ethyl-1,3-benzothiazol-2-yl)amino]-6-(2-methylpropyl)-2-pyrimidinyl]amino}-3-methylbenzenesulfonamide;
4-{[4-[(6-ethyl-1,3-benzothiazol-2-yl)amino]-6-(phenylmethyl)-2-pyrimidinyl]amino}-3-methylbenzenesulfonamide;
4-{[4-[(6-ethyl-1,3-benzothiazol-2-yl)amino]-6-(phenylmethyl)-2-pyrimidinyl]amino}benzenesulfonamide;
4-{[4-[(6-ethyl-1,3-benzothiazol-2-yl)amino]-6-(phenylmethyl)-2-pyrimidinyl]amino}benzoic acid;
(4-{[4-{[6-(1-methylethyl)-1,3-benzothiazol-2-yl]amino}-6-(phenylmethyl)-2-pyrimidinyl]amino}phenyl)acetic acid;
(4-{[4-{[6-(1,1-dimethylethyl)-1,3-benzothiazol-2-yl]amino}-6-(phenylmethyl)-2-pyrimidinyl]amino}phenyl)acetic acid;
methyl[(4-{[4-[(6-ethyl-1,3-benzothiazol-2-yl)amino]-6-(phenylmethyl)-2-pyrimidinyl]amino}phenyl)oxy]acetate;
(4-{[4-{[6-(cyanomethyl)-1,3-benzothiazol-2-yl]amino}-6-(phenylmethyl)-2-pyrimidinyl]amino}phenyl)acetic acid;
(3-fluoro-4-{[4-[(6-methyl-1,3-benzothiazol-2-yl)amino]-6-(phenylmethyl)-2-pyrimidinyl]amino}phenyl)acetic acid;
3-(4-{[4-[(5-chloro[1,3]thiazolo[5,4-b]pyridin-2-yl)amino]-6-(phenylmethyl)-2-pyrimidinyl]amino}phenyl)propanoic acid;
(4-{[4-{[6-(methyloxy)-1,3-benzothiazol-2-yl]amino}-6-(phenylmethyl)-2-pyrimidinyl]amino}phenyl)acetic acid;
2-(4-{[4-{[6-(methyloxy)-1,3-benzothiazol-2-yl]amino}-6-(phenylmethyl)-2-pyrimidinyl]amino}phenyl)ethanol;
4-{[4-{[6-(methyloxy)-1,3-benzothiazol-2-yl]amino}-6-(phenylmethyl)-2-pyrimidinyl]amino}phenol;
6-{[4-[(6-ethyl-1,3-benzothiazol-2-yl)amino]-6-(phenylmethyl)-2-pyrimidinyl]amino}-2-naphthalenecarboxylic acid;
(3-fluoro-4-{[4-{[5-(methyloxy)[1,3]thiazolo[5,4-b]pyridin-2-yl]amino}-6-(phenylmethyl)-2-pyrimidinyl]amino}phenyl)acetic acid;
2-(4-{[4-{[5-(methyloxy)[1,3]thiazolo[5,4-b]pyridin-2-yl]amino}-6-(phenylmethyl)-2-pyrimidinyl]amino}phenyl)cyclopropanecarboxylic acid;
trans-4-{[4-(1,3-benzothiazol-2-ylamino)-6-(phenylmethyl)-2-pyrimidinyl]amino}cyclohexanol;
trans-4-{[4-{[6-(methyloxy)-1,3-benzothiazol-2-yl]amino}-6-(phenylmethyl)-2-pyrimidinyl]amino}cyclohexanol;
4-(2-{[4-{[6-(methyloxy)-1,3-benzothiazol-2-yl]amino}-6-(phenylmethyl)-2-pyrimidinyl]amino}ethyl)benzenesulfonamide;
3-(4-{[4-{[6-(methyloxy)-1,3-benzothiazol-2-yl]amino}-6-(phenylmethyl)-2-pyrimidinyl]amino}phenyl)propanoic acid;
2-{[2-[(trans-4-hydroxycyclohexyl)amino]-6-(phenylmethyl)-4-pyrimidinyl]amino}-1,3-benzothiazole-6-carboxylic acid;
(2-{[2-[(trans-4-hydroxycyclohexyl)amino]-6-(phenylmethyl)-4-pyrimidinyl]amino}-1,3-benzothiazol-6-yl)acetic acid;
3-(2-{[2-[(trans-4-hydroxycyclohexyl)amino]-6-(phenylmethyl)-4-pyrimidinyl]amino}-1,3-benzothiazol-6-yl)propanoic acid;
2-(2-{[2-[(trans-4-hydroxycyclohexyl)amino]-6-(phenylmethyl)-4-pyrimidinyl]amino}-1,3-benzothiazol-6-yl)acetamide;
3-(2-{[2-[(trans-4-hydroxycyclohexyl)amino]-6-(phenylmethyl)-4-pyrimidinyl]amino}-1,3-benzothiazol-6-yl)propanamide;
trans-4-{[4-{[6-(2-hydroxyethyl)-1,3-benzothiazol-2-yl]amino}-6-(phenylmethyl)-2-pyrimidinyl]amino}cyclohexanol;
trans-4-{[4-{[6-(hydroxymethyl)-1,3-benzothiazol-2-yl]amino}-6-(phenylmethyl)-2-pyrimidinyl]amino}cyclohexanol;
trans-4-{[4-{[6-(3-hydroxypropyl)-1,3-benzothiazol-2-yl]amino}-6-(phenylmethyl)-2-pyrimidinyl]amino}cyclohexanol;
trans-4-{[4-[(6-amino-1,3-benzothiazol-2-yl)amino]-6-(phenylmethyl)-2-pyrimidinyl]amino}cyclohexanol;
N-(2-{[2-[(trans-4-hydroxycyclohexyl)amino]-6-(phenylmethyl)-4-pyrimidinyl]amino}-1,3-benzothiazol-6-yl)acetamide;
methyl (2-{[2-[(trans-4-hydroxycyclohexyl)amino]-6-(phenylmethyl)-4-pyrimidinyl]amino}-1,3-benzothiazol-6-yl)carbamate;
N-(2-{[2-[(trans-4-hydroxycyclohexyl)amino]-6-(phenylmethyl)-4-pyrimidinyl]amino}-1,3-benzothiazol-6-yl)methanesulfonamide;
1-(2-{[2-[(trans-4-hydroxycyclohexyl)amino]-6-(phenylmethyl)-4-pyrimidinyl]amino}-1,3-benzothiazol-6-yl)-2-azetidinone;
1-(2-{[2-[(trans-4-hydroxycyclohexyl)amino]-6-(phenylmethyl)-4-pyrimidinyl]amino}-1,3-benzothiazol-6-yl)-2-pyrrolidinone;
1-(2-{[2-[(trans-4-hydroxycyclohexyl)amino]-6-(phenylmethyl)-4-pyrimidinyl]amino}-1,3-benzothiazol-6-yl)-2-piperidinone;
1-(2-{[2-[(trans-4-hydroxycyclohexyl)amino]-6-(phenylmethyl)-4-pyrimidinyl]amino}-1,3-benzothiazol-6-yl)-2,5-pyrrolidinedione;
1-(2-{[2-[(trans-4-hydroxycyclohexyl)amino]-6-(phenylmethyl)-4-pyrimidinyl]amino}-1,3-benzothiazol-6-yl)-2-imidazolidinone;
3-(2-{[2-[(trans-4-hydroxycyclohexyl)amino]-6-(phenylmethyl)-4-pyrimidinyl]amino}-1,3-benzothiazol-6-yl)-2,4-imidazolidinedione;
3-(2-{[2-[(trans-4-hydroxycyclohexyl)amino]-6-(phenylmethyl)-4-pyrimidinyl]amino}-1,3-benzothiazol-6-yl)-1,3-oxazolidin-2-one;
trans-4-{[4-{[5-(dimethylamino)[1,3]thiazolo[5,4-b]pyridin-2-yl]amino}-6-(phenylmethyl)-2-pyrimidinyl]amino}cyclohexanol;
3-(4-{[4-{[5-(dimethylamino)[1,3]thiazolo[5,4-b]pyridin-2-yl]amino}-6-(phenylmethyl)-2-pyrimidinyl]amino}phenyl)propanoic acid;
3-(4-{[4-{[5-(dimethylamino)[1,3]thiazolo[5,4-b]pyridin-2-yl]amino}-6-(phenylmethyl)-2-pyrimidinyl]amino}phenyl)-N,N-bis(2-hydroxyethyl)propanamide;
trans-4-{[4-{[5-(4-morpholinyl)[1,3]thiazolo[5,4-b]pyridin-2-yl]amino}-6-(phenylmethyl)-2-pyrimidinyl]amino}cyclohexanol;
trans-4-[(4-(phenylmethyl)-6-{[5-(1-pyrrolidinyl)[1,3]thiazolo[5,4-b]pyridin-2-yl]amino}-2-pyrimidinyl)amino]cyclohexanol;
trans-4-[(4-(phenylmethyl)-6-{[5-(1-piperidinyl)[1,3]thiazolo[5,4-b]pyridin-2-yl]amino}-2-pyrimidinyl)amino]cyclohexanol;
trans-4-[(4-(phenylmethyl)-6-{[5-(1-piperazinyl)[1,3]thiazolo[5,4-b]pyridin-2-yl]amino}-2-pyrimidinyl)amino]cyclohexanol;
trans-4-{[4-({5-[(2-hydroxyethyl)(methyl)amino][1,3]thiazolo[5,4-b]pyridin-2-yl}amino)-6-(phenylmethyl)-2-pyrimidinyl]amino}cyclohexanol;
trans-4-{[4-({5-[(3-hydroxypropyl)(methyl)amino][1,3]thiazolo[5,4-b]pyridin-2-yl}amino)-6-(phenylmethyl)-2-pyrimidinyl]amino}cyclohexanol;
3-[(2-{[2-[(trans-4-hydroxycyclohexyl)amino]-6-(phenylmethyl)-4-pyrimidinyl]amino}[1,3]thiazolo[5,4-b]pyridin-5-yl)(methyl)amino]-1,2-propanediol;
1-(2-{[2-[(trans-4-hydroxycyclohexyl)amino]-6-(phenylmethyl)-4-pyrimidinyl]amino}[1,3]thiazolo[5,4-b]pyridin-5-yl)-4-piperidinol;
trans-4-{[4-{[5-(4-amino-1-piperidinyl)[1,3]thiazolo[5,4-b]pyridin-2-yl]amino}-6-(phenylmethyl)-2-pyrimidinyl]amino}cyclohexanol;
trans-4-{[4-({5-[(3R)-3-amino-1-pyrrolidinyl][1,3]thiazolo[5,4-b]pyridin-2-yl}amino)-6-(phenylmethyl)-2-pyrimidinyl]amino}cyclohexanol;
trans-4-{[4-({5-[(3S)-3-amino-1-pyrrolidinyl][1,3]thiazolo[5,4-b]pyridin-2-yl}amino)-6-(phenylmethyl)-2-pyrimidinyl]amino}cyclohexanol;
trans-4-{[4-({5-[4-(2-hydroxyethyl)-1-piperazinyl][1,3]thiazolo[5,4-b]pyridin-2-yl}amino)-6-(phenylmethyl)-2-pyrimidinyl]amino}cyclohexanol;
trans-4-{[4-({5-[4-(2-aminoethyl)-1-piperazinyl][1,3]thiazolo[5,4-b]pyridin-2-yl}amino)-6-(phenylmethyl)-2-pyrimidinyl]amino}cyclohexanol;
3-(4-{[4-{[5-(4-morpholinyl)[1,3]thiazolo[5,4-b]pyridin-2-yl]amino}-6-(phenylmethyl)-2-pyrimidinyl]amino}phenyl)propanoic acid;
N²-{4-[3-(4-morpholinyl)-3-oxopropyl]phenyl}-N⁴-[5-(4-morpholinyl)[1,3]thiazolo[5,4-b]pyridin-2-yl]-6-(phenylmethyl)-2,4-pyrimidinediamine;
3-{4-[(4-(phenylmethyl)-6-{[5-(1-piperazinyl)[1,3]thiazolo[5,4-b]pyridin-2-yl]amino}-2-pyrimidinyl)amino]phenyl}propanoic acid;
trans-4-[(4-[difluoro(4-fluorophenyl)methyl]-6-{[5-(methyloxy)[1,3]thiazolo[5,4-b]pyridin-2-yl]amino}-2-pyrimidinyl)amino]cyclohexanol;
trans-4-[(4-(1,1-difluoroethyl)-6-{[6-(methyloxy)-1,3-benzothiazol-2-yl]amino}-2-pyrimidinyl)amino]cyclohexanol;
N2-(trans-4-aminocyclohexyl)-6-[difluoro(4-fluorophenyl)methyl]-N4-[6-(methyloxy)-1,3-benzothiazol-2-yl]-2,4-pyrimidinediamine;
trans-4-[(4-[difluoro(4-fluorophenyl)methyl]-6-{[6-(methyloxy)-1,3-benzothiazol-2-yl]amino}-2-pyrimidinyl)amino]cyclohexanol;
trans-4-({4-[difluoro(4-fluorophenyl)methyl]-6-[(6-methyl-1,3-benzothiazol-2-yl)amino]-2-pyrimidinyl}amino)cyclohexanol;
trans-4-({4-[(6-bromo-1,3-benzothiazol-2-yl)amino]-6-[difluoro(4-fluorophenyl)methyl]-2-pyrimidinyl}amino)cyclohexanol;
trans-4-[(4-(1,1-difluoroethyl)-6-{[6-(trifluoromethyl)-1,3-benzothiazol-2-yl]amino}-2-pyrimidinyl)amino]cyclohexanol;
trans-4-({4-(1,3-benzothiazol-2-ylamino)-6-[difluoro(4-fluorophenyl)methyl]-2-pyrimidinyl}amino)cyclohexanol;
trans-4-{[4-(1,3-benzothiazol-2-ylamino)-6-(1,1-difluoroethyl)-2-pyrimidinyl]amino}cyclohexanol;
4-[(4-[difluoro(4-fluorophenyl)methyl]-6-{[6-(methyloxy)-1,3-benzothiazol-2-yl]amino}-2-pyrimidinyl)amino]-1-butanol;
N2-(4-aminobutyl)-6-[difluoro(4-fluorophenyl)methyl]-N4-[6-(methyloxy)-1,3-benzothiazol-2-yl]-2,4-pyrimidinediamine;
6-[difluoro(4-fluorophenyl)methyl]-N4-[6-(methyloxy)-1,3-benzothiazol-2-yl]-N2-{[4-(methylsulfonyl)-2-morpholinyl]methyl}-2,4-pyrimidinediamine;
trans-4-[(4-[difluoro(4-fluorophenyl)methyl]-6-{[6-(ethyloxy)-1,3-benzothiazol-2-yl]amino}-2-pyrimidinyl)amino]cyclohexanol;
3-[(4-[difluoro(4-fluorophenyl)methyl]-6-{[6-(methyloxy)-1,3-benzothiazol-2-yl]amino}-2-pyrimidinyl)amino]-1-propanol;
trans-4-[(4-[difluoro(4-fluorophenyl)methyl]-6-{[6-(trifluoromethyl)-1,3-benzothiazol-2-yl]amino}-2-pyrimidinyl)amino]cyclohexanol;
6-[difluoro(4-fluorophenyl)methyl]-N4-[6-(methyloxy)-1,3-benzothiazol-2-yl]-N2-{[1-(methylsulfonyl)-3-piperidinyl]methyl}-2,4-pyrimidinediamine;
{4-[(4-[difluoro(4-fluorophenyl)methyl]-6-{[5-(methyloxy)[1,3]thiazolo[5,4-b]pyridin-2-yl]amino}-2-pyrimidinyl)amino]phenyl}acetic acid;
4-[(4-[difluoro(4-fluorophenyl)methyl]-6-{[6-(methyloxy)-1,3-benzothiazol-2-yl]amino}-2-pyrimidinyl)amino]benzenesulfonamide;
2-{4-[(4-[difluoro(4-fluorophenyl)methyl]-6-{[6-(methyloxy)-1,3-benzothiazol-2-yl]amino}-2-pyrimidinyl)amino]phenyl}ethanol;
N-({4-[(4-[difluoro(4-fluorophenyl)methyl]-6-{[6-(ethyloxy)-1,3-benzothiazol-2-yl]amino}-2-pyrimidinyl)amino]phenyl}sulfonyl)acetamide; or
   a salt thereof.

In another embodiment, the compound of the invention is:
trans-4-{[4-[(6-methyl-1,3-benzothiazol-2-yl)amino]-6-(phenylmethyl)-2-pyrimidinyl]amino}cyclohexanol;
trans-4-{[4-[(6-chloro-1,3-benzothiazol-2-yl)amino]-6-(phenylmethyl)-2-pyrimidinyl]amino}cyclohexanol;
trans-4-{[4-[(6-bromo-1,3-benzothiazol-2-yl)amino]-6-(phenylmethyl)-2-pyrimidinyl]amino}cyclohexanol;
trans-4-{[4-[(6-fluoro-1,3-benzothiazol-2-yl)amino]-6-(phenylmethyl)-2-pyrimidinyl]amino}cyclohexanol;
trans-4-{[4-[(6-ethyl-1,3-benzothiazol-2-yl)amino]-6-(phenylmethyl)-2-pyrimidinyl]amino}cyclohexanol;
trans-4-{[4-{[5-(methyloxy)[1,3]thiazolo[5,4-b]pyridin-2-yl]amino}-6-(phenylmethyl)-2-pyrimidinyl]amino}cyclohexanol;
trans-4-{[4-[(5-chloro[1,3]thiazolo[5,4-b]pyridin-2-yl)amino]-6-(phenylmethyl)-2-pyrimidinyl]amino}cyclohexanol;
trans-4-{[4-(phenylmethyl)-6-([1,3]thiazolo[5,4-b]pyridin-2-ylamino)-2-pyrimidinyl]amino}cyclohexanol;
trans-4-{[4-[(6-nitro-1,3-benzothiazol-2-yl)amino]-6-(phenylmethyl)-2-pyrimidinyl]amino}cyclohexanol;
(2-{[2-[(trans-4-hydroxycyclohexyl)amino]-6-(phenylmethyl)-4-pyrimidinyl]amino}-1,3-benzothiazol-6-yl)acetonitrile;
2-hydroxy-5-(2-{[4-[(6-methyl-1,3-benzothiazol-2-yl)amino]-6-(phenylmethyl)-2-pyrimidinyl]amino}ethyl)benzenesulfonamide;
4-{[4-[(6-methyl-1,3-benzothiazol-2-yl)amino]-6-(phenylmethyl)-2-pyrimidinyl]amino}-1-butanol;
5-(2-{[4-[(6-ethyl-1,3-benzothiazol-2-yl)amino]-6-(phenylmethyl)-2-pyrimidinyl]amino}ethyl)-2-hydroxybenzenesulfonamide;
4-(2-{[4-[(6-methyl-1,3-benzothiazol-2-yl)amino]-6-(phenylmethyl)-2-pyrimidinyl]amino}ethyl)benzenesulfonamide;
5-{[4-{[5-(methyloxy)[1,3]thiazolo[5,4-b]pyridin-2-yl]amino}-6-(phenylmethyl)-2-pyrimidinyl]amino}-1-pentanol;
trans-4-{[4-[(5-ethyl[1,3]thiazolo[5,4-b]pyridin-2-yl)amino]-6-(phenylmethyl)-2-pyrimidinyl]amino}cyclohexanol;
2-{[2-[(trans-4-hydroxycyclohexyl)amino]-6-(phenylmethyl)-4-pyrimidinyl]amino}-1,3-benzothiazole-6-carbonitrile;
ethyl (2-{[2-[(trans-4-hydroxycyclohexyl)amino]-6-(phenylmethyl)-4-pyrimidinyl]amino}-1,3-benzothiazol-6-yl)acetate;
ethyl 3-(2-{[2-[(trans-4-hydroxycyclohexyl)amino]-6-(phenylmethyl)-4-pyrimidinyl]amino}-1,3-benzothiazol-6-yl)propanoate;
trans-4-{[4-{[6-(1-methylethyl)-1,3-benzothiazol-2-yl]amino}-6-(phenylmethyl)-2-pyrimidinyl]amino}cyclohexanol;
4-(2-{[4-[(6-ethyl-1,3-benzothiazol-2-yl)amino]-6-(2-methylpropyl)-2-pyrimidinyl]amino}ethyl)benzenesulfonamide;
4-(2-{[4-[(6-ethyl-1,3-benzothiazol-2-yl)amino]-6-(phenylmethyl)-2-pyrimidinyl]amino}ethyl)benzenesulfonamide;
trans-4-{[4-[(6-iodo-1,3-benzothiazol-2-yl)amino]-6-(phenylmethyl)-2-pyrimidinyl]amino}cyclohexanol;
5-(2-{[4-[(6-ethyl-1,3-benzothiazol-2-yl)amino]-6-(2-methylpropyl)-2-pyrimidinyl]amino}ethyl)-2-hydroxybenzenesulfonamide;
(1R,3S)-3-{[4-[(6-ethyl-1,3-benzothiazol-2-yl)amino]-6-(phenylmethyl)-2-pyrimidinyl]amino}cyclopentanecarboxylic acid;
trans-4-{[4-{[6-(methylsulfonyl)-1,3-benzothiazol-2-yl]amino}-6-(phenylmethyl)-2-pyrimidinyl]amino}cyclohexanol;
trans-4-{[4-[(7-bromo-1,3-benzothiazol-2-yl)amino]-6-(phenylmethyl)-2-pyrimidinyl]amino}cyclohexanol;
3-(4-{[4-{[5-(methyloxy)[1,3]thiazolo[5,4-b]pyridin-2-yl]amino}-6-(phenylmethyl)-2-pyrimidinyl]amino}phenyl)propanoic acid;
3-(4-{[4-[(5-ethyl[1,3]thiazolo[5,4-b]pyridin-2-yl)amino]-6-(phenylmethyl)-2-pyrimidinyl]amino}phenyl)propanoic acid;
(4-{[4-[(6-ethyl-1,3-benzothiazol-2-yl)amino]-6-(phenylmethyl)-2-pyrimidinyl]amino}-3-fluorophenyl)acetic acid;
3-[(4-{[4-[(6-methyl-1,3-benzothiazol-2-yl)amino]-6-(phenylmethyl)-2-pyrimidinyl]amino}phenyl)oxy]propanoic acid;
(4-{[4-{[6-(cyanomethyl)-1,3-benzothiazol-2-yl]amino}-6-(phenylmethyl)-2-pyrimidinyl]amino}phenyl)acetic acid;
(3-fluoro-4-{[4-[(6-methyl-1,3-benzothiazol-2-yl)amino]-6-(phenylmethyl)-2-pyrimidinyl]amino}phenyl)acetic acid;
3-(4-{[4-[(5-chloro[1,3]thiazolo[5,4-b]pyridin-2-yl)amino]-6-(phenylmethyl)-2-pyrimidinyl]amino}phenyl)propanoic acid;
(4-{[4-{[6-(methyloxy)-1,3-benzothiazol-2-yl]amino}-6-(phenylmethyl)-2-pyrimidinyl]amino}phenyl)acetic acid;
trans-4-{[4-(1,3-benzothiazol-2-ylamino)-6-(phenylmethyl)-2-pyrimidinyl]amino}cyclohexanol;
trans-4-{[4-{[6-(methyloxy)-1,3-benzothiazol-2-yl]amino}-6-(phenylmethyl)-2-pyrimidinyl]amino}cyclohexanol;
4-(2-{[4-{[6-(methyloxy)-1,3-benzothiazol-2-yl]amino}-6-(phenylmethyl)-2-pyrimidinyl]amino}ethyl)benzenesulfonamide;
3-(4-{[4-{[6-(methyloxy)-1,3-benzothiazol-2-yl]amino}-6-(phenylmethyl)-2-pyrimidinyl]amino}phenyl)propanoic acid;
3-(2-{[2-[(trans-4-hydroxycyclohexyl)amino]-6-(phenylmethyl)-4-pyrimidinyl]amino}-1,3-benzothiazol-6-yl)propanoic acid;
2-(2-{[2-[(trans-4-hydroxycyclohexyl)amino]-6-(phenylmethyl)-4-pyrimidinyl]amino}-1,3-benzothiazol-6-yl)acetamide;
3-(2-{[2-[(trans-4-hydroxycyclohexyl)amino]-6-(phenylmethyl)-4-pyrimidinyl]amino}-1,3-benzothiazol-6-yl)propanamide;
trans-4-{[4-{[6-(2-hydroxyethyl)-1,3-benzothiazol-2-yl]amino}-6-(phenylmethyl)-2-pyrimidinyl]amino}cyclohexanol;
trans-4-{[4-{[6-(hydroxymethyl)-1,3-benzothiazol-2-yl]amino}-6-(phenylmethyl)-2-pyrimidinyl]amino}cyclohexanol;
trans-4-{[4-{[6-(3-hydroxypropyl)-1,3-benzothiazol-2-yl]amino}-6-(phenylmethyl)-2-pyrimidinyl]amino}cyclohexanol;
trans-4-{[4-[(6-amino-1,3-benzothiazol-2-yl)amino]-6-(phenylmethyl)-2-pyrimidinyl]amino}cyclohexanol;
1-(2-{[2-[(trans-4-hydroxycyclohexyl)amino]-6-(phenylmethyl)-4-pyrimidinyl]amino}-1,3-benzothiazol-6-yl)-2-azetidinone;
1-(2-{[2-[(trans-4-hydroxycyclohexyl)amino]-6-(phenylmethyl)-4-pyrimidinyl]amino}-1,3-benzothiazol-6-yl)-2-pyrrolidinone;
1-(2-{[2-[(trans-4-hydroxycyclohexyl)amino]-6-(phenylmethyl)-4-pyrimidinyl]amino}-1,3-benzothiazol-6-yl)-2,5-pyrrolidinedione;
1-(2-{[2-[(trans-4-hydroxycyclohexyl)amino]-6-(phenylmethyl)-4-pyrimidinyl]amino}-1,3-benzothiazol-6-yl)-2-imidazolidinone;
3-(2-{[2-[(trans-4-hydroxycyclohexyl)amino]-6-(phenylmethyl)-4-pyrimidinyl]amino}-1,3-benzothiazol-6-yl)-2,4-imidazolidinedione;
3-(2-{[2-[(trans-4-hydroxycyclohexyl)amino]-6-(phenylmethyl)-4-pyrimidinyl]amino}-1,3-benzothiazol-6-yl)-1,3-oxazolidin-2-one;
trans-4-{[4-{[5-(4-morpholinyl)[1,3]thiazolo[5,4-b]pyridin-2-yl]amino}-6-(phenylmethyl)-2-pyrimidinyl]amino}cyclohexanol;
trans-4-[(4-(phenylmethyl)-6-{[5-(1-piperazinyl)[1,3]thiazolo[5,4-b]pyridin-2-yl]amino}-2-pyrimidinyl)amino]cyclohexanol;
trans-4-{[4-({5-[(2-hydroxyethyl)(methyl)amino][1,3]thiazolo[5,4-b]pyridin-2-yl}amino)-6-(phenylmethyl)-2-pyrimidinyl]amino}cyclohexanol;
trans-4-{[4-({5-[(3-hydroxypropyl)(methyl)amino][1,3]thiazolo[5,4-b]pyridin-2-yl}amino)-6-(phenylmethyl)-2-pyrimidinyl]amino}cyclohexanol;
3-[(2-{[2-[(trans-4-hydroxycyclohexyl)amino]-6-(phenylmethyl)-4-pyrimidinyl]amino}[1,3]thiazolo[5,4-b]pyridin-5-yl)(methyl)amino]-1,2-propanediol;
1-(2-{[2-[(trans-4-hydroxycyclohexyl)amino]-6-(phenylmethyl)-4-pyrimidinyl]amino}[1,3]thiazolo[5,4-b]pyridin-5-yl)-4-piperidinol;
trans-4-{[4-{[5-(4-amino-1-piperidinyl)[1,3]thiazolo[5,4-b]pyridin-2-yl]amino}-6-(phenylmethyl)-2-pyrimidinyl]amino}cyclohexanol;
3-(4-{[4-{[5-(4-morpholinyl)[1,3]thiazolo[5,4-b]pyridin-2-yl]amino}-6-(phenylmethyl)-2-pyrimidinyl]amino}phenyl)propanoic acid;
3-{4-[(4-(phenylmethyl)-6-{[5-(1-piperazinyl)[1,3]thiazolo[5,4-b]pyridin-2-yl]amino}-2-pyrimidinyl)amino]phenyl}propanoic acid; or
   a salt thereof.

In another embodiment, the compound of the invention is:
trans-4-{[4-{[5-(methyloxy)[1,3]thiazolo[5,4-b]pyridin-2-yl]amino}-6-(phenylmethyl)-2-pyrimidinyl]amino}cyclohexanol;
trans-4-{[4-(phenylmethyl)-6-([1,3]thiazolo[5,4-b]pyridin-2-ylamino)-2-pyrimidinyl]amino}cyclohexanol;
trans-4-{[4-[(6-nitro-1,3-benzothiazol-2-yl)amino]-6-(phenylmethyl)-2-pyrimidinyl]amino}cyclohexanol;
(2-{[2-[(trans-4-hydroxycyclohexyl)amino]-6-(phenylmethyl)-4-pyrimidinyl]amino}-1,3-benzothiazol-6-yl)acetonitrile;
5-{[4-{[5-(methyloxy)[1,3]thiazolo[5,4-b]pyridin-2-yl]amino}-6-(phenylmethyl)-2-pyrimidinyl]amino}-1-pentanol;
trans-4-{[4-[(5-ethyl[1,3]thiazolo[5,4-b]pyridin-2-yl)amino]-6-(phenylmethyl)-2-pyrimidinyl]amino}cyclohexanol;
2-{[2-[(trans-4-hydroxycyclohexyl)amino]-6-(phenylmethyl)-4-pyrimidinyl]amino}-1,3-benzothiazole-6-carbonitrile;
3-(4-{[4-[(5-ethyl[1,3]thiazolo[5,4-b]pyridin-2-yl)amino]-6-(phenylmethyl)-2-pyrimidinyl]amino}phenyl)propanoic acid;
3-(2-{[2-[(trans-4-hydroxycyclohexyl)amino]-6-(phenylmethyl)-4-pyrimidinyl]amino}-1,3-benzothiazol-6-yl)propanamide;
trans-4-{[4-{[6-(2-hydroxyethyl)-1,3-benzothiazol-2-yl]amino}-6-(phenylmethyl)-2-pyrimidinyl]amino}cyclohexanol;
trans-4-{[4-{[6-(hydroxymethyl)-1,3-benzothiazol-2-yl]amino}-6-(phenylmethyl)-2-pyrimidinyl]amino}cyclohexanol;
1-(2-{[2-[(trans-4-hydroxycyclohexyl)amino]-6-(phenylmethyl)-4-pyrimidinyl]amino}-1,3-benzothiazol-6-yl)-2-azetidinone;
1-(2-{[2-[(trans-4-hydroxycyclohexyl)amino]-6-(phenylmethyl)-4-pyrimidinyl]amino}-1,3-benzothiazol-6-yl)-2-pyrrolidinone;
1-(2-{[2-[(trans-4-hydroxycyclohexyl)amino]-6-(phenylmethyl)-4-pyrimidinyl]amino}-1,3-benzothiazol-6-yl)-2,5-pyrrolidinedione;
3-(2-{[2-[(trans-4-hydroxycyclohexyl)amino]-6-(phenylmethyl)-4-pyrimidinyl]amino}-1,3-benzothiazol-6-yl)-2,4-imidazolidinedione;
3-(2-{[2-[(trans-4-hydroxycyclohexyl)amino]-6-(phenylmethyl)-4-pyrimidinyl]amino}-1,3-benzothiazol-6-yl)-1,3-oxazolidin-2-one;
3-[(2-{[2-[(trans-4-hydroxycyclohexyl)amino]-6-(phenylmethyl)-4-pyrimidinyl]amino}[1,3]thiazolo[5,4-b]pyridin-5-yl)(methyl)amino]-1,2-propanediol; or
   a salt thereof.

In a further embodiment, the compound of the invention is:
1-(2-{[2-[(trans-4-hydroxycyclohexyl)amino]-6-(phenylmethyl)-4-pyrimidinyl]amino}-1,3-benzothiazol-6-yl)-2,5-pyrrolidinedione;
trans-4-[(4-(phenylmethyl)-6-{[5-(1H-pyrazol-4-yl)[1,3]thiazolo[5,4-b]pyridin-2-yl]amino}-2-pyrimidinyl)amino]cyclohexanol;
5-(2-{[2-[(trans-4-hydroxycyclohexyl)amino]-6-(1-piperidinylmethyl)-4-pyrimidinyl]amino}-1,3-benzothiazol-6-yl)-3-pyridinecarbonitrile;
5-(2-{[2-[(trans-4-hydroxycyclohexyl)amino]-6-(4-morpholinylmethyl)-4-pyrimidinyl]amino}-1,3-benzothiazol-6-yl)-3-pyridinecarbonitrile;
5-[2-({6-{[(2R,6S)-2,6-dimethyl-4-morpholinyl]methyl}-2-[(trans-4-hydroxycyclohexyl)amino]-4-pyrimidinyl}amino)-1,3-benzothiazol-6-yl]-3-pyridinecarbonitrile;
5-[2-({6-[(3,3-difluoro-1-piperidinyl)methyl]-2-[(trans-4-hydroxycyclohexyl)amino]-4-pyrimidinyl}amino)-1,3-benzothiazol-6-yl]-3-pyridinecarbonitrile;
3-[2-({6-{[(2R,6S)-2,6-dimethyl-4-morpholinyl]methyl}-2-[(trans-4-hydroxycyclohexyl)amino]-4-pyrimidinyl}amino)-1,3-benzothiazol-6-yl]-1,3-oxazolidin-2-one;
3-[2-({6-[(3,3-difluoro-1-piperidinyl)methyl]-2-[(trans-4-hydroxycyclohexyl)amino]-4-pyrimidinyl}amino)-1,3-benzothiazol-6-yl]-1,3-oxazolidin-2-one;
trans-4-[(4-(1-piperidinylmethyl)-6-{[6-(1H-pyrazol-4-yl)-1,3-benzothiazol-2-yl]amino}-2-pyrimidinyl)amino]cyclohexanol;
trans-4-[(4-(4-morpholinylmethyl)-6-{[6-(1H-pyrazol-4-yl)-1,3-benzothiazol-2-yl]amino}-2-pyrimidinyl)amino]cyclohexanol;
trans-4-[(4-{[(2R,6S)-2,6-dimethyl-4-morpholinyl]methyl}-6-{[6-(1H-pyrazol-4-yl)-1,3-benzothiazol-2-yl]amino}-2-pyrimidinyl)amino]cyclohexanol;
trans-4-[(4-[(3,3-difluoro-1-piperidinyl)methyl]-6-{[6-(1H-pyrazol-4-yl)-1,3-benzothiazol-2-yl]amino}-2-pyrimidinyl)amino]cyclohexanol; or
   a salt thereof.

### TERMS AND DEFINITIONS

“Alkyl” refers to a saturated hydrocarbon chain having the specified number of member atoms. C₁₋₈alkyl refers to an alkyl group having from 1 to 8 member atoms. For example, C₁₋₆alkyl refers to an alkyl group having from 1 to 6 member atoms, for example 1 to 4 member atoms. Alkyl groups may be optionally substituted with one or more substituents as defined herein. Alkyl groups may be straight or branched. Representative branched alkyl groups have one, two, or three branches. Alkyl includes methyl, ethyl, propyl (n-propyl and isopropyl), butyl (n-butyl, isobutyl, and t-butyl), pentyl (n-pentyl, isopentyl, and neopentyl), and hexyl. Alkyl also includes heptyl, octyl and 1,1,3,3-tetramethylbutyl.

“Alkenyl” refers to a hydrocarbon chain having the specified number of member atoms and at least one double bond. For example, C₂₋₆alkenyl refers to an alkenyl group having from 2 to 6 member atoms, for example 2 to 4 member atoms. Alkenyl groups may be optionally substituted with one or more substituents as defined herein. Alkenyl groups may be straight or branched. Alkenyl includes ethenyl, 2-propenyl, 3-butenyl, 2-butenyl, 2-pentenyl, 3-pentenyl, 3-methyl-2-butenyl, 3-methylbut-2-enyl, 3-hexenyl and 1,1-dimethylbut-2-enyl.

“Cycloalkyl” refers to a saturated hydrocarbon ring having the specified number of member atoms. Cycloalkyl groups are monocyclic ring systems. For example, C₃₋₆cycloalkyl refers to a cycloalkyl group having from 3 to 6 member atoms. Cycloalkyl groups may be optionally substituted with one or more substituents as defined herein. Cycloalkyl includes cyclopropyl, cyclobutyl, cyclopentyl, and cyclohexyl.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” refers to products whose enantiomeric excess is 99% ee or greater.

“Half-life” (or “half-lives”) refers to the time required for half of a quantity of a substance to be converted to another chemically distinct species in vitro or in vivo.

“Halo” refers to the halogen radical fluoro, chloro, bromo, or iodo.

“Heteroaryl”, unless otherwise defined, refers to an aromatic ring containing 1 or 2 heteroatoms as member atoms in the ring. Heteroaryl groups containing more than one heteroatom may contain different heteroatoms. Heteroaryl groups may be optionally substituted with one or more substituents as defined herein. The heteroaryl groups herein are monocyclic ring systems having 5 or 6 member atoms. Heteroaryl includes pyrrolyl, furanyl, pyrazolyl, imidazolyl, oxazolyl, isoxazolyl, pyridinyl, pyrimidinyl, pyridazinyl and pyrazinyl.

“Heteroatom” refers to a nitrogen, sulphur, or oxygen atom.

“Heterocyclyl”, unless otherwise defined, refers to a saturated or unsaturated ring containing 1 or 2 heteroatoms as member atoms in the ring. However, heterocyclyl rings are not aromatic. In certain embodiments, heterocyclyl is saturated. In other embodiments, heterocyclyl is unsaturated but not aromatic. Heterocyclyl groups containing more than one heteroatom may contain different heteroatoms. The heterocyclyl groups herein are monocyclic ring systems having 4, 5 or 6 member atoms. Heterocyclyl groups may be optionally substituted with one or more substituents as defined herein. Heterocyclyl includes azetidinyl, pyrrolidinyl, pyrazolidinyl, imidazolinyl, oxazolidinyl, isoxazolidinyl, tetrahydropyranyl, dihydropyranyl, pyranyl, 1,3-dioxanyl, 1,4-dioxanyl, piperidinyl, piperazinyl and morpholinyl. Heterocyclyl also includes tetrahydrothienyl, tetrahydrothiopyranyl, thiazolidinyl, isothiazolidinyl and thiomorpholinyl. In one embodiment, heterocyclyl is azetidinyl, pyrrolidinyl, imidazolinyl, oxazolidinyl, piperidinyl, piperazinyl or morpholinyl.

“Member atoms” refers to the atom or atoms that form a chain or ring. Where more than one member atom is present in a chain and within a ring, each member atom is covalently bound to an adjacent member atom in the chain or ring. Atoms that make up a substituent group on a chain or ring are not member atoms in the chain or ring.

“Optionally substituted” indicates that a group, such as heteroaryl, may be unsubstituted or substituted with one or more substituents as defined herein.

“Substituted” in reference to a group indicates that a hydrogen atom attached to a member atom within a group is replaced. It should be understood that the term “substituted” includes the implicit provision that such substitution be in accordance with the permitted valence of the substituted atom and the substituent and that the substitution results in a stable compound (i.e. one that does not spontaneously undergo transformation such as by rearrangement, cyclization, or elimination). In certain embodiments, a single atom may be substituted with more than one substituent as long as such substitution is in accordance with the permitted valence of the atom. Suitable substituents are defined herein for each substituted or optionally substituted group.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein the symbols and conventions used in these processes, schemes and examples are consistent with those used in the contemporary scientific literature, for example, the Journal of the American Chemical Society or the Journal of Biological Chemistry. Unless otherwise noted, all starting materials were obtained from commercial suppliers and used without further purification. Specifically, the following abbreviations may be used in the examples and throughout the specification:

DIPEA diisopropylethylamine
Et ethyl
HPLC high performance liquid chromatography
iPr isopropyl
LCMS liquid chromatography-mass spectrometry
MDAP mass-directed autopreparative HPLC
Me methyl
min minutes
mg milligrams
mL millilitres
mM millimolar
mmol millimoles
m/z mass/charge ratio
NMR nuclear magnetic resonance
Pr n-propyl
Rt retention time
tBu tertiary butyl
TFA trifluoroacetic acid
THF tetrahydrofuran
HPLC ultra performance liquid chromatography
UV ultraviolet

All references to brine are to a saturated aqueous solution of NaCl.

Included within the scope of the “compounds of the invention” are all solvates (including hydrates), complexes, polymorphs, prodrugs, radiolabelled derivatives, stereoisomers and optical isomers of the compounds of formula (I) and salts thereof.

The compounds of the invention may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically acceptable solvates may be formed wherein solvent molecules are incorporated into the crystalline lattice during crystallization. Solvates may involve nonaqueous solvents such as ethanol, isopropanol, DMSO, acetic acid, ethanolamine, and EtOAc, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The skilled artisan will further appreciate that certain compounds of the invention that exist in crystalline form, including the various solvates thereof, may exhibit polymorphism (i.e. the capacity to occur in different crystalline structures). These different crystalline forms are typically known as “polymorphs”. The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

The invention also includes isotopically-labelled compounds, which are identical to the compounds of formula (I) and salts thereof, but for the fact that one or more atoms are replaced by an atom having an atomic mass or mass number different from the atomic mass or mass number most commonly found in nature. Examples of isotopes that can be incorporated into the compounds of the invention include isotopes of hydrogen, carbon, nitrogen, oxygen and fluorine, such as 3H, 11C, 14C and 18F.

The compounds according to formula (I) may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in formula (I), or in any chemical structure illustrated herein, is not specified the structure is intended to encompass any stereoisomer and all mixtures thereof. Thus, compounds according to formula (I) containing one or more chiral center may be used as racemic mixtures, enantiomerically enriched mixtures, or as enantiomerically pure individual stereoisomers.

Individual stereoisomers of a compound according to formula (I) which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The compounds according to formula (I) may also contain centers of geometric asymmetry. Where the stereochemistry of a center of geometric asymmetry present in formula (I), or in any chemical structure illustrated herein, is not specified, the structure is intended to encompass the trans geometric isomer, the cis geometric isomer, and all mixtures thereof. Likewise, all tautomeric forms are also included in formula (I) whether such tautomers exist in equilibrium or predominately in one form.

It is to be understood that the references herein to compounds of formula (I) and salts thereof covers the compounds of formula (I) as free acids or free bases, or as salts thereof, for example as pharmaceutically acceptable salts thereof. Thus, in one embodiment, the invention is directed to compounds of formula (I) as the free acid or free base. In another embodiment, the invention is directed to compounds of formula (I) and salts thereof. In a further embodiment, the invention is directed to compounds of formula (I) and pharmaceutically acceptable salts thereof.

The skilled artisan will appreciate that pharmaceutically acceptable salts of the compounds according to formula (I) may be prepared. Indeed, in certain embodiments of the invention, pharmaceutically acceptable salts of the compounds according to formula (I) may be preferred over the respective free base or free acid because such salts impart greater stability or solubility to the molecule thereby facilitating formulation into a dosage form. Accordingly, the invention is further directed to compounds of formula (I) and pharmaceutically acceptable salts thereof.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

Salts and solvates having non-pharmaceutically acceptable counter-ions or associated solvents are within the scope of the present invention, for example, for use as intermediates in the preparation of other compounds of formula (I) and their pharmaceutically acceptable salts. Thus one embodiment of the invention embraces compounds of formula (I) and salts thereof.

In certain embodiments, compounds according to formula (I) may contain an acidic functional group. Suitable pharmaceutically-acceptable salts include salts of such acidic functional groups. Representative salts include pharmaceutically acceptable metal salts such as sodium, potassium, lithium, calcium, magnesium, aluminum, and zinc salts; carbonates and bicarbonates of a pharmaceutically acceptable metal cation such as sodium, potassium, lithium, calcium, magnesium, aluminum, and zinc; pharmaceutically acceptable organic primary, secondary, and tertiary amines including aliphatic amines, aromatic amines, aliphatic diamines, and hydroxy alkylamines such as methylamine, ethylamine, 2-hydroxyethylamine, diethylamine, TEA, ethylenediamine, ethanolamine, diethanolamine, and cyclohexylamine.

In certain embodiments, compounds according to formula (I) may contain a basic functional group and are therefore capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, naphthoate, hydroxynaphthoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

### Compound Preparation

The compounds of the invention may be made by a variety of methods, including standard chemistry. Any previously defined variable will continue to have the previously defined meaning unless otherwise indicated. Illustrative general synthetic methods are set out below and then specific compounds of the invention are prepared in the Examples section.

### Process a

Compounds of formula (I) wherein R¹ to R⁶ and X are as defined above, and salts thereof, may be prepared by a process comprising reaction of a compound of formula (II)

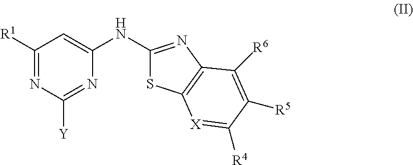

wherein R¹, R⁴, R⁵, R⁶ and X are as defined above and Y is halo such as fluoro or chloro, —SOCH₃, —SO₂CH₃, —SOCH₂Ph or —SO₂CH₂Ph, with an amine of formula (III)

HNR²R³ (III)

wherein R² and R³ as defined above.

When the compound of formula (III) is an aliphatic amine, the process may be carried out under microwave irradiation, in a suitable solvent such as isopropanol, optionally in the presence of a suitable base such as N-ethyldiisopropylamine, and at a suitable temperature such as 120-180° C.

When the compound of formula (III) is an aromatic amine, the process may be carried out under microwave irradiation, in a suitable solvent such as acetonitrile or DMSO, in the presence of catalytic aqueous HCl, and at a suitable temperature such as 140-170° C. Alternatively, the process may be carried out by heating in a suitable solvent such as acetonitrile, in the presence of 4-toluenesulfonic acid, and at a suitable temperature such as 140-160° C.

Compounds of formula (II) wherein Y is halo such as chloro or fluoro, may be prepared by a process comprising reaction of a compound of formula (IV)

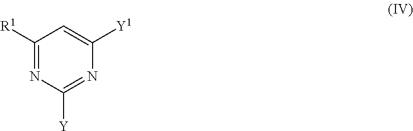

wherein R¹ is —CR⁷R⁸R⁹ wherein R⁷ and R⁸ are both hydrogen and Y and Y¹ are fluoro, or R¹ is —CR⁷R⁸R⁹ wherein R⁷ and R⁸ are both fluoro and Y and Y¹ are halo such as chloro or fluoro, with a compound of formula (V)

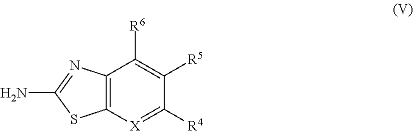

wherein R⁴ to R⁶ and X are as defined above. The process may be carried out in a suitable solvent such as tetrahydrofuran, in the presence of a suitable base such as sodium hydride or lithium hexamethyldisilazide, and at a suitable temperature such as 0° C. or −78° C. then allowing the reaction mixture to warm to ambient temperature.

Compounds of formula (IV) wherein R¹ is —CR⁷R⁸R⁹ wherein R⁷ and R⁸ are both hydrogen and Y and Y¹ are fluoro, may be prepared by a process comprising reaction of the compound of formula (VI)

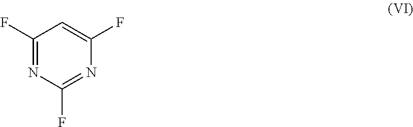

with a Grignard reagent of formula (VII)

R⁹R⁸R⁷CMgCl (VII)

wherein R⁷ and R⁸ are both hydrogen and R⁹ is as defined above. The process may be carried out in the presence of a suitable solvent such as tetrahydrofuran and at a suitable temperature such as −78° C. to −65° C. For compounds of formula (VII) wherein R⁹ is isobutyl, catalytic iron (III) (acac)₃ may be added prior to addition of the Grignard reagent.

Compounds of formula (IV) wherein R¹ is —CR⁷R⁸R⁹ wherein R⁷ and R⁸ are both fluoro and Y and Y¹ are chloro, may be prepared by a process comprising reaction of the compound of formula (VIII)

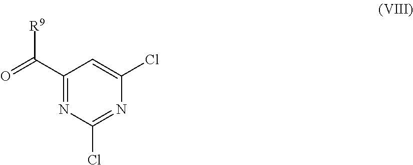

with diethylaminosulfur trifluoride in a suitable solvent such as dichloromethane and at a suitable temperature such as room temperature.

Compounds of formula (VIII) may be prepared by a process comprising reaction of the compound of formula (IX)

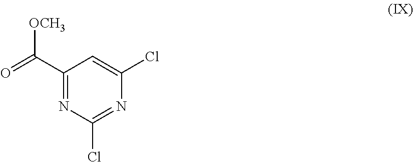

with a Grignard reagent of formula (X)

R⁹MgCl (X)

wherein R⁹ is as defined above. The process may be carried out in the presence of a suitable solvent such as tetrahydrofuran and at a suitable temperature such as −78° C.

Compounds of formula (V) may be prepared by a process comprising reaction of a compound of formula (XII)

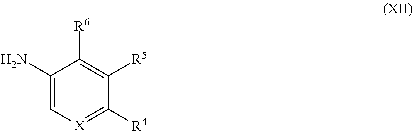

wherein R⁴ to R⁶ and X are as defined above, with potassium thiocyanate and bromine, in a suitable solvent such as acetic acid and at a suitable temperature such as 0° C. then allowing the reaction mixture to warm to ambient temperature.

Alternatively, compounds of formula (V) may be prepared by a process comprising reaction of a compound of formula (XIII)

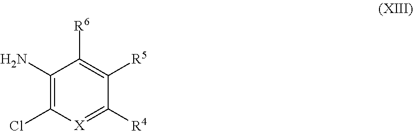

wherein R⁴ to R⁶ are as defined above and X is —N—, with potassium thiocyanate in the presence of aqueous hydrochloric acid. The process may be carried out under microwave irradiation, in a suitable solvent such as ethanol and at a suitable temperature such as 130° C.

Compounds of formula (XIII) may be prepared by a process comprising reaction of a compound of formula (XIV)

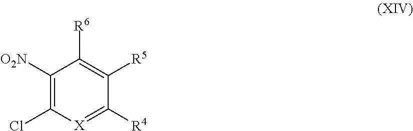

wherein R⁴ to R⁶ are as defined above and X is —N—, with tin (II) chloride in a suitable solvent such as ethanol and at a suitable temperature such as 50° C.

Compounds of formula (XIV) may be prepared by a process comprising reaction of a compound of formula (XV)

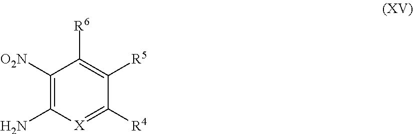

wherein R⁴ to R⁶ are as defined above and X is —N—, with sodium nitrite in concentrated hydrochloric acid, at a suitable temperature such as −15° C.

Compounds of formula (XV) may be prepared by a process comprising reaction of a a compound of formula (XVI)

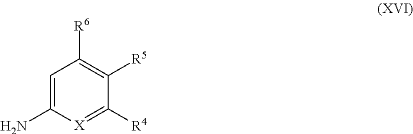

wherein R⁴ to R⁶ are as defined above and X is —N—, with concentrated sulphuric acid and concentrated nitric acid, at a suitable temperature such as 0° C. to 50° C. then allowing the reaction mixture to stir at ambient temperature.

Compounds of formula (II) wherein Y is —SOCH₃ or —SO₂CH₃, may be prepared by a process comprising reaction of a compound of formula (XVII)

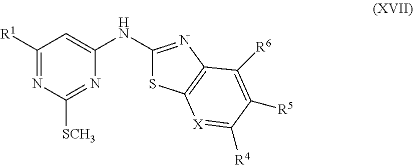

wherein R¹, R⁴ to R⁶ and X are as defined above, with a suitable oxidising agent such as Oxone®, in a suitable solvent such as dimethyl formamide and at a suitable temperature such as room temperature.

Compounds of formula (XVII) may be prepared by a process comprising reaction of a compound of formula (XVIII)

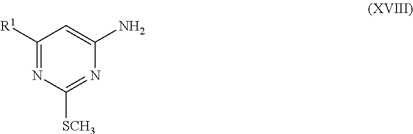

wherein R¹ is as defined above, with a compound of formula (XIX)

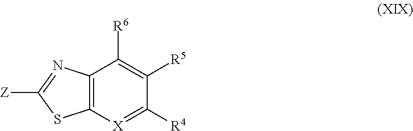

wherein R⁴ to R⁶ and X are as defined above and Z is halo such as chloro. The process may be carried out in a suitable solvent such as tetrahydrofuran, in the presence of a suitable base such as sodium hydride, and at a suitable temperature such as 60° C.

Compounds of formula (XVIII) may be prepared by a process comprising reaction of a compound of formula (XX)

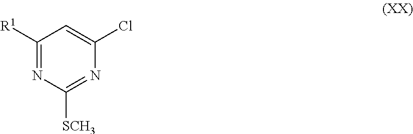

wherein R¹ is as defined above, with aqueous ammonia. The process may be carried out under microwave irradiation in a suitable solvent such as isopropanol and at a suitable temperature such as 150° C.

Compounds of formula (XX) may be prepared by a process comprising reaction of the compound of formula (XXI)

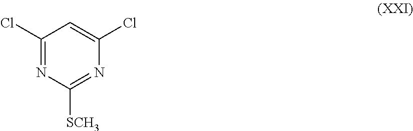

with a Grignard reagent of formula (XXII)

R¹MgCl (XXII)

wherein R¹ is as defined above. The process may be carried out in the presence of a suitable solvent such as tetrahydrofuran, and at a suitable temperature such as −78° C.

### Process b

Compounds of formula (I) wherein R¹, R², R³, R⁵, R⁶ and X are as defined above and R⁴ is C₁₋₆alkyl substituted by —OH, —CO₂H or —CONH₂, and salts thereof, may also be prepared by a process which comprises reduction, hydrolysis or amination of a compound of formula (XXIII)

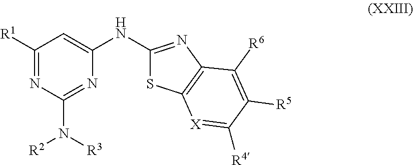

wherein R¹, R², R³, R⁵, R⁶ and X are as defined above and R^{4′} is C₁₋₆alkyl substituted by —CO₂R²⁴ wherein R²⁴ is C₁₋₆alkyl.

For compounds of formula (I) wherein R¹, R², R³, R⁵, R⁶ and X are as defined above and R⁴ is C₁₋₆alkyl substituted by —OH, a compound of formula (XXIII) wherein R^{4′} is C₁₋₅alkyl substituted by —CO₂R²⁴ wherein R²⁴ is C₁₋₆alkyl, may be reduced using a reducing agent such as lithium aluminium hydride, in a suitable solvent such as tetrahydrofuran, and at a suitable temperature such as 0° C.

For compounds of formula (I) wherein R¹, R², R³, R⁵, R⁶ and X are as defined above and R⁴ is C₁₋₆alkyl substituted by —CO₂H, a compound of formula (XXIII) wherein R^{4′} is C₁₋₆alkyl substituted by —CO₂R²⁴ wherein R²⁴ is C₁₋₆alkyl, may be hydrolysed under microwave irradiation using aqueous ammonia at a suitable temperature such as 100° C.

For compounds of formula (I) wherein R¹, R², R³, R⁵, R⁶ and X are as defined above and R⁴ is C₁₋₆alkyl substituted by —CONH₂, a compound of formula (XXIII) wherein R^{4′} is C₁₋₆alkyl substituted by —CO₂R²⁴ wherein R²⁴ is C₁₋₆alkyl, may be aminated under microwave irradiation using ammonia in methanol at a suitable temperature such as 150° C.

### Process c

Compounds of formula (I) wherein R¹ to R⁶ and X are as defined above, and salts thereof, may also be prepared by a process comprising final stage modification of one compound of formula (I), or a salt thereof, into another compound of formula (I), or a salt thereof. Suitable functional group transformations for converting one compound of formula (I) into another compound of formula (I) are well known in the art and are described in, for instance, Comprehensive Heterocyclic Chemistry II, eds. A. R. Katritzky, C. W. Rees and E. F. V. Scriven (Pergamon Press, 1996), Comprehensive Organic Functional Group Transformations, eds. A. R. Katritzky, O. Meth-Cohn and C. W. Rees (Elsevier Science Ltd., Oxford, 1995), Comprehensive Organic Chemistry, eds. D. Barton and W. D. Ollis (Pergamon Press, Oxford, 1979), and Comprehensive Organic Transformations, R. C. Larock (VCH Publishers Inc., New York, 1989).

For example, compounds of formula (I) wherein R⁴ is halo such as chloro may be reacted with a suitable amine compound to produce compounds of formula (I) wherein R⁴ is —NR¹⁶R¹⁷.

Compounds of formula (I) wherein R¹, R², R³, R⁵ and R⁶ are as defined above and R⁴ is —NR¹⁶R¹⁷ wherein R¹⁶ and R¹⁷, together with the nitrogen atom to which they are attached, are linked to form a 5-membered heterocyclyl wherein the 5-membered heterocyclyl optionally contains an oxygen atom and is optionally substituted by one or two oxo substituents may be prepared by a process comprising reacting a compound of formula (I) wherein R¹, R², R³, R⁵ and R⁶ are as defined above and R⁴ is halo such as chloro or bromo, with a 5-membered heterocyclyl containing a nitrogen atom and optionally an oxygen atom wherein the 5-membered heterocyclyl is optionally substituted by one or two oxo substituents. Suitable reaction conditions include treatment with copper (I) iodide and N,N′dimethylethylene diamine in the presence of a suitable base such as caesium carbonate, in a suitable solvent such as DMF, and at a suitable temperature such as 110-120° C. for example about 110° C.

Compounds of formula (I) wherein R⁴ is —NO₂ may be reduced to produce compounds of formula (I) wherein R⁴ is —NH₂. Suitable reduction conditions include treatment with hydrogen in the presence of a suitable catalyst such as palladium on activated carbon, and in a suitable solvent such as tetrahydrofuran.

Compounds of formula (I) wherein R⁴ is —NH₂ may be modified to produce further compounds of formula (I) wherein R⁴ is —NR¹⁶R¹⁷. For example, compounds of formula (I) wherein R¹, R², R³, R⁵ and R⁶ are as defined above and R⁴ is 1,1-dioxido-2-isothiazolidinyl may be prepared by a process comprising reacting a compound of formula (I) wherein R¹, R², R³, R⁵ and R⁶ are as defined above and R⁴ is —NH₂, with 3-chloro-1-propanesulfonyl chloride. Suitable reaction conditions include reaction in the presence of DMAP and a suitable base such as N-ethyldiisopropylamine, in a suitable solvent such as tetrahydrofuran, and at a suitable temperature such as about 0° C.

Compounds of formula (I) wherein R², R³, R⁵ and R⁶ are as defined above, R⁴ is halo such as chloro or bromo and R¹ is —CH₂NR²⁵R²⁶ may be prepared by a process comprising reaction of a compound of formula (I) wherein R², R³, R⁵ and R⁶ are as defined above, R⁴ is halo such as chloro or bromo and R¹ is —CH₂OH with methanesulphonyl chloride followed by a suitable amine of formula HNR²⁵R²⁶.

Certain compounds of formula (I) wherein R¹ to R⁶ are as defined above, and salts thereof, may be prepared by a process comprising a Suzuki coupling. For example, certain compounds of formula (I) wherein R¹ to R⁶ are as defined above, and salts thereof, may be prepared by a process comprising coupling of a compound of formula (I) wherein R¹, R², R³, R⁵ and R⁶ are as defined above and R⁴ is halo such as chloro or bromo, with a suitable boronic acid or boronic ester.

### Process d

Compounds of formula (I) wherein R¹ to R⁶ are as defined above, and salts thereof, may be prepared by a process comprising Suzuki coupling of a compound of formula (XXIV)

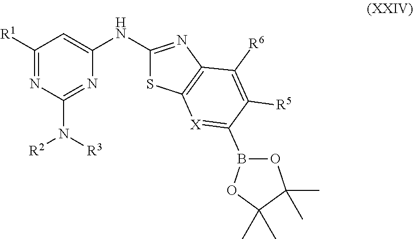

wherein R¹, R², R³, R⁵ and R⁶ are as defined above, with a suitable halide.

Suitable conditions for Suzuki coupling include microwave irradiation, in the presence of a suitable palladium catalyst such as tetrakis(triphenylphosphine)palladium (0), 1,1′-bis(diphenylphosphino)ferrocene-palladium(II) dichloride dichloromethane adduct or 2′-(dimethylamino)-2-biphenyl-palladium (II) chloride dinorbornylphosphine complex, in a suitable solvent such as aqueous 1,4-dioxane, in the presence of a suitable base such as caesium carbonate or potassium phosphate, at a suitable temperature such as between 100-150° C., and for a suitable time such as 30-90 minutes.

Compounds of formula (XXIV) wherein R¹, R², R³, R⁵ and R⁶ are as defined above may be prepared from compounds of formula (I) wherein R¹, R², R³, R⁵ and R⁶ are as defined above and R⁴ is halo such as chloro or bromo, by treatment with 4,4,4′,4′,5,5,5′,5′-octamethyl-2,2′-bi-1,3,2-dioxaborolane under microwave irradiation, in the presence of a suitable palladium catalyst such as 1,1′-bis(diphenylphosphino)ferrocene-palladium(II)dichloride dichloromethane adduct, in a suitable solvent such as tetrahydrofuran, in the presence of a suitable base such as potassium acetate, and at a suitable temperature such as between 110-170° C. for example about 120° C.

### Process e

Compounds of formula (I) wherein R², R³, R⁴, R⁵ and R⁶ are as defined above and R¹ is —CH₂NR²⁵R²⁶ may be prepared by a process comprising reaction of a compound of formula (XXV)

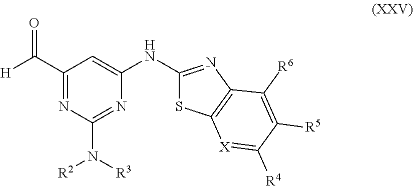

wherein R², R³, R⁴, R⁵ and R⁶ are as defined above, with an amine of formula (III) as defined above followed by treatment with sodium triacetoxyborohydride.

Suitable conditions include stirring in the presence of a suitable solvent such as tetrahydrofuran and dichloromethane, and at a suitable temperature such as room temperature, for example about 20° C.

### Process f

Compounds of formula (I) wherein R², R³, R⁴, R⁵ and R⁶ are as defined above and R¹ is —CH₂NR²⁵R²⁶ wherein R²⁵ and R²⁶, together with the nitrogen atom to which they are attached, are linked to form 2-piperazinone may be prepared by a process comprising reaction of a compound of formula (XXVI)

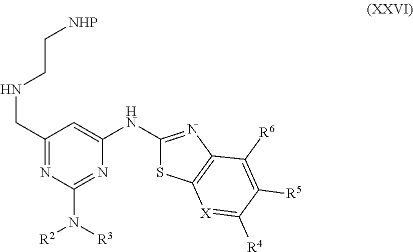

wherein R², R³, R⁴, R⁵ and R⁶ are as defined above and P is a protecting group such as Boc (t-butoxycarbonyl), with chloroacetyl chloride.

Suitable conditions include stirring in the presence of a suitable solvent such as tetrahydrofuran or dichloromethane, in the presence of a suitable base such as N-ethyldiisopropylamine and at a suitable temperature such as room temperature, for example about 20° C., followed by treatment with trifluoroacetic acid and work up using saturate aqueous sodium bicarbonate.

### Process g

Compounds of formula (I) wherein R², R³, R⁴, R⁵ and R⁶ are as defined above and R¹ is 6-methyl-3(2H)-pyridazinone may be prepared by a process comprising reaction of a compound of formula (XXV) as defined above, with 6-methyl-4,5-dihydro-3(2H)-pyridazinone.

Suitable conditions include heating in the presence of a suitable solvent such as ethanol, a suitable base such as potassium hydroxide, and at a suitable temperature such between 50-70° C., for example about 60° C.

Thus, in one embodiment, the invention provides a process for preparing a compound comprising:

a) reaction of a compound of formula (II)

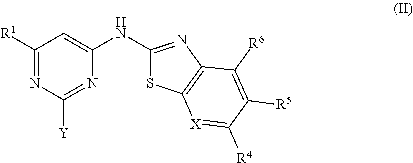

wherein R¹, R⁴, R⁵, R⁶ and X are as defined above and Y is halo, —SOCH₃ or —SO₂CH₃, with an amine of formula (III)

HNR²R³ (III)

wherein R² and R³ as defined above,
b) for compounds of formula (I) wherein R¹, R², R³, R⁵, R⁶ and X are as defined above and R⁴ is C₁₋₆alkyl substituted by —OH, —CO₂H or —CONH₂, and salts thereof, reduction, hydrolysis or amination of a compound of formula (XXIII)

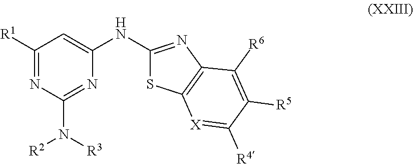

wherein R¹, R², R³, R⁵, R⁶ and X are as defined above and R^{4′} is C₁₋₆alkyl substituted by —CO₂R²⁴ wherein R²⁴ is C₁₋₆alkyl,
c) final stage modification of one compound of formula (I), or a salt thereof, into another compound of formula (I), or a salt thereof,
d) Suzuki coupling of a compound of formula (XXIV)

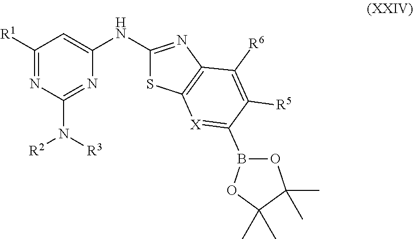

wherein R¹, R², R³, R⁵ and R⁶ are as defined above, with a suitable halide,
e) for compounds of formula (I) wherein R², R³, R⁴, R⁵ and R⁶ are as defined above and R¹ is —CH₂NR²⁵R²⁶, reaction of a compound of formula (XXV)

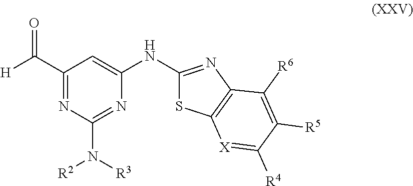

wherein R², R³, R⁴, R⁵ and R⁶ are as defined above, with an amine of formula (III) as defined above followed by treatment with sodium triacetoxyborohydride,
f) for compounds of formula (I) wherein R², R³, R⁴, R⁵ and R⁶ are as defined above and R¹ is —CH₂NR²⁵R²⁶ wherein R²⁵ and R²⁶, together with the nitrogen atom to which they are attached, are linked to form 2-piperazinone, reaction of a compound of formula (XXVI)

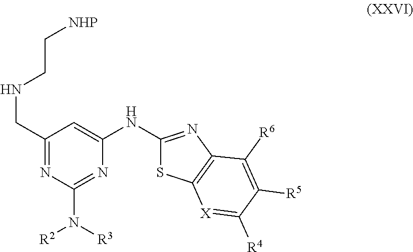

wherein R², R³, R⁴, R⁵ and R⁶ are as defined above and P is a protecting group such as Boc (t-butoxycarbonyl), with chloroacetyl chloride, or
g) for compounds of formula (I) wherein R², R³, R⁴, R⁵ and R⁶ are as defined above and R¹ is 6-methyl-3(2H)-pyridazinone, reaction of a compound of formula (XXV) as defined above, with 6-methyl-4,5-dihydro-3(2H)-pyridazinone.

### Methods of Use

The compounds of the invention are inhibitors of kinase activity, in particular Itk activity. Compounds which are Itk inhibitors may be useful in the treatment of disorders wherein the underlying pathology is (at least in part) attributable to inappropriate Itk activity, such as asthma. “Inappropriate Itk activity” refers to any Itk activity that deviates from the normal Itk activity expected in a particular patient. Inappropriate Itk may take the form of, for instance, an abnormal increase in activity, or an aberration in the timing and or control of Itk activity. Such inappropriate activity may result then, for example, from overexpression or mutation of the protein kinase leading to inappropriate or uncontrolled activation. Accordingly, in another aspect the invention is directed to methods of treating such disorders.

Such disorders include respiratory diseases including asthma, chronic obstructive pulmonary disease (COPD) and bronchitis; allergic diseases including allergic rhinitis and atopic dermatitis; autoimmune diseases including rheumatoid arthritis, multiple sclerosis, psoriasis, type I diabetes, T cell mediated hypersensitivities, Guillain-Barre Syndrome and Hashimoto's thyroiditis; transplant rejection; graft versus host disease; inflammatory disorders including conjunctivitis, contact dermatitis, inflammatory bowel disease and chronic inflammation; HIV; aplastic anemia; and pain including inflammatory pain.

The methods of treatment of the invention comprise administering a safe and effective amount of a compound of formula (I) or a pharmaceutically acceptable salt thereof to a patient in need thereof. Individual embodiments of the invention include methods of treating any one of the above-mentioned disorders by administering a safe and effective amount of a compound of formula (I) or a pharmaceutically acceptable salt thereof to a patient in need thereof.

As used herein, “treat” in reference to a disorder means: (1) to ameliorate or prevent the disorder or one or more of the biological manifestations of the disorder, (2) to interfere with (a) one or more points in the biological cascade that leads to or is responsible for the disorder or (b) one or more of the biological manifestations of the disorder, (3) to alleviate one or more of the symptoms or effects associated with the disorder, or (4) to slow the progression of the disorder or one or more of the biological manifestations of the disorder.

As indicated above, “treatment” of a disorder includes prevention of the disorder. The skilled artisan will appreciate that “prevention” is not an absolute term. In medicine, “prevention” is understood to refer to the prophylactic administration of a drug to substantially diminish the likelihood or severity of a disorder or biological manifestation thereof, or to delay the onset of such disorder or biological manifestation thereof.

As used herein, “safe and effective amount” in reference to a compound of formula (I) or a pharmaceutically acceptable salt thereof or other pharmaceutically-active agent means an amount of the compound sufficient to treat the patient's condition but low enough to avoid serious side effects (at a reasonable benefit/risk ratio) within the scope of sound medical judgment. A safe and effective amount of a compound will vary with the particular compound chosen (e.g. consider the potency, efficacy, and half-life of the compound); the route of administration chosen; the disorder being treated; the severity of the disorder being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human (including adults and children) or other animal. In one embodiment, “patient” refers to a human.

The compounds of formula (I) or pharmaceutically acceptable salts thereof may be administered by any suitable route of administration, including both systemic administration and topical administration. Systemic administration includes oral administration, parenteral administration, transdermal administration and rectal administration. Parenteral administration refers to routes of administration other than enteral or transdermal, and is typically by injection or infusion. Parenteral administration includes intravenous, intramuscular, and subcutaneous injection or infusion. Topical administration includes application to the skin as well as intraocular, otic, intravaginal, inhaled and intranasal administration. Inhalation refers to administration into the patient's lungs whether inhaled through the mouth or through the nasal passages. In one embodiment, the compounds of formula (I) or pharmaceutically acceptable salts thereof may be administered orally. In another embodiment, the compounds of formula (I) or pharmaceutically acceptable salts thereof may be administered topically. In another embodiment, the compounds of formula (I) or pharmaceutically acceptable salts thereof may be administered by inhalation. In a further embodiment, the compounds of formula (I) or pharmaceutically acceptable salts thereof may be administered intranasally.

The compounds of formula (I) or pharmaceutically acceptable salts thereof may be administered once or according to a dosing regimen wherein a number of doses are administered at varying intervals of time for a given period of time. For example, doses may be administered one, two, three, or four times per day. In one embodiment, a dose is administered once per day. In a further embodiment, a dose is administered twice per day. Doses may be administered until the desired therapeutic effect is achieved or indefinitely to maintain the desired therapeutic effect. Suitable dosing regimens for a compound of formula (I) or a pharmaceutically acceptable salt thereof depend on the pharmacokinetic properties of that compound, such as absorption, distribution, and half-life, which can be determined by the skilled artisan. In addition, suitable dosing regimens, including the duration such regimens are administered, for a compound of formula (I) or a pharmaceutically acceptable salt thereof depend on the disorder being treated, the severity of the disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages may vary depending upon the particular route of administration chosen. Typical daily dosages for oral administration range from 0.001 mg to 50 mg per kg of total body weight, for example from 1 mg to 10 mg per kg of total body weight. For example, daily dosages for oral administration may be from 0.5 mg to 2 g per patient, such as 10 mg to 1 g per patient.

Additionally, the compounds of formula (I) may be administered as prodrugs. As used herein, a “prodrug” of a compound of formula (I) is a functional derivative of the compound which, upon administration to a patient, eventually liberates the compound of formula (I) in vivo. Administration of a compound of formula (I) as a prodrug may enable the skilled artisan to do one or more of the following: (a) modify the onset of the activity of the compound in vivo; (b) modify the duration of action of the compound in vivo; (c) modify the transportation or distribution of the compound in vivo; (d) modify the solubility of the compound in vivo; and (e) overcome a side effect or other difficulty encountered with the compound. Typical functional derivatives used to prepare prodrugs include modifications of the compound that are chemically or enzymatically cleavable in vivo. Such modifications, which include the preparation of phosphates, amides, esters, thioesters, carbonates, and carbamates, are well known to those skilled in the art.

The invention thus provides a method of treating a disorder mediated by inappropriate Itk activity comprising administering a safe and effective amount of a compound of formula (I) or a pharmaceutically acceptable salt thereof to a patient in need thereof.

In one embodiment, the disorder mediated by inappropriate Itk activity is selected from the group consisting of respiratory diseases (including asthma, chronic obstructive pulmonary disease (COPD) and bronchitis); allergic diseases (including allergic rhinitis and atopic dermatitis); autoimmune diseases (including rheumatoid arthritis, multiple sclerosis, psoriasis, type I diabetes, T cell mediated hypersensitivities, Guillain-Barre Syndrome and Hashimoto's thyroiditis); transplant rejection; graft versus host disease; inflammatory disorders (including conjunctivitis, contact dermatitis, inflammatory bowel disease and chronic inflammation); HIV; aplastic anemia; and pain including inflammatory pain.

In one embodiment, the disorder mediated by inappropriate Itk activity is a respiratory disease. In a further embodiment, the disorder mediated by inappropriate Itk activity is asthma.

In one embodiment, the invention provides a compound of formula (I) or a pharmaceutically acceptable salt thereof for use in medical therapy. In another embodiment, the invention provides a compound of formula (I) or a pharmaceutically acceptable salt thereof for use in the treatment of a disorder mediated by inappropriate Itk activity. In another embodiment, the invention provides the use of a compound of formula (I) or a pharmaceutically acceptable salt thereof in the manufacture of a medicament for use in the treatment of a disorder mediated by inappropriate Itk activity. In a further embodiment, the invention provides a pharmaceutical composition for the treatment or prophylaxis of a disorder mediated by inappropriate Itk activity comprising a compound of formula (I) or a pharmaceutically acceptable salt thereof.

### Compositions

The compounds of formula (I) and pharmaceutically acceptable salts thereof will normally, but not necessarily, be formulated into pharmaceutical compositions prior to administration to a patient. Accordingly, in another aspect the invention is directed to pharmaceutical compositions comprising a compound of formula (I) or a pharmaceutically acceptable salt thereof and one or more pharmaceutically acceptable excipients. In a further aspect the invention is directed to pharmaceutical compositions for the treatment or prophylaxis of a disorder mediated by inappropriate Itk activity comprising a compound of formula (I) or a pharmaceutically acceptable salt thereof.

The pharmaceutical compositions of the invention may be prepared and packaged in bulk form wherein a safe and effective amount of a compound of formula (I) or a pharmaceutically acceptable salt thereof can be extracted and then given to the patient such as with powders or syrups. Alternatively, the pharmaceutical compositions of the invention may be prepared and packaged in unit dosage form wherein each physically discrete unit contains a compound of formula (I) or a pharmaceutically acceptable salt thereof. When prepared in unit dosage form, the pharmaceutical compositions of the invention typically may contain, for example, from 0.5 mg to 1 g, or from 1 mg to 700 mg, or from 5 mg to 100 mg of a compound of formula (I) or a pharmaceutically acceptable salt thereof.

The pharmaceutical compositions of the invention typically contain one compound of formula (I) or a pharmaceutically acceptable salt thereof.

As used herein, “pharmaceutically acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of formula (I) or a pharmaceutically acceptable salt thereof when administered to a patient and interactions which would result in pharmaceutical compositions that are not pharmaceutically acceptable are avoided. In addition, each excipient must of course be pharmaceutically acceptable eg of sufficiently high purity.

The compound of formula (I) or a pharmaceutically acceptable salt thereof and the pharmaceutically acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols, solutions, and dry powders; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of formula (I) or pharmaceutically acceptable salts thereof once administered to the patient from one organ, or portion of the body, to another organ, or portion of the body. Certain pharmaceutically acceptable excipients may be chosen for their ability to enhance patient compliance.

Suitable pharmaceutically-acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other excipients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

Accordingly, in another aspect the invention is directed to process for the preparation of a pharmaceutical composition comprising a compound of formula (I) or a pharmaceutically acceptable salt thereof and one or more pharmaceutically-acceptable excipients which comprises mixing the ingredients. A pharmaceutical composition comprising a compound of formula (I) or a pharmaceutically acceptable salt thereof may be prepared by, for example, admixture at ambient temperature and atmospheric pressure.

In one embodiment, the compounds of formula (I) or pharmaceutically acceptable salts thereof will be formulated for oral administration. In another embodiment, the compounds of formula (I) or pharmaceutically acceptable salts thereof will be formulated for inhaled administration. In a further embodiment, the compounds of formula (I) or pharmaceutically acceptable salts thereof will be formulated for intranasal administration.

In one aspect, the invention is directed to a solid oral dosage form such as a tablet or capsule comprising a safe and effective amount of a compound of formula (I) or a pharmaceutically acceptable salt thereof and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

Where appropriate, dosage unit formulations for oral administration can be microencapsulated. The composition can also be prepared to prolong or sustain the release as for example by coating or embedding particulate material in polymers, wax or the like.

The compounds of formula (I) or pharmaceutically acceptable salts thereof may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds of formula (I) or pharmaceutically acceptable salts thereof may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

In another aspect, the invention is directed to a liquid oral dosage form. Oral liquids such as solution, syrups and elixirs can be prepared in dosage unit form so that a given quantity contains a predetermined amount of a compound of formula (I) or a pharmaceutically acceptable salt thereof. Syrups can be prepared by dissolving the compound of formula (I) or a pharmaceutically acceptable salt thereof in a suitably flavored aqueous solution, while elixirs are prepared through the use of a non-toxic alcoholic vehicle. Suspensions can be formulated by dispersing the compound of formula (I) or a pharmaceutically acceptable salt thereof in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

In another aspect, the invention is directed to a dosage form adapted for administration to a patient by inhalation. For example, as a dry powder, an aerosol, a suspension, or a solution composition.

Dry powder compositions for delivery to the lung by inhalation typically comprise a compound of formula (I) or a pharmaceutically acceptable salt thereof as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides. The finely divided powder may be prepared by, for example, micronisation and milling. Generally, the size-reduced (eg micronised) compound can be defined by a D₅₀ value of about 1 to about 10 microns (for example as measured using laser diffraction).

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Alternatively, the dry powder may be presented in capsules (e.g. gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 20 μg-10 mg of the compound of formula (I) or a pharmaceutically acceptable salt thereof.

Aerosols may be formed by suspending or dissolving a compound of formula (I) or a pharmaceutically acceptable salt thereof in a liquified propellant. Suitable propellants include halocarbons, hydrocarbons, and other liquified gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of formula (I) or a pharmaceutically acceptable salt thereof will typically be administered to a patient via a metered dose inhaler (MDI). Such devices are known to those skilled in the art.

The aerosol may contain additional pharmaceutically-acceptable excipients typically used with MDIs such as surfactants, lubricants, cosolvents and other excipients to improve the physical stability of the formulation, to improve valve performance, to improve solubility, or to improve taste.

There is thus provided as a further aspect of the invention a pharmaceutical aerosol formulation comprising a compound of formula (I) or a pharmaceutically acceptable salt thereof and a fluorocarbon or hydrogen-containing chlorofluorocarbon as propellant, optionally in combination with a surfactant and/or a cosolvent.

According to another aspect of the invention, there is provided a pharmaceutical aerosol formulation wherein the propellant is selected from 1,1,1,2-tetrafluoroethane, 1,1,1,2,3,3,3-heptafluoro-n-propane and mixtures thereof.

The formulations of the invention may be buffered by the addition of suitable buffering agents.

Capsules and cartridges for use in an inhaler or insufflator, of for example gelatine, may be formulated containing a powder mix for inhalation of a compound of formula (I) or a pharmaceutically acceptable salt thereof and a suitable powder base such as lactose or starch. Each capsule or cartridge may generally contain from 20 μg to 10 mg of the compound of formula (I) or pharmaceutically acceptable salt thereof.

Alternatively, the compound of formula (I) or pharmaceutically acceptable salt thereof may be presented without excipients such as lactose.

The proportion of the active compound of formula (I) or pharmaceutically acceptable salt thereof in the local compositions according to the invention depends on the precise type of formulation to be prepared but will generally be within the range of from 0.001 to 10% by weight. Generally, for most types of preparations, the proportion used will be within the range of from 0.005 to 1%, for example from 0.01 to 0.5%. However, in powders for inhalation or insufflation the proportion used will normally be within the range of from 0.1 to 5%.

Aerosol formulations are preferably arranged so that each metered dose or “puff” of aerosol contains from 20 μg to 10 mg, preferably from 20 μg to 2000 μg, more preferably from about 20 μg to 500 μg of a compound of formula (I). Administration may be once daily or several times daily, for example 2, 3, 4 or 8 times, giving for example 1, 2 or 3 doses each time. The overall daily dose with an aerosol will be within the range from 100 μg to 10 mg, preferably from 200 μg to 2000 μg. The overall daily dose and the metered dose delivered by capsules and cartridges in an inhaler or insufflator will generally be double that delivered with aerosol formulations.

In the case of suspension aerosol formulations, the particle size of the particulate (e.g., micronised) drug should be such as to permit inhalation of substantially all the drug into the lungs upon administration of the aerosol formulation and will thus be less than 100 microns, desirably less than 20 microns, and in particular in the range of from 1 to 10 microns, such as from 1 to 5 microns, more preferably from 2 to 3 microns.

The formulations of the invention may be prepared by dispersal or dissolution of the medicament and a compound of formula (I) or a pharmaceutically acceptable salt thereof in the selected propellant in an appropriate container, for example, with the aid of sonication or a high-shear mixer. The process is desirably carried out under controlled humidity conditions.

The chemical and physical stability and the pharmaceutical acceptability of the aerosol formulations according to the invention may be determined by techniques well known to those skilled in the art. Thus, for example, the chemical stability of the components may be determined by HPLC assay, for example, after prolonged storage of the product. Physical stability data may be gained from other conventional analytical techniques such as, for example, by leak testing, by valve delivery assay (average shot weights per actuation), by dose reproducibility assay (active ingredient per actuation) and spray distribution analysis.

The stability of the suspension aerosol formulations according to the invention may be measured by conventional techniques, for example, by measuring flocculation size distribution using a back light scattering instrument or by measuring particle size distribution by cascade impaction or by the “twin impinger” analytical process. As used herein reference to the “twin impinger” assay means “Determination of the deposition of the emitted dose in pressurised inhalations using apparatus A” as defined in British Pharmacopaeia 1988, pages A204-207, Appendix XVII C. Such techniques enable the “respirable fraction” of the aerosol formulations to be calculated. One method used to calculate the “respirable fraction” is by reference to “fine particle fraction” which is the amount of active ingredient collected in the lower impingement chamber per actuation expressed as a percentage of the total amount of active ingredient delivered per actuation using the twin impinger method described above.

The term “metered dose inhaler” or MDI means a unit comprising a can, a secured cap covering the can and a formulation metering valve situated in the cap. MDI system includes a suitable channelling device. Suitable channelling devices comprise for example, a valve actuator and a cylindrical or cone-like passage through which medicament may be delivered from the filled canister via the metering valve to the nose or mouth of a patient such as a mouthpiece actuator.

MDI canisters generally comprise a container capable of withstanding the vapour pressure of the propellant used such as a plastic or plastic-coated glass bottle or preferably a metal can, for example, aluminium or an alloy thereof which may optionally be anodised, lacquer-coated and/or plastic-coated (for example incorporated herein by reference WO96/32099 wherein part or all of the internal surfaces are coated with one or more fluorocarbon polymers optionally in combination with one or more non-fluorocarbon polymers), which container is closed with a metering valve. The cap may be secured onto the can via ultrasonic welding, screw fitting or crimping. MDIs taught herein may be prepared by methods of the art (e.g. see Byron, above and WO96/32099). Preferably the canister is fitted with a cap assembly, wherein a drug-metering valve is situated in the cap, and said cap is crimped in place.

In one embodiment of the invention the metallic internal surface of the can is coated with a fluoropolymer, more preferably blended with a non-fluoropolymer. In another embodiment of the invention the metallic internal surface of the can is coated with a polymer blend of polytetrafluoroethylene (PTFE) and polyethersulfone (PES). In a further embodiment of the invention the whole of the metallic internal surface of the can is coated with a polymer blend of polytetrafluoroethylene (PTFE) and polyethersulfone (PES).

The metering valves are designed to deliver a metered amount of the formulation per actuation and incorporate a gasket to prevent leakage of propellant through the valve. The gasket may comprise any suitable elastomeric material such as, for example, low density polyethylene, chlorobutyl, bromobutyl, EPDM, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak plc, UK (e.g. BK300, BK357) and 3M-Neotechnic Ltd, UK (e.g. Spraymiser™).

In various embodiments, the MDIs may also be used in conjunction with other structures such as, without limitation, overwrap packages for storing and containing the MDIs, including those described in U.S. Pat. Nos. 6,119,853; 6,179,118; 6,315,112; 6,352,152; 6,390,291; and 6,679,374, as well as dose counter units such as, but not limited to, those described in U.S. Pat. Nos. 6,360,739 and 6,431,168.

Conventional bulk manufacturing methods and machinery well known to those skilled in the art of pharmaceutical aerosol manufacture may be employed for the preparation of large-scale batches for the commercial production of filled canisters. Thus, for example, in one bulk manufacturing method for preparing suspension aerosol formulations a metering valve is crimped onto an aluminium can to form an empty canister. The particulate medicament is added to a charge vessel and liquefied propellant together with the optional excipients is pressure filled through the charge vessel into a manufacturing vessel. The drug suspension is mixed before recirculation to a filling machine and an aliquot of the drug suspension is then filled through the metering valve into the canister. In one example bulk manufacturing method for preparing solution aerosol formulations a metering valve is crimped onto an aluminium can to form an empty canister. The liquefied propellant together with the optional excipients and the dissolved medicament is pressure filled through the charge vessel into a manufacturing vessel.

In an alternative process, an aliquot of the liquefied formulation is added to an open canister under conditions which are sufficiently cold to ensure the formulation does not vaporise, and then a metering valve crimped onto the canister.

Typically, in batches prepared for pharmaceutical use, each filled canister is check-weighed, coded with a batch number and packed into a tray for storage before release testing.

Suspensions and solutions comprising a compound of formula (I) or a pharmaceutically acceptable salt thereof may also be administered to a patient via a nebulizer. The solvent or suspension agent utilized for nebulization may be any pharmaceutically-acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically-acceptable excipients may be added to the suspension or solution. The compound of formula (I) or pharmaceutically acceptable salt thereof may be stabilized by the addition of an inorganic acid, e.g., hydrochloric acid, nitric acid, sulphuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize the compound of formula (I) or pharmaceutically acceptable salt thereof. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

In a further aspect, the invention is directed to a dosage form adapted for intranasal administration.

Formulations for administration to the nose may include pressurised aerosol formulations and aqueous formulations administered to the nose by pressurised pump. Formulations which are non-pressurised and adapted to be administered topically to the nasal cavity are of particular interest. Suitable formulations contain water as the diluent or carrier for this purpose. Aqueous formulations for administration to the lung or nose may be provided with conventional excipients such as buffering agents, tonicity modifying agents and the like. Aqueous formulations may also be administered to the nose by nebulisation.

The compounds of formula (I) or pharmaceutically acceptable salts thereof may be formulated as a fluid formulation for delivery from a fluid dispenser, for example a fluid dispenser having a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO05/044354, the entire content of which is hereby incorporated herein by reference. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. In one embodiment, the fluid dispenser is of the general type illustrated in FIGS. 30-40 of WO05/044354.

Pharmaceutical compositions adapted for intranasal administration wherein the carrier is a solid include a coarse powder having a particle size for example in the range 20 to 500 microns which is administered by rapid inhalation through the nasal passage from a container of the powder held close up to the nose. Suitable compositions wherein the carrier is a liquid, for administration as a nasal spray or as nasal drops, include aqueous or oil solutions of the compound of formula (I) or a pharmaceutically acceptable salt thereof.

Pharmaceutical compositions adapted for transdermal administration may be presented as discrete patches intended to remain in intimate contact with the epidermis of the patient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils.

Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents, suspending agents or preservatives.

Topical preparations may be administered by one or more applications per day to the affected area; over skin areas occlusive dressings may advantageously be used. Continuous or prolonged delivery may be achieved by an adhesive reservoir system.

For treatments of the eye or other external tissues, for example mouth and skin, the compositions may be applied as a topical ointment or cream. When formulated in an ointment, the compound of formula (I) or a pharmaceutically acceptable salt thereof may be employed with either a paraffinic or a water-miscible ointment base. Alternatively, the compound of formula (I) or pharmaceutically acceptable salt thereof may be formulated in a cream with an oil-in-water cream base or a water-in-oil base.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The compound and pharmaceutical formulations according to the invention may be used in combination with or include one or more other therapeutic agents, for example selected from anti-inflammatory agents, anticholinergic agents (particularly an M₁/M₂/M₃ receptor antagonist), β₂-adrenoreceptor agonists, antiinfective agents, such as antibiotics or antivirals, or antihistamines. The invention thus provides, in a further aspect, a combination comprising a compound of formula (I) or a pharmaceutically acceptable salt thereof together with one or more other therapeutically active agents, for example selected from an anti-inflammatory agent, such as a corticosteroid or an NSAID, an anticholinergic agent, a β₂β-adrenoreceptor agonist, an antiinfective agent, such as an antibiotic or an antiviral, or an antihistamine. One embodiment of the invention encompasses combinations comprising a compound of formula (I) or a pharmaceutically acceptable salt thereof together with a β₂β-adrenoreceptor agonist, and/or an anticholinergic, and/or a PDE-4 inhibitor, and/or an antihistamine.

One embodiment of the invention encompasses combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimise the activity and/or stability and/or physical characteristics, such as solubility, of the therapeutic ingredient. It will be clear also that, where appropriate, the therapeutic ingredients may be used in optically pure form.

In one embodiment, the invention encompasses a combination comprising a compound of formula (I) or a pharmaceutically acceptable salt thereof together with a β₂β-adrenoreceptor agonist.

Examples of β₂β-adrenoreceptor agonists include salmeterol (which may be a racemate or a single enantiomer such as the R-enantiomer), salbutamol (which may be a racemate or a single enantiomer such as the R-enantiomer), formoterol (which may be a racemate or a single duastereomer such as the R,R-diastereomer), salmefamol, fenoterol carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt or free base of salbutamol or the fumarate salt of formoterol. In one embodiment, long-acting β₂β-adrenoreceptor agonists, for example, compounds which provide effective bronchodilation for about 12 hrs or longer, are preferred.

Other β₂β-adrenoreceptor agonists include those described in WO 02/066422, WO 02/070490, WO 02/076933, WO 03/024439, WO 03/072539, WO 03/091204, WO 04/016578, WO 2004/022547, WO 2004/037807, WO 2004/037773, WO 2004/037768, WO 2004/039762, WO 2004/039766, WO01/42193 and WO03/042160.

Examples of β₂β-adrenoreceptor agonists include:
3-(4-{[6-({(2R)-2-hydroxy-2-[4-hydroxy-3-(hydroxymethyl)phenyl]ethyl}amino) hexyl]oxy}butyl)benzenesulfonamide;
3-(3-{[7-({(2R)-2-hydroxy-2-[4-hydroxy-3-hydroxymethyl)phenyl]ethyl}-amino) heptyl]oxy}propyl)benzenesulfonamide;
4-{(1R)-2-[(6-{2-[(2,6-dichlorobenzyl)oxy]ethoxy}hexyl)amino]-1-hydroxyethyl}-2-(hydroxymethyl)phenol;
4-{(1R)-2-[(6-{4-[3-(cyclopentylsulfonyl)phenyl]butoxy}hexyl)amino]-1-hydroxyethyl}-2-(hydroxymethyl)phenol;
N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;
N-2{2-[4-(3-phenyl-4-methoxyphenyl)aminophenyl]ethyl}-2-hydroxy-2-(8-hydroxy-2(1H)-quinolinon-5-yl)ethylamine; and
5-[(R)-2-(2-{4-[4-(2-amino-2-methyl-propoxy)-phenylamino]-phenyl}-ethylamino)-1-hydroxy-ethyl]-8-hydroxy-1H-quinolin-2-one.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

Suitable anti-inflammatory agents include corticosteroids. Suitable corticosteroids which may be used in combination with the compounds of formula (I) or pharmaceutically acceptable salts thereof are those oral and inhaled corticosteroids and their pro-drugs which have anti-inflammatory activity. Examples include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), 6α,9α-difluoro-1,0-hydroxy-16α-methyl-3-oxo-17α-propionyloxy-androsta-1,4-diene-17β-carbothioic acid S-(2-oxo-tetrahydro-furan-3S-yl) ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (for example the 17-propionate ester or the 17,21-dipropionate ester), budesonide, flunisolide, mometasone esters (for example mometasone furoate), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. Preferred corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester. In one embodiment the corticosteroid is 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Examples of corticosteroids may include those described in WO2002/088167, WO2002/100879, WO2002/12265, WO2002/12266, WO2005/005451, WO2005/005452, WO2006/072599 and WO2006/072600.

Non-steroidal compounds having glucocorticoid agonism that may possess selectivity for transrepression over transactivation and that may be useful in combination therapy include those covered in the following patents: WO03/082827, WO98/54159, WO04/005229, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651 and WO03/08277. Further non-steroidal compounds are covered in: WO2006/000401, WO2006/000398 and WO2006/015870.

Examples of anti-inflammatory agents include non-steroidal anti-inflammatory drugs (NSAID's).

Examples of NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example montelukast), iNOS inhibitors, tryptase and elastase inhibitors, beta-2 integrin antagonists and adenosine receptor agonists or antagonists (e.g. adenosine 2a agonists), cytokine antagonists (for example chemokine antagonists, such as a CCR3 antagonist) or inhibitors of cytokine synthesis, or 5-lipoxygenase inhibitors. An iNOS (inducible nitric oxide synthase inhibitor) is preferably for oral administration. Examples of iNOS inhibitors include those disclosed in WO93/13055, WO98/30537, WO02/50021, WO95/34534 and WO99/62875. Examples of CCR3 inhibitors include those disclosed in WO02/26722.

In one embodiment, the invention provides the use of the compounds of formula (I) in combination with a phosphodiesterase 4 (PDE4) inhibitor, especially in the case of a formulation adapted for inhalation. The PDE4-specific inhibitor useful in this aspect of the invention may be any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

Compounds include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep., 1996; this patent and the compounds it discloses are incorporated herein in full by reference.

Other compounds include AWD-12-281 from Elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

Further compounds are disclosed in the published international patent application WO04/024728 (Glaxo Group Ltd), WO04/056823 (Glaxo Group Ltd) and WO04/103998 (Glaxo Group Ltd) (e.g. Example 399 or 544 disclosed therein). Further compounds are also disclosed in WO2005/058892, WO2005/090348, WO2005/090353, and WO2005/090354, all in the name of Glaxo Group Limited.

Examples of anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO01/04118. Exemplary compounds for oral administration include pirenzepine (CAS 28797-61-7), darifenacin (CAS 133099-04-4, or CAS 133099-07-7 for the hydrobromide sold under the name Enablex), oxybutynin (CAS 5633-20-5, sold under the name Ditropan), terodiline (CAS 15793-40-5), tolterodine (CAS 124937-51-5, or CAS 124937-52-6 for the tartrate, sold under the name Detrol), otilonium (for example, as the bromide, CAS 26095-59-0, sold under the name Spasmomen), trospium chloride (CAS 10405-02-4) and solifenacin (CAS 242478-37-1, or CAS 242478-38-2 for the succinate also known as YM-905 and sold under the name Vesicare).

Additional compounds are disclosed in WO 2005/037280, WO 2005/046586 and WO 2005/104745, incorporated herein by reference. The present combinations include, but are not limited to:
(3-endo)-3-(2,2-di-2-thienylethenyl)-8,8-dimethyl-8-azoniabicyclo[3.2.1]octane iodide;
(3-endo)-3-(2-cyano-2,2-diphenylethyl)-8,8-dimethyl-8-azoniabicyclo[3.2.1]octane bromide;
4-[hydroxy(diphenyl)methyl]-1-{2-[(phenylmethyl)oxy]ethyl}-1-azoniabicyclo[2.2.2]octane bromide; and
(1R,5S)-3-(2-cyano-2,2-diphenylethyl)-8-methyl-8-{2-[(phenylmethyl)oxy]ethyl}-8-azoniabicyclo[3.2.1]octane bromide.

Other anticholinergic agents include compounds which are disclosed in U.S. patent application 60/487,981 including, for example:
(3-endo)-3-(2,2-di-2-thienylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane 4-methylbenzenesulfonate;
(3-endo)-8,8-dimethyl-3-[2-phenyl-2-(2-thienyl)ethenyl]-8-azoniabicyclo[3.2.1]octane bromide; and/or
(3-endo)-8,8-dimethyl-3-[2-phenyl-2-(2-pyridinyl)ethenyl]-8-azoniabicyclo[3.2.1]octane bromide.

Further anticholinergic agents include compounds which are disclosed in U.S. patent application 60/511,009 including, for example:
(endo)-3-(2-methoxy-2,2-di-thiophen-2-yl-ethyl)-8,8-dimethyl-8-azonia-bicyclo[3.2.1]octane iodide;
3-((endo)-8-methyl-8-aza-bicyclo[3.2.1]oct-3-yl)-2,2-diphenyl-propionitrile;
(endo)-8-methyl-3-(2,2,2-triphenyl-ethyl)-8-aza-bicyclo[3.2.1]octane;
3-((endo)-8-methyl-8-aza-bicyclo[3.2.1]oct-3-yl)-2,2-diphenyl-propionamide;
3-((endo)-8-methyl-8-aza-bicyclo[3.2.1]oct-3-yl)-2,2-diphenyl-propionic acid;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
3-((endo)-8-methyl-8-aza-bicyclo[3.2.1]oct-3-yl)-2,2-diphenyl-propan-1-ol;
N-benzyl-3-((endo)-8-methyl-8-aza-bicyclo[3.2.1]oct-3-yl)-2,2-diphenyl-propionamide;
(endo)-3-(2-carbamoyl-2,2-diphenyl-ethyl)-8,8-dimethyl-8-azonia-bicyclo[3.2.1]octane iodide;
1-benzyl-3-[3-((endo)-8-methyl-8-aza-bicyclo[3.2.1]oct-3-yl)-2,2-diphenyl-propyl]-urea;
1-ethyl-3-[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-acetamide;
N-[3-((endo)-8-methyl-8-aza-bicyclo[3.2.1]oct-3-yl)-2,2-diphenyl-propyl]-benzamide;
3-((endo)-8-methyl-8-aza-bicyclo[3.2.1]oct-3-yl)-2,2-di-thiophen-2-yl-propionitrile;
(endo)-3-(2-cyano-2,2-di-thiophen-2-yl-ethyl)-8,8-dimethyl-8-azonia-bicyclo[3.2.1]octane iodide;
N-[3-((endo)-8-methyl-8-aza-bicyclo[3.2.1]oct-3-yl)-2,2-diphenyl-propyl]-benzenesulfonamide;
[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-methanesulfonamide; and/or
(endo)-3-{2,2-diphenyl-3-[(1-phenyl-methanoyl)-amino]-propyl}-8,8-dimethyl-8-azonia-bicyclo[3.2.1]octane bromide.

Further compounds include:
(endo)-3-(2-methoxy-2,2-di-thiophen-2-yl-ethyl)-8,8-dimethyl-8-azonia-bicyclo[3.2.1]octane iodide;

(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
(endo)-3-(2-carbamoyl-2,2-diphenyl-ethyl)-8,8-dimethyl-8-azonia-bicyclo[3.2.1]octane iodide;
(endo)-3-(2-cyano-2,2-di-thiophen-2-yl-ethyl)-8,8-dimethyl-8-azonia-bicyclo[3.2.1]octane iodide; and/or
(endo)-3-{(2,2-diphenyl-3-[(1-phenyl-methanoyl)-amino]-propyl}-8,8-dimethyl-8-azonia-bicyclo[3.2.1]octane bromide.

In one embodiment the invention provides a combination comprising a compound of formula (I) or a pharmaceutically acceptable salt thereof together with an H1 antagonist. Examples of H1 antagonists include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. In a further embodiment the invention provides a combination comprising a compound of formula (I) or a pharmaceutically acceptable salt thereof together with an H3 antagonist (and/or inverse agonist). Examples of H3 antagonists include, for example, those compounds disclosed in WO2004/035556 and in WO2006/045416. Other histamine receptor antagonists which may be used in combination with the compounds of the present invention include antagonists (and/or inverse agonists) of the H4 receptor, for example, the compounds disclosed in Jablonowski et al., J. Med. Chem. 46:3957-3960 (2003).

The invention thus provides, in a further aspect, a combination comprising a compound of formula (I) or a pharmaceutically acceptable salt thereof together with a PDE4 inhibitor.

The invention thus provides, in a further aspect, a combination comprising a compound of formula (I) or a pharmaceutically acceptable salt thereof together with a β₂-adrenoreceptor agonist.

The invention thus provides, in a further aspect, a combination comprising a compound of formula (I) or a pharmaceutically acceptable salt thereof together with a corticosteroid.

The invention thus provides, in a further aspect, a combination comprising a compound of formula (I) or a pharmaceutically acceptable salt thereof together with a non-steroidal GR agonist.

The invention thus provides, in a further aspect, a combination comprising a compound of formula (I) or a pharmaceutically acceptable salt thereof together with an anticholinergic.

The invention thus provides, in a further aspect, a combination comprising a compound of formula (I) or a pharmaceutically acceptable salt thereof together with an antihistamine.

The invention thus provides, in a further aspect, a combination comprising a compound of formula (I) or a pharmaceutically acceptable salt thereof together with a PDE4 inhibitor and a β₂-adrenoreceptor agonist.

The invention thus provides, in a further aspect, a combination comprising a compound of formula (I) or a pharmaceutically acceptable salt thereof together with an anticholinergic and a PDE-4 inhibitor.

The combinations referred to above may conveniently be presented for use in the form of a pharmaceutical composition and thus pharmaceutical compositions comprising a combination as defined above together with a pharmaceutically acceptable diluent or carrier represent a further aspect of the invention.

The individual compounds of such combinations may be administered either sequentially or simultaneously in separate or combined pharmaceutical formulations. In one embodiment, the individual compounds will be administered simultaneously in a combined pharmaceutical formulation. Appropriate doses of known therapeutic agents will readily be appreciated by those skilled in the art.

The invention thus provides, in a further aspect, a pharmaceutical composition comprising a combination of a compound of formula (I) or a pharmaceutically acceptable salt thereof together with another therapeutically active agent.

The invention thus provides, in a further aspect, a pharmaceutical composition comprising a combination of a compound of formula (I) or a pharmaceutically acceptable salt thereof together with a PDE4 inhibitor.

The invention thus provides, in a further aspect, a pharmaceutical composition comprising a combination of a compound of formula (I) or a pharmaceutically acceptable salt thereof together with a β₂-adrenoreceptor agonist.

The invention thus provides, in a further aspect, a pharmaceutical composition comprising a combination of a compound of formula (I) or a pharmaceutically acceptable salt thereof together with a corticosteroid.

The invention thus provides, in a further aspect, a pharmaceutical composition comprising a combination of a compound of formula (I) or a pharmaceutically acceptable salt thereof together with a non-steroidal GR agonist.

The invention thus provides, in a further aspect, a pharmaceutical composition comprising a combination of a compound of formula (I) or a pharmaceutically acceptable salt thereof together with an anticholinergic.

The invention thus provides, in a further aspect, a pharmaceutical composition comprising a combination of a compound of formula (I) or a pharmaceutically acceptable salt thereof together with an antihistamine.

The invention thus provides, in a further aspect, a pharmaceutical composition comprising a combination of a compound of formula (I) or a pharmaceutically acceptable salt thereof together with a PDE4 inhibitor and a β₂-adrenoreceptor agonist.

The invention thus provides, in a further aspect, a pharmaceutical composition comprising a combination of a compound of formula (I) or a pharmaceutically acceptable salt thereof together with an anticholinergic and a PDE4 inhibitor.

The invention will now be illustrated by way of the following non-limiting examples.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

### General Methods

Unless stated otherwise, starting materials were commercially available. All solvents and commercial reagents were of laboratory grade and were used as received.

In the examples ¹H NMR spectra were recorded on a Bruker DRX 400 (400 MHz) instrument. The following abbreviations have been used: s, singlet; d, doublet; t, triplet; Hz, Hertz

Unless stated otherwise, flash chromatography was carried out using pre-packed Biotage “Isolute” flash silica cartridges on a Biotage “Flashmaster 2” system.

The following methods were used for LCMS (liquid chromatography-mass spectral) analysis:

### LCMS Method A:

The analysis was conducted on an Acquity UPLC BEH C18 column (50 mm×2.1 mm internal diameter 1.7 μm packing diameter) at 40° C.

The solvents employed were:

A=0.1% v/v solution of formic acid in water.
B=0.1% v/v solution of formic acid in acetonitrile.

The gradient employed was as follows:

| Time | Flow Rate | | |
|---|---|---|---|
| (minutes) | (mL/min) | % A | % B |
| 0 | 1 | 97 | 3 |
| 1.5 | 1 | 0 | 100 |
| 1.9 | 1 | 0 | 100 |
| 2.0 | 1 | 97 | 3 |

The UV detection was an averaged signal from wavelength of 210 nm to 350 nm and mass spectra were recorded on a mass spectrometer using alternate-scan positive and negative mode electrospray ionization.

### LCMS Method B:

The analysis was conducted on an XBridge C18 column (50 mm×4.6 mm internal diameter 3.5 μm packing diameter) at 30° C.

The solvents employed were:

A=10 mM ammonium bicarbonate in water adjusted to pH 10 with ammonia solution.
B=acetonitrile.

The typical gradient employed was as follows:

| Time | Flow Rate | | |
|---|---|---|---|
| (minutes) | (mL/min) | % A | % B |
| 0 | 3 | 99 | 1 |
| 0.1 | 3 | 99 | 1 |
| 4.0 | 3 | 3 | 97 |
| 5.0 | 3 | 3 | 97 |

The UV detection was an averaged signal from wavelength of 210 nm to 350 nm and mass spectra were recorded on a mass spectrometer using alternate-scan positive and negative mode electrospray ionization.

The following illustrates the mobile phases and gradients used when compounds underwent purification by mass-directed autopreparative HPLC.

### Mass-Directed Autopreparative HPLC (Formic Acid Modifier)

The HPLC analysis was conducted on a Sunfire C18 column (150 mm×30 mm internal diameter, 5 μm packing diameter) at ambient temperature.

The solvents employed were:

A=0.1% v/v solution of formic acid in water.
B=0.1% v/v solution of formic acid in acetonitrile.

### Mass-Directed Autopreparative HPLC (Trifluoroacetic Acid Modifier)

The HPLC analysis was conducted on a Sunfire C18 column (150 mm×30 mm internal diameter, 5 μm packing diameter) at ambient temperature.

The solvents employed were:

A=0.1% v/v solution of trifluoroacetic acid in water.
B=0.1% v/v solution of trifluoroacetic acid in acetonitrile.

### Mass-Directed Autopreparative HPLC (Ammonium Bicarbonate Modifier)

The HPLC analysis was conducted on an XBridge C18 column (150 mm×30 mm internal diameter, 5 μm packing diameter) at ambient temperature.

The solvents employed were:

A=10 mM ammonium bicarbonate in water adjusted to pH 10 with ammonia solution.
B=acetonitrile.

For each of the mass-directed autopreparative purifications, irrespective of the modifier used, the gradient employed was dependent upon the retention time of the particular compound undergoing purification as recorded in the analytical LCMS, and was as follows:

For compounds with an analytical LCMS retention time below 0.6 minutes (LCMS method A) or below 1.5 minutes (LCMS method B) the following gradient was used:

| Time | Flow Rate | | |
|---|---|---|---|
| (minutes) | (mL/min) | % A | % B |
| 0 | 40 | 99 | 1 |
| 1 | 40 | 99 | 1 |
| 10 | 40 | 70 | 30 |
| 11 | 40 | 1 | 99 |
| 15 | 40 | 1 | 99 |

For compounds with an analytical LCMS retention time between 0.6 and 0.9 minutes (LCMS method A) or between 1.5 and 2.2 minutes (LCMS method B) the following gradient was used:

| Time | Flow Rate | | |
|---|---|---|---|
| (minutes) | (mL/min) | % A | % B |
| 0 | 40 | 85 | 15 |
| 1 | 40 | 85 | 15 |
| 10 | 40 | 45 | 55 |
| 11 | 40 | 1 | 99 |
| 15 | 40 | 1 | 99 |

For compounds with an analytical LCMS retention time between 0.9 and 1.2 minutes (LCMS method A) or between 2.2 and 3.0 minutes (LCMS method B) the following gradient was used:

| Time | Flow Rate | | |
|---|---|---|---|
| (minutes) | (mL/min) | % A | % B |
| 0 | 40 | 70 | 30 |
| 1 | 40 | 70 | 30 |
| 10 | 40 | 15 | 85 |
| 11 | 40 | 1 | 99 |
| 15 | 40 | 1 | 99 |

For compounds with an analytical LCMS retention time between 1.2 and 1.4 minutes (LCMS method A) or between 3.0 and 3.6 minutes (LCMS method B) the following gradient was used:

| Time | Flow Rate | | |
|---|---|---|---|
| (minutes) | (mL/min) | % A | % B |
| 0 | 40 | 50 | 50 |
| 1 | 40 | 50 | 50 |
| 10 | 40 | 1 | 99 |
| 11 | 40 | 1 | 99 |
| 15 | 40 | 1 | 99 |

For compounds with an analytical LCMS retention time greater than 1.4 minutes (LCMS method A) or greater than 3.6 minutes (LCMS method B) the following gradient was used:

| Time | Flow Rate | | |
|---|---|---|---|
| (minutes) | (mL/min) | % A | % B |
| 0 | 40 | 20 | 80 |
| 1 | 40 | 20 | 80 |
| 10 | 40 | 1 | 99 |
| 11 | 40 | 1 | 99 |
| 15 | 40 | 1 | 99 |

The UV detection was an averaged signal from wavelength of 210 nm to 350 nm and mass spectra were recorded on a mass spectrometer using alternate-scan positive and negative mode electrospray ionization.

The chemical names were generated using ACD Name Pro version 6.02 from Advanced Chemistry Development, Inc.

### Intermediate 1

### 6-ethyl-N-[2-fluoro-6-(phenylmethyl)-4-pyrimidinyl]-1,3-benzothiazol-2-amine

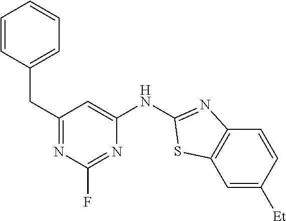

To an ice-cooled solution of 6-ethyl-1,3-benzothiazol-2-amine (130 mg, 0.73 mmol) in tetrahydrofuran (10 mL) under nitrogen was added sodium hydride (58 mg, 60% dispersion in oil, 1.46 mmol) and the mixture was stirred for 15 minutes. A solution of 2,4-difluoro-6-(phenylmethyl)pyrimidine (150 mg, 0.73 mmol) in tetrahydrofuran (1 mL) was added and the mixture allowed to stir and slowly warm to ambient temperature. After 3 hours the mixture was treated with saturated aqueous ammonium chloride (20 mL) and ethyl acetate (20 mL). The organic phase was dried over magnesium sulfate, filtered and evaporated to dryness. Purification by flash chromatography on silica using a gradient elution from 0 to 50% ethyl acetate in cyclohexane afforded the title compound (82 mg, 0.23 mmol, 31% yield) as a white solid. LCMS (Method A): Rt 1.37 minutes; m/z 365 (MH+).

The compounds shown in the table were prepared in an analogous manner to that for 6-ethyl-N-[2-fluoro-6-(phenylmethyl)-4-pyrimidinyl]-1,3-benzothiazol-2-amine by reacting the appropriately substituted 1,3-benzothiazol-2-amine or [1,3]thiazolo[5,4-b]pyridin-2-amine with the appropriate 2,4-difluoropyrimidine:

| | | | | Purification |
|---|---|---|---|---|
| Intermediate | Structure | Name | Analytical Data | Method |
| 2 | 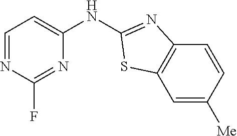 | N-(2-fluoro-4- pyrimidinyl)-6- methyl-1,3- benzothiazol- 2-amine | LCMS (Method A): Rt 1.05 minutes; m/z 261 (MH+) | Chromatography on silica; 0-100% ethyl acetate in cyclohexane |
| 3 | 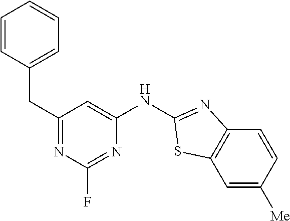 | N-[2-fluoro-6- (phenylmethyl)- 4-pyrimidinyl]- 6-methyl-1,3- benzothiazol- 2-amine | LCMS (Method A): Rt 1.29 minutes; m/z 351 (MH+) | Chromatography on silica; 0-50% ethyl acetate in cyclohexane |
| 4 | 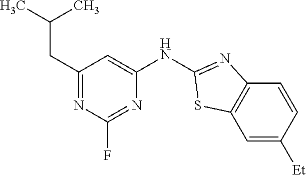 | 6-ethyl-N-[2- fluoro-6-(2- methylpropyl)- 4-pyrimidinyl]- 1,3- benzothiazol- 2-amine | LCMS (Method A): Rt 1.47 minutes; m/z 331 (MH+) | Chromatography on silica; 0-50% ethyl acetate in cyclohexane |
| 5 | 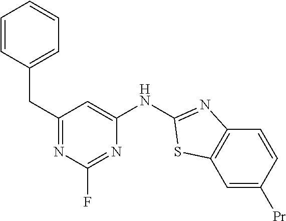 | N-[2-fluoro-6- (phenylmethyl)- 4-pyrimidinyl]- 6-propyl-1,3- benzothiazol- 2-amine | LCMS (Method A): Rt 1.43 minutes; m/z 379 (MH+) | Chromatography on silica; 0-100% ethyl acetate in cyclohexane |
| 6 | 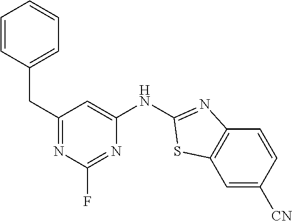 | 2-{[2-fluoro-6- (phenylmethyl)- 4-pyrimidinyl] amino}-1,3- benzothiazole- 6-carbonitrile | LCMS (Method A): Rt 1.19 minutes; m/z 362 (MH+) | Chromatography on silica; 0-100% ethyl acetate in cyclohexane |
| 7 | 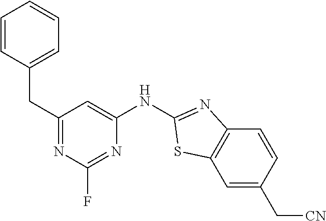 | (2-{[2-fluoro-6- (phenylmethyl)- 4-pyrimidinyl] amino}-1,3- benzothiazol- 6-yl)acetonitrile | LCMS (Method A): Rt 1.13 minutes; m/z 376 (MH+) | Chromatography on silica; 0-50% ethyl acetate in cyclohexane |
| 8 | 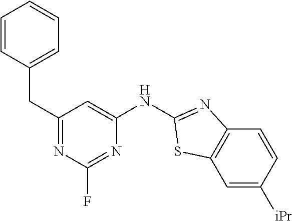 | N-[2-fluoro-6- (phenylmethyl)- 4-pyrimidinyl]- 6-(1- methylethyl)- 1,3- benzothiazol- 2-amine | LCMS (Method A): Rt 1.40 minutes; m/z 379 (MH+) | Chromatography on silica; 0-100% ethyl acetate in cyclohexane |
| 9 | 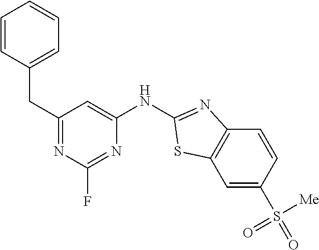 | N-[2-fluoro-6- (phenylmethyl)- 4-pyrimidinyl]- 6-(methyl- sulfonyl)-1,3- benzothiazol- 2-amine | LCMS (Method A): Rt 1.07 minutes; m/z 415 (MH+) | Chromatography on silica; 0-50% ethyl acetate in cyclohexane |
| 10 | 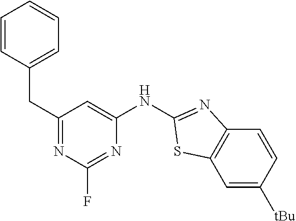 | 6-(1,1- dimethylethyl)- N-[2-fluoro-6- (phenylmethyl)- 4-pyrimidinyl]- 1,3- benzothiazol- 2-amine | LCMS (Method A): Rt 1.44 minutes; m/z 393 (MH+) | Chromatography on silica; 0-100% ethyl acetate in cyclohexane |
| 11 | 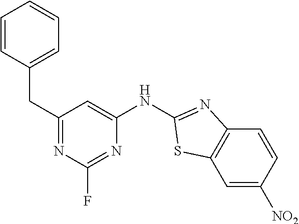 | N-[2-fluoro-6- (phenylmethyl)- 4-pyrimidinyl]- 6-nitro-1,3- benzothiazol- 2-amine | LCMS (Method A): Rt 1.25 minutes; m/z 382 (MH+) | Chromatography on silica; 0-50% ethyl acetate in cyclohexane |
| 12 | 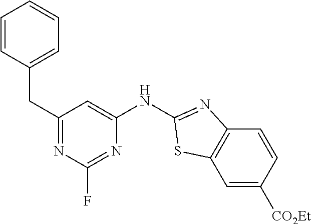 | ethyl 2-{[2- fluoro-6- (phenylmethyl)- 4-pyrimidinyl] amino}-1,3- benzothiazole- 6-carboxylate | LCMS (Method A): Rt 1.29 minutes; m/z 409 (MH+) | Chromatography on silica; 0-50% ethyl acetate in cyclohexane |
| 13 | 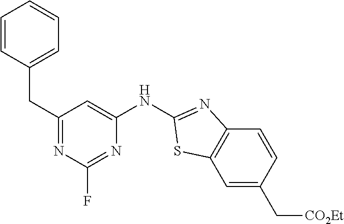 | ethyl (2-{[2- fluoro-6- (phenylmethyl)- 4-pyrimidinyl] amino}-1,3- benzothiazol- 6-yl)acetate | LCMS (Method A): Rt 1.29 minutes; m/z 423 (MH+) | Chromatography on silica; 0-50% ethyl acetate in cyclohexane |
| 14 | 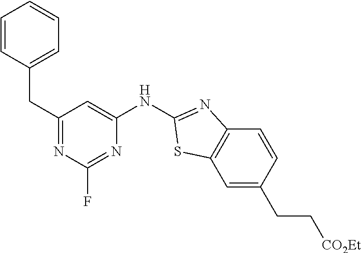 | ethyl 3-(2-{[2- fluoro-6- (phenylmethyl)- 4-pyrimidinyl] amino}-1,3- benzothiazol-6- yl)propanoate | LCMS (Method A): Rt 1.28 minutes; m/z 437 (MH+) | Chromatography on silica; 0-50% ethyl acetate in cyclohexane |
| 15 | 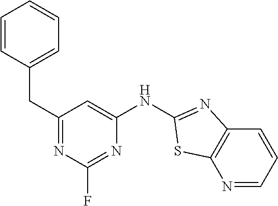 | N-[2-fluoro-6- (phenylmethyl)- 4-pyrimidinyl] [1,3]thiazolo [5,4-b]pyridin- 2-amine | LCMS (Method A): Rt 1.06 minutes; m/z 338 (MH+) | Chromatography on silica; 0-50% ethyl acetate in cyclohexane |
| 16 | 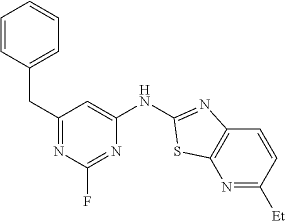 | 5-ethyl-N-[2- fluoro-6- (phenylmethyl)- 4-pyrimidinyl] [1,3]thiazolo [5,4-b]pyridin- 2-amine | LCMS (Method A): Rt 1.20 minutes; m/z 366 (MH+) | Chromatography on silica; 0-50% ethyl acetate in cyclohexane |
| 17 | 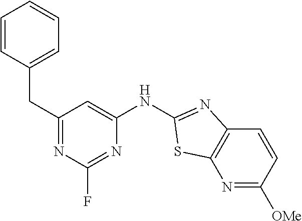 | N-[2-fluoro-6- (phenylmethyl)- 4-pyrimidinyl]- 5-(methyloxy) [1,3]thiazolo [5,4-b]pyridin- 2-amine | LCMS (Method A): Rt 1.24 minutes; m/z 368 (MH+) | Chromatography on silica; 0-50% ethyl acetate in cyclohexane |
| 18 | 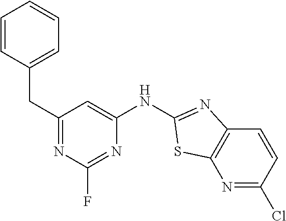 | 5-chloro-N-[2- fluoro-6- (phenylmethyl)- 4-pyrimidinyl] [1,3]thiazolo [5,4-b]pyridin- 2-amine | LCMS (Method A): Rt 1.23 minutes; m/z 372 (MH+) | Impurities precipitated in THF/methanol, filtered, the filtrate was evaporated and dried |
| 19 | 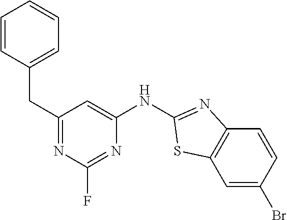 | 6-bromo-N-[2- fluoro-6- (phenylmethyl)- 4-pyrimidinyl]- 1,3- benzothiazol- 2-amine | LCMS (Method A): Rt 1.37 minutes; m/z 415, 417 (MH+) | Chromatography on silica; 0-50% ethyl acetate in cyclohexane |
| 20 | 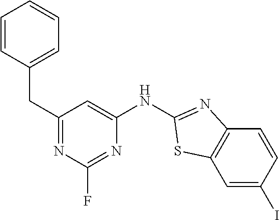 | N-[2-fluoro-6- (phenylmethyl)- 4-pyrimidinyl]- 6-iodo-1,3- benzothiazol- 2-amine | LCMS (Method A): Rt 1.39 minutes; m/z 463 (MH+) | Chromatography on silica; 0-50% ethyl acetate in cyclohexane |
| 21 | 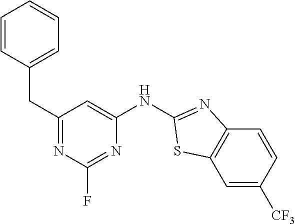 | N-[2-fluoro-6- (phenylmethyl)- 4-pyrimidinyl]- 6-(trifluoro- methyl)-1,3- benzothiazol- 2-amine | LCMS (Method A): Rt 1.36 minutes; m/z 405 (MH+) | Chromatography on silica; 0-25% ethyl acetate in cyclohexane |
| 22 | 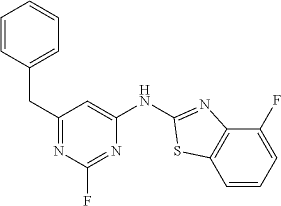 | 4-fluoro-N-[2- fluoro-6- (phenylmethyl)- 4-pyrimidinyl]- 1,3- benzothiazol- 2-amine | LCMS (Method A): Rt 1.26 minutes; m/z 355 (MH+) | Chromatography on silica; 0-50% ethyl acetate in cyclohexane |
| 23 | 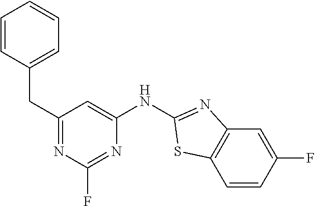 | 5-fluoro-N-[2- fluoro-6- (phenylmethyl)- 4-pyrimidinyl]- 1,3- benzothiazol- 2-amine | LCMS (Method A): Rt 1.27 minutes; m/z 355 (MH+) | Chromatography on silica; 0-25% ethyl acetate in cyclohexane |
| 24 | 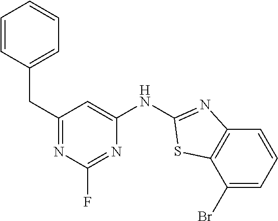 | 7-bromo-N-[2- fluoro-6- (phenylmethyl)- 4-pyrimidinyl]- 1,3- benzothiazol- 2-amine | LCMS (Method A): Rt 1.39 minutes; m/z 415, 417 (MH+) | Chromatography on silica; 0-25% ethyl acetate in cyclohexane |

### Intermediate 25

### N-[2-chloro-6-(1,1-difluoroethyl)-4-pyrimidinyl]-6-(methyloxy)-1,3-benzothiazol-2-amine

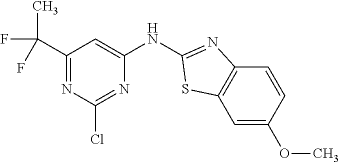

Under a atmosphere of nitrogen, a solution of 6-(methyloxy)-1,3-benzothiazol-2-amine (381 mg, 2.11 mmol) in tetrahydrofuran (5 mL) was cooled to −78° C. and then treated slowly with a solution of lithium hexamethyldisilazide in tetrahydrofuran (1M, 1.8 mL, 1.8 mmol). After 10 minutes a solution of 2,4-dichloro-6-(1,1-difluoroethyl)pyrimidine in tetrahydrofuran (5 mL) was slowly added and the reaction mixture then allowed to warm to ambient temperature over 2 hours. The reaction mixture was treated with aqueous hydrochloric acid (2M) and water and then extracted with chloroform. The organic phase was collected and evaporated to dryness. The product was purified by chromatography on silica using a gradient elution from 0 to 50% ethyl acetate in cyclohexane to afford 160 mg (160 mg, 0.45 mmol, 32% yield) of the title compound. LCMS (Method A): Rt 1.20 minutes; m/z 357 (MH⁺).

The compounds shown in the table were prepared in an analogous manner to that for N-[2-chloro-6-(1,1-difluoroethyl)-4-pyrimidinyl]-6-(methyloxy)-1,3-benzothiazol-2-amine by reacting the appropriately substituted 1,3-benzothiazol-2-amine or [1,3]thiazolo[5,4-b]pyridin-2-amine with the appropriately substituted 2,4-dichloropyrimidine:

| | | | Analytical | Purification |
|---|---|---|---|---|
| Intermediate | Structure | Name | Data | Method |
| 26 | 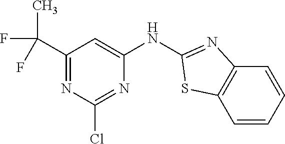 | N-[2-chloro-6- (1,1- difluoroethyl)- 4-pyrimidinyl]- 1,3- benzothiazol- 2-amine | LCMS (Method A): Rt 1.21 minutes; m/z 327 (MH+) | Chromatography on silica; 0-50% ethyl acetate in cyclohexane |
| 27 | 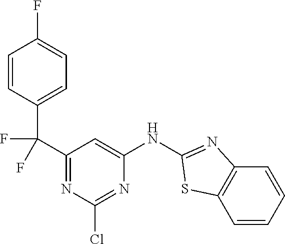 | N-{2-chloro-6- [difluoro(4- fluorophenyl) methyl]-4- pyrimidinyl}- 1,3- benzothiazol- 2-amine | LCMS (Method A): Rt 1.37 minutes; m/z 407 (MH+) | Chromatography on silica; 0-50% ethyl acetate in cyclohexane |
| 28 | 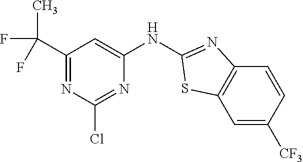 | N-[2-chloro-6- (1,1- difluoroethyl)- 4-pyrimidinyl]- 6-(trifluoro- methyl)- 1,3- benzothiazol- 2-amine | LCMS (Method A): Rt 1.33 minutes; m/z 395 (MH+) | Chromatography on silica; 0-100% ethyl acetate in cyclohexane |
| 29 | 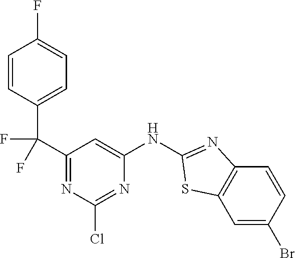 | 6-bromo-N-{2- chloro-6- [difluoro(4- fluorophenyl) methyl]-4- pyrimidinyl}- 1,3- benzothiazol- 2-amine | LCMS (Method A): Rt 1.48 minutes; m/z 485, 487 (MH+) | Chromatography on silica; 0-50% ethyl acetate in cyclohexane |
| 30 | 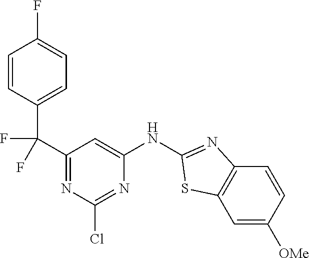 | N-{2-chloro-6- [difluoro(4- fluorophenyl) methyl]-4- pyrimidinyl}-6- (methyloxy)- 1,3- benzothiazol- 2-amine | LCMS (Method A): Rt 1.26 minutes; m/z 437 (MH+) | Chromatography on silica; 0-100% ethyl acetate in cyclohexane |
| 31 | 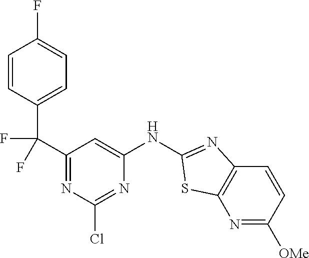 | N-{2-chloro-6- [difluoro(4- fluorophenyl) methyl]-4- pyrimidinyl}-5- (methyloxy) [1,3]thiazolo [5,4-b]pyridin- 2-amine | LCMS (Method A): Rt 1.38 minutes; m/z 438 (MH+) | Chromatography on silica; 0-50% ethyl acetate in cyclohexane |
| 32 | 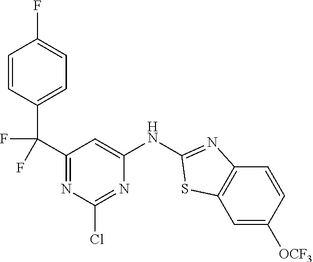 | N-{2-chloro-6- [difluoro(4- fluorophenyl) methyl]-4- pyrimidinyl}-6- [(trifluoro- methyl)oxy]- 1,3- benzothiazol- 2-amine | LCMS (Method A): Rt 1.44 minutes; m/z 491 (MH+) | Chromatography on silica; 0-50% ethyl acetate in cyclohexane |
| 33 | 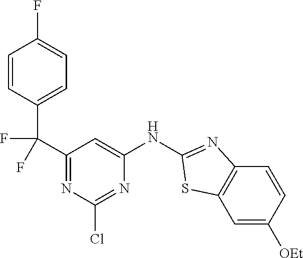 | N-{2-chloro-6- [difluoro(4- fluorophenyl) methyl]-4- pyrimidinyl}-6- (ethyloxy)-1,3- benzothiazol- 2-amine | LCMS (Method A): Rt 1.39 minutes; m/z 451 (MH+) | Chromatography on silica; 0-50% ethyl acetate in cyclohexane |

### Intermediate 34

### 6-(1,1-dimethylethyl)-1,3-benzothiazol-2-amine

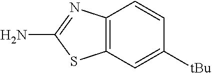

To an ice-cooled, stirred solution of 4-(1,1-dimethylethyl)aniline (2.1 mL, 13.4 mmol) and potassium thiocyanate (5.21 g, 53.6 mmol) in acetic acid (20 mL) under an atmosphere of nitrogen was added a solution of bromine (1.0 mL, 20 mmol) in acetic acid (10 mL) dropwise over 30 minutes. The reaction mixture was stirred at ambient temperature for a further 30 minutes. Water (120 mL) was added to the reaction mixture, which was then heated to 80° C. and cautiously filtered whilst hot. The filtered solid was washed with further hot acetic acid (100 mL). When the combined filtrate had cooled to ambient temperature, aqueous ammonia (28%, 200 mL) was cautiously added resulting in the formation of a precipitate which was subsequently filtered off and dried to afford the title compound (2.60 g, 12.6 mmol, 94% yield) as a white solid. LCMS (Method A): Rt 0.76 minutes; m/z 207 (MH+).

The compound shown in the table was prepared in an analogous manner to that for (2-amino-1,3-benzothiazol-6-yl)acetonitrile by reacting 6-chloro-3-pyridinamine with potassium thiocyanate and bromine:

| | | | | Purifi- |
|---|---|---|---|---|
| Inter- | | | Analytical | cation |
| mediate | Structure | Name | Data | Method |
| 35 | 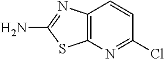 | 5-chloro [1,3] thiazolo [5,4-b] pyridin-2- amine | LCMS (Method A): Rt 0.66 minutes; m/z 186 (MH+) | Filtered off and dried |

### Intermediate 36

### (2-amino-1,3-benzothiazol-6-yl)acetonitrile

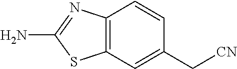

To a stirred solution of (4-aminophenyl)acetonitrile (1 g, 7.57 mmol) and potassium thiocyanate (2.94 g, 30.3 mmol) in acetic acid (20 mL) under an atmosphere of nitrogen was added, dropwise, bromine (0.58 mL, 11.4 mmol) in acetic acid (10 mL). The reaction mixture was stirred at ambient temperature overnight. Water (250 mL) was added to the reaction mixture, which was then made alkaline with the cautious addition of aqueous ammonia (28%). The mixture was extracted with ethyl acetate (2×200 mL). The combined organics were dried over magnesium sulfate, filtered and evaporated to dryness. The product was purified by chromatography on silica using a gradient elution from 0 to 100% ethyl acetate in cyclohexane to afford the title compound (0.88 g, 4.64 mmol, 61% yield). LCMS (Method A): Rt 0.41 minutes; m/z 190 (MH+).

The compounds shown in the table were prepared in an analogous manner to that for (2-amino-1,3-benzothiazol-6-yl)acetonitrile by reacting the appropriately substituted aniline with potassium thiocyanate and bromine:

| | | | Analytical | Purification |
|---|---|---|---|---|
| Intermediate | Structure | Name | Data | Method |
| 37 | 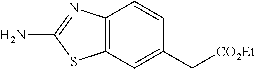 | ethyl (2-amino- 1,3- benzothiazol- 6-yl)acetate | LCMS (Method A): Rt 0.83 minutes; m/z 237 (MH+) | Chromatography on silica; 0-100% ethyl acetate in cyclohexane |
| 38 | 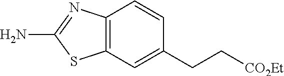 | ethyl 3-(2- amino-1,3- benzothiazol- 6- yl)propanoate | LCMS (Method A): Rt 0.88 minutes; m/z 251 (MH+) | Chromatography on silica; 0-100% ethyl acetate in cyclohexane |
| 39 | 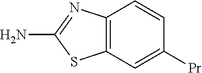 | 6-propyl-1,3- benzothiazol- 2-amine | LCMS (Method A): Rt 0.78 minutes; m/z 193 (MH+) | Chromatography on silica; 0-100% ethyl acetate in cyclohexane |

### Intermediate 40

### 6-ethyl-3-nitro-2-pyridinamine

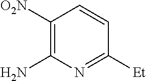

To an ice-cooled solution of 6-ethyl-2-pyridinamine (5 g, 40.9 mmol) in concentrated sulfuric acid (97%) (20 mL, 375 mmol) was slowly added concentrated nitric acid (70%) (1.9 mL, 42.5 mmol). The cooling was removed and the mixture allowed to warm to about 50° C. under a mild exotherm (some bubbling & frothing observed). After the exotherm had subsided the mixture was stirred at ambient temperature and was allowed to stand overnight. The mixture was then poured carefully into stirred, crushed ice (about 500 mL). The resulting solution was treated with aqueous sodium hydroxide (10M) until pH 4-5 was achieved. The precipitated solid was filtered off, washed with water and dried. The product was purified by flash chromatography on silica using a gradient elution from 0 to 100% ethyl acetate in cyclohexane to afford the title compound (1.7 g, 10.2 mmol, 25% yield) (as well as 2.6 g of the later-eluting isomeric by-product, 6-ethyl-5-nitro-2-pyridinamine). LCMS (Method A): Rt 0.76 minutes; m/z 168 (MH+). ¹H NMR (400 MHz, MeOD) δ 1.25 (t, J=7.65 Hz, 3H), 2.69 (q, J=7.70 Hz, 2H), 6.64 (d, J=8.53 Hz, 1H), 8.33 (d, J=8.53 Hz, 1H). (The amine functionality was not seen in this NMR spectrum due to proton exchange with the solvent).

### Intermediate 41

### 2-chloro-6-ethyl-3-nitropyridine

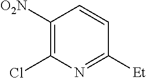

A solution of 6-ethyl-3-nitro-2-pyridinamine (0.8 g, 4.8 mmol) in concentrated hydrochloric acid (37%) (40 mL) was cooled to −15° C. and treated portionwise with sodium nitrite (6.6 g, 96 mmol). The stirred mixture was allowed to warm slowly to ambient temperature and stirred at that temperature for 2 hours. The mixture was then diluted with water (40 mL) and extracted with diethyl ether (3×50 mL). The combined organics were dried over magnesium sulfate, filtered and evaporated. The product was purified by flash chromatography on silica using a gradient elution from 0% to 50% ethyl acetate in cyclohexane to afford the title compound (0.57 g, 3.1 mmol, 64% yield) as a yellow liquid. LCMS (Method A): Rt 1.00 minutes; m/z 187 (MH+).

### Intermediate 42

### 2-chloro-6-ethyl-3-pyridinamine

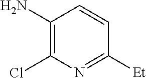

A solution of 2-chloro-6-ethyl-3-nitropyridine (430 mg, 2.3 mmol) in ethanol (5 mL) was treated portionwise over 5 minutes with tin (II) chloride (2.19 g, 11.6 mmol). The resulting solution was heated to 50° C. for 30 minutes and then cooled to ambient temperature. The mixture was then treated cautiously with saturated aqueous sodium bicarbonate (40 mL) followed by ethyl acetate (50 mL). The mixture was filtered and the filtrate separated. The aqueous phase was extracted with more ethyl acetate (50 mL) and the combined organics were dried over magnesium sulfate, filtered and evaporated to dryness to afford the title compound (361 mg, 2.3 mmol, quantitative) as a pale yellow solid. LCMS (Method A): Rt 0.68 minutes; m/z 157 (MH+).

### Intermediate 43

### 5-ethyl[1,3]thiazolo[5,4-b]pyridin-2-amine

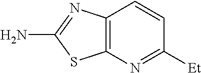

A mixture of 2-chloro-6-ethyl-3-pyridinamine (280 mg, 1.79 mmol) and potassium thiocyanate (348 mg, 3.6 mmol) in ethanol (5 mL) was treated with aqueous hydrochloric acid (2M, 10 drops) and heated in a Biotage “Initiator” microwave at 130° C. for 2 hours. The cooled mixture was partitioned between dichloromethane (40 mL) and water (20 mL) and the organic fraction was collected, dried over magnesium sulfate, filtered and evaporated to dryness. The product was purified by flash chromatography on silica using a gradient elution from 0 to 100% ethyl acetate in cyclohexane to afford the title compound (227 mg, 1.27 mmol, 71% yield). LCMS (Method A): Rt 0.57 minutes; m/z 180 (MH+).

### Intermediate 44

### (2,6-dichloro-4-pyrimidinyl)(4-fluorophenyl)methanone

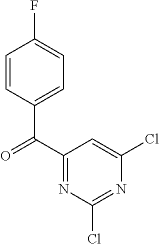

A solution of methyl 2,6-dichloro-4-pyrimidinecarboxylate (12 g, 58.0 mmol) in anhydrous tetrahydrofuran (100 mL) under an atmosphere of nitrogen was cooled to −78° C. and treated dropwise with 4-fluorophenylmagnesium bromide (2M in diethyl ether) (29 mL, 58.0 mmol). The mixture stirred at −78° C. for 30 minutes and then treated with brine (10 mL) and water (10 mL). The mixture was allowed to warm to ambient temperature and then extracted with ethyl acetate (2×30 mL). The combined organics were dried over magnesium sulfate, filtered and evaporated to dryness. The product was purified by flash chromatography on silica using a gradient elution from 0 to 50% ethyl acetate in cyclohexane to afford the title compound (13.6 g, 50.0 mmol, 86% yield) as a white solid. LCMS (Method A): Rt 1.20 minutes; m/z 271 (MH⁺)

### Intermediate 45

### 1-(2,6-dichloro-4-pyrimidinyl)ethanone

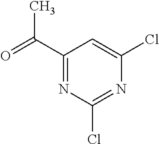

A solution of methyl 2,6-dichloro-4-pyrimidinecarboxylate (15 g, 72.5 mmol) in anhydrous tetrahydrofuran (150 mL) was cooled to −78° C. and treated dropwise with methylmagnesium bromide (1M in tetrahydrofuran) (123 mL, 123 mmol). The mixture was allowed to stir at −78 C for 30 minutes and then treated with brine (60 mL) and water (60 mL). The mixture was allowed to warm to ambient temperature and then extracted with ethyl acetate (2×150 mL). The combined organics were dried over magnesium sulfate, filtered and evaporated to dryness. The product was purified by flash chromatography on silica using a gradient elution from 0 to 100% ethyl acetate in cyclohexane to afford the title compound (8.1 g, 42.4 mmol, 59% yield) as a colourless liquid. LCMS (Method A): Rt 0.92 minutes; m/z 191 (MH⁺) (weak signal). ¹H NMR (400 MHz, CDCl₃); δ 2.71 (3H, s), 7.85 (1H, s).

### Intermediate 46

### 2,4-difluoro-6-(phenylmethyl)pyrimidine

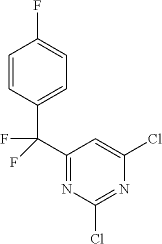

Under an atmosphere of nitrogen, a solution of (2,6-dichloro-4-pyrimidinyl)(4-fluorophenyl)methanone (13.5 g, 49.8 mmol) in dichloromethane (4 mL) was treated with diethylaminosulfur trifluoride (16 mL, 121 mmol) and the mixture was stirred at ambient temperature for 20 hours. The mixture was then cautiously added dropwise to 10 mL of ice/water and the product extracted with dichloromethane (2×10 mL). The combined organic fractions were evaporated to dryness and passed through a short silica (Merck-60) column (cyclohexane as eluent). Product-containing fractions were combined and evaporated to dryness to afford the title compound (12.4 g, 42.3 mmol, 85% yield). LCMS (Method A): Rt 1.20 minutes; no mass ion detected. ¹H NMR (400 MHz, CDCl₃); δ 7.16 (2H, t, J=8.66 Hz), 7.62 (2H, dd, J=8.78 Hz, 5.27 Hz), 7.68 (1H, s).

### Intermediate 47

### 2,4-dichloro-6-(1,1-difluoroethyl)pyrimidine

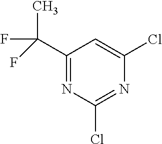

Under a atmosphere of nitrogen, a solution of 1-(2,6-dichloro-4-pyrimidinyl)ethanone (660 mg, 3.5 mmol) in dichloromethane (1 mL) was treated with diethylaminosulfur trifluoride (1 mL, 7.6 mmol) and the mixture was stirred at ambient temperature for 20 hours. The mixture was cautiously added dropwise to 10 mL of ice/water and the product was then extracted with dichloromethane (2×10 mL). The combined organic fractions were evaporated to dryness and passed through a short silica column (cyclohexane as eluent). Product-containing fractions were combined and evaporated to dryness to afford the title compound (692 mg, 3.25 mmol, 94% yield). LCMS (Method A): Rt 1.06 minutes; no mass ion detected. ¹H NMR (400 MHz, CDCl₃); δ 2.71 (3H, t, J=18.82 Hz), 7.61 (1H, s).

### Intermediate 48

### 2,4-difluoro-6-(phenylmethyl)pyrimidine

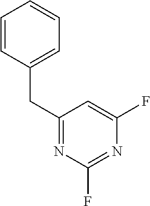

A stirred solution of 2,4,6-trifluoropyrimidine (51 g, 380 mmol) in tetrahydrofuran (750 mL) under nitrogen cooled to −75° C. was treated dropwise with benzylmagnesium chloride (2M in tetrahydrofuran) (190 mL, 380 mmol) over 30 minutes, maintaining the temperature between −65° C. and −75° C. The mixture was stirred at around −75° C. for a further 60 minutes and then quenched by the addition of brine (250 mL). The mixture was allowed to warm to ambient temperature and was then treated with water (300 mL) and ethyl acetate (300 mL). The mixture was separated and the aqueous phase was extracted with more ethyl acetate (300 mL). The combined organics were dried over magnesium sulfate, filtered and evaporated to dryness to afford 78 g of the crude product as a yellow oil. This product was purified by chromatography on silica using a gradient elution from 0 to 25% ethyl acetate in cyclohexane to afford the title compound (61.8 g, 300 mmol, 79% yield) as a pale yellow oil. LCMS (Method A): Rt 0.83 minutes; m/z 207 (MH⁺)

### Intermediate 49

### 2,4-difluoro-6-(2-methylpropyl)pyrimidine

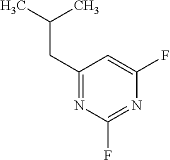

A stirred solution of 2,4,6-trifluoropyrimidine (9 g, 67.1 mmol) in tetrahydrofuran (450 mL) under nitrogen was treated with iron(III)(acac)₃ (200 mg) and cooled to −75° C. To this was added a solution of isobutylmagnesium chloride in tetrahydrofuran (2M, 33.6 mL, 67.1 mmol) dropwise over 15 minutes maintaining the temperature between −65° C. and −75° C. and stirred at −75° C. for a further 1.5 hours. The mixture was treated with brine (200 mL), allowed to warm to ambient temperature and then separated. The aqueous phase was extracted with diethyl ether and the combined organic extracts then dried over magnesium sulfate, filtered and the filtrate evaporated to dryness to afford the title compound (10.8 g, 62.8 mmol, 94%). LCMS (Method A): Rt 1.25 minutes; m/z 173 (MH+)

### Intermediate 50

### 4-chloro-2-(methylthio)-6-(phenylmethyl)pyrimidine

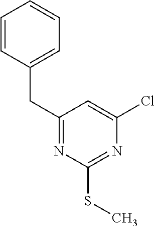

A solution of 4,6-dichloro-2-(methylthio)pyrimidine (10 g, 51.3 mmol) in tetrahydrofuran (250 mL) was cooled to −78° C. and treated dropwise with benzylmagnesium chloride (2M in tetrahydrofuran) (51.3 mL, 103 mmol) over 25 minutes. The mixture was stirred at −78° C. for 4 hours and then quenched by the addition of brine (100 mL). The mixture was allowed to warm to ambient temperature and then treated with water (80 mL), aqueous hydrochloric acid (2M, 30 mL) and ethyl acetate (100 mL). The mixture was separated and the aqueous phase was extracted with more ethyl acetate (150 mL). The combined organics were dried over magnesium sulfate, filtered and evaporated to dryness. The product was purified by flash chromatography on silica, using a gradient elution from 0 to 25% ethyl acetate in cyclohexane to afford the title compound (7.8 g, 31.1 mmol, 61% yield) as a yellow liquid. LCMS (Method A): Rt 1.37 minutes; m/z 251 (MH+)

### Intermediate 51

### 2-(methylthio)-6-(phenylmethyl)-4-pyrimidinamine

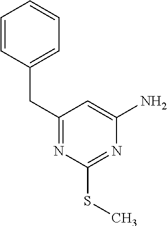

A solution of 4-chloro-2-(methylthio)-6-(phenylmethyl)pyrimidine (800 mg, 3.19 mmol) in isopropanol (10 mL) was treated with concentrated aqueous ammonia (2 mL, 103 mmol) and the mixture was sealed and heated at 150° C. in a Biotage “Initiator” microwave for 2 hours. The mixture was evaporated to dryness, partitioned between dichloromethane (10 mL) and water (10 mL), separated, the organic fraction evaporated to dryness and the product was then purified by flash chromatography on silica using a gradient elution from 0% to 100% ethyl acetate in cyclohexane to afford the title compound (584 mg, 2.52 mmol, 79% yield) as a pale yellow solid. LCMS (Method A): Rt 0.61 minutes; m/z 232 (MH+)

### Intermediate 52

### N-[2-(methylthio)-6-(phenylmethyl)-4-pyrimidinyl]-1,3-benzothiazol-2-amine

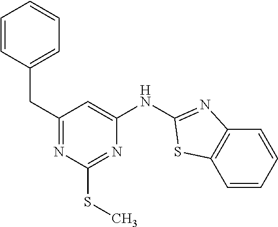

Under an atmosphere of nitrogen, an ice-cooled, stirred solution of 2-(methylthio)-6-(phenylmethyl)-4-pyrimidinamine (500 mg, 2.16 mmol) in tetrahydrofuran (5 mL) was treated with sodium hydride (112 mg, 2.8 mmol). After 20 minutes, the mixture was treated with 2-chloro-1,3-benzothiazole (367 mg, 2.16 mmol) stirred and allowed to warm to ambient temperature over 30 minutes and then heated at 60° C. for 12 hours. The cooled mixture was then treated with saturated aqueous ammonium chloride (20 mL) and dichloromethane (20 mL). The organic phase was collected, evaporated to dryness and the product was purified by mass-directed autopreparative HPLC (ammonium bicarbonate modifier) to afford 140 mg of the title compound as a white solid. LCMS (Method A): Rt 1.31 minutes; m/z 365 (MH+)

The compound shown in the table was prepared in an analogous manner to that for N-[2-(methylthio)-6-(phenylmethyl)-4-pyrimidinyl]-1,3-benzothiazol-2-amine by reacting 2-(methylthio)-6-(phenylmethyl)-4-pyrimidinamine with 2-chloro-6-(methyloxy)-1,3-benzothiazole:

| | | | Analytical | Purification |
|---|---|---|---|---|
| Intermediate | Structure | Name | Data | Method |
| 53 | 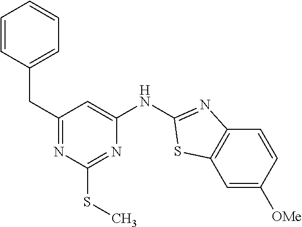 | 6-(methyloxy)-N-[2- (methylthio)-6- (phenylmethyl)-4- pyrimidinyl]-1,3- benzothiazol-2- amine | LCMS (Method A): Rt 1.29 minutes; m/z 395 (MH+) | MDAP, ammonium bicarbonate modifier |

### Intermediate 54

### N-[2-(methylsulfinyl)-6-(phenylmethyl)-4-pyrimidinyl]-1,3-benzothiazol-2-amine

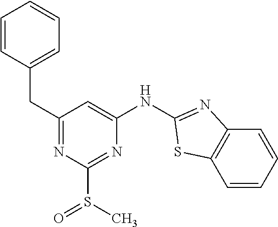

A solution of N-[2-(methylthio)-6-(phenylmethyl)-4-pyrimidinyl]-1,3-benzothiazol-2-amine (120 mg, 0.329 mmol) in N,N-dimethylformamide (8 mL) was treated with a solution of Oxone® (607 mg, 0.988 mmol) in water (3 mL) and the mixture was stirred at ambient temperature for 18 hours. The mixture was treated with dichloromethane (20 mL) and water (20 mL). The organic fraction was separated and evaporated to dryness to afford the title compound (65 mg, 0.17 mmol, 52% yield). LCMS (Method A): Rt 0.98 minutes; m/z 381 (MH+).

### Intermediate 55

### 6-(methyloxy)-N-[2-(methylsulfonyl)-6-(phenylmethyl)-4-pyrimidinyl]-1,3-benzothiazol-2-amine

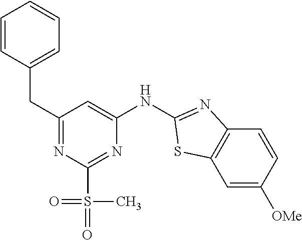

A solution of 6-(methyloxy)-N-[2-(methylthio)-6-(phenylmethyl)-4-pyrimidinyl]-1,3-benzothiazol-2-amine (200 mg, 0.51 mmol) in N,N-dimethylformamide (10 mL) was treated with Oxone® (935 mg, 1.52 mmol) and the mixture was stirred overnight at ambient temperature. Water (20 mL) and dichloromethane (20 mL) were added to the reaction mixture which was then separated. The organic fraction collected and evaporated to dryness to afford the title compound (178 mg, 0.42 mmol, 82% yield) as an off-white solid. This was utilised without further purification. LCMS (Method A): Rt 1.07 minutes; m/z 427 (MH+).

### Intermediate 56

### 2,2,2-trifluoro-N-{2-[4-(methyloxy)phenyl]ethyl}acetamide

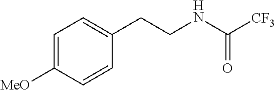

A solution of trifluoroacetic anhydride (93.4 mL, 660 mmol) in anhydrous dichloromethane (100 mL) was added dropwise to a cooled solution of {2-[4-(methyloxy)phenyl]ethyl}amine (50 g, 330 mmol) and triethylamine (92 mL, 660 mmol) in anhydrous dichloromethane (300 mL). Upon addition, the mixture was stirred for 1 hour and then washed sequentially with aqueous hydrochloric acid (2M, 400 mL), aqueous sodium hydrogen carbonate solution (8% w/v, 400 mL) and water (200 mL). The organic fraction was dried over magnesium sulfate, filtered and evaporated to dryness to afford the crude product as a pale yellow solid. The product was purified by recystallisation from cyclohexane to afford the title compound (38 g, 154 mmol, 47% yield) as a white solid. Melting point 73-74° C.

### Intermediate 57

### 2-(methyloxy)-5-{2-[(trifluoroacetyl)amino]ethyl}benzenesulfonyl chloride

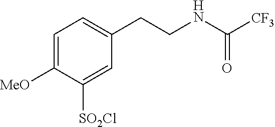

A stirred solution of 2,2,2-trifluoro-N-{2-[4-(methyloxy)phenyl]ethyl}acetamide (30 g, 120 mmol) in dry chloroform (150 mL) was cooled to −5° C. and treated dropwise with chlorosulfonic acid (150 mL, 1500 mmol). The mixture was then stirred at ambient temperature for 30 minutes before being added cautiously to ice/water (300 mL). The organic phase was collected, dried over magnesium sulfate, filtered and evaporated to dryness. The product was purified by crystallisation from chloroform/petroleum ether (60-80° C.) to afford the title compound (35.3 g, 102 mmol, 85% yield) as a white solid. Melting point 96-98° C.

### Intermediate 58

### N-{2-[3-(aminosulfonyl)-4-(methyloxy)phenyl]ethyl}-2,2,2-trifluoroacetamide

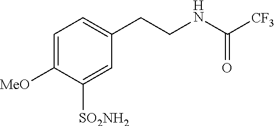

To a mixture of liquid ammonia in anhydrous tetrahydrofuran (100 mL) was added 2-(methyloxy)-5-{2-[(trifluoroacetyl)amino]ethyl}benzenesulfonyl chloride. The mixture was stirred for 3 hours and then evaporated to dryness. The residual solid was stirred in water for 30 minutes and then filtered and dried. The product was purified by recrystallisation from ethyl acetate/petroleum ether to afford the title compound (1.82 g, 95% yield) as a white solid. Melting point 167-168° C.

### Intermediate 59

### 5-(2-aminoethyl)-2-(methyloxy)benzenesulfonamide

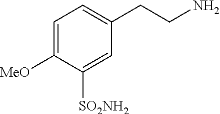

A mixture of N-{2-[3-(aminosulfonyl)-4-(methyloxy)phenyl]ethyl}-2,2,2-trifluoroacetamide (0.5 g, 1.53 mmol) and concentrated aqueous ammonia (10 mL) was heated at reflux for 6.5 hours and then allowed to stand at ambient temperature for a further 2 days. The mixture was filtered and the filtrate evaporated to dryness. The product was purified by recrystallisation from n-propanol and cyclohexane to afford the title compound (0.28 g, 1.2 mmol, 78% yield) as a white solid. Melting point 164-167° C.

### Intermediate 60

### 5-(2-aminoethyl)-2-hydroxybenzenesulfonamide hydrobromide

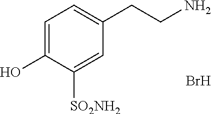

Under an atmosphere of nitrogen, a solution of boron tribromide in dichloromethane (1M, 1.9 mL, 19 mmol) was added dropwise to a cooled, stirred solution of 5-(2-aminoethyl)-2-(methyloxy)benzenesulfonamide (2.3 g, 10 mmol) in anhydrous dichloromethane (70 mL) and the mixture was stirred for 4 hours. Methanol (40 mL) was then added and the mixture was heated at reflux for 1 hour. The resulting solution was evaporated to dryness and the residual solid was recrystallised from a mixture of methanol, diethyl ether and petroleum ether to afford the title compound (1.2 g, 4.0 mmol, 40% yield) as a white, crystalline solid. Melting point 228-230° C.

### Intermediate 61

### diethyl(3-fluoro-4-nitrophenyl)(methyl)propanedioate

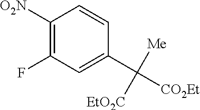

An ice-cooled, stirred solution of a mixture of 2,4-difluoro-1-nitrobenzene (6.28 mL, 57.3 mmol) and diethyl methylpropanedioate (9.97 g, 57.3 mmol) in N,N-dimethylformamide (80 mL) was treated with sodium hydroxide pellets (2.29 g, 57.3 mmol). The mixture was allowed to warm to ambient temperature and then allowed to stand for 3 days. The mixture was treated with aqueous hydrochloric acid (2M, 10 mL), then with water (200 mL) and extracted with diethyl ether (3×100 mL). The combined organics were washed with brine, dried over magnesium sulfate, filtered and evaporated to dryness. The product was purified by chromatography on silica using a gradient elution from 0 to 30% ethyl acetate in hexane to afford the title compound (12.7 g, 40.6 mmol, 71% yield) as a yellow oil. LCMS (Method B): Rt 3.04 minutes; m/z 314 (MH+).

### Intermediate 62

### diethyl(4-amino-3-fluorophenyl)(methyl)propanedioate

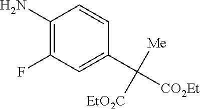

Under an atmosphere of argon, ethanol (300 mL) was cautiously added to a mixture of diethyl(3-fluoro-4-nitrophenyl)(methyl)propanedioate (12.7 g, 40.6 mmol), ammonium formate (5.1 g, 81.1 mmol) and palladium on activated charcoal (10%, 50% wet with water, 2.15 g) and the mixture was heated at 60° C. for 4 hours and at reflux for a further 3 hours. More ammonium formate (5.1 g, 81.1 mmol) was added and the mixture was heated at reflux for a further hour. The reaction mixture was cooled and cautiously filtered. The filtrate was evaporated to dryness to afford the title compound (11.5 g, 40.6 mmol, 100% yield) as a yellow oil. LCMS (Method B): Rt 2.64 minutes; m/z 284 (MH+).

### Intermediate 63

### 2-(4-amino-3-fluorophenyl)propanoic acid

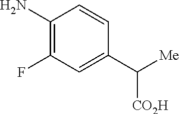

A solution of diethyl(4-amino-3-fluorophenyl)(methyl)propanedioate (10.5 g, 37 mmol) in a mixture of ethanol (250 mL) and water (10 mL) was treated with sodium hydroxide (1.48 g, 37 mmol) and heated at 90° C. for 20 hours. More sodium hydroxide (0.725 g, 18.1 mmol) was added and the mixture heated at 90° C. for a further 7 hours. The cooled mixture was evaporated to dryness and the residue was treated with aqueous hydrochloric acid (2M). The acidic, aqueous solution was washed with diethyl ether (3×100 mL), neutralised to pH 7 with aqueous sodium hydroxide (2M) and extracted with ethyl acetate (3×100 mL). The combined ethyl acetate fractions were dried over magnesium sulfate, filtered and evaporated to dryness. The product was purified by chromatography on a pre-packed reverse-phase C18 cartridge using a gradient elution from 5 to 100% acetonitrile in water to afford the title compound (2.4 g, 13.1 mmol, 35% yield) as a white solid. LCMS (Method B): Rt 1.64 minutes; m/z 184 (MH+).

### Intermediate 64

### 6-bromo-N-[2-(methylthio)-4-pyrimidinyl]-1,3-benzothiazol-2-amine

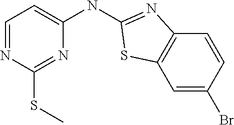

Under an atmosphere of nitrogen, a solution of 2-(methylthio)-4-pyrimidinamine (3 g, 21.25 mmol) in anhydrous tetrahydrofuran (50 mL) at 0° C. was treated with sodium hydride (1.870 g, 46.7 mmol) portionwise. The reaction mixture was stirred for 10 minutes at 0° C. then 6-bromo-2-chloro-1,3-benzothiazole (5.81 g, 23.37 mmol) was added and the reaction mixture was stirred at 65° C. overnight. The cooled reaction mixture was treated with water (200 mL), the precipitated product was filtered and thoroughly dried to afford the title compound (6.92 g, 19.5 mmol, 92% yield). LCMS (Method B): Rt 3.07 minutes; m/z 353,355 (MH+)

### Intermediate 65

### 6-bromo-N-[2-(methylsulfonyl)-4-pyrimidinyl]-1,3-benzothiazol-2-amine

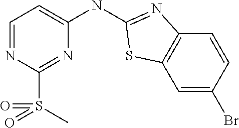

To 6-bromo-N-[2-(methylthio)-4-pyrimidinyl]-1,3-benzothiazol-2-amine (4 g, 11.32 mmol) in anhydrous N,N-dimethylformamide (80 mL) was added Oxone® (20.88 g, 34.0 mmol) portionwise. The reaction mixture was stirred at room temperature for 4 hours. Water (200 mL) was added and the mixture was extracted with dichloromethane (2×200 mL); the combined organics were washed with brine (200 mL) and evaporated to dryness to afford the title compound (2.78 g). LCMS (Method B): Rt 2.18 minutes; m/z 385,387 (MH+)

### Intermediate 66

### trans-4-[(4-{[6-(4,4,5,5-tetramethyl-1,3,2-dioxaborolan-2-yl)-1,3-benzothiazol-2-yl]amino}-2-pyrimidinyl)amino]cyclohexanol

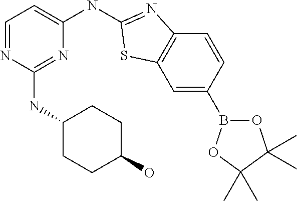

A mixture of 4,4,4′,4′,5,5,5′,5′-octamethyl-2,2′-bi-1,3,2-dioxaborolane (1.399 g, 5.51 mmol), trans-4-({4-[(6-bromo-1,3-benzothiazol-2-yl-amino]-2-pyrimidinyl}amino)cyclohexanol (example 156) (0.965 g, 2.296 mmol), 1,1′-bis(diphenylphosphino)ferrocene (0.127 g, 0.23 mmol), 1,1′-bis(diphenylphosphino)ferrocene-palladium(II)dichloride dichloromethane complex (0.187 g, 0.230 mmol) and potassium acetate (0.676 g, 6.89 mmol) in anhydrous tetrahydrofuran was sealed and heated in a Biotage “Initiator” microwave at 120° C. for 3 hours. The reaction mixture was then partitioned between water (200 mL) and ethyl acetate (200 mL). The aqueous phase was extracted with further ethyl acetate (200 mL) and the combined ethyl acetate fractions were evaporated to dryness. The product was purified by chromatography on silica using a gradient elution from 0 to 100% ethyl acetate in cyclohexane followed by 0 to 20% methanol in dichloromethane to afford the title compound (1.05 g, 92% yield). LCMS (Method A): Rt 0.84 minutes; m/z 468 (MH+).

### Intermediate 67

### 1-[(5-bromo-3-pyridinyl)methyl]-4-methylpiperazine

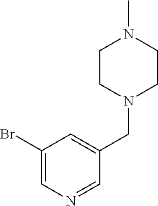

Under an atmosphere of nitrogen, a mixture of 5-bromo-3-pyridinecarbaldehyde (500 mg, 2.69 mmol), 1-methylpiperazine (538 mg, 5.38 mmol) in dichloromethane (5 mL) was treated with sodium triacetoxyborohydride (855 mg, 4.03 mmol) and stirred at room temperature overnight. The reaction mixture was then washed with water, the aqueous phase was extracted with dichloromethane (10 mL) and the combined dichloromethane fractions were evaporated to dryness. The product was purified by chromatography on silica using a gradient elution from 0 to 100% ethyl acetate in cyclohexane followed by 0 to 20% methanol in dichloromethane followed by ion exchange chromatography using an SCX (sulfonic acid) solid-phase extraction cartridge and eluting with methanol followed by 2 molar ammonia in methanol to afford the title compound (293 mg, 40% yield). LCMS (Method C): Rt 1.87 minutes; m/z 270,272 (MH+).

### Intermediate 68

### 4-[(5-bromo-3-pyridinyl)methyl]morpholine

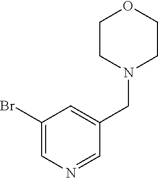

A solution of 3-bromo-5-(chloromethyl)pyridine hydrochloride (500 mg, 2.058 mmol) in methanol (5 mL) was cooled to 0° C. and morpholine (0.36 mL, 4.1 mmol) was added. The reaction mixture was stirred at room temperature overnight and then evaporated to dryness. The product was purified by chromatography on silica using a gradient elution from 0 to 100% ethyl acetate in cyclohexane followed by 0 to 20% methanol in dichloromethane to afford the title compound (217 mg, 0.84 mmol 41% yield). LCMS (Method C): Rt 1.87 minutes; m/z 257,259 (MH+).

### Intermediate 69

### 3-bromo-5-[(1-methylethyl)oxy]pyridine

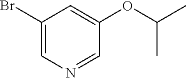

Under an atmosphere of nitrogen, a mixture of 5-bromo-3-pyridinol (5 g, 28.7 mmol), potassium carbonate (5.96 g, 43.1 mmol) and 2-bromopropane (5.30 g, 43.1 mmol) was stirred at 80° C. for 2.5 days. The cooled reaction mixture was partitioned between water (150 mL) and ethyl acetate (150 mL). The aqueous phase was extracted with further ethyl acetate (150 mL) and the combined ethyl acetate extracts were evaporated to dryness. The product was purified by flash chromatography on silica using a gradient elution from 0 to 25% ethyl acetate in cyclohexane to afford the title compound (3.84 g, 17.7 mmol, 62% yield). LCMS (Method B): Rt 2.58 minutes; m/z 216,218 (MH+).

### Intermediate 70

### 3-[(1-methylethyl)oxy]-5-(4,4,5,5-tetramethyl-1,3,2-dioxaborolan-2-yl)pyridine

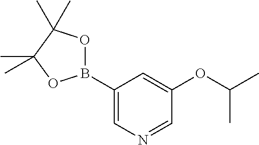

A mixture of 4,4,4′,4′,5,5,5′,5′-octamethyl-2,2′-bi-1,3,2-dioxaborolane (705 mg, 2.78 mmol), 1,1′-bis(diphenylphosphino)ferrocene-palladium(ii)dichloride dichloromethane complex (76 mg, 0.093 mmol), potassium acetate (681 mg, 6.94 mmol) was sealed in a microwave vial and placed under nitrogen via a needle through the septum and applying alternately vacuum and nitrogen. A solution of 3-bromo-5-[(1-methylethyl)oxy]pyridine (500 mg, 2.314 mmol) in anhydrous acetonitrile (10 mL) was added, the solution degassed by alternate application of vacuum and nitrogen. The reaction mixture was then heated in a Biotage “Initiator” microwave at 160° C. for 15 minutes. After cooling, the reaction was filtered and the filtrate evaporated to dryness to give the crude (approximately 60% pure by NMR analysis) title compound (960 mg, >100% yield) which was used subsequently without further purification. LCMS (Method A): Rt 0.65 minutes; m/z 182 (ionizes as the boronic acid).

### Intermediate 71

### {2-[(5-bromo-3-pyridinyl)oxy]ethyl}dimethylamine

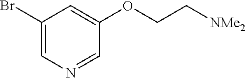

A solution of 2-(dimethylamino)ethanol (0.376 g, 4.22 mmol) in anhydrous N,N-dimethylformamide (10 mL) was cooled to 0° C. then sodium hydride (0.338 g, 8.44 mmol) was added portionwise. The reaction mixture was stirred at room temperature for 45 minutes. 3,5-dibromopyridine (1 g, 4.22 mmol) was added and the reaction mixture was heated at 90° C. overnight. The reaction mixture was allowed to cool to ambient temperature then treated with water (20 mL) and extracted with ethyl acetate (2×20 mL). The combined ethyl acetate extracts were evaporated to dryness. The product was purified by chromatography on silica using a gradient elution from 0 to 100% ethyl acetate in cyclohexane followed by 0 to 20% methanol in dichloromethane to afford the title compound (231 mg, 0.94 mmol, 22% yield). LCMS (Method B): Rt 2.13 minutes; m/z 245,247 (MH+).

### Intermediate 72

### 2-[(5-bromo-3-pyridinyl)oxy]ethanol

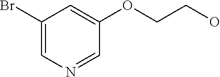

A mixture of 1,3-dioxolan-2-one (1.01 g, 11.5 mmol), 5-bromo-3-pyridinol (1 g, 5.75 mmol) and potassium carbonate (1.19 g, 8.62 mmol) in N,N-dimethylformamide (10 mL) was stirred at 86° C. overnight. The cooled reaction mixture was partitioned between water (20 mL) and ethyl acetate (20 mL). The ethyl acetate extract was evaporated to dryness and the product was purified by chromatography on silica using a gradient elution from 0 to 100% ethyl acetate in cyclohexane followed by 0 to 20% methanol in dichloromethane to afford the title compound (600 mg, 0.28 mmol, 48% yield). LCMS (Method A): Rt 0.63 minutes; m/z 218,220 (MH+).

### Intermediate 73

### 3-bromo-5-(tetrahydro-2H-pyran-4-yloxy)pyridine

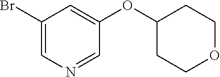

Under an atmosphere of nitrogen, a mixture of tetrahydro-2H-pyran-4-ol (0.88 g, 8.62 mmol), 5-bromo-3-pyridinol (1 g, 5.75 mmol) and triphenylphosphine (2.26 g, 8.62 mmol) in anhydrous toluene (25 mL) was treated with diisopropyl azodicarboxylate (1.676 mL, 8.62 mmol) and then heated at 110° C. overnight. The cooled reaction mixture was washed with water (25 mL) and saturated aqueous sodium bicarbonate solution (25 mL) then evaporated to dryness. The product was purified by chromatography on silica using a gradient elution from 0 to 100% ethyl acetate in cyclohexane followed by ion-exchange chromatography using an aminopropyl solid-phase extraction cartridge and methanol as eluent to afford the title compound (0.932 g, 3.61 mmol, 66% yield). LCMS (Method B): Rt 2.30 minutes; m/z 258,260 (MH+).

### Intermediate 74

### (2R,6S)-4-[(5-bromo-3-pyridinyl)methyl]-2,6-dimethylmorpholine

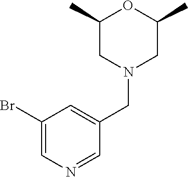

A mixture of 5-bromo-3-pyridinecarbaldehyde (500 mg, 2.69 mmol) and (2R,6S)-2,6-dimethylmorpholine (0.33 mL, 2.69 mmol) in dichloromethane (5 mL) was treated with sodium triacetoxyborohydride (855 mg, 4.03 mmol) and stirred at room temperature under an atmosphere of nitrogen overnight. The reaction mixture was washed with water, the aqueous phase was extracted with dichloromethane (10 ml) and the combined organic extracts were evaporated to dryness. The residue was purified by flash chromatography on silica using a gradient elution from 0-100% ethyl acetate in cyclohexane followed by 0-20% methanol in dichloromethane to afford the title compound (300 mg, 1.05 mmol, 39%). LCMS (Method B): Rt 2.37 minutes; m/z 285, 287 (MH+).

### Intermediate 75

### 2-{[(5-bromo-3-pyridinyl)methyl]amino}ethanol

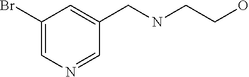

A mixture of 3-bromo-5-(chloromethyl)pyridine hydrochloride (500 mg, 2.06 mmol), 2-aminoethanol (377 mg, 6.17 mmol) and potassium carbonate (284 mg, 2.06 mmol) in acetonitrile (10 mL) was heated at 80° C. for 4 hours. Water (10 mL) was added to the cooled reaction mixture and the mixture was extracted with ethyl acetate (2×20 mL). The combined ethyl acetate extracts were evaporated to dryness. The product was purified by ion exchange chromatography using an SCX (sulfonic acid) solid-phase extraction cartridge and eluting with methanol and then with 2 molar ammonia in methanol to afford the title compound (321 mg, 1.39 mmol, 68% yield). LCMS (Method B): Rt 1.48 minutes; m/z 231,233 (MH+).

### Intermediate 76

### [(5-bromo-3-pyridinyl)methyl][2-(methyloxy)ethyl]amine

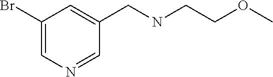

A mixture of 3-bromo-5-(chloromethyl)pyridine hydrochloride (500 mg, 2.06 mmol), 2-(methyloxy)ethanamine (464 mg, 6.17 mmol), potassium carbonate (284 mg, 2.06 mmol) in acetonitrile (10 mL) was heated at 80° C. for 4 hr. Water (10 mL) was added to the cooled reaction mixture and the mixture was extracted with ethyl acetate (2×20 mL). The combined ethyl acetate extracts were evaporated to dryness. The product was purified by ion exchange chromatography using an SCX (sulfonic acid) solid-phase extraction cartridge and eluting with methanol and then with 2 molar ammonia in methanol to afford the title compound (452 mg, 1.84 mmol, 90% yield). LCMS (Method B): Rt 1.80 minutes; m/z 245,247 (MH+).

### Intermediate 77

### 2-(methylthio)-6-(4-morpholinylmethyl)-4(1H)-pyrimidinone

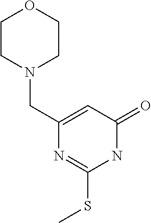

6-(Chloromethyl)-2-(methylthio)-4(1H)-pyrimidinone (1 g, 5.25 mmol) and morpholine (0.46 mL, 5.25 mmol) were dissolved in anhydrous tetrahydrofuran (105 mL) under an atmosphere of nitrogen and heated at 90° C. overnight. The reaction mixture was cooled and diluted with chloroform (200 mL), washed with brine, dried over sodium sulfate, filtered and the solvent was evaporated to dryness. The product was purified by chromatography on silica using a gradient elution from 0% to 15% methanol (+1% triethylamine) in dichloromethane to afford the title compound (0.273 g, 1.13 mmol, 22% yield) as a white solid. NMR (400 MHz, CDCl₃) δ 2.56 (4H, m), 2.57 (3H, s), 3.14 (2H, s), 4.12 (4H, m), 6.43 (1H, s).

### Intermediate 78

### 4-{[6-chloro-2-(methylthio)-4-pyrimidinyl]methyl}morpholine

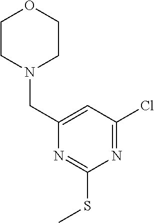

A mixture of 2-(methylthio)-6-(4-morpholinylmethyl)-4(1H)-pyrimidinone (1.03 g, 4.27 mmol) and phosphorus oxychloride (5.57 mL, 59.8 mmol) was heated at 80° C. under an atmosphere of nitrogen overnight. The cooled reaction mixture was poured on to ice (600 g) and stirred for 1 hour. An aqueous solution of sodium hydroxide (2M, 60 mL) was added and the resulting solution was extracted with dichloromethane (3×100 mL). The combined organic phase was evaporated to dryness to afford the title compound (0.63 g, 2.43 mmol, 57% yield). LCMS (Method A): Rt 0.51 minutes; m/z 260 (MH+).

### Intermediate 79

### 2-(methylthio)-6-(4-morpholinylmethyl)-4-pyrimidinamine

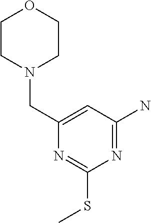

A solution of 4-{[6-chloro-2-(methylthio)-4-pyrimidinyl]methyl}morpholine (500 mg, 1.93 mmol) and concentrated aqueous ammonia (0.16 mL, 8.28 mmol) in isopropanol (3 mL) was sealed and heated in a Biotage “Initiator” microwave at 180° C. for 3 hours. The reaction mixture was evaporated to dryness and the residue was triturated with methanol, filtered and dried to afford the title compound (257 mg, 1.07 mmol, 56% yield). LCMS (Method A): Rt 0.35 minutes; m/z 241 (MH+).

### Intermediate 80

### N-[2-(methylthio)-6-(4-morpholinylmethyl)-4-pyrimidinyl]-6-nitro-1,3-benzothiazol-2-amine

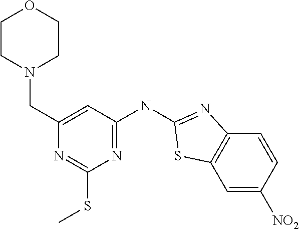

An ice-cooled solution of 2-(methylthio)-6-(4-morpholinylmethyl)-4-pyrimidinamine (250 mg, 1.04 mmol) in tetrahydrofuran (15 mL) was treated with lithium hexamethyldisilazide (1M in tetrahydrofuran, 1.04 mL, 1.04 mmol). After stirring at 0° C. for 20 minutes, a solution of 2-chloro-6-nitro-1,3-benzothiazole (223 mg, 1.04 mmol) in tetrahydrofuran (10 mL) was added dropwise. The reaction mixture was allowed to stir and warm to ambient temperature overnight. The solvent was evaporated to dryness and the residue was treated with saturated aqueous ammonium chloride (50 mL) and ethyl acetate (50 mL). The organic layer was separated, dried over magnesium sulfate, filtered and the solvent evaporated to dryness. The residue was triturated with methanol, filtered and dried to afford the title compound (186 mg, 0.444 mmol, 43% yield). LCMS (Method A): Rt 0.79 minutes; m/z 419 (MH+).

### Intermediate 81

### N-[2-(methylsulfinyl)-6-(4-morpholinylmethyl)-4-pyrimidinyl]-6-nitro-1,3-benzothiazol-2-amine

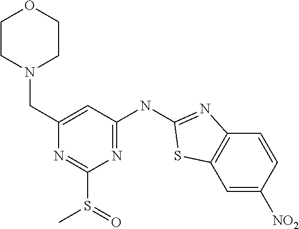

To a stirred solution of N-[2-(methylthio)-6-(4-morpholinylmethyl)-4-pyrimidinyl]-6-nitro-1,3-benzothiazol-2-amine (136 mg, 0.33 mmol) in N,N-dimethylformamide (10 mL) at ambient temperature was added Oxone® (200 mg, 0.33 mmol) portionwise and stirring was continued for 2 hours. Dichloromethane (50 mL) and water (50 mL) were then added to the reaction mixture; the organic layer was separated, washed with brine and the solvent was evaporated to dryness to afford the title compound (80 mg, 0.184 mmol, 57% yield). LCMS (Method A): Rt 0.65 minutes; m/z 435 (MH+).

### Intermediate 82

### ethyl 4,4-bis(ethyloxy)-3-oxobutanoate

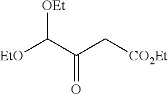

Under an atmosphere of nitrogen, sodium (6.3 g, 274 mmol) was added in 500 mg portions over 1 hour to a solution of ethyl bis(ethyloxy)acetate (30.3 mL, 170 mmol) in ethyl acetate (56 mL) at ambient temperature. There was a slow exotherm to afford a red/brown solution. The solution was heated at 60° C. for 2 hours and then stirred at ambient temperature for 18 hours. The mixture was cautiously treated with ethanol (5 mL) and water (50 mL) and then acidified to pH6 by the addition of 1M aqueous hydrochloric acid (about 160 mL). The mixture was extracted with dichloromethane (3×100 mL) and the combined organics were washed with brine (2×100 mL) and evaporated to dryness to afford the title compound (34.6 g, 159 mmol, 93% yield). ¹H NMR (400 MHz, DMSO-d6) δ 1.06-1.20 (9H, m), 3.46-3.62 (6H, m), 4.08 (2H, q, J=7.6 Hz), 4.77 (1H, s).

### Intermediate 83

### 6-[bis(ethyloxy)methyl]-2-[(phenylmethyl)thio]-4(1H)-pyrimidinone

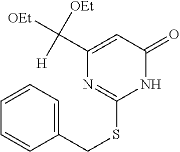

A mixture of ethyl 4,4-bis(ethyloxy)-3-oxobutanoate (34.6 g, 159 mmol) and thiourea (13.27 g, 174 mmol) in ethanol (150 mL) was treated with sodium methoxide (25% in methanol) (36 mL, 159 mmol) and the mixture was heated to reflux for 4 hours and then allowed to stand for 12 hours. Water (150 mL) was added and the stirred mixture was treated with benzyl bromide (18.9 mL, 159 mmol). After 5 minutes the stirring was ceased and a white precipitate formed. After 1 hour the mixture was diluted with water (500 mL) and the precipitated solid was filtered off, washed with water and thoroughly dried to afford the title compound (38.7 g, 121 mmol, 76% yield). LCMS (Method A): Rt 1.03 minutes; m/z 321 (MH+).

### Intermediate 84

### 4-[bis(ethyloxy)methyl]-6-chloro-2-[(phenylmethyl)thio]pyrimidine

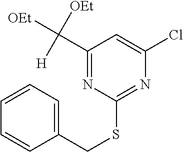

A stirred suspension of 6-[bis(ethyloxy)methyl]-2-[(phenylmethyl)thio]-4(1H)-pyrimidinone (33 g, 103 mmol) in toluene (180 mL) under an atmosphere of nitrogen was cooled in an ice/water bath and treated with N,N-dimethylformamide (16.8 mL, 216 mmol) followed, dropwise, by phosphorus oxychloride (11.5 mL, 124 mmol). The cooled mixture was stirred for 2 hours to afford a pale brown solution whereupon it was cautiously added to a saturated aqueous solution of sodium carbonate (500 mL). The aqueous phase was extracted with ethyl acetate (2×250 mL) and the combined organics were dried over magnesium sulfate, filtered and evaporated to dryness. The residue was re-evaporated twice with toluene (100 mL) to afford the title compound (32.9 g, 97 mmol, 94% yield). LCMS (Method A): Rt 1.44 minutes; m/z 339 (MH+).

### Intermediate 85

### 6-[bis(ethyloxy)methyl]-2-[(phenyl methyl)thio]-4-pyrimidinamine

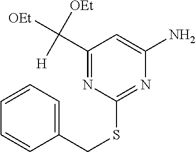

A solution of 4-[bis(ethyloxy)methyl]-6-chloro-2-[(phenylmethyl)thio]pyrimidine (32.9 g, 97 mmol) in ethanol (30 mL) was divided into 6 equal portions and each treated with concentrated aqueous ammonia solution (10 mL) and then sealed and heated in a Biotage Initiator microwave at 150° C. for 45 minutes. The mixtures were combined and evaporated to dryness. The residue was partitioned between ethyl acetate (200 mL) and water (200 mL). The aqueous phase was extracted with more ethyl acetate (2×150 mL) and the combined organics were dried over magnesium sulfate, filtered and evaporated to dryness to afford the title compound (28.8 g, 90 mmol, 93% yield). LCMS (Method A): Rt 1.00 minutes; m/z 320 (MH+).

### Intermediate 86

### N-{6-[bis(ethyloxy)methyl]-2-[(phenylmethyl)thio]-4-pyrimidinyl}-6-bromo-1,3-benzothiazol-2-amine

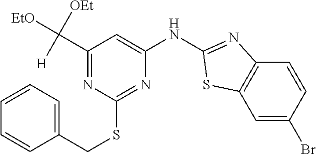

In an atmosphere of nitrogen, an ice-cooled solution of a mixture of 6-[bis(ethyloxy)methyl]-2-[(phenylmethyl)thio]-4-pyrimidinamine (10.8 g, 33.8 mmol) and 6-bromo-2-chloro-1,3-benzothiazole (8.82 g, 35.5 mmol) in dry dimethylformamide (200 mL) was treated portionwise over 5 minutes with sodium hydride (60% w/w in oil) (2.70 g, 67.6 mmol) and the mixture stirred with cooling for 3 hours. The mixture was treated with aqueous ammonium chloride (5%, 200 mL) and ethyl acetate (400 mL). The organic phase was dried over magnesium sulfate, filtered and evaporated to dryness to afford the title compound (17.82 g, 33.5 mmol, 99% yield). LCMS (Method A): Rt 1.53 minutes; m/z 531,533 (MH+).

### Intermediate 87

### N-{6-[bis(ethyloxy)methyl]-2-[(phenylmethyl)sulfonyl]-4-pyrimidinyl}-6-bromo-1,3-benzothiazol-2-amine

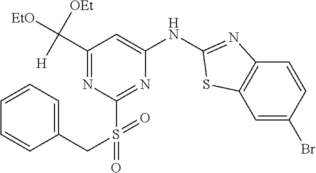

A solution of N-{6-[bis(ethyloxy)methyl]-2-[(phenylmethyl)thio]-4-pyrimidinyl}-6-bromo-1,3-benzothiazol-2-amine (18 g, 33.9 mmol) in dimethylformamide (100 mL) was treated with Oxone® (62.5 g, 102 mmol) and the mixture was stirred at ambient temperature overnight. The mixture was added to water (300 mL) and extracted with chloroform (2×250 mL). The combined organics were evaporated to dryness to afford the title compound (18.8 g, 33.4 mmol, 99% yield). LCMS (Method A): Rt 1.27 minutes; m/z 563,565 (MH+).

### Intermediate 88

### trans-4-({4-[bis(ethyloxy)methyl]-6-[(6-bromo-1,3-benzothiazol-2-yl)amino]-2-pyrimidinyl}amino)cyclohexanol

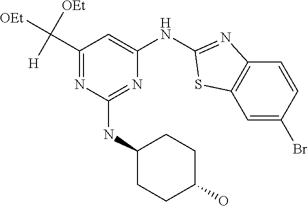

A mixture of N-{6-[bis(ethyloxy)methyl]-2-[(phenylmethyl)sulfonyl]-4-pyrimidinyl}-6-bromo-1,3-benzothiazol-2-amine (17.8 g, 31.6 mmol) and trans-4-aminocyclohexanol (10.91 g, 95 mmol) in isopropanol (10 mL) was sealed and heated in a Biotage “Initiator” microwave at 150° C. for 1 hour. The reaction mixture was added to water (500 mL), treated with saturated aqueous sodium bicarbonate (250 mL), stirred and filtered off. The filtered solid was washed with water and dried to afford the title compound (14.5 g, 27.8 mmol, 88% yield). LCMS (Method A): Rt 0.92 minutes; m/z 522,524 (MH+).

### Intermediate 89

### 6-[(6-bromo-1,3-benzothiazol-2-yl)amino]-2-[(trans-4-hydroxycyclohexyl)amino]-4-pyrimidinecarbaldehyde

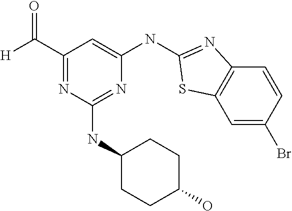

A solution of trans-4-({4-[bis(ethyloxy)methyl]-6-[(6-bromo-1,3-benzothiazol-2-yl)amino]-2-pyrimidinyl}amino)cyclohexanol (15 g, 28.7 mmol) in tetrahydrofuran (200 mL) was treated with aqueous hydrochloric acid (5M, 200 mL, 1 mol) and the mixture was heated at reflux for 6 hours. The mixture was evaporated to dryness and the residue partitioned between dichloromethane (+5% methanol) (200 mL) and saturated aqueous sodium bicarbonate (150 mL). The organic phase was dried over magnesium sulfate, filtered and evaporated to dryness to afford the title compound (7.7 g, 17.2 mmol, 60% yield). LCMS (Method A): Rt 0.72 minutes; m/z 448,450 (MH+).

### Intermediate 90

### N-{6-[bis(ethyloxy)methyl]-2-[(phenylmethyl)thio]-4-pyrimidinyl}[1,3]thiazolo[5,4-b]pyridin-2-amine

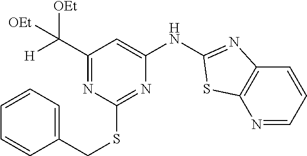

In an atmosphere of nitrogen, an ice-cooled solution of a mixture of 6-[bis(ethyloxy)methyl]-2-[(phenylmethyl)thio]-4-pyrimidinamine (15 g, 47.0 mmol) and 2-bromo[1,3]thiazolo[5,4-b]pyridine (10.60 g, 49.3 mmol) in dry N,N-dimethylformamide (200 mL) was treated portionwise over 5 minutes with sodium hydride (60% w/w in oil) (3.76 g, 94 mmol) and the mixture was stirred with cooling for 3 hours and at ambient temperature for a further 2 hours. The mixture was treated cautiously with water (250 mL) and the precipitated material was filtered off. The filtered solid was washed with water and dried to afford the title compound (19.6 g, 43.2 mmol, 92% yield). LCMS (Method A): Rt 1.32 minutes; m/z 454 (MH+).

### Intermediate 91

### N-{6-[bis(ethyloxy)methyl]-2-[(phenylmethyl)sulfonyl]-4-pyrimidinyl}[1,3]thiazolo[5,4-b]pyridin-2-amine

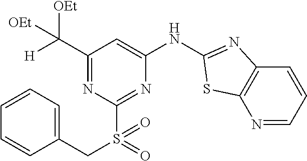

A solution of N-{6-[bis(ethyloxy)methyl]-2-[(phenyl methyl)thio]-4-pyrimidinyl}[1,3]thiazolo[5,4-b]pyridin-2-amine (18 g, 39.7 mmol) in N,N-dimethylformamide (200 mL) was treated with Oxone® (62.5 g, 102 mmol) and the mixture was stirred at ambient temperature overnight. The mixture was added to water (300 mL) and extracted with chloroform (2×250 mL). The combined organics were evaporated to dryness to afford the title compound (18.8 g, 33.4 mmol, 99% yield). LCMS (Method A): Rt 1.07 minutes; m/z 486 (MH+).

### Intermediate 92

### trans-4-{[4-[bis(ethyloxy)methyl]-6-([1,3]thiazolo[5,4-b]pyridin-2-ylamino)-2-pyrimidinyl]amino}cyclohexanol

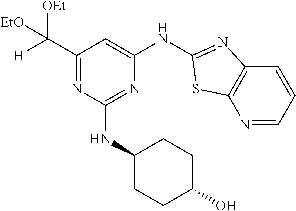

A mixture of N-{6-[bis(ethyloxy)methyl]-2-[(phenylmethyl)sulfonyl]-4-pyrimidinyl}[1,3]thiazolo[5,4-b]pyridin-2-amine (18.4 g, 37.9 mmol) and trans-4-aminocyclohexanol (13.09 g, 114 mmol) in 1,4-dioxane (150 mL) was heated at 100° for 8 hours. The cooled mixture was treated with water (300 mL) and stirred for 30 minutes. The mixture was filtered and the filtered solid was washed with water and thoroughly dried to afford the title compound (14.3 g, 32.2 mmol, 85% yield). LCMS (Method A): Rt 0.73 minutes; m/z 445 (MH+)

### Intermediate 93

### 2-[(trans-4-hydroxycyclohexyl)amino]-6-([1,3]thiazolo[5,4-b]pyridin-2-ylamino)-4-pyrimidinecarbaldehyde

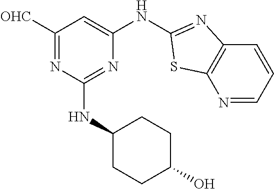

A solution of trans-4-{[4-[bis(ethyloxy)methyl]-6-([1,3]thiazolo[5,4-b]pyridin-2-ylamino)-2-pyrimidinyl]amino}cyclohexanol (14 g, 31.5 mmol) in tetrahydrofuran (150 mL) was treated with aqueous hydrochloric acid (5M) (150 mL, 750 mmol) and the mixture was heated at reflux for 6 hours. The mixture was evaporated to dryness and the residue was partitioned between ethyl acetate (200 mL) and saturated aqueous sodium bicarbonate (150 mL). The organic phase was dried over magnesium sulfate, filtered and evaporated to dryness to afford the title compound as the hydrate (9.88 g, 26.7 mmol, 85% yield). LCMS (Method A): Rt 0.51 minutes; m/z 389 (MH+).

### Intermediate 94

### 2-[(phenylmethyl)thio]-6-([1,3]thiazolo[5,4-b]pyridin-2-ylamino)-4-pyrimidinecarbaldehyde

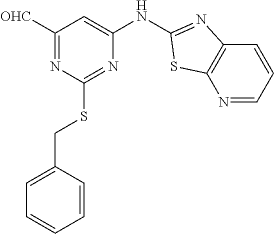

A solution of N-{6-[bis(ethyloxy)methyl]-2-[(phenyl methyl)thio]-4-pyrimidinyl}[1,3]thiazolo[5,4-b]pyridin-2-amine (4.3 g, 9.48 mmol) in tetrahydrofuran (50 mL) was treated with aqueous hydrochloric acid (5M) (50 mL, 250 mmol) and the mixture was heated at reflux for 6 hours. The mixture was evaporated to dryness and the residue partitioned between dichloromethane (+5% methanol) (200 mL) and saturated aqueous sodium bicarbonate (150 mL). The organic phase was dried over magnesium sulfate, filtered and evaporated to afford the title compound (2.6 g, 6.85 mmol, 72.3% yield). LCMS (Method A): Rt 1.17 minutes; m/z 380 (MH+) & Rt 0.93 minutes; m/z 398 (hydrate, MH+).

### Intermediate 95

### N-[2-[(phenylmethyl)thio]-6-(1-piperidinylmethyl)-4-pyrimidinyl][1,3]thiazolo[5,4-b]pyridin-2-amine

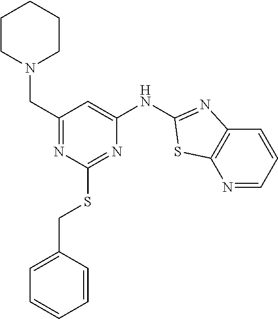

A mixture of 2-[(phenylmethyl)thio]-6-([1,3]thiazolo[5,4-b]pyridin-2-ylamino)-4-pyrimidinecarbaldehyde (2 g, 5.27 mmol), piperidine (0.57 mL, 5.80 mmol) and sodium triacetoxyborohydride (2.57 g, 12.12 mmol) in dichloromethane (25 mL) was stirred at ambient temperature for 1 hour. The reaction mixture was then stirred with saturated aqueous sodium bicarbonate (25 mL) for 20 minutes. The organic layer was then evaporated to dryness to afford the title compound (2.1 g, 4.68 mmol, 89% yield). LCMS (Method A): Rt 0.85 minutes; m/z 449 (MH+)

### Intermediates 96 and 97

### N-[2-[(phenylmethyl)sulfonyl]-6-(1-piperidinyl methyl)-4-pyrimidinyl][1,3]thiazolo[5,4-b]pyridin-2-amine and N-[2-[(phenylmethyl)sulfinyl]-6-(1-piperidinylmethyl)-4-pyrimidinyl][1,3]thiazolo[5,4-b]pyridin-2-amine

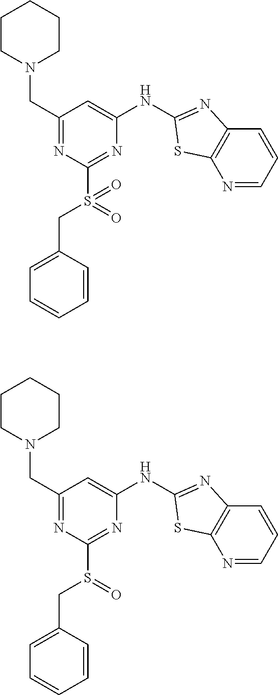

A solution of N-[2-[(phenylmethyl)thio]-6-(1-piperidinylmethyl)-4-pyrimidinyl][1,3]thiazolo[5,4-b]pyridin-2-amine (2.1 g, 4.68 mmol) in N,N-dimethylformamide was treated with Oxone® (7.19 g, 11.70 mmol). Once all the starting material had been consumed (by LCMS analysis) the reaction was treated with sodium bicarbonate and left to stir for 20 minutes. This was then added to dichloromethane (300 mL). The organic phase was collected, dried over magnesium sulfate and evaporated to dryness. The product was then purified via chromatography on silica using a gradient elution from 0 to 30% methanol in dichloromethane). Fractions containing the sulfoxide and/or the sulfone were pooled together and evaporated to afford a mixture of the title compounds (955 mg. approximately 42% yield). LCMS (Method A): Rt 0.69 minutes; m/z 465 (sulfoxide, MH+) & Rt 0.73 minutes; m/z 481 (sulfone, MH+).

### Intermediate 98

### 2,4-difluoro-6-methylpyrimidine

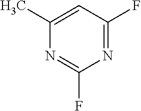

A mixture of anhydrous potassium fluoride (50 g, 861 mmol), 2,4-dichloro-6-methylpyrimidine (25 g, 153 mmol) and cis-dicyclohexano-18-crown-6 (0.9 g, 2.416 mmol) was treated with tetraglyme (60 mL) and the mixture was heated under nitrogen at 150° C. for 16 hours. The product was then distilled directly from the reaction mixture to afford 2,4-difluoro-6-methylpyrimidine (8 g, 61.5 mmol, 40.1% yield) as a colourless oil. Bp 50-60° C. at 12 mbar. LCMS (Method A): Rt 0.62 minutes; m/z 131 (MH+)

### Intermediate 99

### 5-chloro-N-(2-fluoro-6-methyl-4-pyrimidinyl)[1,3]thiazolo[5,4-b]pyridin-2-amine

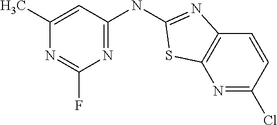

Under an atmosphere of nitrogen, a solution of 2,4-difluoro-6-methylpyrimidine (4.5 g, 34.6 mmol) in dry N,N-dimethylformamide (50 mL) was treated with 5-chloro[1,3]thiazolo[5,4-b]pyridin-2-amine (6.42 g, 34.6 mmol) and the mixture was cooled with an ice/water bath. Sodium hydride (60% in oil) (2.77 g, 69.2 mmol) was added portion wise and the mixture was stirred whilst being allowed to slowly warm to ambient temperature overnight. The mixture was treated cautiously with aqueous ammonium chloride solution (5%, 20 mL) and water (100 mL). The mixture was then extracted with chloroform (2×150 mL) and the combined organic fraction was evaporated to dryness. The product was purified by flash chromatography on silica using a gradient elution from 0% to 100% ethyl acetate in cyclohexane to afford the title compound (4.2 g, 14.20 mmol, 41.1% yield). LCMS (Method A): Rt 1.02 minutes; m/z 296 (MH+).

### Intermediate 100

### 6-[(methyloxy)methyl]-2-(methylthio)-4-pyrimidinyl trifluoromethanesulfonate

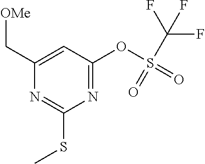

Under an atmosphere of nitrogen an ice-cooled mixture of 6-[(methyloxy)methyl]-2-(methylthio)-4-pyrimidinol (16.23 g, 87 mmol) and N,N-diisopropylethylamine (45.7 mL, 261 mmol) in dichloromethane (200 mL) was stirred and treated dropwise with trifluoromethanesulfonic anhydride (21.7 mL, 131 mmol). The cooling was removed and the reaction mixture was stirred for 1 hour at ambient temperature. Water (200 mL) was added and the organic layer was collected, dried over magnesium sulfate, filtered and evaporated to dryness. The residue was taken up in diethyl ether and then filtered. The filtrate was evaporated to dryness and the product was purified by silica chromatography using a gradient elution from 0-50% ethyl acetate in cyclohexane to afford the title compound (26.1 g, 82 mmol, 94% yield). LCMS (Method A): Rt 1.22 minutes; m/z 319 (MH+).

### Intermediate 101

### 6-[(methyloxy)methyl]-2-(methylthio)-4-pyrimidinamine

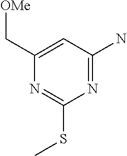

To a solution of 6-[(methyloxy)methyl]-2-(methylthio)-4-pyrimidinyl trifluoromethanesulfonate (26.05 g, 82 mmol) in acetonitrile (200 mL) were added N,N-diisopropylethylamine (20.3 mL, 123 mmol) and (1,1,3,3-tetramethylbutyl)amine (20.5 mL, 123 mmol). The reaction mixture was then stirred and heated at 100° C. for 7 hours. The mixture was then evaporated to dryness, taken up in ethyl acetate (100 mL) and washed with a saturated sodium bicarbonate solution (3×100 mL). The organic phase was dried over magnesium sulfate and evaporated to dryness. Trifluoroacetic acid (200 mL, 2596 mmol) was added and the mixture was stirred at ambient temperature under an atmosphere of nitrogen for 2 hours and then heated at 50° C. for 3 hours. The mixture was evaporated to dryness. The residue was taken up in dichloromethane and washed with saturated aqueous sodium bicarbonate. The organic layer was collected, dried over magnesium sulfate, filtered and evaporated to dryness. The residue was dissolved in methanol, added to a 70 g aminopropyl ion-exchange column and eluted with methanol. The product-containing fractions were combined and evaporated to dryness to afford the title compound (14 g, 76 mmol, 92% yield). LCMS (Method A): Rt 0.36 minutes; m/z 186 (MH+).

### Intermediate 102

### 6-bromo-N-[6-[(methyloxy)methyl]-2-(methylthio)-4-pyrimidinyl]-1,3-benzothiazol-2-amine

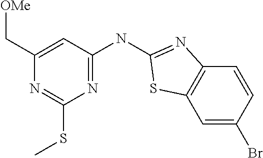

Under an atmosphere of nitrogen, an ice-cooled, stirred solution of 6-[(methyloxy)methyl]-2-(methylthio)-4-pyrimidinamine (14 g, 76 mmol) in N,N-dimethylformamide (500 mL) was treated with sodium hydride (60% in mineral oil) (6.05 g, 151 mmol) portionwise. The reaction mixture was stirred with cooling for 10 minutes and then treated with a solution of 2-chloro-6-bromobenzothiazole (18.78 g, 76 mmol) in DMF (200 mL). The reaction mixture was allowed to warm to ambient temperature and was stirred overnight. The reaction mixture was then treated with saturated aqueous ammonium chloride (200 mL). Water (300 mL) was added and the precipitated product was filtered off and dried to afford the title compound (27.6 g, 69.5 mmol, 92% yield). LCMS (Method A): Rt 1.27 minutes; m/z 397,399 (MH+).

### Intermediate 103

### 6-bromo-N-[6-[(methyloxy)methyl]-2-(methylsulfonyl)-4-pyrimidinyl]-1,3-benzothiazol-2-amine

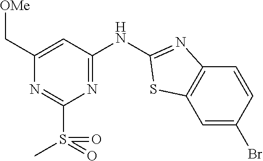

Under an atmosphere of nitrogen, a stirred suspension of 6-bromo-N-[6-[(methyloxy)methyl]-2-(methylthio)-4-pyrimidinyl]-1,3-benzothiazol-2-amine (27.6 g, 69.5 mmol) in N,N-dimethylformamide (1 L) was treated with Oxone® (128.2 g, 208 mmol) portionwise. The mixture was stirred at room temperature for 1.5 hours. The reaction was then treated with water (1 L) resulting in a solid precipitate which was filtered off, washed with water and thoroughly dried to afford the title compound (22.02 g, 51.3 mmol, 73.8% yield). LCMS (Method A): Rt 0.89 minutes; m/z 429,431 (MH+).

### Intermediate 104

### {6-[(6-bromo-1,3-benzothiazol-2-yl)amino]-2-[(trans-4-hydroxycyclohexyl)amino]-4-pyrimidinyl}methyl methanesulfonate

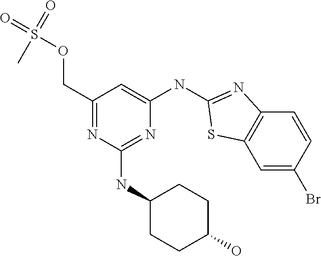

To an ice-cooled, stirred solution of trans-4-{[4-[(6-bromo-1,3-benzothiazol-2-yl)amino]-6-(hydroxymethyl)-2-pyrimidinyl]amino}cyclohexanol (5 g, 11.10 mmol) and N-ethyldiisopropylamine (3.9 mL, 22.2 mmol) in tetrahydrofuran (200 mL) was added methanesulfonyl chloride (2.16 mL, 27.8 mmol) dropwise. The reaction mixture was allowed to warm to ambient temperature and stirred for 1 hour. Saturated aqueous sodium bicarbonate (100 mL) was added followed by water (200 mL) and ethyl acetate (200 mL). The organic phase was collected, dried over magnesium sulfate, filtered and evaporated to dryness to afford the crude (61% by LCMS) title compound (4.5 g, 8.52 mmol, 77% yield) which was used subsequently without further purification. LCMS (Method A): Rt 0.91 minutes; m/z 528,530 (MH+).

### Example 1

### trans-4-{[4-[(6-methyl-1,3-benzothiazol-2-yl)amino]-6-(phenylmethyl)-2-pyrimidinyl]amino}cyclohexanol

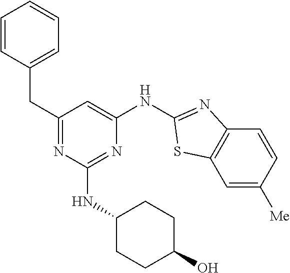

A mixture of N-[2-fluoro-6-(phenylmethyl)-4-pyrimidinyl]-6-methyl-1,3-benzothiazol-2-amine (200 mg, 0.57 mmol), trans-4-aminocyclohexanol (131 mg, 1.14 mmol) and N-ethyldiisopropylamine (0.299 mL, 1.71 mmol) in isopropanol (7 mL) was sealed and heated in a Biotage “Initiator” microwave at 160° C. for 15 minutes. After cooling, the resulting precipitate was filtered off, washed with acetonitrile (14 mL) and dried to afford the title compound (170 mg, 0.382 mmol, 66.8% yield) as a white solid. LCMS (Method A): Rt 1.13 minutes; m/z 447 (MH+).

The compounds shown in the table were prepared in an analogous manner to that for trans-4-{[4-[(6-methyl-1,3-benzothiazol-2-yl)amino]-6-(phenylmethyl)-2-pyrimidinyl]amino}cyclohexanol by reacting the appropriately-substituted 2-fluoropyrimidine with the appropriate amine:

| | | | Analytical | Purification |
|---|---|---|---|---|
| Example | Structure | Name | Data | Method |
| 2 | 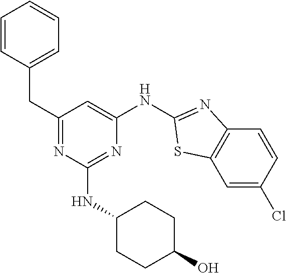 | trans-4-{[4-[(6- chloro-1,3- benzothiazol-2- yl)amino]-6- (phenylmethyl)-2- pyrimidinyl]amino} cyclohexanol | LCMS (Method A): Rt 0.91 minutes; m/z 466 (MH⁺) | Precipitated product filtered and washed with methanol |
| 3 | 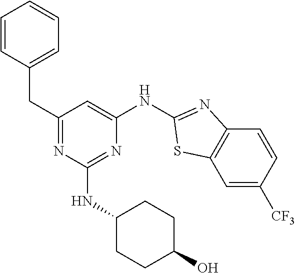 | trans-4-[(4- (phenylmethyl)-6- {[6- (trifluoromethyl)- 1,3-benzothiazol- 2-yl]amino}-2- pyrimidinyl)amino] cyclohexanol | LCMS (Method A): Rt 0.95 minutes; m/z 500 (MH⁺) | MDAP, ammonium bicarbonate modifier |
| 4 | 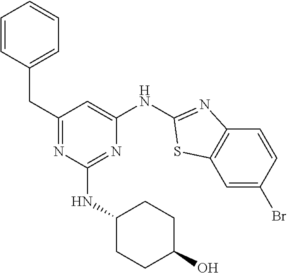 | trans-4-{[4-[(6- bromo-1,3- benzothiazol-2- yl)amino]-6- (phenylmethyl)-2- pyrimidinyl]amino} cyclohexanol | LCMS (Method B): Rt 3.03 minutes; m/z 510, 512 (MH⁺) | MDAP, ammonium bicarbonate modifier |
| 5 | 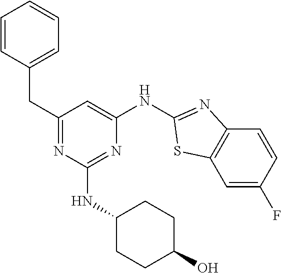 | trans-4-{[4-[(6- fluoro-1,3- benzothiazol-2- yl)amino]-6- (phenylmethyl)-2- pyrimidinyl]amino} cyclohexanol | LCMS (Method B): Rt 2.82 minutes; m/z 450 (MH⁺) | MDAP, ammonium bicarbonate modifier |
| 6 | 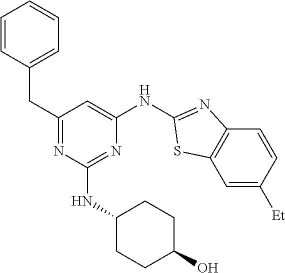 | trans-4-{[4-[(6- ethyl-1,3- benzothiazol-2- yl)amino]-6- (phenylmethyl)-2- pyrimidinyl]amino} cyclohexanol | LCMS (Method A): Rt 0.92 minutes; m/z 460 (MH⁺) | MDAP, ammonium bicarbonate modifier |
| 7 | 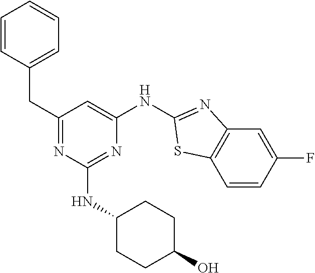 | trans-4-{[4-[(5- fluoro-1,3- benzothiazol-2- yl)amino]-6- (phenylmethyl)-2- pyrimidinyl]amino} cyclohexanol | LCMS (Method B): Rt 2.84 minutes; m/z 450 (MH⁺) | MDAP, ammonium bicarbonate modifier |
| 8 | 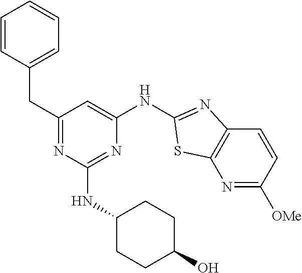 | trans-4-{[4-{[5- (methyloxy)[1,3] thiazolo[5,4- b]pyridin-2- yl]amino}-6- (phenylmethyl)-2- pynmidinyl]amino} cyclohexanol | LCMS (Method B): Rt 2.66 minutes; m/z 463 (MH⁺) | MDAP, ammonium bicarbonate modifier |
| 9 | 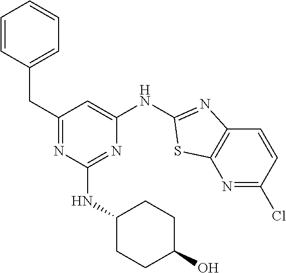 | trans-4-{[4-[(5- chloro[1,3]thiazolo [5,4-b]pyridin-2- yl)amino]-6- (phenylmethyl)-2- pyrimidinyl]amino} cyclohexanol | LCMS (Method A): Rt 0.79 minutes; m/z 467 (MH⁺) | Precipitated product filtered and washed with isopropanol |
| 10 | 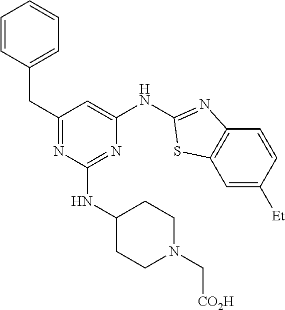 | (4-{[4-[(6-ethyl- 1,3-benzothiazol- 2-yl)amino]-6- (phenylmethyl)-2- pyrimidinyl] amino}-1- piperidinyl)acetic acid | LCMS (Method A): Rt 0.72 minutes; m/z 503 (MH⁺) | MDAP, formic acid modifier |
| 11 | 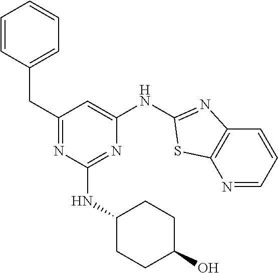 | trans-4-{[4- (phenylmethyl)-6- ([1,3]thiazolo[5,4- b]pyridin-2- ylamino)-2- pyrimidinyl]amino} cyclohexanol | LCMS (Method A): Rt 0.71 minutes; m/z 433 (MH⁺) | MDAP, TFA modifier |
| 12 | 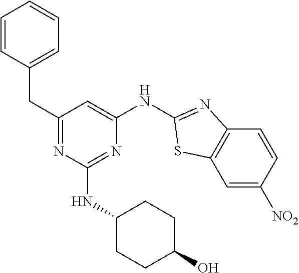 | trans-4-{[4-[(6- nitro-1,3- benzothiazol-2- yl)amino]-6- (phenylmethyl)-2- pyrimidinyl]amino} cyclohexanol | LCMS (Method A): Rt 0.86 minutes; m/z 477 (MH⁺) | Precipitated product filtered and washed with acetonitrile |
| 13 | 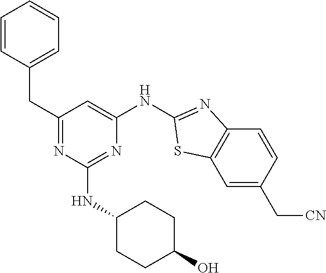 | (2-{[2-[(trans-4- hydroxy- cyclohexyl) amino]-6- (phenylmethyl)-4- pyrimidinyl] amino}1,3- benzothiazol- 6-yl)acetonitrile | LCMS (Method A): Rt 0.73 minutes; m/z 471 (MH⁺) | MDAP, ammonium bicarbonate modifier |
| 14 | 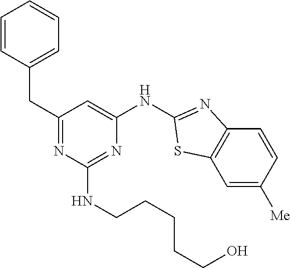 | 5-{[4-[(6-methyl- 1,3-benzothiazol- 2-yl)amino]-6- (phenylmethyl)-2- pyrimidinyl] amino}-1-pentanol | LCMS (Method A): Rt 0.84 minutes; m/z 434 (MH+) | MDAP, TFA modifier |
| 15 | 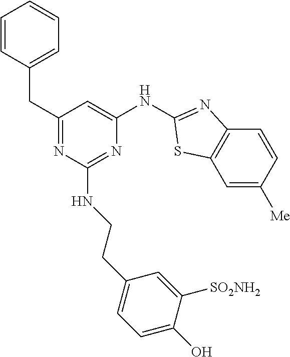 | 2-hydroxy-5-(2- {[4-[(6-methyl-1,3- benzothiazol-2- yl)amino]-6- (phenylmethyl)-2- pyrimidinyl]amino} ethyl)benzene- sulfonamide | LCMS (Method A): Rt 0.83 minutes; m/z 547 (MH+) | MDAP, TFA modifier |
| 16 | 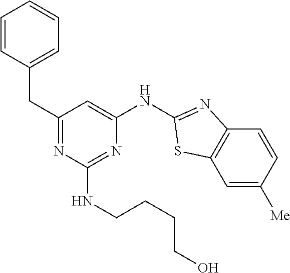 | 4-{[4-[(6-methyl- 1,3-benzothiazol- 2-yl)amino]-6- (phenylmethyl)-2- pyrimidinyl] amino}-1-butanol | LCMS (Method A): Rt 0.81 minutes; m/z 420 (MH+) | MDAP, TFA modifier |
| 17 | 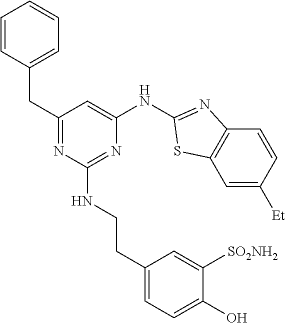 | 5-(2-{[4-[(6-ethyl- 1,3-benzothiazol- 2-yl)amino]-6- (phenylmethyl)-2- pyrimidinyl]amino} ethyl)-2- hydroxybenzene- sulfonamide | LCMS (Method A): Rt 0.88 minutes; m/z 561 (MH+) | MDAP, TFA modifier |
| 18 | 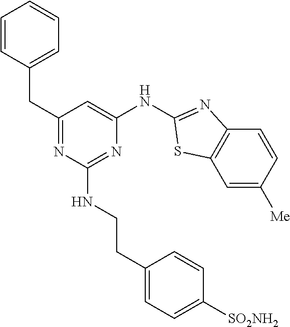 | 4-(2-{[4-[(6- methyl-1,3- benzothiazol-2- yl)amino]-6- (phenylmethyl)-2- pyrimidinyl]amino} ethyl)benzene- sulfonamide | LCMS (Method A): Rt 0.86 minutes; m/z 531 (MH+) | MDAP, TFA modifier |
| 19 | 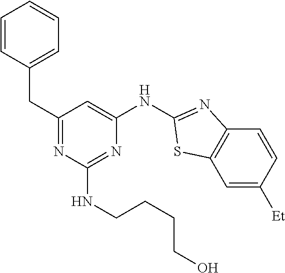 | 4-{[4-[(6-ethyl-1,3- benzothiazol-2- yl)amino]-6- (phenylmethyl)-2- pyrimidinyl] amino}-1-butanol | LCMS (Method A): Rt 0.87 minutes; m/z 434 (MH+) | MDAP, TFA modifier |
| 20 | 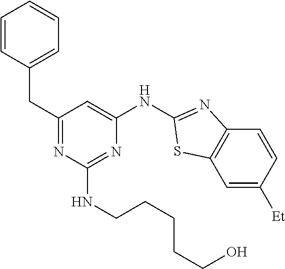 | 5-{[4-[(6-ethyl-1,3- benzothiazol-2- yl)amino]-6- (phenylmethyl)-2- pyrimidinyl] amino}-1-pentanol | LCMS (Method A): Rt 0.89 minutes; m/z 448 (MH+) | MDAP, TFA modifier |
| 21 | 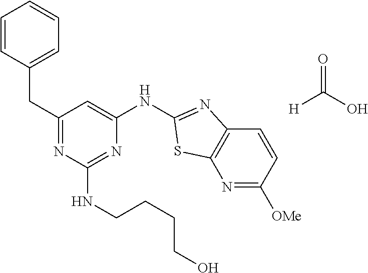 | 4-{[4-{[5- (methyloxy)[1,3] thiazolo[5,4- b]pyridin-2- yl]amino}-6- (phenylmethyl)-2- pyrimidinyl] amino}-1-butanol formate | LCMS (Method A): Rt 0.76 minutes; m/z 437 (MH+) | MDAP, formic acid modifier |
| 22 | 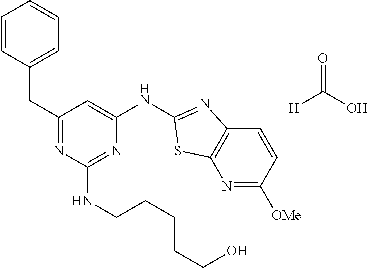 | 5-{[4-{[5- (methyloxy)[1,3] thiazolo[5,4- b]pyridin-2- yl]amino}-6- (phenylmethyl)-2- pyrimidinyl] amino}-1-pentanol formate | LCMS (Method A): Rt 0.78 minutes; m/z 451 (MH+) | MDAP, formic acid modifier |
| 23 | 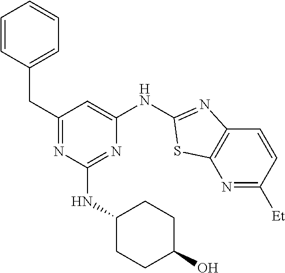 | trans-4-{[4-[(5- ethyl[1,3]thiazolo [5,4-b]pyridin-2- yl)amino]-6- (phenylmethyl)-2- pyrimidinyl]amino} cyclohexanol | LCMS (Method A): Rt 0.77 minutes; m/z 461 (MH+) | MDAP, formic acid modifier |
| 24 | 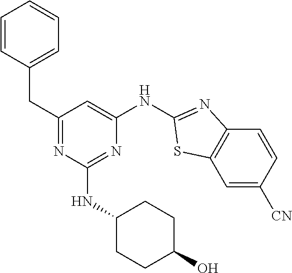 | 2-{[2-[(trans-4- hydroxy- cyclohexyl)amino]- 6-(phenylmethyl)- 4-pyrimidinyl] amino}-1,3- benzothiazole-6- carbonitrile | LCMS (Method A): Rt 0.81 minutes; m/z 457 (MH+) | MDAP, ammonium bicarbonate modifier |
| 25 | 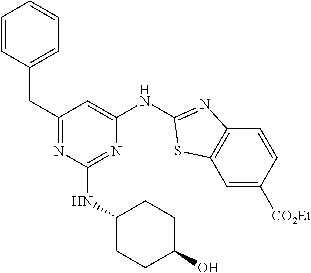 | ethyl 2-{[2-[(trans- 4-hydroxy- cyclohexyl) amino]-6- (phenylmethyl)-4- pyrimidinyl] amino}-1,3- benzothiazole-6- carboxylate | LCMS (Method A): Rt 0.84 minutes; m/z 504 (MH+) | Precipitated product filtered and washed with acetonitrile |
| 26 | 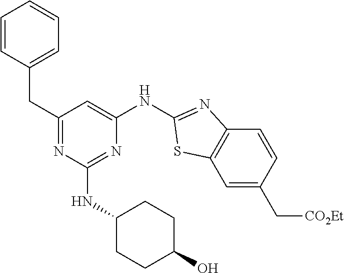 | ethyl (2-{[2- [(trans-4- hydroxy- cyclohexyl)amino]- 6-(phenylmethyl)- 4-pyrimidinyl] amino}-1,3- benzothiazol- 6-yl)acetate | LCMS (Method A): Rt 1.12 minutes; m/z 518 (MH+) | MDAP, ammonium bicarbonate modifier |
| 27 | 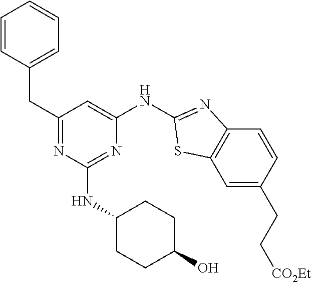 | ethyl 3-(2-{[2- [(trans-4-hydroxy- cyclohexyl) amino]-6- (phenylmethyl)-4- pyrimidinyl] amino}-1,3- benzothiazol- 6-yl)propanoate | LCMS (Method A): Rt 0.82 minutes; m/z 532 (MH+) | Precipitated product filtered and washed with acetonitrile |
| 28 | 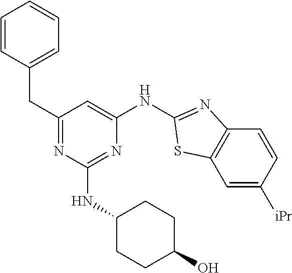 | trans-4-{[4-{[6-(1- methylethyl)-1,3- benzothiazol-2- yl]amino}-6- (phenylmethyl)-2- pyrimidinyl]amino} cyclohexanol | LCMS (Method A): Rt 0.90 minutes; m/z 474 (MH+) | MDAP, ammonium bicarbonate modifier |
| 29 | 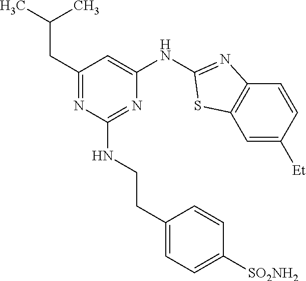 | 4-(2-{[4-[(6-ethyl- 1,3-benzothiazol- 2-yl)amino]-6-(2- methylpropyl)-2- pyrimidinyl]amino} ethyl)benzene- sulfonamide | LCMS (Method A): Rt 0.86 minutes; m/z 511 (MH+) | MDAP, TFA modifier |
| 30 | 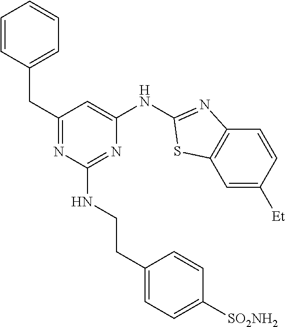 | 4-(2-{[4-[(6-ethyl- 1,3-benzothiazol- 2-yl)amino]-6- (phenylmethyl)-2- pyrimidinyl]amino} ethyl)benzene- sulfonamide | LCMS (Method A): Rt 0.91 minutes; m/z 545 (MH+) | MDAP, TFA modifier |
| 31 | 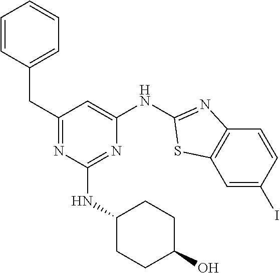 | trans-4-{[4-[(6- iodo-1,3- benzothiazol-2- yl)amino]-6- (phenylmethyl)-2- pyrimidinyl]amino} cyclohexanol | LCMS (Method A): Rt 0.88 minutes; m/z 558 (MH+) | MDAP, ammonium bicarbonate modifier |
| 32 | 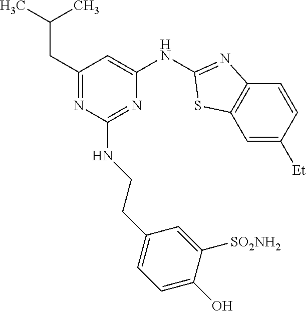 | 5-(2-{[4-[(6-ethyl- 1,3-benzothiazol- 2-yl)amino]-6-(2- methylpropyl)-2- pyrimidinyl]amino} ethyl)-2- hydroxybenzene- sulfonamide | LCMS (Method A): Rt 0.84 minutes; m/z 527 (MH+) | MDAP, TFA modifier |
| 33 | 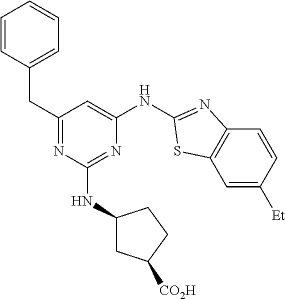 | (1R,3S)-3-{[4-[(6- ethyl-1,3- benzothiazol-2- yl)amino]-6- (phenylmethyl)-2- pyrimidinyl]amino} cyclopentane- carboxylic acid | LCMS (Method A): Rt 0.93 minutes; m/z 474 (MH+) | MDAP, formic acid modifier |
| 34 | 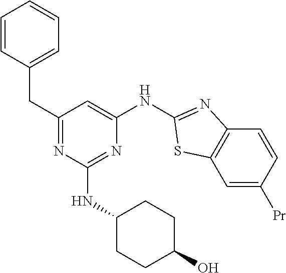 | trans-4-({4- (phenylmethyl)-6- [(6-propyl-1,3- benzothiazol-2- yl)amino]-2- pyrimidinyl}amino) cyclohexanol | LCMS (Method B): Rt 2.25 minutes; m/z 474 (MH+) | MDAP, ammonium bicarbonate modifier |
| 35 | 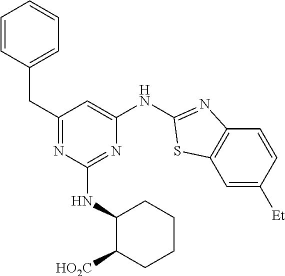 | (1R,2S)-2-{[4-[(6- ethyl-1,3- benzothiazol-2- yl)amino]-6- (phenylmethyl)-2- pyrimidinyl]amino} cyclohexane- carboxylic acid | LCMS (Method A): Rt 1.00 minutes; m/z 488 (MH+) | MDAP, formic acid modifier |
| 36 | 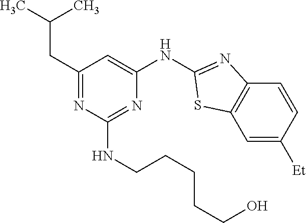 | 5-{[4-[(6-ethyl-1,3- benzothiazol-2- yl)amino]-6-(2- methylpropyl)-2- pyrimidinyl] amino}-1-pentanol | LCMS (Method A): Rt 0.86 minutes; m/z 414 (MH+) | MDAP, TFA modifier |
| 37 | 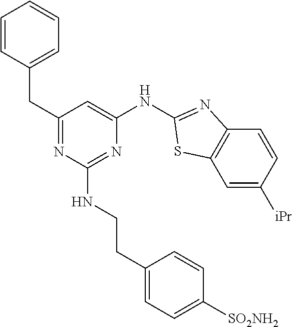 | 4-(2-{[4-{[6-(1- methylethyl)-1,3- benzothiazol-2- yl]amino}-6- (phenylmethyl)-2- pyrimidinyl]amino} ethyl)benzene- sulfonamide | LCMS (Method A): Rt 0.98 minutes; m/z 559 (MH+) | Precipitated product filtered and washed with acetonitrile |
| 38 | 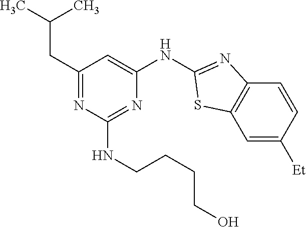 | 4-{[4-[(6-ethyl-1,3- benzothiazol-2- yl)amino]-6-(2- methylpropyl)-2- pyrimidinyl] amino}-1-butanol | LCMS (Method A): Rt 0.83 minutes; m/z 400 (MH+) | MDAP, TFA modifier |
| 39 | 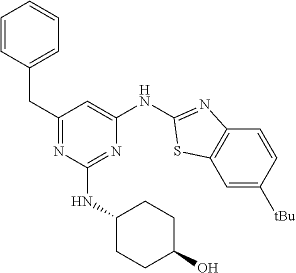 | trans-4-{[4-{[6- (1,1- dimethylethyl)- 1,3-benzothiazol- 2-yl]amino}-6- (phenylmethyl)-2- pyrimidinyl]amino} cyclohexanol | LCMS (Method A): Rt 0.98 minutes; m/z 488 (MH+) | MDAP, ammonium bicarbonate modifier |
| 40 | 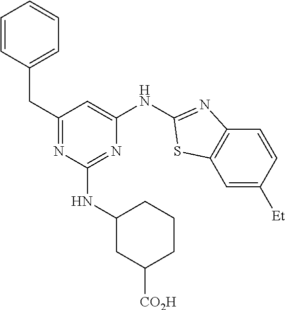 | 3-{[4-[(6-ethyl-1,3- benzothiazol-2- yl)amino]-6- (phenylmethyl)-2- pyrimidinyl]amino} cyclohexane- carboxylic acid | LCMS (Method A): Rt 0.93 minutes; m/z 488 (MH+) | MDAP, TFA modifier |
| 41 | 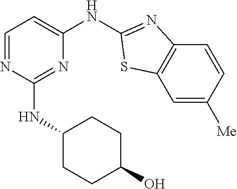 | trans-4-({4-[(6- methyl-1,3- benzothiazol-2- yl)amino]-2- pyrimidinyl}amino) cyclohexanol | LCMS (Method A): Rt 0.66 minutes; m/z 356 (MH+) | MDAP, ammonium bicarbonate modifier |
| 42 | 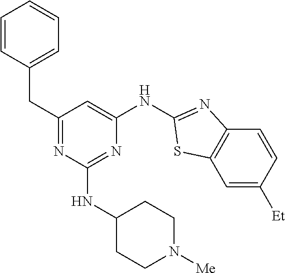 | N⁴-6-ethyl-1,3- benzothiazol-2- yl)-N²-(1-methyl- 4-piperidinyl)-6- (phenylmethyl)- 2,4- pyrimidinediamine | LCMS (Method A): Rt 0.68 minutes; m/z 459 (MH+) | MDAP, ammonium bicarbonate modifier |
| 43 | 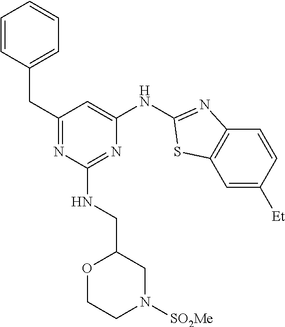 | N⁴-(6-ethyl-1,3- benzothiazol-2- yl)-N²-{[4- (methylsulfonyl)- 2-morpholinyl] methyl}-6- (phenylmethyl)- 2,4- pyrimidinediamine | LCMS (Method A): Rt 0.91 minutes; m/z 539 (MH+) | MDAP, ammonium bicarbonate modifier |
| 44 | 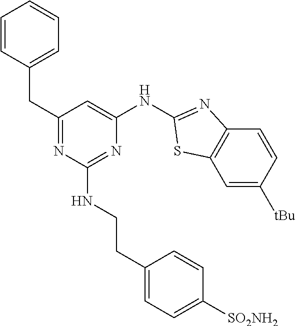 | 4-(2-{[4-{[6-(1,1- dimethylethyl)- 1,3-benzothiazol- 2-yl]amino}-6- (phenylmethyl)-2- pyrimidinyl]amino} ethyl)benzene- sulfonamide | LCMS (Method A): Rt 1.17 minutes; m/z 573 (MH+) | Precipitated product filtered and washed with acetonitrile |
| 45 | 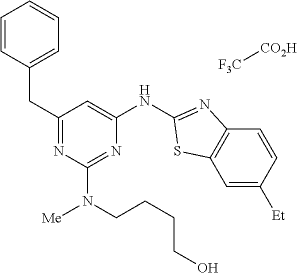 | 4-[[4-[(6-ethyl-1,3- benzothiazol-2- yl)amino]-6- (phenylmethyl)-2- pyrimidinyl] (methyl)amino]-1- butanol trifluoroacetate | LCMS (Method A): Rt 0.98 minutes; m/z 448 (MH+) | MDAP, formic acid modifier |
| 46 | 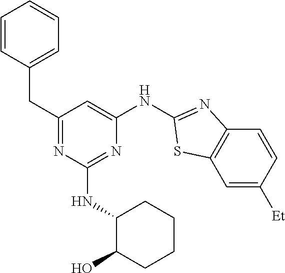 | (1R,2R)-2-{[4-[(6- ethyl-1,3- benzothiazol-2- yl)amino]-6- (phenylmethyl)-2- pyrimidinyl]amino} cyclohexanol | LCMS (Method A): Rt 0.97 minutes; m/z 460 (MH+) | MDAP, formic acid modifier |
| 47 | 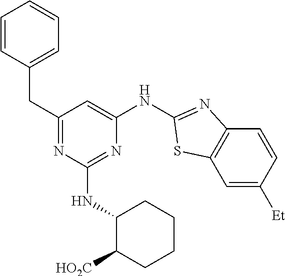 | (1R,2R)-2-{[4-[(6- ethyl-1,3- benzothiazol-2- yl)amino]-6- (phenylmethyl)-2- pyrimidinyl]amino} cyclohexane- carboxylic acid | LCMS (Method A): Rt 0.98 minutes; m/z 488 (MH+) | MDAP, formic acid modifier |
| 48 | 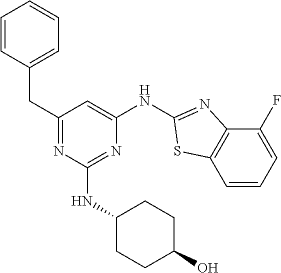 | trans-4-{[4-[(4- fluoro-1,3- benzothiazol-2- yl)amino]-6- (phenylmethyl)-2- pyrimidinyl]amino} cyclohexanol | LCMS (Method A): Rt 0.78 minutes; m/z 450 (MH+) | MDAP, ammonium bicarbonate modifier |
| 49 | 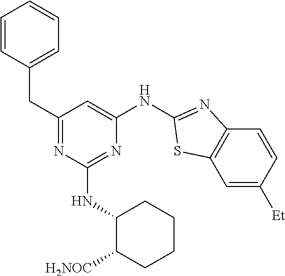 | (1S,2R)-2-{[4-[(6- ethyl-1,3- benzothiazol-2- yl)amino]-6- (phenylmethyl)-2- pyrimidinyl]amino} cyclohexane- carboxamide | LCMS (Method A): Rt 0.94 minutes; m/z 487 (MH+) | MDAP, formic acid modifier |
| 50 | 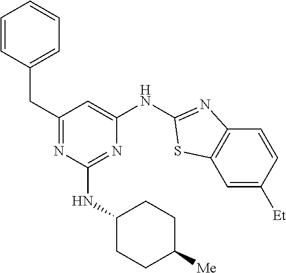 | N⁴-(6-ethyl-1,3- benzothiazol-2- yl)-N²-(trans-4- methylcyclohexyl)- 6-(phenylmethyl)- 2,4- pyrimidinediamine | LCMS (Method A): Rt 0.77 minutes; m/z 458 (MH+) | MDAP, ammonium bicarbonate modifier |
| 51 | 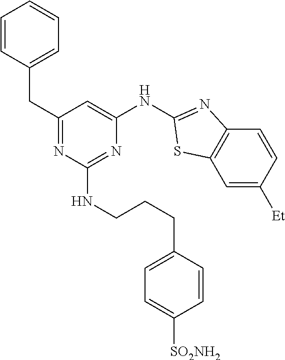 | 4-(3-{[4-[(6-ethyl- 1,3-benzothiazol- 2-yl)amino]-6- (phenylmethyl)-2- pyrimidinyl]amino} propyl)benzene- sulfonamide | LCMS (Method A): Rt 0.92 minutes; m/z 559 (MH+) | MDAP, TFA modifier |
| 52 | 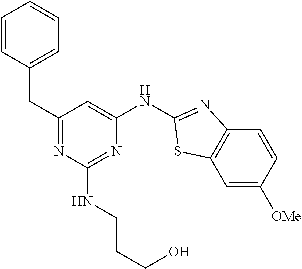 | 3-{[4-{[6- (methyloxy)-1,3- benzothiazol-2- yl]amino}-6- (phenylmethyl)-2- pyrimidinyl] amino}-1-propanol | LCMS (Method A): Rt 0.95 minutes; m/z 422 (MH+) | MDAP, ammonium bicarbonate modifier |
| 53 | 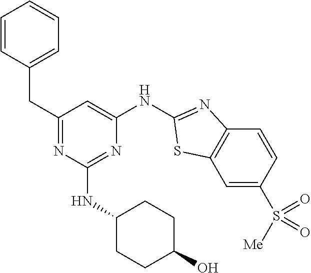 | trans-4-{[4-{[6- (methylsulfonyl)- 1,3-benzothiazol- 2-yl]amino}-6- (phenylmethyl)-2- pyrimidinyl]amino} cyclohexanol | LCMS (Method A): Rt 0.73 minutes; m/z 510 (MH+) | MDAP, ammonium bicarbonate modifier |
| 54 | 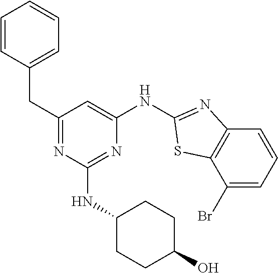 | trans-4-{[4-[(7- bromo-1,3- benzothiazol-2- yl)amino]-6- (phenylmethyl)-2- pyrimidinyl]amino} cyclohexanol | LCMS (Method A): Rt 0.80 minutes; m/z 510, 512 (MH+) | MDAP, ammonium bicarbonate modifier |

### Example 55

### 3-(4-{[4-[(6-ethyl-1,3-benzothiazol-2-yl)amino]-6-(phenylmethyl)-2-pyrimidinyl]amino}phenyl)propanoic acid

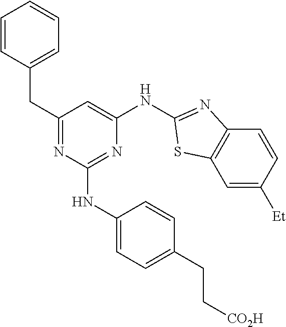

A mixture of 6-ethyl-N-[2-fluoro-6-(phenylmethyl)-4-pyrimidinyl]-1,3-benzothiazol-2-amine (110 mg, 0.302 mmol) and 3-(4-aminophenyl)propanoic acid (100 mg, 0.604 mmol) in acetonitrile (4 mL) was treated with aqueous hydrochloric acid (2M, 3 drops) and the mixture was sealed and heated in a Biotage “Initiator” microwave at 145° C. for 75 minutes. The cooled mixture was filtered and the filtered solid was washed with acetonitrile and dried. The product was purified by mass-directed autopreparative HPLC (ammonium bicarbonate modifier) to afford of the title compound (154 mg, 0.30 mmol, 82%). LCMS (Method A): Rt 1.07 minutes; m/z 510 (MH+).

The compounds shown in the table were prepared in an analogous manner to that for 3-(4-{[4-[(6-ethyl-1,3-benzothiazol-2-yl)amino]-6-(phenylmethyl)-2-pyrimidinyl]amino}phenyl)propanoic acid by reacting the appropriately-substituted 2-fluoropyrimidine with the appropriately-substituted aniline:

| | | | | Purification |
|---|---|---|---|---|
| Example | Structure | Name | Analytical Data | Method |
| 56 | 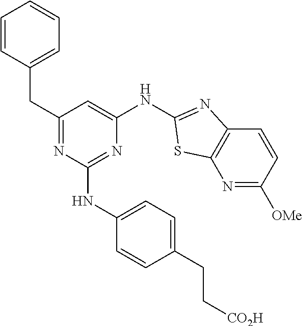 | 3-(4-{[4-{[5- (methyloxy)[1,3] thiazolo[5,4- b]pyridin-2- yl]amino}-6- (phenylmethyl)- 2- pyrimidinyl]amino} phenyl)propanoic acid | LCMS (Method A): Rt 1.01 minutes; m/z 513 (MH+) | MDAP, ammonium bicarbinate modifier |
| 57 | 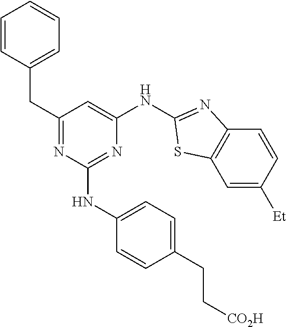 | 3-(4-{[4-[(6-ethyl- 1,3- benzothiazol-2- yl)amino]-6- (phenylmethyl)- 2- pyrimidinyl]amino} phenyl)propanoic acid | LCMS (Method A): Rt 1.15 minutes; m/z 510 (MH+) | MDAP, ammonium bicarbonate modifier |
| 58 | 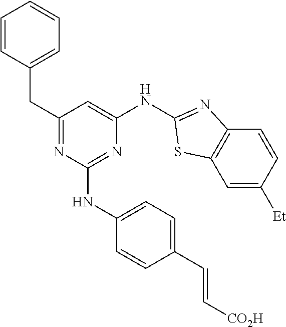 | (2E)-3-(4-{[4-[(6- ethyl-1,3- benzothiazol-2- yl)amino]-6- (phenylmethyl)- 2- pyrimidinyl]amino} phenyl)-2- propenoic acid | LCMS (Method A): Rt 1.23 minutes; m/z 508 (MH+) | MDAP, ammonium bicarbonate modifier |
| 59 | 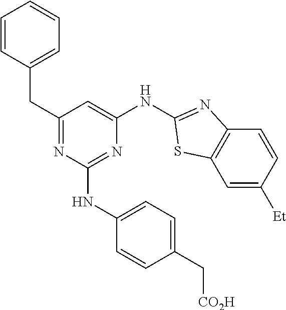 | (4-{[4-[(6-ethyl- 1,3- benzothiazol-2- yl)amino]-6- (phenylmethyl)- 2- pyrimidinyl]amino} phenyl)acetic acid | LCMS (Method A): Rt 1.05 minutes; m/z 496 (MH+) | Precipitated product filtered and washed with methanol |
| 60 | 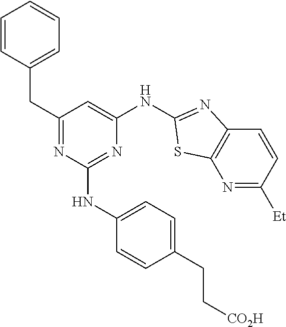 | 3-(4-{[4-[(5- ethyl[1,3]thiazolo [5,4-b]pyridin-2- yl)amino]-6- (phenylmethyl)- 2- pyrimidinyl]amino} phenyl)propanoic acid | LCMS (Method A): Rt 1.06 minutes; m/z 511 (MH+) | MDAP, ammonium bicarbonate modifier |
| 61 | 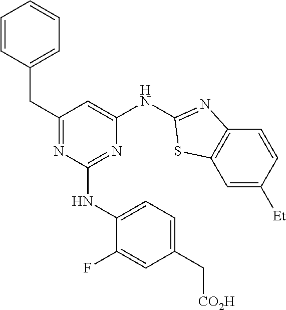 | (4-{[4-[(6-ethyl- 1,3- benzothiazol-2- yl)amino]-6- (phenylmethyl)- 2- pyrimidinyl]amino}-3- fluorophenyl)acetic acid | LCMS (Method A): Rt 1.12 minutes; m/z 514 (MH+) | MDAP, TFA modifier |
| 62 | 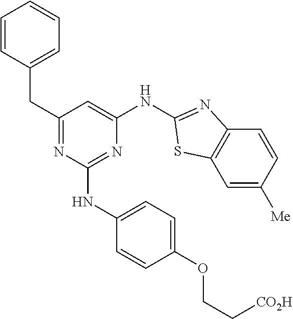 | 3-[(4-{[4-[(6- methyl-1,3- benzothiazol-2- yl)amino]-6- (phenylmethyl)- 2- pyrimidinyl]amino} phenyl)oxy]propanoic acid | LCMS (Method A): Rt 0.95 minutes; m/z 512 (MH+) | MDAP, TFA modifier |
| 63 | 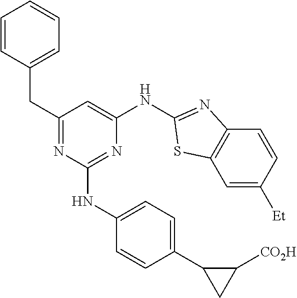 | 2-(4-{[4-[(6-ethyl- 1,3- benzothiazol-2- yl)amino]-6- (phenylmethyl)- 2- pyrimidinyl]amino} phenyl)cyclopropanecarboxylic acid | LCMS (Method A): Rt 1.10 minutes; m/z 522 (MH+) | MDAP, TFA modifier |
| 64 | 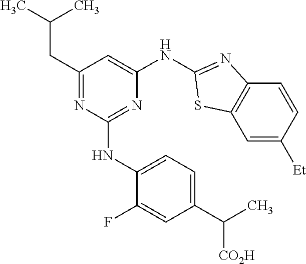 | 2-(4-{[4-[(6-ethyl- 1,3- benzothiazol-2- yl)amino]-6-(2- methylpropyl)-2- pyrimidinyl]amino}-3- fluorophenyl)propanoic acid | LCMS (Method A): Rt 1.04 minutes; m/z 494 (MH+) | MDAP, TFA modifier |
| 65 | 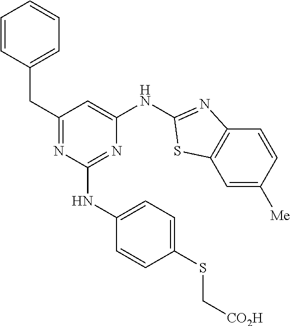 | [(4-{[4-[(6- methyl-1,3- benzothiazol-2- yl)amino]-6- (phenylmethyl)- 2- pyrimidinyl]amino} phenyl)thio]acetic acid | LCMS (Method A): Rt 1.11 minutes; m/z 514 (MH+) | MDAP, TFA modifier |
| 66 | 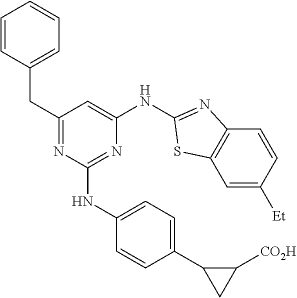 | 2-(4-{[4-[(6-ethyl- 1,3- benzothiazol-2- yl)amino]-6- (phenylmethyl)- 2- pyrimidinyl]amino} phenyl)cyclopropanecarboxylic acid | LCMS (Method A): Rt 1.04 minutes; m/z 522 (MH+) | MDAP, TFA modifier |
| 67 | 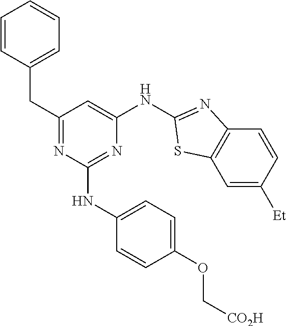 | [(4-{[4-[(6-ethyl- 1,3- benzothiazol-2- yl)amino]-6- (phenylmethyl)- 2- pyrimidinyl]amino} phenyl)oxy]acetic acid | LCMS (Method A): Rt 1.04 minutes; m/z 512 (MH+) | MDAP, ammonium bicarbonate modifier |
| 68 | 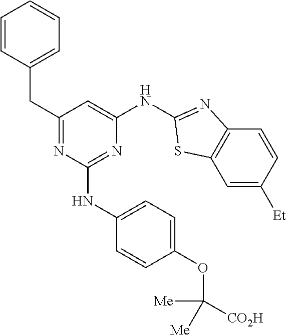 | 2-[(4-{[4-[(6- ethyl-1,3- benzothiazol-2- yl)amino]-6- (phenylmethyl)- 2- pyrimidinyl]amino} phenyl)oxy]-2- methylpropanoic acid | LCMS (Method A): Rt 1.12 minutes; m/z 540 (MH+) | MDAP, TFA modifier |
| 69 | 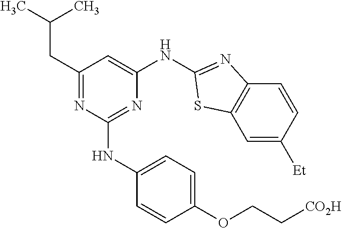 | 3-[(4-{[4-[(6- ethyl-1,3- benzothiazol-2- yl)amino]-6-(2- methylpropyl)-2- pyrimidinyl]amino} phenyl)oxy]propanoic acid | LCMS (Method A): Rt 0.90 minutes; m/z 492 (MH+) | MDAP, TFA modifier |
| 70 | 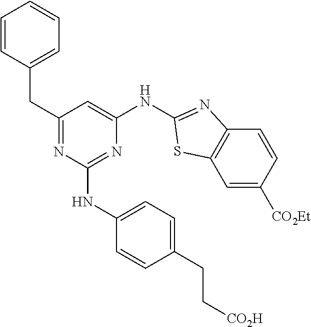 | 3-(4-{[4-({6- [(ethyloxy)carbonyl]-1,3- benzothiazol-2- yl}amino)-6- (phenylmethyl)- 2- pyrimidinyl]amino} phenyl)propanoic acid | LCMS (Method A): Rt 1.01 minutes; m/z 554 (MH+) | Precipitated product filtered and washed with acetonitrile |
| 71 | 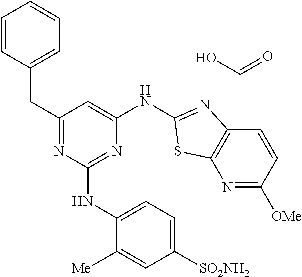 | 3-methyl-4-{[4-{[5- (methyloxy)[1,3] thiazolo[5,4- b]pyridin-2- yl]amino}-6- (phenylmethyl)- 2- pyrimidinyl]amino} benzenesulfonamide formate | LCMS (Method A): Rt 0.96 minutes; m/z 534 (MH+) | MDAP, formic acid modifier |
| 72 | 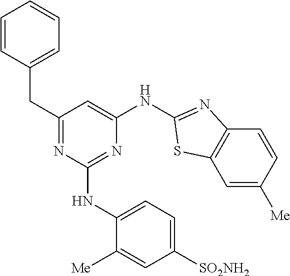 | 3-methyl-4-{[4- [(6-methyl-1,3- benzothiazol-2- yl)amino]-6- (phenylmethyl)-2- pyrimidinyl]amino} benzenesulfonamide | LCMS (Method A): Rt 0.99 minutes; m/z 517 (MH+) | MDAP, TFA modifier |
| 73 | 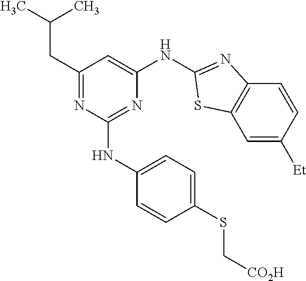 | [(4-{[4-[(6-ethyl- 1,3- benzothiazol-2- yl)amino]-6-(2- methylpropyl)-2- pyrimidinyl]amino} phenyl)thio]acetic acid | LCMS (Method A): Rt 1.03 minutes; m/z 494 (MH+) | MDAP, TFA modifier |
| 74 | 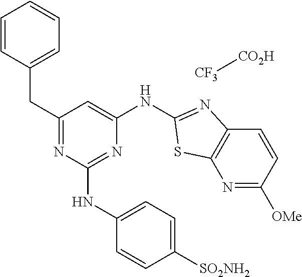 | 4-{[4-{[5- (methyloxy)[1,3] thiazolo[5,4- b]pyridin-2- yl]amino}-6- (phenylmethyl)-2- pyrimidinyl]amino} benzenesulfonamide trifluoroacetate | LCMS (Method A): Rt 1.06 minutes; m/z 520 (MH+) | MDAP, TFA modifier |
| 75 | 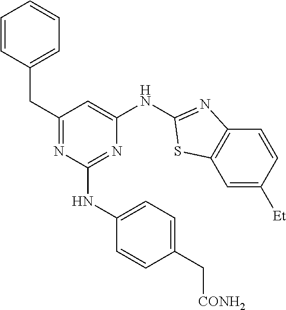 | 2-(4-{[4-[(6-ethyl- 1,3- benzothiazol-2- yl)amino]-6- (phenylmethyl)-2- pyrimidinyl]amino} phenyl)acetamide | LCMS (Method A): Rt 0.98 minutes; m/z 495 (MH+) | MDAP, ammonium bicarbonate modifier |
| 76 | 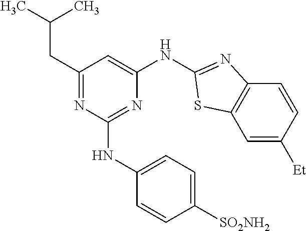 | 4-{[4-[(6-ethyl- 1,3- benzothiazol-2- yl)amino]-6-(2- methylpropyl)-2- pyrimidinyl]amino} benzenesulfonamide | LCMS (Method A): Rt 1.09 minutes; m/z 483 (MH+) | MDAP, TFA modifier |
| 77 | 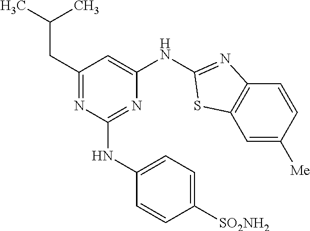 | 4-{[4-[(6-methyl- 1,3- benzothiazol-2- yl)amino]-6-(2- methylpropyl)-2- pyrimidinyl]amino} benzenesulfonamide | LCMS (Method A): Rt 1.10 minutes; m/z 469 (MH+) | MDAP, TFA modifier |
| 78 | 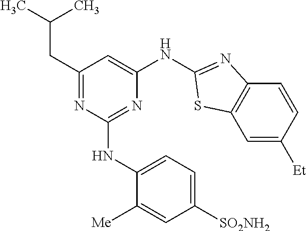 | 4-{[4-[(6-ethyl- 1,3- benzothiazol-2- yl)amino]-6-(2- methylpropyl)-2- pyrimidinyl]amino}-3- methylbenzenesulfonamide | LCMS (Method A): Rt 0.91 minutes; m/z 497 (MH+) | MDAP, TFA modifier |
| 79 | 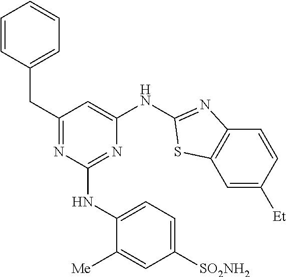 | 4-{[4-[(6-ethyl- 1,3- benzothiazol-2- yl)amino]-6- (phenylmethyl)-2- pyrimidinyl]amino}-3- methylbenzenesulfonamide | LCMS (Method A): Rt 1.04 minutes; m/z 531 (MH+) | MDAP, TFA modifier |
| 80 | 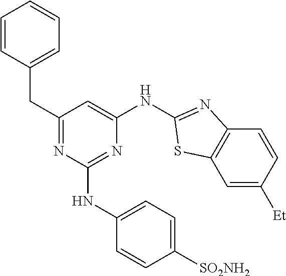 | 4-{[4-[(6-ethyl- 1,3- benzothiazol-2- yl)amino]-6- (phenylmethyl)-2- pyrimidinyl]amino} benzenesulfonamide | LCMS (Method A): Rt 1.16 minutes; m/z 517 (MH+) | MDAP, TFA modifier |
| 81 | 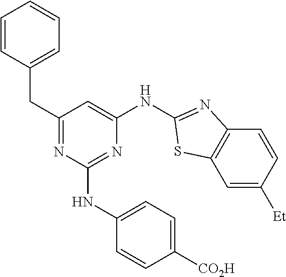 | 4-{[4-[(6-ethyl- 1,3- benzothiazol-2- yl)amino]-6- (phenylmethyl)-2- pyrimidinyl]amino} benzoic acid | LCMS (Method A): Rt 1.19 minutes; m/z 482 (MH+) | MDAP, ammonium bicarbonate modifier |
| 82 | 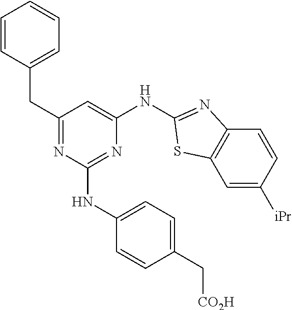 | (4-{[4-{[6-(1- methylethyl)-1,3- benzothiazol-2- yl]amino}-6- (phenylmethyl)-2- pyrimidinyl]amino} phenyl)acetic acid | LCMS (Method A): Rt 1.11 minutes; m/z 510 (MH+) | MDAP, ammonium bicarbonate modifier |
| 83 | 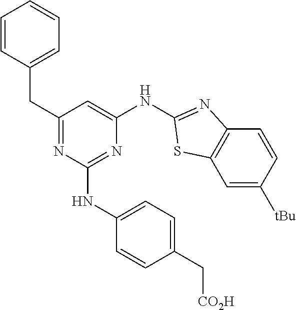 | (4-{[4-{[6-(1,1- dimethylethyl)- 1,3- benzothiazol-2- yl]amino}-6- (phenylmethyl)-2- pyrimidinyl]amino} phenyl)acetic acid | LCMS (Method A): Rt 1.21 minutes; m/z 524 (MH+) | MDAP, ammonium bicarbonate modifier |
| 84 | 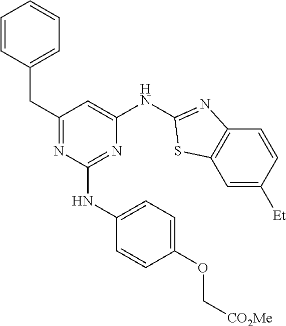 | methyl [(4-{[4- [(6-ethyl-1,3- benzothiazol-2- yl)amino]-6- (phenylmethyl)-2- pyrimidinyl]amino} phenyl)oxy]acetate | LCMS (Method A): Rt 1.17 minutes; m/z 526 (MH+) | MDAP, ammonium bicarbonate modifier |
| 85 | 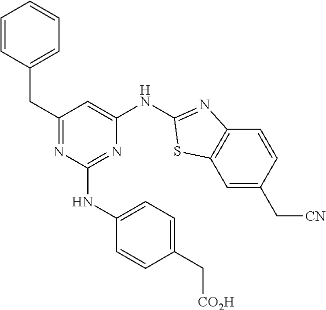 | (4-{[4-{[6- (cyanomethyl)- 1,3- benzothiazol-2- yl]amino}-6- (phenylmethyl)-2- pyrimidinyl]amino} phenyl)acetic acid | LCMS (Method A): Rt 0.93 minutes; m/z 507 (MH+) | MDAP, ammonium bicarbonate modifier |
| 86 | 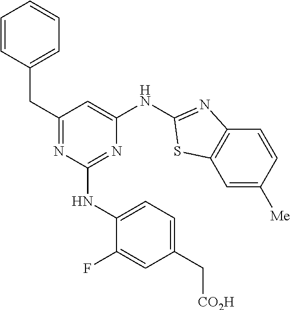 | (3-fluoro-4-{[4- [(6-methyl-1,3- benzothiazol-2- yl)amino]-6- (phenylmethyl)-2- pyrimidinyl]amino} phenyl)acetic acid | LCMS (Method A): Rt 1.05 minutes; m/z 500 (MH+) | MDAP, TFA modifier |
| 87 | 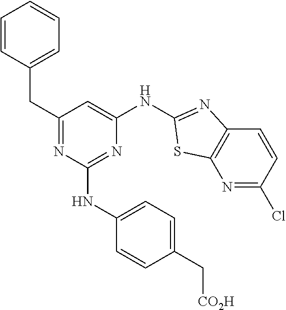 | 3-(4-{[4-[(5- chloro[1,3]thiazolo [5,4-b]pyridin-2- yl)amino]-6- (phenylmethyl)-2- pyrimidinyl]amino} phenyl)propanoic acid | LCMS (Method A): Rt 1.08 minutes; m/z 518 (MH+) | Precipitated product filtered and washed with acetonitrile and methanol |
| 88 | 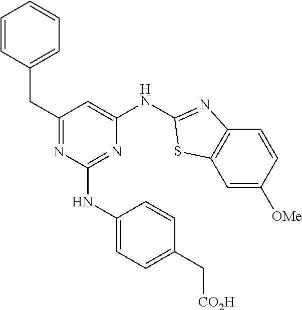 | (4-{[4-{[6- (methyloxy)-1,3- benzothiazol-2- yl]amino}-6- (phenylmethyl)-2- pyrimidinyl]amino} phenyl)acetic acid | LCMS (Method A): Rt 0.95 minutes; m/z 498 (MH+) | MDAP, ammonium bicarbonate modifier |
| 89 | 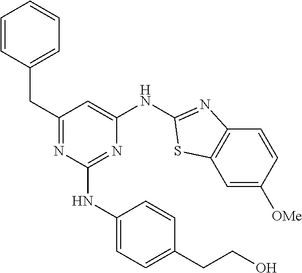 | 2-(4-{[4-{[6- (methyloxy)-1,3- benzothiazol-2- yl]amino}-6- (phenylmethyl)-2- pyrimidinyl]amino} phenyl)ethanol | LCMS (Method A): Rt 0.95 minutes; m/z 484 (MH+) | MDAP, ammonium bicarbonate modifier |
| 90 | 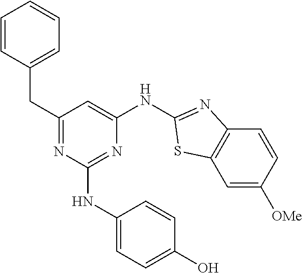 | 4-{[4-{[6- (methyloxy)-1,3- benzothiazol-2- yl]amino}-6- (phenylmethyl)-2- pyrimidinyl]amino} phenol | LCMS (Method A): Rt 0.87 minutes; m/z 456 (MH+) | MDAP, ammonium bicarbonate modifier |
| 91 | 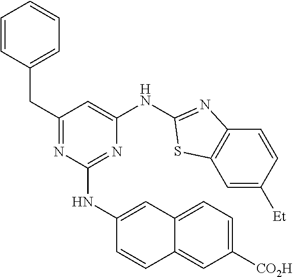 | 6-{[4-[(6-ethyl- 1,3- benzothiazol-2- yl)amino]-6- (phenylmethyl)-2- pyrimidinyl]amino}-2- naphthalenecarboxylic acid | LCMS (Method A): Rt 1.24 minutes; m/z 532 (MH+) | MDAP, TFA modifier |
| 92 | 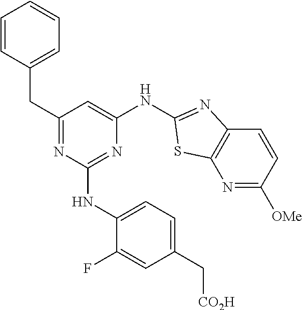 | (3-fluoro-4-{[4- {[5- (methyloxy)[1,3] thiazolo[5,4- b]pyridin-2- yl]amino}-6- (phenylmethyl)-2- pyrimidinyl]amino} phenyl)acetic acid | LCMS (Method B): Rt 2.68 minutes; m/z 517 (MH+) | MDAP, formic acid modifier |
| 93 | 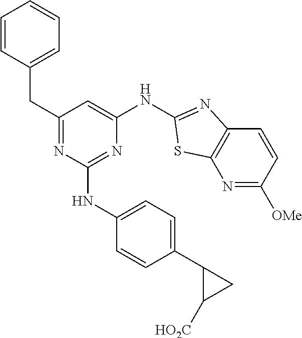 | 2-(4-{[4-{[5- (methyloxy)[1,3] thiazolo[5,4- b]pyridin-2- yl]amino}-6- (phenylmethyl)-2- pyrimidinyl]amino} phenyl)cyclopropanecarboxylic acid | LCMS (Method B): Rt 2.69 minutes; m/z 525 (MH+) | MDAP, formic acid modifier |

### Example 94

### trans-4-{[4-(1,3-benzothiazol-2-ylamino)-6-(phenylmethyl)-2-pyrimidinyl]amino}cyclohexanol

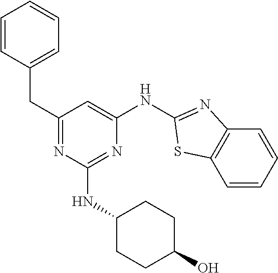

A mixture of N-[2-(methylsulfinyl)-6-(phenylmethyl)-4-pyrimidinyl]-1,3-benzothiazol-2-amine (60 mg, 0.158 mmol) and trans-4-aminocyclohexanol (60 mg, 0.521 mmol) in isopropanol (2 mL) was heated in a Biotage “Initiator” microwave at 160° C. for 4 hours. The cooled reaction mixture was evaporated to dryness and the product purified by mass-directed autopreparative HPLC (ammonium bicarbonate modifier) to afford the title compound (49 mg, 0.114 mmol, 72.0% yield) as an off-white solid. LCMS (Method B): Rt 2.73 minutes; m/z 432 (MH+).

### Example 95

### trans-4-{[4-{[6-(methyloxy)-1,3-benzothiazol-2-yl]amino}-6-(phenylmethyl)-2-pyrimidinyl]amino}cyclohexanol

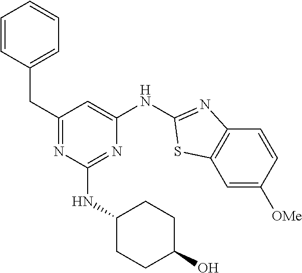

A mixture of 6-(methyloxy)-N-[2-(methylsulfonyl)-6-(phenylmethyl)-4-pyrimidinyl]-1,3-benzothiazol-2-amine (30 mg, 0.070 mmol) and trans-4-aminocyclohexanol (24.30 mg, 0.211 mmol) in isopropanol (2 mL) was sealed and heated in a Biotage “Initiator” microwave at 130° C. for 2 hours. The mixture was evaporated to dryness and the product purified by mass-directed autopreparative HPLC (ammonium bicarbonate modifier) to afford the title compound (10.2 mg, 31.4% yield) as a white solid. LCMS (Method A): Rt 0.83 minutes; m/z 462 (MH+).

The compound shown in the table was prepared in an analogous manner to that for trans-4-{[4-{[6-(methyloxy)-1,3-benzothiazol-2-yl]amino}-6-(phenylmethyl)-2-pyrimidinyl]amino}cyclohexanol by reacting 6-(methyloxy)-N-[2-(methylsulfonyl)-6-(phenylmethyl)-4-pyrimidinyl]-1,3-benzothiazol-2-amine with 4-(2-aminoethyl)benzenesulfonamide:

| | | | Analytical | Purification |
|---|---|---|---|---|
| Example | Structure | Name | Data | Method |
| 96 | 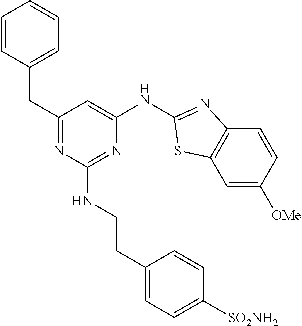 | 4-(2-{[4-{[6- (methyloxy)-1,3- benzothiazol-2- yl]amino}-6- (phenylmethyl)-2- pyrimidinyl]amino} ethyl)benzene- sulfonamide | LCMS (Method A): Rt 0.80 minutes; m/z 547 (MH+) | Precipitated product filtered and washed with methanol |

### Example 97

### 3-(4-{[4-{[6-(methyloxy)-1,3-benzothiazol-2-yl]amino}-6-(phenylmethyl)-2-pyrimidinyl]amino}phenyl)propanoic acid

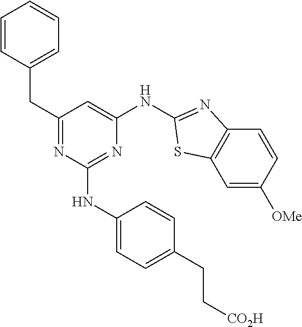

A mixture of 3-(4-aminophenyl)propanoic acid (12.4 mg, 0.075 mmol) and 6-(methyloxy)-N-[2-(methylsulfonyl)-6-(phenylmethyl)-4-pyrimidinyl]-1,3-benzothiazol-2-amine (16 mg, 0.038 mmol) in acetonitrile (2 mL) was treated with 2M aqueous hydrochloric acid (2 drops) and then sealed and heated in a Biotage “Initiator” microwave at 145° C. for 60 minutes. The cooled mixture was evaporated to dryness and the product was purified by mass-directed autopreparative HPLC (ammonium bicarbonate modifier) to afford the title compound (21 mg, 0.038 mmol, 65.5% yield) as a white solid. LCMS (Method A): Rt 0.95 minutes; m/z 512 (MH+).

### Example 98

### 2-{[2-[(trans-4-hydroxycyclohexyl)amino]-6-(phenylmethyl)-4-pyrimidinyl]amino}-1,3-benzothiazole-6-carboxylic acid

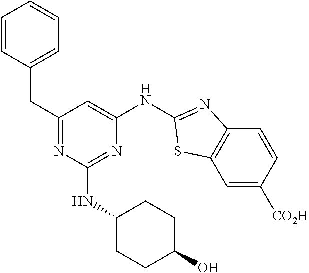

A mixture of ethyl 2-{[2-[(trans-4-hydroxycyclohexyl)amino]-6-(phenylmethyl)-4-pyrimidinyl]amino}-1,3-benzothiazole-6-carboxylate (150 mg, 0.298 mmol) and concentrated aqueous ammonia (2 mL, 51.4 mmol) was sealed and heated in a Biotage “Initiator” microwave at 100° C. for 30 minutes. The reaction mixture was evaporated to dryness and the product was purified by mass-directed autopreparative HPLC (ammonium bicarbonate modifier) to afford the title compound (13 mg, 0.027 mmol, 9.2% yield) as a white solid. LCMS (Method A): Rt 0.70 minutes; m/z 476 (MH+).

The compounds shown in the table were prepared in an analogous manner to that for 2-{[2-[(trans-4-hydroxycyclohexyl)amino]-6-(phenylmethyl)-4-pyrimidinyl]amino}-1,3-benzothiazole-6-carboxylic acid by reacting the appropriate carboxylic ester with aqueous ammonia:

| | | | Analytical | Purification |
|---|---|---|---|---|
| Example | Structure | Name | Data | Method |
| 99 | 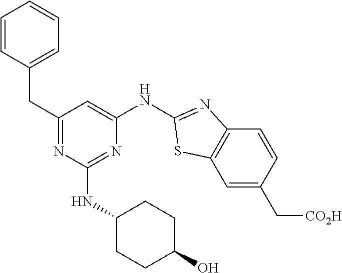 | (2-{[2-[(trans-4- hydroxycyclohexyl) amino]-6-(phenylmethyl)- 4-pyrimidinyl]amino}-1,3- benzothiazol-6-yl)acetic acid | LCMS (Method A): Rt 0.89 minutes; m/z 490 (MH+) | MDAP, ammonium bicarbonate modifier |
| 100 | 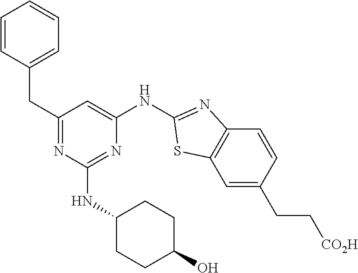 | 3-(2-{[2-[(trans-4- hydroxycyclohexyl) amino]-6-(phenylmethyl)- 4-pyrimidinyl]amino}-1,3- benzothiazol-6- yl)propanoic acid | LCMS (Method A): Rt 0.72 minutes; m/z 504 (MH+) | MDAP, ammonium bicarbonate modifier |

### Example 101

### 2-(2-{[2-[(trans-4-hydroxycyclohexyl)amino]-6-(phenylmethyl)-4-pyrimidinyl]amino}-1,3-benzothiazol-6-yl)acetamide

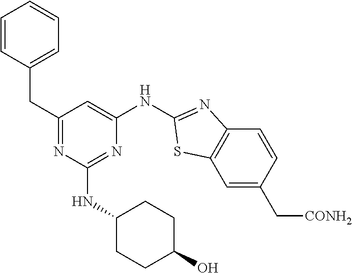

A mixture of ethyl (2-{[2-[(trans-4-hydroxycyclohexyl)amino]-6-(phenylmethyl)-4-pyrimidinyl]amino}-1,3-benzothiazol-6-yl)acetate (50 mg, 0.097 mmol) and a solution of ammonia in methanol (7M, 2 mL, 14 mmol) was sealed and heated in a Biotage “Initiator” microwave at 150° C. for 7 hours. The cooled reaction mixture was evaporated to dryness and the product was purified by mass-directed autopreparative HPLC (ammonium bicarbonate modifier) to afford the title compound (30 mg, 0.061 mmol, 63.6% yield) as a white solid. LCMS (Method A): Rt 0.64 minutes; m/z 489 (MH+).

The compound shown in the table was prepared in an analogous manner to that for 2-(2-{[2-[(trans-4-hydroxycyclohexyl)amino]-6-(phenylmethyl)-4-pyrimidinyl]amino}-1,3-benzothiazol-6-yl)acetamide by reacting ethyl 3-(2-{[2-[(trans-4-hydroxycyclohexyl)amino]-6-(phenylmethyl)-4-pyrimidinyl]amino}-1,3-benzothiazol-6-yl)propanoate with ammonia in methanol:

| | | | Analytical | Purification |
|---|---|---|---|---|
| Example | Structure | Name | Data | Method |
| 102 | 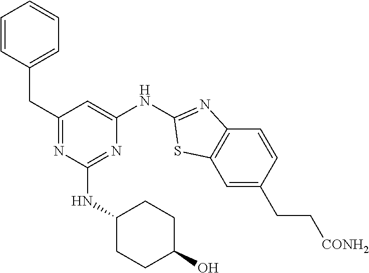 | 3-(2-{[2-[(trans-4- hydroxycyclohexyl) amino]-6- (phenylmethyl)-4- pyrimidinyl]amino}- 1,3-benzothiazol-6- yl)propanamide | LCMS (Method A): Rt 0.67 minutes; m/z 503 (MH+) | MDAP, ammonium bicarbonate modifier |

### Example 103

trans-4-{[4-{[6-(2-hydroxyethyl)-1,3-benzothiazol-2-yl]amino}-6-(phenylmethyl)-2-pyrimidinyl]amino}cyclohexanol

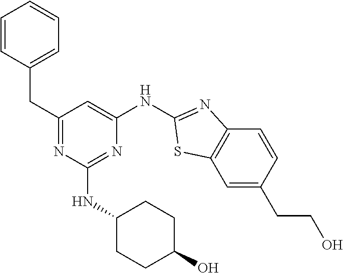

To an ice-cooled, stirred solution of ethyl (2-{[2-[(trans-4-hydroxycyclohexyl)amino]-6-(phenylmethyl)-4-pyrimidinyl]amino}-1,3-benzothiazol-6-yl)acetate (60 mg, 0.116 mmol) in tetrahydrofuran (2 mL) under an atmosphere of nitrogen was added dropwise a solution of lithium aluminium hydride (1.0M in diethyl ether) (0.12 mL, 0.12 mmol). The reaction mixture was stirred at 0° C. for 30 minutes then treated cautiously with water (10 mL) and then extracted with ethyl acetate (2×10 mL). The organic fractions were combined, evaporated to dryness and the product was purified using mass-directed autopreparative HPLC (ammonium bicarbonate modifier) to afford the title compound (21 mg, 0.044 mmol, 38% yield) as a white solid. LCMS (Method A): Rt 0.68 minutes; m/z 476 (MH+).

The compounds shown in the table were prepared in an analogous manner to that for trans-4-{[4-{[6-(2-hydroxyethyl)-1,3-benzothiazol-2-yl]amino}-6-(phenylmethyl)-2-pyrimidinyl]amino}cyclohexanol by reacting the appropriate carboxylic ester with lithium aluminium hydride:

| | | | Analytical | Purification |
|---|---|---|---|---|
| Example | Structure | Name | Data | Method |
| 104 | 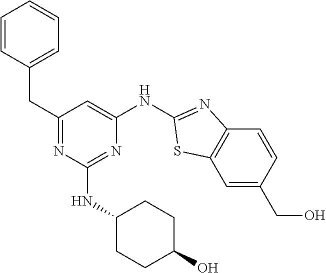 | trans-4-{[4-{[6- (hydroxymethyl)-1,3- benzothiazol-2- yl]amino}-6- (phenylmethyl)-2- pyrimidinyl]amino} cyclohexanol | LCMS (Method A): Rt 0.66 minutes; m/z 462 (MH+) | MDAP, ammonium bicarbonate modifier |
| 105 | 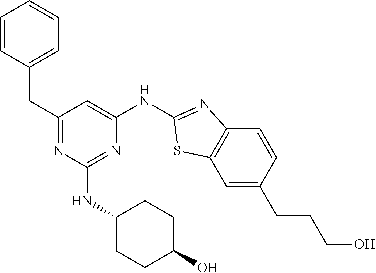 | trans-4-{[4-{[6-(3- hydroxypropyl)-1,3- benzothiazol-2- yl]amino}-6- (phenylmethyl)-2- pyrimidinyl]amino} cyclohexanol | LCMS (Method A): Rt 0.71 minutes; m/z 490 (MH+) | MDAP, ammonium bicarbonate modifier |

### Example 106

### trans-4-{[4-[(6-amino-1,3-benzothiazol-2-yl)amino]-6-(phenylmethyl)-2-pyrimidinyl]amino}cyclohexanol

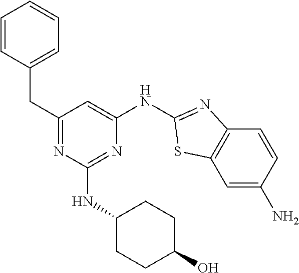

A mixture of trans-4-{[4-[(6-nitro-1,3-benzothiazol-2-yl)amino]-6-(phenylmethyl)-2-pyrimidinyl]amino}cyclohexanol (1.38 g, 2.90 mmol) and palladium on activated carbon (10% wt (dry basis), (˜50% water)) (150 mg) in tetrahydrofuran (50 mL) was stirred in an atmosphere of hydrogen for 24 hours at ambient temperature. The reaction mixture was then filtered through celite which was subsequently washed with more tetrahydrofuran. The combined filtrate was concentrated in vacuo to afford the title compound (1.17 g, 2.63 mmol, 91% yield) as a pale yellow solid. LCMS (Method A): Rt 0.60 minutes; m/z 447 (MH+).

### Example 107

### N-(2-{[2-[(trans-4-hydroxycyclohexyl)amino]-6-(phenylmethyl)-4-pyrimidinyl]amino}-1,3-benzothiazol-6-yl)acetamide

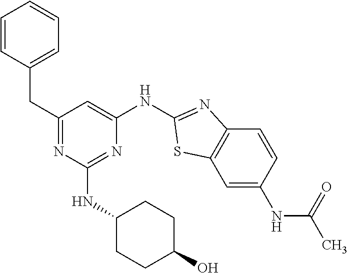

Acetyl chloride (0.008 mL, 0.112 mmol) was added to an ice-cooled, stirred solution of trans-4-{[4-[(6-amino-1,3-benzothiazol-2-yl)amino]-6-(phenylmethyl)-2-pyrimidinyl]amino}cyclohexanol (50 mg, 0.112 mmol) and N-ethyldiisopropylamine (0.020 mL, 0.112 mmol) in tetrahydrofuran (2 mL). The solution was stirred for 15 minutes at 0° C. and then passed through a 1 g aminopropyl solid-phase extraction cartridge, eluting with methanol. Product-containing fractions were combined, evaporated to dryness and the product was then purified by mass-directed autopreparative HPLC (formic acid modifier) to afford the title compound (7 mg, 0.014 mmol, 13% yield) as a white solid. LCMS (Method A): Rt 0.80 minutes; m/z 489 (MH+).

### Example 108

### methyl (2-{[2-[(trans-4-hydroxycyclohexyl)amino]-6-(phenylmethyl)-4-pyrimidinyl]amino}-1,3-benzothiazol-6-yl)carbamate

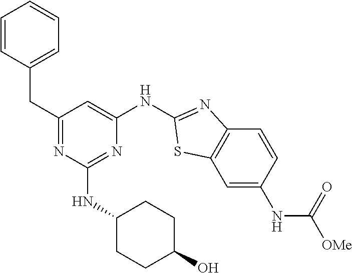

Methyl chloroformate (0.035 mL, 0.448 mmol) was added to an ice-cooled, stirred solution of trans-4-{[4-[(6-amino-1,3-benzothiazol-2-yl)amino]-6-(phenylmethyl)-2-pyrimidinyl]amino}cyclohexanol (200 mg, 0.448 mmol) and N-ethyldiisopropylamine (0.078 mL, 0.448 mmol) in tetrahydrofuran (2 mL). The solution was stirred for 15 minutes at 0° C. and then passed through a 10 g aminopropyl solid-phase extraction cartridge, eluting with methanol. The product-containing fractions were combined, evaporated to dryness and the product was purified by mass-directed autopreparative HPLC (ammonium bicarbonate modifier) to afford the title compound (220 mg, 0.436 mmol, 97% yield) as a yellow solid. LCMS (Method A): Rt 0.83 minutes; m/z 505 (MH+).

### Example 109

### N-(2-{[2-[(trans-4-hydroxycyclohexyl)amino]-6-(phenylmethyl)-4-pyrimidinyl]amino}-1,3-benzothiazol-6-yl)methanesulfonamide

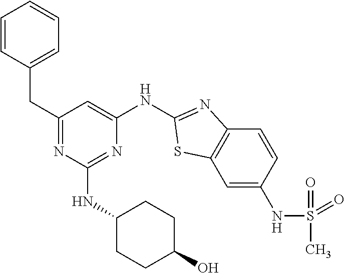

Methanesulfonyl chloride (0.0087 mL, 0.112 mmol) was added to an ice-cooled, stirred solution of trans-4-{[4-[(6-amino-1,3-benzothiazol-2-yl)amino]-6-(phenylmethyl)-2-pyrimidinyl]amino}cyclohexanol (50 mg, 0.112 mmol) and N-ethyldiisopropylamine (0.020 mL, 0.112 mmol) in tetrahydrofuran (2 mL). The solution was stirred for 15 minutes at 0° C. and then passed through a 1 g aminopropyl solid-phase extraction cartridge, eluting with methanol. The product-containing fractions were combined, evaporated to dryness and the product was purified by mass-directed autopreparative HPLC (formic acid modifier) to afford the title compound (20 mg, 0.038 mmol, 34% yield) as a white solid. LCMS (Method A): Rt 0.82 minutes; m/z 525 (MH+).

### Example 110

### 1-(2-{[2-[(trans-4-hydroxycyclohexyl)amino]-6-(phenylmethyl)-4-pyrimidinyl]amino}-1,3-benzothiazol-6-yl)-2-azetidinone

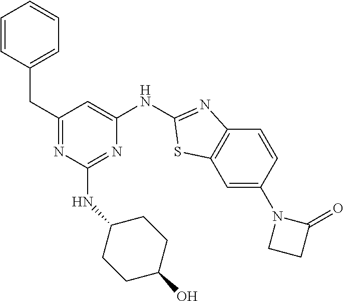

To an ice-cooled, stirred solution of trans-4-{[4-[(6-amino-1,3-benzothiazol-2-yl)amino]-6-(phenylmethyl)-2-pyrimidinyl]amino}cyclohexanol (50 mg, 0.112 mmol) and N-ethyldiisopropylamine (0.020 mL, 0.112 mmol) in tetrahydrofuran (2 mL) was added, dropwise, 3-bromopropanoyl chloride (1.13 mL, 0.112 mmol) and the reaction mixture was stirred for 5 minutes. The reaction mixture was then treated portion-wise with potassium tert-butoxide (50 mg, 0.446 mmol). The mixture was then treated with water (2 mL) and brine (20 mL) and the product extracted with ethyl acetate (2×40 mL). The combined organics were dried over magnesium sulfate, filtered and evaporated to dryness. The product was purified by mass-directed autopreparative HPLC (ammonium bicarbonate modifier) to afford the crude title compound (30 mg) (purity about 70%, contaminated with a by-product of identical mass assumed to be the corresponding acrylamide, N-(2-{[2-[(trans-4-hydroxycyclohexyl)amino]-6-(phenylmethyl)-4-pyrimidinyl]amino}-1,3-benzothiazol-6-yl-2-propenamide). The crude solid was dissolved in isopropanol (2 mL) and treated with N-ethyldiisopropylamine (0.020 mL, 0.112 mmol) and glycine (8.40 mg, 0.112 mmol. The reaction mixture was sealed and heated in a Biotage “Initiator” microwave at 180° C. for 1 hr. The reaction mixture was evaporated to dryness and the product was purified by mass-directed autopreparative HPLC (ammonium bicarbonate modifier) to afford the title compound (9.8 mg, 0.020 mmol, 17% yield) as a white solid. LCMS (Method A): Rt 0.77 minutes; m/z 501 (MH+).

### Example 111

### 1-(2-{[2-[(trans-4-hydroxycyclohexyl)amino]-6-(phenylmethyl)-4-pyrimidinyl]amino}-1,3-benzothiazol-6-yl)-2-pyrrolidinone

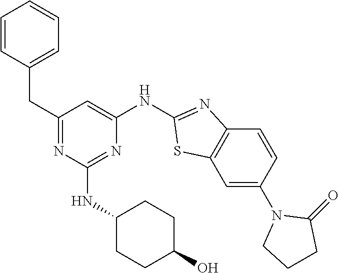

To an ice-cooled, stirred solution of trans-4-{[4-[(6-amino-1,3-benzothiazol-2-yl)amino]-6-(phenylmethyl)-2-pyrimidinyl]amino}cyclohexanol (50 mg, 0.112 mmol) and N-ethyldiisopropylamine (0.020 mL, 0.112 mmol) in tetrahydrofuran (2 mL) was added a solution of 4-bromobutanoyl chloride (0.013 mL, 0.112 mmol) in tetrahydrofuran (1 mL). The reaction mixture was then treated portion-wise over 2 hours with potassium tert-butoxide (50 mg, 0.446 mmol). The mixture was then treated with water (2 mL) and brine (20 mL) and the product extracted with ethyl acetate (2×20 mL). The combined organics were dried over magnesium sulfate, filtered and evaporated to dryness. The product was purified by mass-directed autopreparative HPLC (formic acid modifier) to afford the title compound (20 mg, 0.039 mmol, 34.7% yield) as a yellow solid. LCMS (Method A): Rt 0.73 minutes; m/z 515 (MH+).

The compound shown in the table was prepared in an analogous manner to that for 1-(2-{[2-[(trans-4-hydroxycyclohexyl)amino]-6-(phenylmethyl)-4-pyrimidinyl]amino}-1,3-benzothiazol-6-yl)-2-pyrrolidinone by reacting trans-4-{[4-[(6-amino-1,3-benzothiazol-2-yl)amino]-6-(phenylmethyl)-2-pyrimidinyl]amino}cyclohexanol with 5-bromopentanoyl chloride:

| | | | Analytical | Purification |
|---|---|---|---|---|
| Example | Structure | Name | Data | Method |
| 112 | 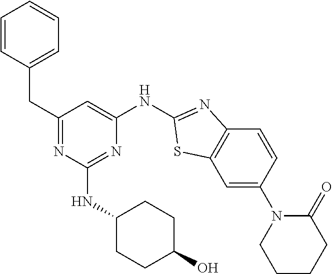 | 1-(2-{[2-[(trans-4- hydroxycyclohexyl) amino]-6- (phenylmethyl)-4- pyrimidinyl]amino}- 1,3-benzothiazol-6- yl)-2-piperidinone | LCMS (Method A): Rt 0.75 minutes; m/z 529 (MH+) | MDAP, formic acid modifier |

### Example 113

### 1-(2-{[2-[(trans-4-hydroxycyclohexyl)amino]-6-(phenylmethyl)-4-pyrimidinyl]amino}-1,3-benzothiazol-6-yl)-2,5-pyrrolidinedione

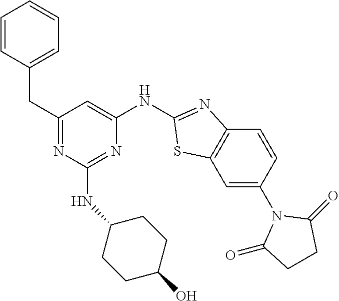

A mixture of trans-4-{[4-[(6-amino-1,3-benzothiazol-2-yl)amino]-6-(phenylmethyl)-2-pyrimidinyl]amino}cyclohexanol (50 mg, 0.112 mmol) and succinic anhydride (11.20 mg, 0.112 mmol) in acetonitrile (2 mL) was sealed and heated in a Biotage “Initiator” microwave at 150° C. for 1 hour. The cooled mixture was then treated with 2 drops of concentrated hydrochloric acid, sealed and heated at 150° C. in the microwave for a further hour. The reaction mixture was then evaporated to dryness and the product was purified by mass-directed autopreparative HPLC (formic acid modifier) to afford the title compound (23 mg, 0.044 mmol, 39% yield) as a white solid. LCMS (Method A): Rt 0.70 minutes; m/z 529 (MH+).

### Example 114

### 1-(2-{[2-[(trans-4-hydroxycyclohexyl)amino]-6-(phenylmethyl)-4-pyrimidinyl]amino}-1,3-benzothiazol-6-yl)-2-imidazolidinone

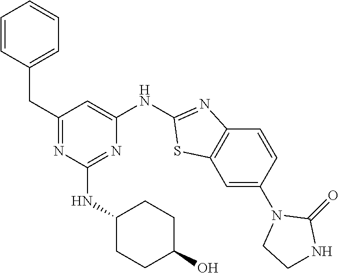

To an ice-cooled, stirred solution of trans-4-{[4-[(6-amino-1,3-benzothiazol-2-yl)amino]-6-(phenylmethyl)-2-pyrimidinyl]amino}cyclohexanol (50 mg, 0.112 mmol) and N-ethyldiisopropylamine (0.020 mL, 0.112 mmol) in tetrahydrofuran (2 mL) was added dropwise 2-chloroethyl isocyanate (24 mg, 0.224 mmol) and the reaction mixture was stirred for 5 minutes. The reaction mixture was then sealed and heated in a Biotage “Initiator” microwave at 160° C. for 1 hour. The reaction mixture was then evaporated to dryness and the product was purified by mass-directed autopreparative HPLC (formic acid modifier) to afford the title compound (23 mg, 0.044 mmol, 39% yield). LCMS (Method A): Rt 0.72 minutes; m/z 516 (MH+).

### Example 115

### 3-(2-{[2-[(trans-4-hydroxycyclohexyl)amino]-6-(phenylmethyl)-4-pyrimidinyl]amino}-1,3-benzothiazol-6-yl)-2,4-imidazolidinedione

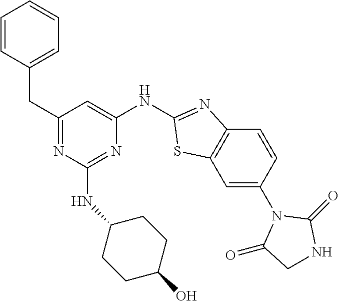

To an ice-cooled, stirred solution of trans-4-{[4-[(6-amino-1,3-benzothiazol-2-yl)amino]-6-(phenylmethyl)-2-pyrimidinyl]amino}cyclohexanol (50 mg, 0.112 mmol) and N-ethyldiisopropylamine (0.020 mL, 0.112 mmol) in tetrahydrofuran (2 mL) was added dropwise ethyl isocyanoacetate (14 mg, 0.112 mmol) and the reaction mixture was stirred for 5 minutes. The reaction mixture was then sealed and heated in a Biotage “Initiator” microwave at 100° C. for 30 minutes. The reaction mixture was then evaporated to dryness and the product was purified by mass-directed autopreparative HPLC (formic acid modifier) to afford the title compound (28 mg, 0.053 mmol, 47% yield) as a white solid. LCMS (Method A): Rt 0.66 minutes; m/z 530 (MH+).

### Example 116

### 3-(2-{[2-[(trans-4-hydroxycyclohexyl)amino]-6-(phenylmethyl)-4-pyrimidinyl]amino}-1,3-benzothiazol-6-yl)-1,3-oxazolidin-2-one

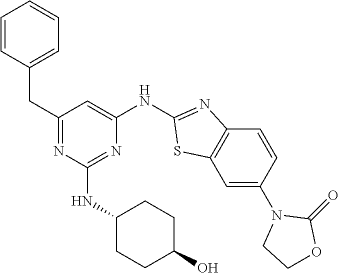

To an ice-cooled, stirred solution of trans-4-{[4-[(6-amino-1,3-benzothiazol-2-yl)amino]-6-(phenylmethyl)-2-pyrimidinyl]amino}cyclohexanol (50 mg, 0.112 mmol) and N-ethyldiisopropylamine (0.020 mL, 0.112 mmol) in tetrahydrofuran (2 mL) was added a solution of 2-bromoethyl chloroformate (0.012 mL, 0.112 mmol) in tetrahydrofuran (1 mL). The reaction mixture was then treated portion-wise over 2 hours with potassium tert-butoxide (50 mg, 0.446 mmol). The mixture was then treated with water (2 mL) and brine (20 mL) and the product extracted with ethyl acetate (2×20 mL). The combined organics were dried over magnesium sulfate, filtered and evaporated to dryness. The product was purified by mass-directed autopreparative HPLC (formic acid modifier) to afford the title compound (33 mg, 0.065 mmol, 60% yield) as a pale yellow solid. LCMS (Method A): Rt 0.75 minutes; m/z 517 (MH+).

### Example 117

### trans-4-{[4-{[5-(dimethylamino)[1,3]thiazolo[5,4-b]pyridin-2-yl]amino}-6-(phenylmethyl)-2-pyrimidinyl]amino}cyclohexanol

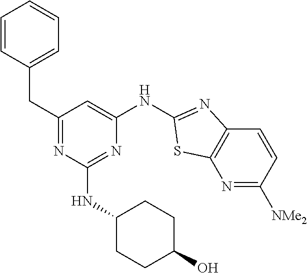

Trans-4-{[4-[(5-chloro[1,3]thiazolo[5,4-b]pyridin-2-yl)amino]-6-(phenylmethyl)-2-pyrimidinyl]amino}cyclohexanol (25 mg, 0.054 mmol) was treated with a solution of diethanolamine (200 mg, 1.902 mmol) in N,N-dimethylformamide (1 mL) and the mixture was sealed and heated in a Biotage “Initiator” microwave at 160° C. for 6 hours. The product was purified by mass-directed autopreparative HPLC (ammonium bicarbonate modifier) to afford the title compound (10.1 mg, 0.021 mmol, 40%) as a white solid. LCMS (Method A): Rt 0.81 minutes; m/z 476 (MH+).

The compounds shown in the table were prepared in an analogous manner to that for trans-4-{[4-{[5-(dimethylamino)[1,3]thiazolo[5,4-b]pyridin-2-yl]amino}-6-(phenylmethyl)-2-pyrimidinyl]amino}cyclohexanol by reacting 3-(4-{[4-[(5-chloro[1,3]thiazolo[5,4-b]pyridin-2-yl)amino]-6-(phenylmethyl)-2-pyrimidinyl]amino}phenyl)propanoic acid with diethanolamine:

| | | | Analytical | Purification |
|---|---|---|---|---|
| Example | Structure | Name | Data | Method |
| 118 | 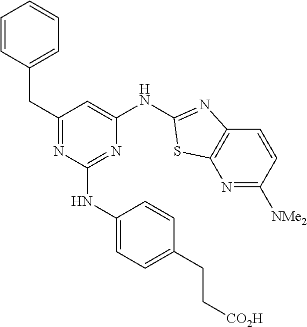 | 3-(4-{[4-{[5- (dimethylamino) [1,3]thiazolo[5,4- b]pyridin-2- yl]amino}-6- (phenylmethyl)-2- pyrimidinyl]amino} phenyl)propanoic acid | LCMS (Method A): Rt 0.97 minutes; m/z 526 (MH+) | MDAP, ammonium bicarbonate modifier |
| 119 | 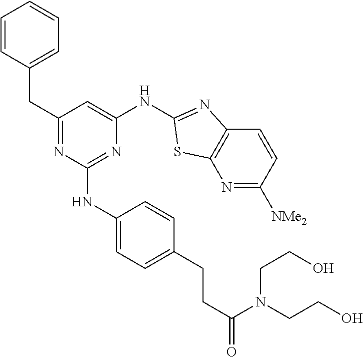 | 3-(4-{[4-{[5- (dimethylamino) [1,3]thiazolo[5,4- b]pyridin-2- yl]amino}-6- (phenylmethyl)-2- pyrimidinyl]amino} phenyl)-N,N-bis(2- hydroxyethyl) propanamide | LCMS (Method A): Rt 0.83 minutes; m/z 613 (MH+) | MDAP, ammonium bicarbonate modifier |

### Example 120

### trans-4-{[4-{[5-(4-morpholinyl)[1,3]thiazolo[5,4-b]pyridin-2-yl]amino}-6-(phenylmethyl)-2-pyrimidinyl]amino}cyclohexanol

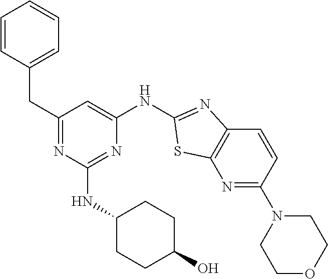

A mixture of trans-4-{[4-[(5-chloro[1,3]thiazolo[5,4-b]pyridin-2-yl)amino]-6-(phenylmethyl)-2-pyrimidinyl]amino}cyclohexanol (25 mg, 0.054 mmol) and 1 mL of morpholine was sealed and heated in a Biotage “Initiator” microwave at 160° C. for 6 hours. The product was purified by mass-directed autopreparative HPLC (ammonium bicarbonate modifier) to afford the title compound (14.5 mg, 0.028 mmol, 52%) as a white solid. LCMS (Method A): Rt 0.86 minutes; m/z 518 (MH+).

The compounds shown in the table were prepared in an analogous manner to that for trans-4-{[4-{[5-(4-morpholinyl)[1,3]thiazolo[5,4-b]pyridin-2-yl]amino}-6-(phenylmethyl)-2-pyrimidinyl]amino}cyclohexanol by reacting trans-4-{[4-[(5-chloro[1,3]thiazolo[5,4-b]pyridin-2-yl)amino]-6-(phenylmethyl)-2-pyrimidinyl]amino}cyclohexanol with the appropriate amine:

| | | | Analytical | Purification |
|---|---|---|---|---|
| Example | Structure | Name | Data | Method |
| 121 | 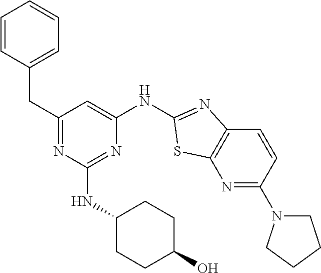 | trans-4-[(4- (phenylmethyl)-6-{[5- (1-pyrrolidinyl) [1,3]thiazolo [5,4-b]pyridin-2- yl]amino}-2- pyrimidinyl)amino] cyclohexanol | LCMS (Method A): Rt 0.82 minutes; m/z 502 (MH+) | MDAP, ammonium bicarbonate modifier |
| 122 | 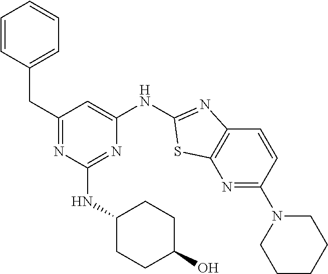 | trans-4-[(4- (phenylmethyl)-6-{[5- (1-piperidinyl) [1,3]thiazolo [5,4-b]pyridin-2- yl]amino}-2- pyrimidinyl)amino] cyclohexanol | LCMS (Method A): Rt 0.89 minutes; m/z 516 (MH+) | MDAP, ammonium bicarbonate modifier |
| 123 | 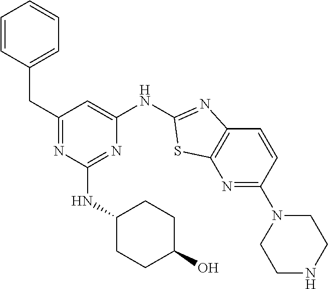 | trans-4-[(4- (phenylmethyl)-6-{[5- (1-piperazinyl) [1,3]thiazolo [5,4-b]pyridin-2- yl]amino}-2- pyrimidinyl)amino] cyclohexanol | LCMS (Method A): Rt 0.57 minutes; m/z 517 (MH+) | MDAP, ammonium bicarbonate modifier |
| 124 | 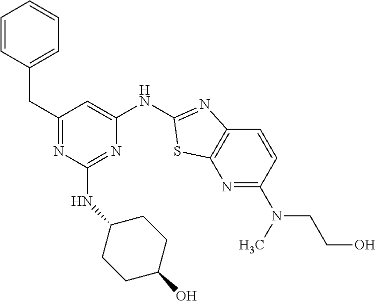 | trans-4-{[4-({5-[(2- hydroxyethyl)(methyl) amino][1,3]thiazolo[5,4- b]pyridin-2-yl}amino)- 6-(phenylmethyl)-2- pyrimidinyl]amino} cyclohexanol | LCMS (Method A): Rt 0.73 minutes; m/z 506 (MH+) | MDAP, ammonium bicarbonate modifier |
| 125 | 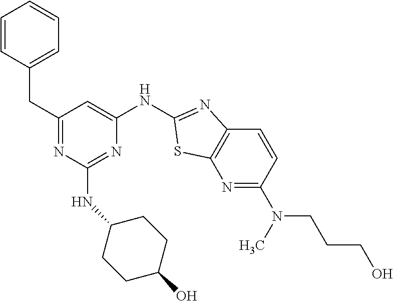 | trans-4-{[4-({5-[(3- hydroxypropyl)(methyl) amino][1,3]thiazolo[5, 4-b]pyridin-2- yl}amino)-6- (phenylmethyl)-2- pyrimidinyl]amino} cyclohexanol | LCMS (Method A): Rt 0.76 minutes; m/z 520 (MH+) | MDAP, ammonium bicarbonate modifier |
| 126 | 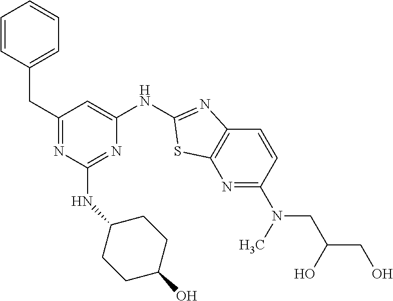 | 3-[(2-{[2-[(trans-4- hydroxycyclohexyl) amino]-6-(phenylmethyl)- 4-pyrimidinyl]amino} [1,3]thiazolo[5,4-b] pyridin-5-yl)(methyl) amino]-1,2-propanediol | LCMS (Method A): Rt 0.75 minutes; m/z 536 (MH+) | MDAP, ammonium bicarbonate modifier |
| 127 | 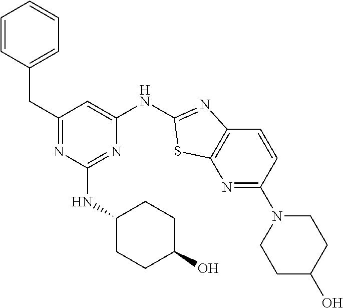 | 1-(2-{[2-[(trans-4- hydroxycyclohexyl) amino]-6-(phenylmethyl)- 4-pyrimidinyl]amino} [1,3]thiazolo[5,4-b] pyridin-5-yl)-4- piperidinol | LCMS (Method A): Rt 0.71 minutes; m/z 532 (MH+) | MDAP, ammonium bicarbonate modifier |
| 128 | 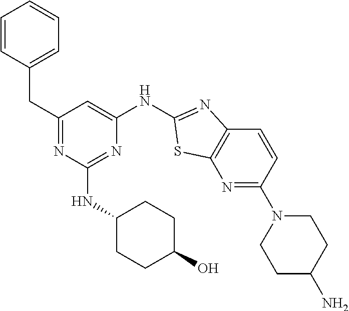 | trans-4-{[4-{[5-(4- amino-1- piperidinyl)[1,3]thiazolo [5,4-b]pyridin-2- yl]amino}-6- (phenylmethyl)-2- pyrimidinyl]amino} cyclohexanol | LCMS (Method A): Rt 0.59 minutes; m/z 531 (MH+) | MDAP, ammonium bicarbonate modifier |
| 129 | 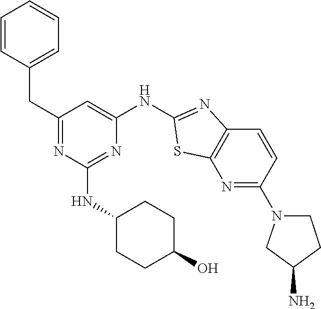 | trans-4-{[4-({5-[(3R)-3- amino-1- pyrrolidinyl][1,3] thiazolo[5,4-b]pyridin-2- yl}amino)-6- (phenylmethyl)-2- pyrimidinyl]amino} cyclohexanol | LCMS (Method A): Rt 0.58 minutes; m/z 517 (MH+) | MDAP, ammonium bicarbonate modifier |
| 130 | 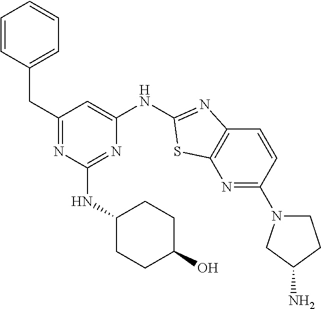 | trans-4-{[4-({5-[(3S)-3- amino-1- pyrrolidinyl][1,3]thiazolo [5,4-b]pyridin-2- yl}amino)-6- (phenylmethyl)-2- pyrimidinyl]amino} cyclohexanol | LCMS (Method A): Rt 0.57 minutes; m/z 517 (MH+) | MDAP, ammonium bicarbonate modifier |
| 131 | 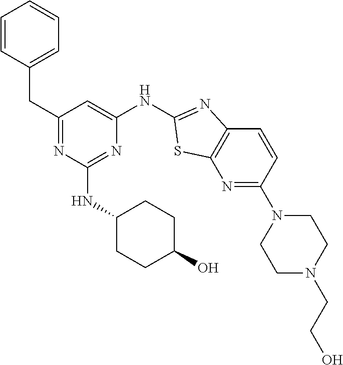 | trans-4-{[4-({5-[4-(2- hydroxyethyl)-1- piperazinyl][1,3]thiazolo [5,4-b]pyridin-2- yl}amino)-6- (phenylmethyl)-2- pyrimidinyl]amino} cyclohexanol | LCMS (Method A): Rt 0.57 minutes; m/z 561 (MH+) | MDAP, ammonium bicarbonate modifier |
| 132 | 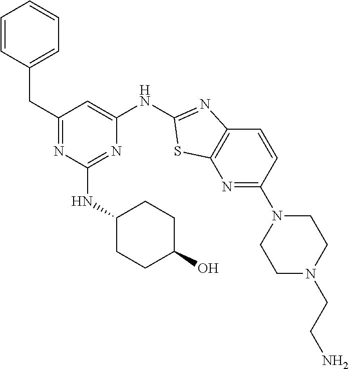 | trans-4-{[4-({5-[4-(2- aminoethyl)-1- piperazinyl][1,3]thiazolo [5,4-b]pyridin-2- yl}amino)-6- (phenylmethyl)-2- pyrimidinyl]amino} cyclohexanol | LCMS (Method A): Rt 0.53 minutes; m/z 560 (MH+) | MDAP, ammonium bicarbonate modifier |

### Examples 133 and 134

3-(4-{[4-{[5-(4-morpholinyl)[1,3]thiazolo[5,4-b]pyridin-2-yl]amino}-6-(phenylmethyl)-2-pyrimidinyl]amino}phenyl)propanoic acid and N²-{4-[3-(4-morpholinyl)-3-oxopropyl]phenyl}-N⁴-[5-(4-morpholinyl)[1,3]thiazolo[5,4-b]pyridin-2-yl]-6-(phenylmethyl)-2,4-pyrimidinediamine

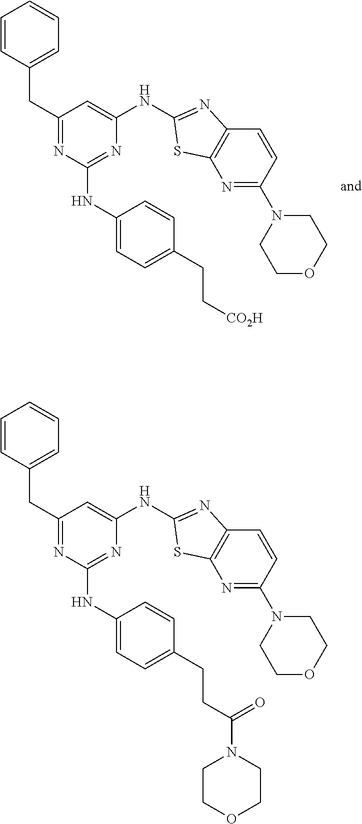

A mixture of 3-(4-{[4-[(5-chloro[1,3]thiazolo[5,4-b]pyridin-2-yl)amino]-6-(phenylmethyl)-2-pyrimidinyl]amino}phenyl)propanoic acid (50 mg, 0.097 mmol) and morpholine (1 mL, 11.48 mmol) was sealed and heated in a Biotage “Initiator” microwave at 130° C. for 4 hours. The products were purified by mass-directed autopreparative HPLC (ammonium bicarbonate modifier) to afford the title compounds.

### Example 133

(4.3 mg, 8%), white solid. LCMS (Method A): Rt 0.93 minutes; m/z 568 (MH+).

### Example 134

(5.2 mg, 5%), white solid. LCMS (Method A): Rt 0.93 minutes; m/z 637 (MH+).

The compound shown in the table was prepared in an analogous manner to that for 3-(4-{[4-{[5-(4-morpholinyl)[1,3]thiazolo[5,4-b]pyridin-2-yl]amino}-6-(phenylmethyl)-2-pyrimidinyl]amino}phenyl)propanoic acid by reacting 3-(4-{[4-[(5-chloro[1,3]thiazolo[5,4-b]pyridin-2-yl)amino]-6-(phenylmethyl)-2-pyrimidinyl]amino}phenyl)propanoic acid with piperazine:

| | | | Analytical | Purification |
|---|---|---|---|---|
| Example | Structure | Name | Data | Method |
| 135 | 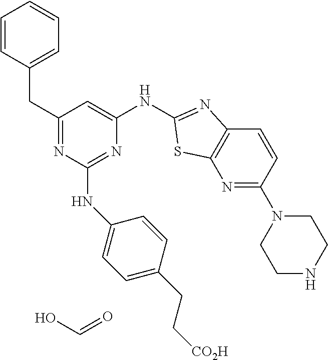 | 3-{4-[(4- (phenylmethyl)-6-{[5- (1-piperazinyl) [1,3]thiazolo [5,4-b]pyridin-2- yl]amino}-2- pyrimidinyl)amino] phenyl}propanoic acid formate | LCMS (Method A): Rt 0.74 minutes; m/z 567 (MH+) | MDAP, ammonium bicarbonate modifier |

### Example 136

### trans-4-[(4-[difluoro(4-fluorophenyl)methyl]-6-{[5-(methyloxy)[1,3]thiazolo[5,4-b]pyridin-2-yl]amino}-2-pyrimidinyl)amino]cyclohexanol

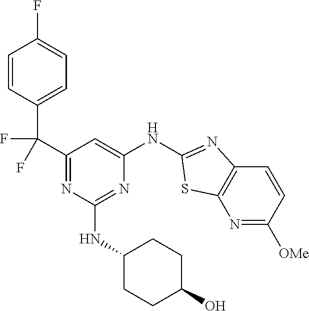

A mixture of N-{2-chloro-6-[difluoro(4-fluorophenyl)methyl]-4-pyrimidinyl}-5-(methyloxy)[1,3]thiazolo[5,4-b]pyridin-2-amine (224 mg, 0.512 mmol), trans-4-aminocyclohexanol (118 mg, 1.023 mmol) and N-ethyldiisopropylamine (0.178 mL, 1.023 mmol) in isopropanol (5 mL) was heated in a Biotage “Initiator” microwave at 170° C. for 2.5 hours. The product was purified by mass-directed autopreparative HPLC (formic acid modifier) to afford the title compound. LCMS (Method A): Rt 1.15 minutes; m/z 517 (MH+).

The compounds shown in the table were prepared in an analogous manner to that for trans-4-[(4-[difluoro(4-fluorophenyl)methyl]-6-{[5-(methyloxy)[1,3]thiazolo[5,4-b]pyridin-2-yl]amino}-2-pyrimidinyl)amino]cyclohexanol by reacting the appropriately substituted 2-chloropyrimidine with the appropriate amine:

| | | | | Purification |
|---|---|---|---|---|
| Example | Structure | Name | Analytical Data | Method |
| 137 | 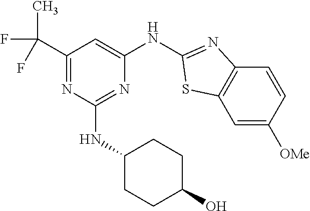 | trans-4-[(4-(1,1- difluoroethyl)-6- {[6-(methyloxy)- 1,3- benzothiazol-2- yl]amino}-2- pyrimidinyl)amino] cyclohexanol | LCMS (Method A): Rt 0.96 minutes; m/z 436 (MH+) | MDAP, formic acid modifier |
| 138 | 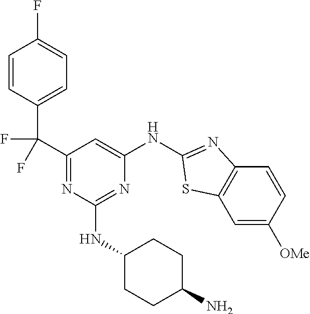 | N²-(trans-4- aminocyclohexyl)- 6-[difluoro(4- fluorophenyl)methyl]- N⁴-[6- (methyloxy)-1,3- benzothiazol-2- yl]-2,4- pyrimidinediamine | LCMS (Method A): Rt 0.89 minutes; m/z 515 (MH+) | MDAP, ammonium bicarbonate modifier |
| 139 | 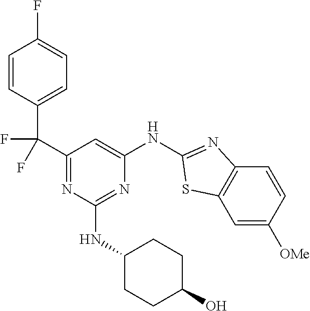 | trans-4-[(4- [difluoro(4- fluorophenyl)methyl]- 6-{[6- (methyloxy)-1,3- benzothiazol-2- yl]amino}-2- pyrimidinyl)amino] cyclohexanol | LCMS (Method A): Rt 0.89 minutes; m/z 516 (MH+) | MDAP, formic acid modifier |
| 140 | 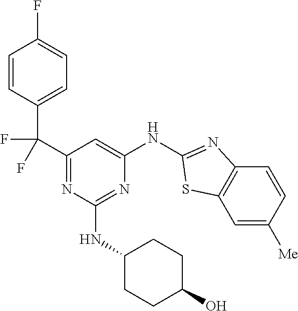 | trans-4-({4- [difluoro(4- fluorophenyl)methyl]- 6-[(6-methyl- 1,3- benzothiazol-2- yl)amino]-2- pyrimidinyl}amino) cyclohexanol | LCMS (Method A): Rt 1.17 minutes; m/z 500 (MH+) | MDAP, formic acid modifier |
| 141 | 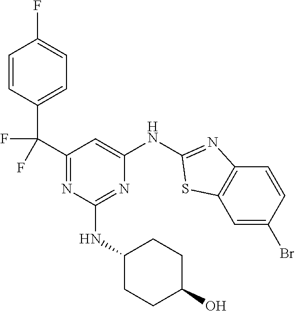 | trans-4-({4-[(6- bromo-1,3- benzothiazol-2- yl)amino]-6- [difluoro(4- fluorophenyl)methyl]-2- pyrimidinyl}amino) cyclohexanol | LCMS (Method A): Rt 1.21 minutes; m/z 564,566 (MH+) | MDAP, formic acid modifier |
| 142 | 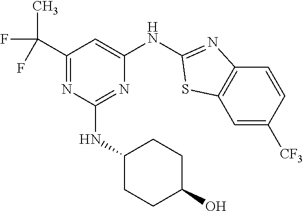 | trans-4-[(4-(1,1- difluoroethyl)-6-{[6- (trifluoromethyl)- 1,3- benzothiazol-2- yl]amino}-2- pyrimidinyl)amino] cyclohexanol | LCMS (Method A): Rt 1.12 minutes; m/z 474 (MH+) | MDAP, formic acid modifier |
| 143 | 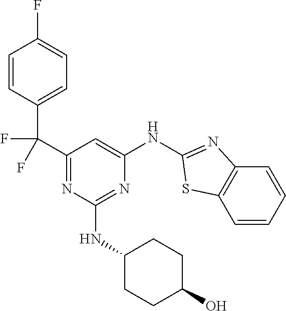 | trans-4-({4-(1,3- benzothiazol-2- ylamino)-6- [difluoro(4- fluorophenyl)methyl]-2- pyrimidinyl}amino) cyclohexanol | LCMS (Method A): Rt 1.09 minutes; m/z 466 (MH+) | MDAP, formic acid modifier |
| 144 | 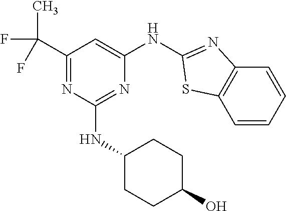 | trans-4-{[4-(1,3- benzothiazol-2- ylamino)-6-(1,1- difluoroethyl)-2- pyrimidinyl]amino} cyclohexanol | LCMS (Method A): Rt 0.96 minutes; m/z 406 (MH+) | MDAP, formic acid modifier |
| 145 | 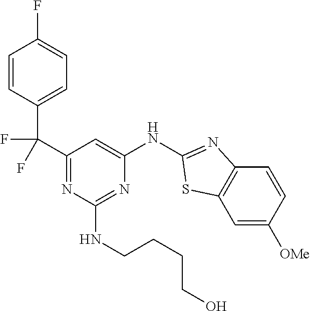 | 4-[(4-[difluoro(4- fluorophenyl)methyl]-6- {[6-(methyloxy)-1,3- benzothiazol-2- yl]amino}-2- pyrimidinyl)amino]- 1-butanol | LCMS (Method A): Rt 1.14 minutes; m/z 490 (MH+) | MDAP, formic acid modifier |
| 146 | 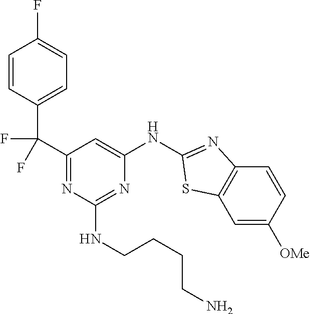 | N²-(4- aminobutyl)-6- [difluoro(4- fluorophenyl)methyl]- N⁴-[6-(methyloxy)-1,3- benzothiazol-2-yl]-2,4- pyrimidinediamine | LCMS (Method A): Rt 1.08 minutes; m/z 489 (MH+) | MDAP, formic acid modifier |
| 147 | 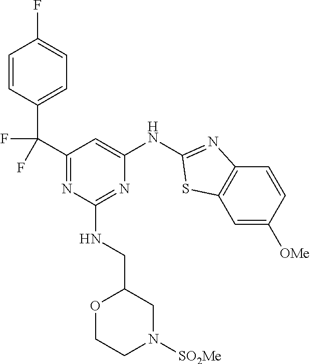 | 6-[difluoro(4- fluorophenyl)methyl]- N⁴-[6-(methyloxy)-1,3- benzothiazol-2- yl]-N²-{[4- (methylsulfonyl)-2- morpholinyl]methyl}-2,4- pyrimidinediamine | LCMS (Method A): Rt 1.11 minutes; m/z 595 (MH+) | MDAP, formic acid modifier |
| 148 | 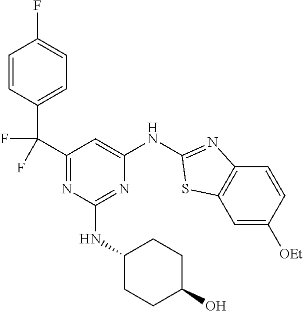 | trans-4-[(4- [difluoro(4- fluorophenyl)methyl]- 6-{[6-(ethyloxy)-1,3- benzothiazol-2- yl]amino}-2- pyrimidinyl)amino] cyclohexanol | LCMS (Method A): Rt 1.17 minutes; m/z 530 (MH+) | MDAP, formic acid modifier |
| 149 | 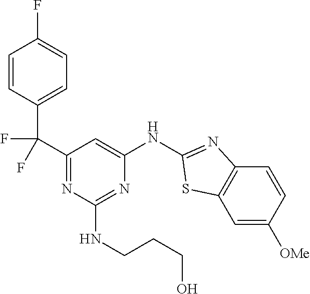 | 3-[(4-[difluoro(4- fluorophenyl)methyl]- 6-{[6-(methyloxy)-1,3- benzothiazol-2- yl]amino}-2- pyrimidinyl)amino]- 1-propanol | LCMS (Method A): Rt 1.12 minutes; m/z 476 (MH+) | MDAP, ammonium bicarbonate modifier |
| 150 | 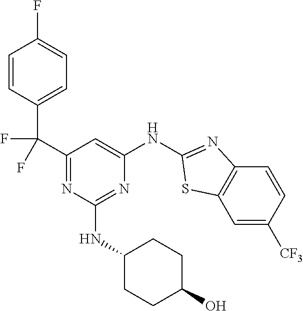 | trans-4-[(4- [difluoro(4- fluorophenyl)methyl]- 6-{[6-(trifluoromethyl)- 1,3-benzothiazol-2- yl]amino}-2- pyrimidinyl)amino] cyclohexanol | LCMS (Method A): Rt 1.23 minutes; m/z 554 (MH+) | MDAP, formic acid modifier |
| 151 | 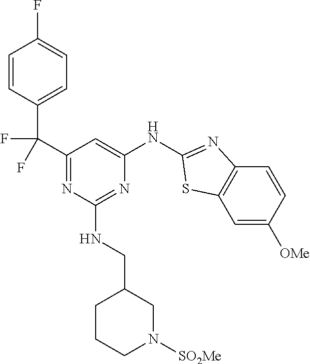 | 6-[difluoro(4- fluorophenyl)methyl]- N⁴-[6-(methyloxy)-1,3- benzothiazol-2- yl]-N²-{[1- (methylsulfonyl)-3- piperidinyl]methyl}-2,4- pyrimidinediamine | LCMS (Method A): Rt 1.19 minutes; m/z 593 (MH+) | MDAP, ammonium bicarbonate modifier |

### Example 152

### {4-[(4-[difluoro(4-fluorophenyl)methyl]-6-{[6-(methyloxy)-1,3-benzothiazol-2-yl]amino}-2-pyrimidinyl)amino]phenyl}acetic acid

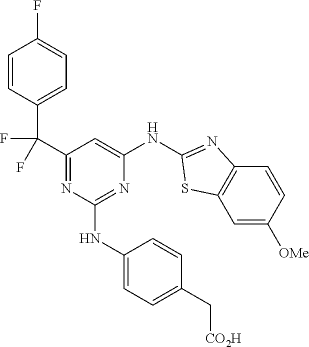

A mixture of N-{2-chloro-6-[difluoro(4-fluorophenyl)methyl]-4-pyrimidinyl}-6-(methyloxy)-1,3-benzothiazol-2-amine (50 mg, 0.114 mmol) and (4-aminophenyl)acetic acid (32.3 mg, 0.28 mmol) in DMSO (0.8 mL) was treated with aqueous hydrochloric acid (2M, 2 drops), sealed and heated in a Biotage “Initiator” microwave at 170° C. for 2 hours. The product was purified by mass-directed autopreparative HPLC (ammonium bicarbonate modifier) to afford the title compound (19 mg, 0.034 mmol, 30% yield). LCMS (Method A): Rt 1.15 minutes; m/z 552 (MH+).

The compounds shown in the table were prepared in an analogous manner to that for {4-[(4-[difluoro(4-fluorophenyl)methyl]-6-{[5-(methyloxy)[1,3]thiazolo[5,4-b]pyridin-2-yl]amino}-2-pyrimidinyl)amino]phenyl}acetic acid by reacting N-{2-chloro-6-[difluoro(4-fluorophenyl)methyl]-4-pyrimidinyl}-6-(methyloxy)-1,3-benzothiazol-2-amine with the appropriate aniline:

| | | | | Purification |
|---|---|---|---|---|
| Example | Structure | Name | Analytical Data | Method |
| 153 | 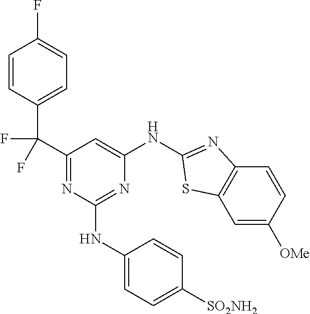 | 4-[(4-[difluoro(4- fluorophenyl)methyl]- 6-{[6-(methyloxy)-1,3- benzothiazol-2- yl]amino}-2- pyrimidinyl)amino] benzenesulfonamide | LCMS (Method A): Rt 1.10 minutes; m/z 573 (MH+) | MDAP, ammonium bicarbonate modifier |
| 154 | 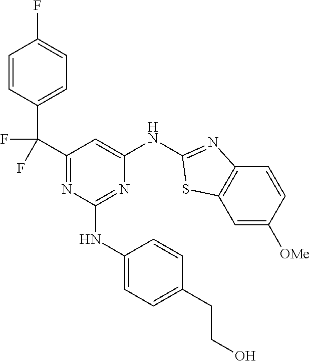 | 2-{4-[(4-[difluoro(4- fluorophenyl)methyl]- 6-{[6-(methyloxy)-1,3- benzothiazol-2- yl]amino}-2- pyrimidinyl)amino] phenyl}ethanol | LCMS (Method A): Rt 1.18 minutes; m/z 538 (MH+) | MDAP, ammonium bicarbonate modifier |

### Example 155

### N-({4-[(4-[difluoro(4-fluorophenyl)methyl]-6-{[6-(ethyloxy)-1,3-benzothiazol-2-yl]amino}-2-pyrimidinyl)amino]phenyl}sulfonyl)acetamide

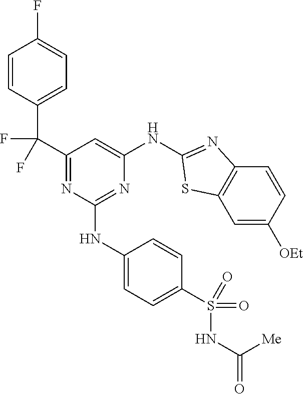

A mixture of N-{2-chloro-6-[difluoro(4-fluorophenyl)methyl]-4-pyrimidinyl}-6-(ethyloxy)-1,3-benzothiazol-2-amine (100 mg, 0.222 mmol), N-[(4-aminophenyl)sulfonyl]acetamide (95 mg, 0.444 mmol) and 4-toluenesulfonic acid monohydrate (50.6 mg, 0.266 mmol) in acetonitrile (3 mL) was sealed and heated at 120° C. for 4 hours in a Biotage “Initiator” microwave. The product was purified by mass-directed autopreparative HPLC (formic acid modifier) to afford the title compound (21 mg, 0.033 mmol, 20% yield) as a white solid. LCMS (Method A): Rt 1.19 minutes; m/z 629 (MH+).

### Example 156

### trans-4-({4-[(6-bromo-1,3-benzothiazol-2-yl)amino]-2-pyrimidinyl}amino)cyclohexanol

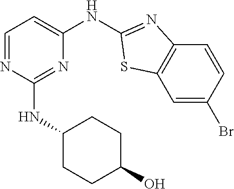

A mixture of 6-bromo-N-[2-(methylsulfonyl)-4-pyrimidinyl]-1,3-benzothiazol-2-amine (1.19 g, 3.09 mmol), trans-4-aminocyclohexanol (0.712 g, 6.18 mmol) and diisopropylethylamine (1.62 mL, 9.27 mmol) in isopropanol (10 mL) was sealed and heated in a Biotage “Initiator” microwave at 130° C. for 3 hours. The cooled reaction mixture was filtered and the filtered solid was dried to afford the title compound (2.38 g, 5.67 mmol, 64% yield). LCMS (Method B): Rt 2.41 minutes; m/z 420,422 (MH+).

### Example 157

### trans-4-({4-[(6-{5-[(1-methylethyl)oxy]-3-pyridinyl}-1,3-benzothiazol-2-yl)amino]-2-pyrimidinyl}amino)cyclohexanol

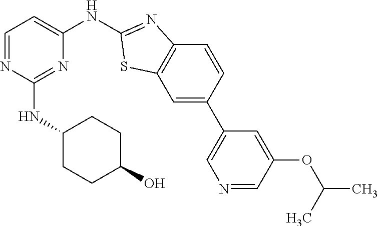

A mixture of 3-[(1-methylethyl)oxy]-5-(4,4,5,5-tetramethyl-1,3,2-dioxaborolan-2-yl)pyridine (125 mg, 0.476 mmol), trans-4-({4-[(6-bromo-1,3-benzothiazol-2-yl)amino]-2-pyrimidinyl}amino)cyclohexanol (100 mg, 0.238 mmol), tetrakis(triphenylphosphine)-palladium(0) (82 mg, 0.071 mmol), caesium carbonate (233 mg, 0.714 mmol) in 1,4-dioxane (2 mL) and water (0.5 mL) was sealed and heated in a Biotage “Initiator” microwave at 130° C. for 30 minutes. The cooled reaction mixture was evaporated to dryness and the product was purified by mass-directed automated preparative HPLC (ammonium bicarbonate modifier) followed by ion exchange chromatography using an SCX (sulfonic acid) solid-phase extraction cartridge and eluting with methanol followed by 2M ammonia in methanol to afford the title compound (4.8 mg, 4% yield). LCMS (Method B): Rt 2.48 minutes; m/z 477 (MH+).

### Example 158

### trans-4-({4-[(6-{5-[(2-hydroxyethyl)oxy]-3-pyridinyl}-1,3-benzothiazol-2-yl)amino]-2-pyrimidinyl}amino)cyclohexanol

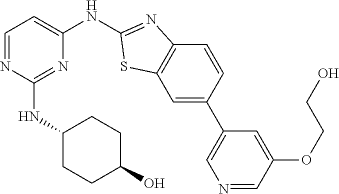

A mixture of trans-4-[(4-{[6-(4,4,5,5-tetramethyl-1,3,2-dioxaborolan-2-yl)-1,3-benzothiazol-2-yl]amino}-2-pyrimidinyl)amino]cyclohexanol (100 mg, 0.214 mmol), 2-[(5-bromo-3-pyridinyl)oxy]ethanol (46.7 mg, 0.214 mmol), tetrakis(triphenylphosphine)palladium(0) (74.2 mg, 0.064 mmol) and caesium carbonate (209 mg, 0.642 mmol) in 1,4-dioxane (2 mL) and water (0.5 mL) was sealed and heated in a Biotage “Initiator” microwave at 130° C. for 30 minutes. The reaction mixture was then loaded onto a C18 column (5 g) (primed with 0.1% trifluoroacetic acid in acetonitrile) and eluted with 0.1% trifluoroacetic acid in acetonitrile). Product-containing fractions were evaporated to dryness and the product was purified by mass-directed automated preparative HPLC (ammonium bicarbonate modifier) to afford the title compound (5 mg, 5% yield). LCMS (Method B): Rt 1.94 minutes; m/z 479 (MH+).

The compounds shown in the table were prepared in an analogous manner to that for trans-4-({4-[(6-{5-[(2-hydroxyethyl)oxy]-3-pyridinyl}-1,3-benzothiazol-2-yl)amino]-2-pyrimidinyl}amino)cyclohexanol by reacting trans-4-[(4-{[6-(4,4,5,5-tetramethyl-1,3,2-dioxaborolan-2-yl-1,3-benzothiazol-2-yl]amino}-2-pyrimidinyl)amino]cyclohexanol with the appropriate aryl bromide:

| | | | | Purification |
|---|---|---|---|---|
| Example | Structure | Name | Analytical Data | Method |
| 159 | 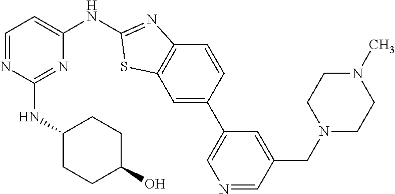 | trans-4-({4-[(6-{5-[(4- methyl-1- piperazinyl)methyl]-3- pyridinyl}-1,3- benzothiazol-2- yl)amino]-2- pyrimidinyl}amino)cyclohexanol | LCMS (Method A): Rt 0.45 minutes; m/z 531 (MH+) | MDAP, ammonium bicarbonate modifier |
| 160 | 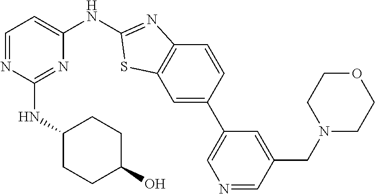 | trans-4-{[4-({6-[5-(4- morpholinylmethyl)-3- pyridinyl]-1,3- benzothiazol-2- yl}amino)-2- pyrimidinyl]amino}cyclohexanol | LCMS (Method A): Rt 0.47 minutes; m/z 518 (MH+) | MDAP, ammonium bicarbonate modifier |
| 161 | 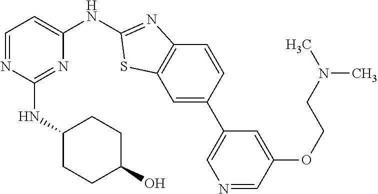 | trans-4-[(4-{[6-(5-{[2- (dimethylamino)ethyl]oxy}- 3-pyridinyl)-1,3- benzothiazol-2- yl]amino}-2- pyrimidinyl)amino]cyclohexanol | LCMS (Method A): Rt 0.46 minutes; m/z 506 (MH+) | MDAP, ammonium bicarbonate modifier |
| 162 | 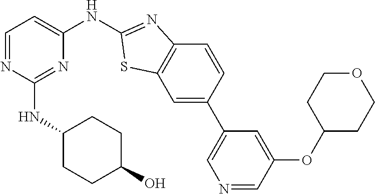 | trans-4-{[4-({6-[5- (tetrahydro-2H-pyran- 4-yloxy)-3-pyridinyl]- 1,3-benzothiazol-2- yl}amino)-2- pyrimidinyl]amino}cyclohexanol | LCMS (Method A): Rt 0.61 minutes; m/z 519 (MH+) | MDAP, ammonium bicarbonate modifier |
| 163 | 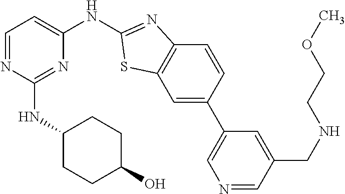 | trans-4-{[4-({6-[5-({[2- (methyloxy)ethyl]amino} methyl)-3-pyridinyl]- 1,3-benzothiazol-2- yl}amino)-2- pyrimidinyl]amino}cyclohexanol | LCMS (Method A): Rt 0.47 minutes; m/z 506 (MH+) | MDAP, ammonium bicarbonate modifier |
| 164 | 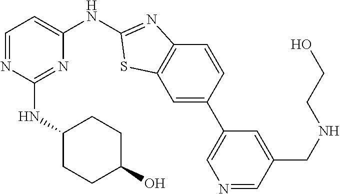 | trans-4-[(4-{[6-(5-{[(2- hydroxyethyl)amino]methyl}- 3-pyridinyl)-1,3- benzothiazol-2- yl]amino}-2- pyrimidinyl)amino]cyclohexanol | LCMS (Method A): Rt 0.45 minutes; m/z 492 (MH+) | MDAP, ammonium bicarbonate modifier |
| 165 | 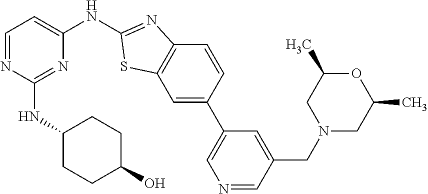 | trans-4-[(4-{[6-(5- {[(2R,6S)-2,6- dimethyl-4- morpholinyl]methyl}-3- pyridinyl)-1,3- benzothiazol-2- yl]amino}-2- pyrimidinyl)amino]cyclohexanol | LCMS (Method A): Rt 0.51 minutes; m/z 546 (MH+) | MDAP, ammonium bicarbonate modifier |

### Example 166

### trans-4-({4-[(5-chloro[1,3]thiazolo[5,4-b]pyridin-2-yl)amino]-6-methyl-2-pyrimidinyl}amino)cyclohexanol

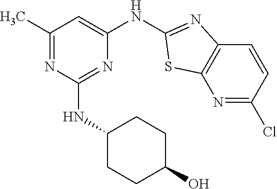

A mixture of 5-chloro-N-(2-fluoro-6-methyl-4-pyrimidinyl)[1,3]thiazolo[5,4-b]pyridin-2-amine (8.8 g, 29.8 mmol) and trans-4-aminocyclohexanol (10.28 g, 89 mmol) was treated with ethylene glycol (20 mL) and the mixture was heated at 150° C. for 4 hours. The cooled mixture was added to water (200 mL) and stirred for 30 minutes. The precipitated product was filtered off, washed with water and dried to afford trans-4-({4-[(5-chloro[1,3]thiazolo[5,4-b]pyridin-2-yl)amino]-6-methyl-2-pyrimidinyl}amino)cyclohexanol (9.35 g, 23.92 mmol, 80% yield). LCMS (Method A): Rt 0.65 minutes; m/z 391 (MH+)

### Example 167

### trans-4-[(4-(phenylmethyl)-6-{[5-(1H-pyrazol-4-yl)[1,3]thiazolo[5,4-b]pyridin-2-yl]amino}-2-pyrimidinyl)amino]cyclohexanol

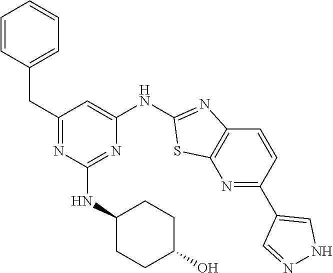

A mixture of trans-4-{[4-[(5-chloro[1,3]thiazolo[5,4-b]pyridin-2-yl)amino]-6-(phenylmethyl)-2-pyrimidinyl]amino}cyclohexanol (32 mg, 0.069 mmol), 4-(4,4,5,5-tetramethyl-1,3,2-dioxaborolan-2-yl)-1H-pyrazole (29.3 mg, 0.151 mmol), tetrakis(triphenylphosphine)-palladium(0) (4.04 mg, 0.0035 mmol), caesium carbonate (59.4 mg, 0.182 mmol) in 1,4-dioxane (2.4 mL) and water (0.6 mL) was heated in a sealed tube in a Biotage “Initiator” microwave at 150° C. for 20 minutes. Additional 4-(4,4,5,5-tetramethyl-1,3,2-dioxaborolan-2-yl)-1H-pyrazole (29.3 mg, 0.151 mmol) and tetrakis(triphenylphosphine)palladium(0) (4.04 mg, 0.0035 mmol) was added, the reaction was degassed for 15 minutes and then heated in the microwave at 150° C. for 20 minutes. Additional 1,1-dimethylethyl 4-(4,4,5,5-tetramethyl-1,3,2-dioxaborolan-2-yl)-1H-pyrazole-1-carboxylate (44.3 mg, 0.151 mmol) and tetrakis(triphenylphosphine)-palladium(0) (4.04 mg, 0.0035 mmol) were added, the reaction was degassed for 15 minutes then heated in the microwave at 150° C. for 1 hour. Additional 1,1-dimethylethyl 4-(4,4,5,5-tetramethyl-1,3,2-dioxaborolan-2-yl)-1H-pyrazole-1-carboxylate (44.3 mg, 0.151 mmol) and tetrakis(triphenylphosphine)palladium(0) (4.04 mg, 0.0035 mmol) were added and the reaction was heated in the microwave at 150° C. for 1.5 hours. The reaction mixture was then partitioned between water (10 mL) and ethyl acetate (10 mL). The aqueous phase was extracted with further ethyl acetate (10 mL). The aqueous phase was filtered and the filtered solid dissolved in a mixture of methanol and dichloromethane, combined with the ethyl acetate extracts and evaporated to dryness. The product was purified by mass-directed automated preparative HPLC (ammonium bicarbonate modifier) to afford the title compound (9.7 mg, 0.019 mmol, 15%). LCMS (Method B): Rt 1.98 minutes; m/z 499 (MH+).

### Example 168

### trans-4-[(4-(phenylmethyl)-6-{[6-(1H-pyrazol-4-yl)-1,3-benzothiazol-2-yl]amino}-2-pyrimidinyl)amino]cyclohexanol

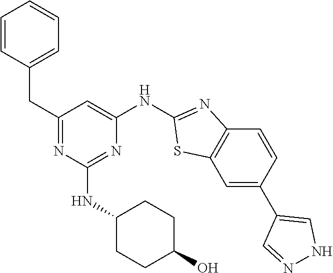

A mixture of 1,1-dimethylethyl 4-(4,4,5,5-tetramethyl-1,3,2-dioxaborolan-2-yl)-1H-pyrazole-1-carboxylate (192 mg, 0.652 mmol), trans-4-{[4-[(6-bromo-1,3-benzothiazol-2-yl)amino]-6-(phenylmethyl)-2-pyrimidinyl]amino}cyclohexanol (131 mg, 0.257 mmol), caesium carbonate (251 mg, 0.770 mmol), 1,1′-bis(diphenylphosphino)ferrocene-palladium(II) dichloride dichloromethane adduct (10.48 mg, 0.013 mmol) was sealed and heated in a Biotage “Initiator” microwave at 150° C. for 30 minutes. Tetrakis(triphenylphosphine)palladium(0) (15 mg, 0.013 mmol), 1,1-dimethylethyl 4-(4,4,5,5-tetramethyl-1,3,2-dioxaborolan-2-yl)-1H-pyrazole-1-carboxylate (192 mg, 0.652 mmol) and water (0.6 mL) were added and the reaction mixture was heated at 150° C. for a further 30 minutes. More tetrakis(triphenylphosphine)palladium(0) (15 mg, 0.013 mmol) and 1,1-dimethylethyl 4-(4,4,5,5-tetramethyl-1,3,2-dioxaborolan-2-yl)-1H-pyrazole-1-carboxylate (192 mg, 0.652 mmol) were added and the reaction mixture heated for a further 1 hour at 150° C. The reaction mixture was then partitioned between water (20 mL) and ethyl acetate (20 mL). The aqueous phase was extracted with ethyl acetate (20 mL) and the combined ethyl acetate extracts were evaporated to dryness. The product was purified by mass-directed automated preparative HPLC (ammonium bicarbonate modifier) to afford the title compound (46 mg, 0.09 mmol, 36%). LCMS (Method B): Rt 2.48 minutes; m/z 498 (MH+).

### Example 169

### N²-(trans-4-aminocyclohexyl)-6-(phenylmethyl)-N⁴-[5-(1H-pyrazol-4-yl)[1,3]thiazolo[5,4-b]pyridin-2-yl]-2,4-pyrimidinediamine

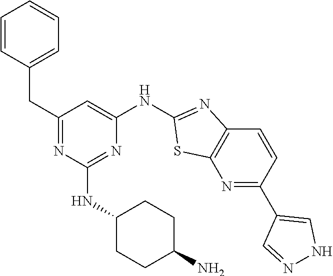

A mixture of 1,1-dimethylethyl 4-(4,4,5,5-tetramethyl-1,3,2-dioxaborolan-2-yl)-1H-pyrazole-1-carboxylate (303 mg, 1.03 mmol), N²-(trans-4-aminocyclohexyl)-N⁴-(5-chloro[1,3]thiazolo[5,4-b]pyridin-2-yl)-6-(phenylmethyl)-2,4-pyrimidinediamine (189 mg, 0.406 mmol), tetrakis(triphenylphosphine)palladium(0) (141 mg, 0.122 mmol), caesium carbonate (396 mg, 1.22 mmol) in 1,4-dioxane (8 mL) and water (2 mL) was heated in a sealed tube in a Biotage “Initiator” microwave at 150° C. for 1 hour. Further 1,1-dimethylethyl 4-(4,4,5,5-tetramethyl-1,3,2-dioxaborolan-2-yl)-1H-pyrazole-1-carboxylate (303 mg, 1.03 mmol) and tetrakis(triphenylphosphine)palladium(0) (141 mg, 0.122 mmol) were added and the reaction mixture heated in the microwave at 150° C. for 1 hour. The reaction mixture was partitioned between water (50 mL) and ethyl acetate (50 mL) and the aqueous phase extracted with further ethyl acetate (50 mL). The aqueous phase was filtered and the recovered solid was combined with the ethyl acetate extracts and evaporated to dryness. The residue was triturated with dichloromethane, filtered and the filtered solid was purified by mass-directed automated preparative HPLC (ammonium bicarbonate modifier) to afford the title compound (30 mg, 0.06 mmol, 15%). LCMS (Method B): Rt 2.32 minutes; m/z 498 (MH+).

### Example 170

### trans-4-{[4-{[6-(3-methyl-1H-pyrazol-4-yl)-1,3-benzothiazol-2-yl]amino}-6-(phenylmethyl)-2-pyrimidinyl]amino}cyclohexanol trifluoroacetate (salt)

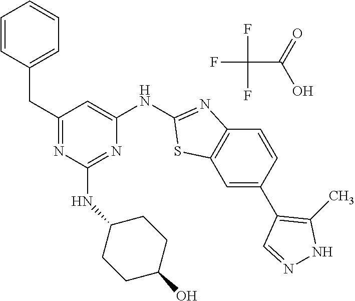

A mixture of trans-4-{[4-[(6-bromo-1,3-benzothiazol-2-yl)amino]-6-(phenylmethyl)-2-pyrimidinyl]amino}cyclohexanol (50 mg, 0.1 mmol), 3-methyl-4-(4,4,5,5-tetramethyl-1,3,2-dioxaborolan-2-yl-1H-pyrazole (42 mg, 0.2 mmol), 2′-(dimethylamiono)-2-biphenyl-palladium(II) chloride dinorbornylphosphine complex (2.2 mg, 0.004 mmol) and potassium phosphate (0.15 mmol, 32 mg) in 1,4-dioxane (0.8 mL) and water (0.2 mL) was sealed and heated in a CEM “Discover” microwave at 110° C. for 20 minutes. After cooling additional 3-methyl-4-(4,4,5,5-tetramethyl-1,3,2-dioxaborolan-2-yl)-1H-pyrazole (0.2 mmol, 42 mg) and 2′-(dimethylamiono)-2-biphenyl-palladium(ii) chloride dinorbornylphosphine complex (2.2 mg, 0.004 mmol) was added and the vessel sealed and heated at 110° C. for an additional 30 minutes, then at 135° C. for 5 minutes, then at 140° C. for a further 5 minutes. The reaction mixture was then loaded onto a C18 solid-phase extraction cartridge (pre-conditioned with acetonitrile/0.1% trifluoroacetic acid) and the cartridge was eluted with acetonitrile/0.1% trifluoroacetic acid. The product-containing fractions were evaporated to dryness and the product was purified using mass-directed automated preparative HPLC (TFA modifier) to afford the title compound (1.4 mg, 0.0027 mmol, 3% yield). LCMS (Method A): Rt 0.68 minutes; m/z 512 (MH+).

### Example 171

### trans-4-[(4-(phenylmethyl)-6-{[6-(4-pyridinyl)-1,3-benzothiazol-2-yl]amino}-2-pyrimidinyl)amino]cyclohexanol formate (salt)

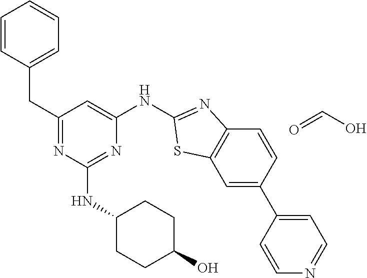

A mixture of trans-4-{[4-[(6-bromo-1,3-benzothiazol-2-yl)amino]-6-(phenylmethyl)-2-pyrimidinyl]amino}cyclohexanol (50 mg, 0.1 mmol), 4-pyridinylboronic acid (25 mg, 0.2 mmol), 2′-(dimethylamiono)-2-biphenyl-palladium(II) chloride dinorbornylphosphine complex (2.2 mg, 0.004 mmol) and potassium phosphate (32 mg, 0.15 mmol) in 1,4-dioxane (0.8 mL) and water (0.2 mL) was sealed and heated in a CEM “Discover” microwave at 110° C. for 20 minutes. After cooling additional 2′-(dimethylamiono)-2-biphenyl-palladium(ii) chloride dinorbornylphosphine complex (2.242 mg, 0.004 mmol) was added and the mixture was heated at 110° C. for an additional 30 minutes. The reaction mixture was then loaded onto C18 solid-phase extraction cartridge (pre-conditioned with acetonitrile/0.1% trifluoroacetic acid) and the cartridge was eluted with acetonitrile/0.1% trifluoroacetic acid. The product-containing fractions were evaporated to dryness and the product was purified using mass-directed automated preparative HPLC (TFA modifier) followed by purification using mass-directed automated preparative HPLC (formic acid modifier) to afford the title compound (6.5 mg, 0.013 mmol, 13% yield). LCMS (Method A): Rt 0.56 minutes; m/z 509 (MH+).

The compounds shown in the table were prepared in an analogous manner to that for trans-4-[(4-(phenylmethyl)-6-{[6-(4-pyridinyl)-1,3-benzothiazol-2-yl]amino}-2-pyrimidinyl)amino]cyclohexanol formate by reacting trans-4-{[4-[(6-bromo-1,3-benzothiazol-2-yl)amino]-6-(phenylmethyl)-2-pyrimidinyl]amino}cyclohexanol with the appropriate boronic acid or boronic ester:

| | | | | Purification |
|---|---|---|---|---|
| Example | Structure | Name | Analytical Data | Method |
| 172 | 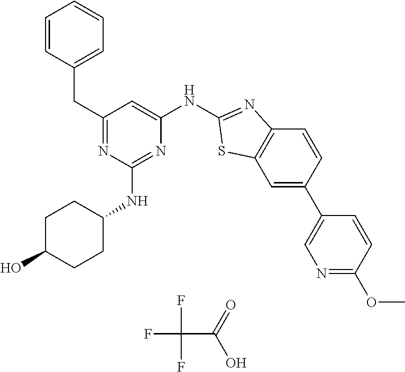 | trans-4-{[4-({6-[6- (methyloxy)-3- pyridinyl]-1,3- benzothiazol-2- yl}amino)-6- (phenylmethyl)-2- pyrimidinyl]amino}cyclohexanol trifluoroacetate (salt) | LCMS (Method A): Rt 0.82 minutes; m/z 539 (MH+) | MDAP, trifluoroacetic acid modifier |
| 173 | 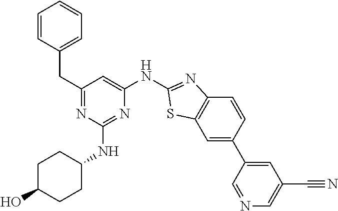 | 5-(2-{[2-[(trans-4- hydroxycyclohexyl)amino]- 6-(phenylmethyl)- 4-pyrimidinyl]amino}- 1,3-benzothiazol-6-yl)- 3-pyridinecarbonitrile | LCMS (Method A): Rt 0.81 minutes; m/z 534 (MH+) | MDAP, ammonium bicarbonate modifier |
| 174 | 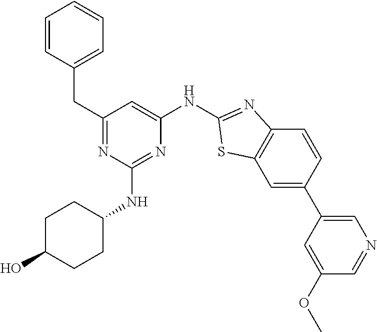 | trans-4-{[4-({6-[5- (methyloxy)-3- pyridinyl]-1,3- benzothiazol-2- yl}amino)-6- (phenylmethyl)-2- pyrimidinyl]amino}cyclohexanol | LCMS (Method A): Rt 0.74 minutes; m/z 539 (MH+) | MDAP, ammonium bicarbonate modifier |
| 175 | 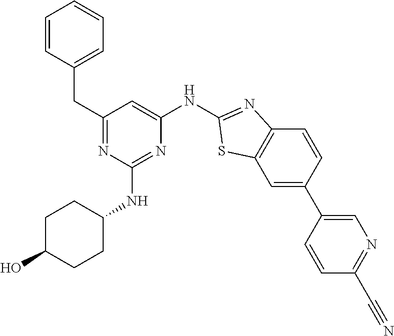 | 5-(2-{[2-[(trans-4- hydroxycyclohexyl)amino]- 6-(phenylmethyl)- 4-pyrimidinyl]amino}- 1,3-benzothiazol-6-yl)- 2-pyridinecarbonitrile | LCMS (Method A): Rt 0.84 minutes; m/z 534 (MH+) | MDAP, ammonium bicarbonate modifier |

### Example 176

### trans-4-{[4-{[6-(4-isoxazolyl)-1,3-benzothiazol-2-yl]amino}-6-(phenylmethyl)-2-pyrimidinyl]amino}cyclohexanol

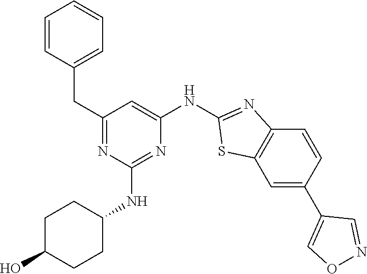

A mixture of trans-4-{[4-[(6-bromo-1,3-benzothiazol-2-yl)amino]-6-(phenylmethyl)-2-pyrimidinyl]amino}cyclohexanol (50 mg, 0.1 mmol), 4-(4,4,5,5-tetramethyl-1,3,2-dioxaborolan-2-yl)isoxazole (39 mg, 0.2 mmol), 2′-(dimethylamiono)-2-biphenyl-palladium(II) chloride dinorbornylphosphine complex (2.2 mg, 0.004 mmol) and potassium phosphate (42 mg, 0.2 mmol) in 1,4-dioxane (0.8 mL) and water (0.2 mL) was sealed and heated in an Anton Parr microwave at 100° C. for 20 minutes. After cooling additional 2′-(dimethylamiono)-2-biphenyl-palladium(ii) chloride dinorbornylphosphine complex (2.2 mg, 0.004 mmol) and 4-(4,4,5,5-tetramethyl-1,3,2-dioxaborolan-2-yl)isoxazole (39 mg, 0.2 mmol) were added and the mixture was heated in a CEM “Discover” microwave at 135° C. for an additional 5 minutes. The reaction mixture was then loaded onto a C18 solid-phase extraction cartridge (pre-conditioned with acetonitrile/0.1% trifluoroacetic acid) and the cartridge was eluted with acetonitrile/0.1% trifluoroacetic acid. The product-containing fractions were evaporated to dryness and the product was purified using mass-directed automated preparative HPLC (ammonium bicarbonate modifier) to afford the title compound (1.15 mg, 0.002 mmol, 2.2% yield). LCMS (Method A): Rt 0.79 minutes; m/z 499 (MH+).

### Example 177

### 3-(2-{[2-[(trans-4-hydroxycyclohexyl)amino]-6-(phenylmethyl)-4-pyrimidinyl]amino}[1,3]thiazolo[5,4-b]pyridin-5-yl)-1,3-oxazolidin-2-one

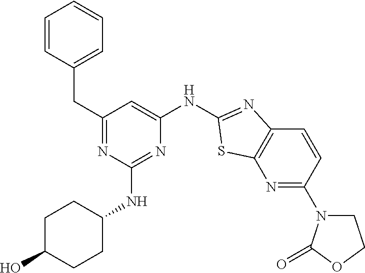

Under an atmosphere of nitrogen, a mixture of trans-4-{[4-[(5-chloro[1,3]thiazolo[5,4-b]pyridin-2-yl)amino]-6-(phenylmethyl)-2-pyrimidinyl]amino}cyclohexanol (80 mg, 0.171 mmol), 1,3-oxazolidin-2-one (30 mg, 0.345 mmol), caesium carbonate (167 mg, 0.514 mmol) and copper(1) iodide (98 mg, 0.514 mmol) in dry N,N-dimethylformamide (5 mL) was treated with N,N′-dimethylethylenediamine (0.073 mL, 0.685 mmol) and the mixture was heated at 120° C. for 6 hours. The cooled mixture was filtered and the filtrate was evaporated to dryness. The product was purified by mass-directed automated preparative HPLC (formic acid modifier) to afford the title compound (3.4 mg, 0.0066 mmol, 3.8% yield). LCMS (Method A): Rt 0.72 minutes; m/z 518 (MH+).

### Example 178

### trans-4-{[4-{[6-(1,1-dioxido-2-isothiazolidinyl)-1,3-benzothiazol-2-yl]amino}-6-(phenylmethyl)-2-pyrimidinyl]amino}cyclohexanol

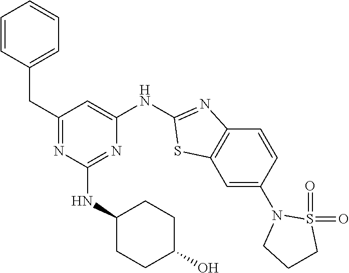

3-Chloro-1-propanesulfonyl chloride (1.361 mL, 0.112 mmol) was added dropwise to an ice-cooled, stirred solution of trans-4-{[4-[(6-amino-1,3-benzothiazol-2-yl)amino]-6-(phenylmethyl)-2-pyrimidinyl]amino}cyclohexanol (50 mg, 0.112 mmol), diisopropylethylamine (0.020 mL, 0.112 mmol) and N,N-dimethyl-4-pyridinamine (6.84 mg, 0.056 mmol) in tetrahydrofuran (2 mL). The solution was stirred for 5 minutes before potassium tert-butoxide (50 mg, 0.446 mmol) was added portionwise over 5 minutes. The reaction mixture was treated with water (2 mL) and brine (20 mL) and then extracted twice with ethyl acetate (2×20 mL). The product was purified by mass-directed automated preparative HPLC (formic acid modifier) to afford the title compound (8.5 mg, 0.015 mmol, 14% yield). LCMS (Method B): Rt 2.86 minutes; m/z 551 (MH+).

### Example 179

### N²-(trans-4-aminocyclohexyl)-N⁴-(5-chloro[1,3]thiazolo[5,4-b]pyridin-2-yl)-6-(phenylmethyl)-2,4-pyrimidinediamine

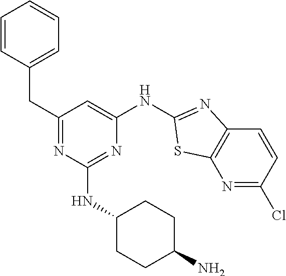

A mixture of 5-chloro-N-[2-fluoro-6-(phenylmethyl)-4-pyrimidinyl][1,3]thiazolo[5,4-b]pyridin-2-amine (150 mg, 0.403 mmol), trans-1,4-cyclohexanediamine (184 mg, 1.614 mmol) in isopropanol (2 mL) was sealed and heated in a Biotage “Initiator” microwave at 130° C. for 1 hour. The cooled reaction mixture was filtered and the recovered solid was washed with isopropanol to give the title compound (189 mg, 0.41 mmol, 100% yield) as a white solid. LCMS (Method B): Rt 2.53 minutes; m/z 466 (MH+).

### Example 180

### trans-4-({4-[(6-bromo-1,3-benzothiazol-2-yl)amino]-6-[(methyloxy)methyl]-2-pyrimidinyl}amino)cyclohexanol

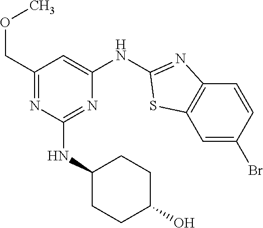

To a stirred suspension of 6-bromo-N-[6-[(methyloxy)methyl]-2-(methylsulfonyl)-4-pyrimidinyl]-1,3-benzothiazol-2-amine (21.5 g, 50.1 mmol) and diisopropylethylamine (26.2 mL, 150 mmol) in 1,4-dioxane (200 mL) at ambient temperature was added trans-4-aminocyclohexanol (17.30 g, 150 mmol). The reaction mixture was heated to reflux until LCMS indicated reaction completion. Water (300 mL) was added and the mixture was filtered. The recovered solid was washed with more water (200 mL) and dried thoroughly to afford the title compound (21.16 g, 45.6 mmol, 91% yield). LCMS (Method A) Rt: 0.78 minutes; m/z: 464, 466 (MH+).

### Example 181

### trans-4-{[4-[(6-bromo-1,3-benzothiazol-2-yl)amino]-6-(hydroxymethyl)-2-pyrimidinyl]amino}cyclohexanol

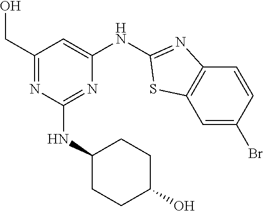

To a suspension of trans-4-({4-[(6-bromo-1,3-benzothiazol-2-yl)amino]-6-[(methyloxy)methyl]-2-pyrimidinyl}amino)cyclohexanol (50 mg, 0.108 mmol) in dichloromethane (5 mL) at 0° C. was added a solution of boron tribromide (1M in dichloromethane) (0.11 mL, 0.108 mmol) dropwise. The solution was stirred at 0° C. for 15 minutes then left to reach ambient temperature whereupon the reaction mixture was stirred for 2 hours. The reaction mixture was then cooled to 0° C. and water (10 mL) was added dropwise. The mixture was separated and the aqueous phase extracted with dichloromethane (10 mL). The organic fractions were combined, dried over magnesium sulfate, filtered and evaporated to dryness. The product was purified by mass-directed automated preparative HPLC (ammonium bicarbonate modifier) to afford the title compound (19.33 g, 42.9 mmol, 94% yield). LCMS (Method A): Rt 0.70 minutes; m/z 450, 452 (MH+).

### Example 182

### trans-4-({4-(4-morpholinylmethyl)-6-[(6-nitro-1,3-benzothiazol-2-yl)amino]-2-pyrimidinyl}amino)cyclohexanol

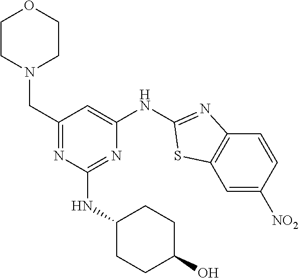

A mixture of N-[2-(methylsulfinyl)-6-(4-morpholinylmethyl)-4-pyrimidinyl]-6-nitro-1,3-benzothiazol-2-amine (80 mg, 0.184 mmol), trans-4-aminocyclohexanol (27.6 mg, 0.239 mmol) and diisopropylethylamine (0.096 mL, 0.552 mmol) in isopropanol (4 mL) was sealed and heated in a Biotage “Initiator” microwave at 150° C. for 1 hour. The solvent was removed under vacuum and the product was purified by mass-directed automated preparative HPLC (ammonium bicarbonate modifier) to afford the title compound (3.3 mg, 4% yield) as a yellow solid. LCMS (Method B): Rt 2.22 minutes; m/z 486 (MH+).

### Examples 183 and 184

trans-4-({4-[(6-bromo-1,3-benzothiazol-2-yl)amino]-6-[cis-(3,5-dimethyl-4-morpholinyl)methyl]-2-pyrimidinyl}amino)cyclohexanol and trans-4-({4-[(6-bromo-1,3-benzothiazol-2-yl)amino]-6-[trans-(3,5-dimethyl-4-morpholinyl)methyl]-2-pyrimidinyl}amino)cyclohexanol

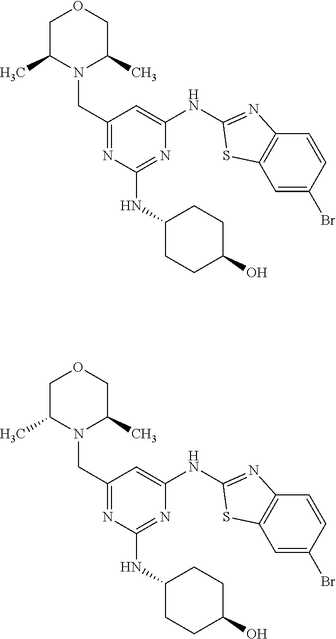

A mixture of 6-[(6-bromo-1,3-benzothiazol-2-yl)amino]-2-[(trans-4-hydroxycyclohexyl)amino]-4-pyrimidinecarbaldehyde (30 mg, 0.067 mmol) and 3,5-dimethylmorpholine (0.020 mL, 0.163 mmol) in tetrahydrofuran (1 mL) was stirred at ambient temperature for 30 minutes. The mixture was then treated with dichloromethane (3 mL) and sodium triacetoxyborohydride (45.3 mg, 0.203 mmol) and stirred for 18 hours. The mixture was then treated with methanol (0.5 mL) and saturated aqueous sodium bicarbonate (5 mL) and then separated. The aqueous phase was extracted with chloroform (+10% methanol) (2×5 mL) and the combined organic phase was evaporated to dryness. The product was purified by mass-directed automated preparative HPLC (ammonium bicarbonate modifier) to afford the isomeric title compounds. First isomer (7.2 mg, 0.013 mmol, 20% yield). LCMS (Method B): Rt 2.65 minutes; m/z 547,549 (MH+); second isomer (8.9 mg, 0.016 mmol, 24% yield). LCMS (Method B): Rt 2.75 minutes; m/z 547,549 (MH+).

### Example 185

### trans-4-{[4-[(6-bromo-1,3-benzothiazol-2-yl)amino]-6-(1-piperidinylmethyl)-2-pyrimidinyl]amino}cyclohexanol

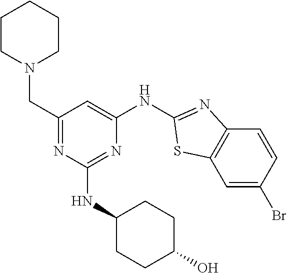

To a stirred suspension of 6-[(6-bromo-1,3-benzothiazol-2-yl)amino]-2-[(trans-4-hydroxycyclohexyl)amino]-4-pyrimidinecarbaldehyde (1.68 g, 3.75 mmol) in tetrahydrofuran (50 mL) was added piperidine (0.4 mL, 4.50 mmol) dropwise. The reaction mixture was stirred at room temperature for 30 minutes, followed by the addition of dichloromethane (150 mL) and sodium triacetoxyborohydride (1.25 g, 5.62 mmol) portionwise. The reaction mixture was stirred at room temperature for 20 minutes, then treated with saturated aqueous sodium bicarbonate and stirred rapidly for 1 hour. The mixture was separated and the aqueous phase was extracted twice with dichloromethane. The combined organic phase was dried over magnesium sulfate, filtered and evaporated to dryness to afford the title compound (1.912 g, 3.69 mmol, 99% yield). LCMS (Method A): Rt 0.77 minutes; m/z 517, 519 (MH+).

### Example 186

### trans-4-({4-[(6-bromo-1,3-benzothiazol-2-yl)amino]-6-[(diethylamino)methyl]-2-pyrimidinyl}amino)cyclohexanol

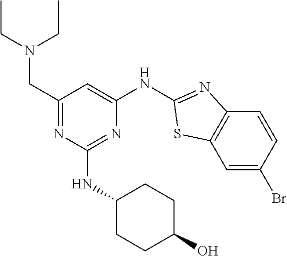

A mixture of 6-[(6-bromo-1,3-benzothiazol-2-yl)amino]-2-[(trans-4-hydroxycyclohexyl)amino]-4-pyrimidinecarbaldehyde (30 mg, 0.067 mmol) and diethylamine (0.014 mL, 0.134 mmol) in tetrahydrofuran (1 mL) was stirred at ambient temperature for 30 minutes. The mixture was treated with dichloromethane (3 mL) and sodium triacetoxyborohydride (45.3 mg, 0.203 mmol) and stirred for 18 hours. The mixture was then treated with methanol (0.5 mL) and saturated aqueous sodium bicarbonate (5 mL) and then separated. The aqueous phase was extracted with chloroform (+10% methanol) (2×5 mL) and the combined organic phase was blown to dryness with a stream of nitrogen. The product was purified by mass-directed automated preparative HPLC (ammonium bicarbonate modifier) to afford the title compound (14.2 mg, 0.028 mmol, 42% yield). LCMS (Method A): Rt 0.80 minutes; m/z 505,507 (MH+)

The compounds shown in the table were prepared in an analogous manner to that for trans-4-([4-[(6-bromo-1,3-benzothiazol-2-yl-amino]-6-[(diethylamino)methyl]-2-pyrimidinyl]amino)cyclohexanol by reacting 6-[(6-bromo-1,3-benzothiazol-2-yl)amino]-2-[(trans-4-hydroxycyclohexyl)amino]-4-pyrimidinecarbaldehyde with the appropriate amine:

| | | | | Purification |
|---|---|---|---|---|
| Example | Structure | Name | Analytical Data | Method |
| 187 | 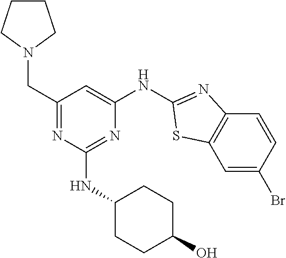 | trans-4-{[4-[(6-bromo- 1,3-benzothiazol-2- yl)amino]-6-(1- pyrrolidinylmethyl)-2- pyrimidinyl]amino}cyclohexanol | LCMS (Method A): Rt 0.77 minutes; m/z 503,505 (MH+) | MDAP, ammonium bicarbonate modifier |
| 188 | 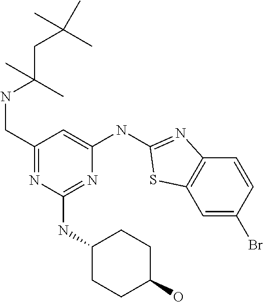 | trans-4-[(4-[(6-bromo- 1,3-benzothiazol-2- yl)amino]-6-{[(1,1,3,3- tetramethylbutyl)amino]methyl}-2- pyrimidinyl)amino]cyclohexanol | LCMS (Method A): Rt 0.92 minutes; m/z 561,563 (MH+) | MDAP, ammonium bicarbonate modifier |

### Example 189

### trans-4-{[4-[(6-bromo-1,3-benzothiazol-2-yl)amino]-6-(4-morpholinylmethyl)-2-pyrimidinyl]amino}cyclohexanol

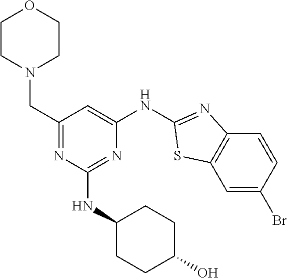

To a stirred solution of {6-[(6-bromo-1,3-benzothiazol-2-yl)amino]-2-[(trans-4-hydroxycyclohexyl)amino]-4-pyrimidinyl}methyl methanesulfonate (1.10 g, 2.08 mmol) and diisopropylethylamine (0.73 mL, 4.16 mmol) in tetrahydrofuran under nitrogen was added morpholine (0.48 mL, 6.24 mmol) dropwise. The reaction mixture was heated at 50° C. for 2 hours. Water (50 mL) was added to the reaction mixture, followed by brine (50 mL) and the mixture was extracted with ethyl acetate (2×100 mL). The organic fractions were combined, dried over magnesium sulfate, filtered and evaporated to dryness. The product was purified by chromatography on silica using a gradient elution from 0 to 15% methanol in dichloromethane to afford the title compound (370 mg, 0.712 mmol, 34% yield). LCMS (Method A): Rt 0.73 minutes; m/z 519,521 (MH+).

The compounds shown in the table were prepared in an analogous manner to that for trans-4-{[4-[(6-bromo-1,3-benzothiazol-2-yl)amino]-6-(4-morpholinylmethyl)-2-pyrimidinyl]amino}cyclohexanol by reacting {6-[(6-bromo-1,3-benzothiazol-2-yl)amino]-2-[(trans-4-hydroxycyclohexyl)amino]-4-pyrimidinyl}methyl methanesulfonate with the appropriate amine:

| | | | | Purification |
|---|---|---|---|---|
| Example | Structure | Name | Analytical Data | Method |
| 190 | 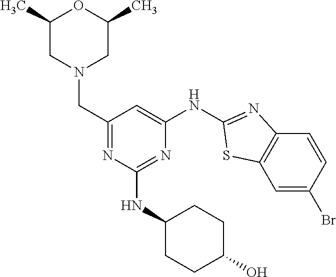 | trans-4-[(4-[(6- bromo-1,3- benzothiazol-2- yl)amino]-6- {[(2R,6S)-2,6- dimethyl-4- morpholinyl]methyl}- 2-pyrimidinyl)amino]cyclo- hexanol | LCMS (Method A): Rt 0.81 minutes; m/z 547,549 (MH+). | Silica chromatography (0-15% methanol +1% triethylamine in dichloromethane |
| 191 | 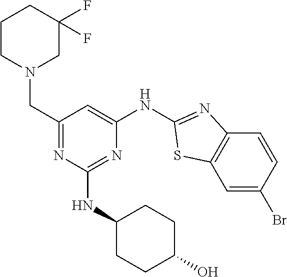 | trans-4-({4-[(6- bromo-1,3- benzothiazol-2- yl)amino]-6-[(3,3- difluoro-1- piperidinyl)methyl]- 2-pyrinidinyl}amino)cyclo- hexanol | LCMS (Method A): Rt 0.88 minutes; m/z 553,555 (MH+). | MDAP, formic acid modifier |

### Example 192

### 5-(2-{[2-[(trans-4-hydroxycyclohexyl)amino]-6-(1-piperidinylmethyl)-4-pyrimidinyl]amino}-1,3-benzothiazol-6-yl)-3-pyridinecarbonitrile

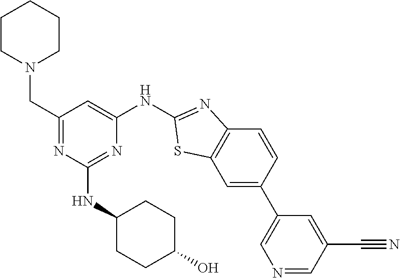

A mixture of trans-4-{[4-[(6-bromo-1,3-benzothiazol-2-yl)amino]-6-(1-piperidinylmethyl)-2-pyrimidinyl]amino}cyclohexanol (100 mg, 0.193 mmol), 5-(4,4,5,5-tetramethyl-1,3,2-dioxaborolan-2-yl)-3-pyridinecarbonitrile (89 mg, 0.386 mmol), tetrakis(triphenylphosphine)Pd(0) (44.7 mg, 0.039 mmol) and potassium phosphate (61.5 mg, 0.290 mmol) in 1,4-dioxane (7 mL) and water (2 mL) was sealed and heated in a Biotage “Initiator” microwave at 100° C. for 1 hour. The reaction mixture was then added to water (50 mL) and the crude product filtered off and dried. The product was purified by mass-directed automated preparative HPLC (ammonium bicarbonate modifier) to afford the title compound (6.1 mg, 0.011 mmol, 5.8% yield). LCMS (Method A) Rt: 0.72 minutes; m/z: 541 (MH+).

The compounds shown in the table were prepared in an analogous manner to that for 5-(2-{[2-[(trans-4-hydroxycyclohexyl)amino]-6-(1-piperidinylmethyl)-4-pyrimidinyl]amino}-1,3-benzothiazol-6-yl)-3-pyridinecarbonitrile by reacting 5-(4,4,5,5-tetramethyl-1,3,2-dioxaborolan-2-yl)-3-pyridinecarbonitrile with the appropriate bromide:

| | | | Analytical | Purification |
|---|---|---|---|---|
| Example | Structure | Name | Data | Method |
| 193 | 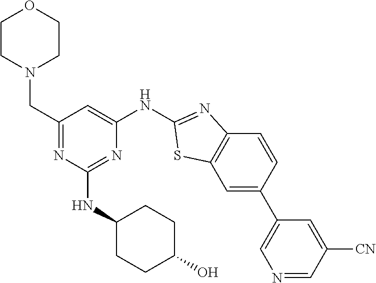 | 5-(2-{[2-[(trans-4- hydroxycyclohexyl) amino]-6-(4- morpholinyl- methyl)-4- pyrimidinyl]amino}- 1,3-benzothiazol-6- yl)-3- pyridinecarbonitrile | LCMS (Method A) Rt: 0.69 minutes; m/z: 543 (MH+). | MDAP, ammonium bicarbonate modifier |
| 194 | 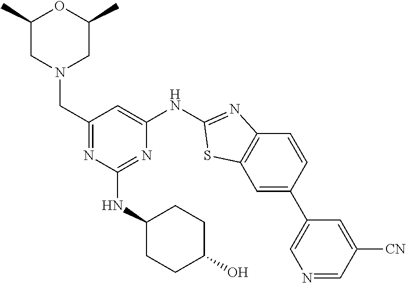 | 5-[2-({6-{[(2R,6S)- 2,6-dimethyl-4- morpholinyl]methyl}- 2-[(trans-4- hydroxycyclohexyl) amino]-4- pyrimidinyl}amino)- 1,3-benzothiazol-6- yl]-3- pyridinecarbonitrile | LCMS (Method A) Rt: 0.75 minutes; m/z: 571 (MH+). | MDAP, ammonium bicarbonate modifier |
| 195 | 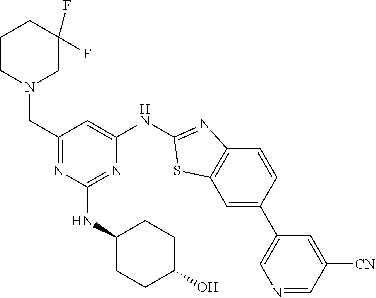 | 5-[2-({6-[(3,3- difluoro-1- piperidinyl)methyl]- 2-[(trans-4- hydroxycyclohexyl) amino]-4- pyrimidinyl}amino)- 1,3-benzothiazol-6- yl]-3- pyridinecarbonitrile | LCMS (Method A) Rt: 0.78 minutes; m/z: 577 (MH+). | MDAP, ammonium bicarbonate modifier |

### Example 196

### trans-4-{[4-[(6-{5-[(1-methylethyl)oxy]-3-pyridinyl}-1,3-benzothiazol-2-yl)amino]-6-(4-morpholinylmethyl)-2-pyrimidinyl]amino}cyclohexanol

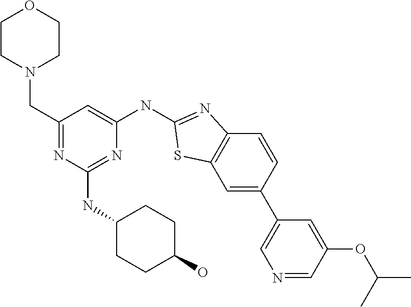

A mixture of 3-[(1-methylethyl)oxy]-5-(4,4,5,5-tetramethyl-1,3,2-dioxaborolan-2-yl)pyridine (101 mg, 0.385 mmol), trans-4-{[4-[(6-bromo-1,3-benzothiazol-2-yl)amino]-6-(4-morpholinylmethyl)-2-pyrimidinyl]amino}cyclohexanol (100 mg, 0.193 mmol), tetrakis(triphenylphosphine)palladium(0) (66.7 mg, 0.058 mmol), caesium carbonate (188 mg, 0.578 mmol) in 1,4-dioxane (2 mL) and water (0.5 mL) was sealed and heated in a Biotage “Initiator” microwave at 130° C. for 30 minutes. The cooled reaction mixture was evaporated to dryness. The product was purified by mass-directed automated preparative HPLC (formic acid modifier) to afford the title compound (20 mg, 0.035 mmol, 18% yield). LCMS (Method A): Rt 0.71 minutes; m/z 576 (MH+).

The compound shown in the table was prepared in an analogous manner to that for trans-4-{[4-[(6-{5-[(1-methylethyl)oxy]-3-pyridinyl}-1,3-benzothiazol-2-yl)amino]-6-(4-morpholinylmethyl)-2-pyrimidinyl]amino}cyclohexanol by reacting 3-[(1-methylethyl)oxy]-5-(4,4,5,5-tetramethyl-1,3,2-dioxaborolan-2-yl)pyridine with the appropriate aryl bromide:

| | | | | Purification |
|---|---|---|---|---|
| Example | Structure | Name | Analytical Data | Method |
| 197 | 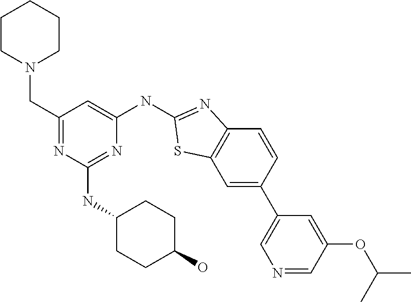 | trans-4-{[4-[(6-{5- [(1- methylethyl)oxy]-3- pyridinyl}-1,3- benzothiazol-2- yl)amino]-6-(1- piperidinylmethyl)- 2-pyrimidinyl]amino}cyclo- hexanol | LCMS (Method A): Rt: 0.74 minutes; m/z 574 (MH+). | MDAP, formic acid modifier |
| 198 | 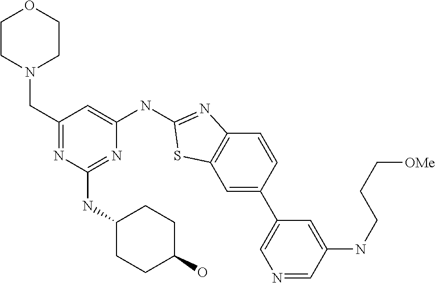 | trans-4-{[4-({6-[5- ({[2- (methyloxy)ethyl]amino}methyl)- 3-pyridinyl]-1,3- benzothiazol-2- yl}amino)-6-(4- morpholinylmethyl)- 2- pyrimidinyl]amino}cyclohexanol | LCMS (Method A): Rt: 0.50 minutes; m/z 605 (MH+). | MDAP, formic acid modifier |

### Example 199

### 1-(2-{[2-[(trans-4-hydroxycyclohexyl)amino]-6-(1-piperidinylmethyl)-4-pyrimidinyl]amino}-1,3-benzothiazol-6-yl)-2,5-pyrrolidinedione

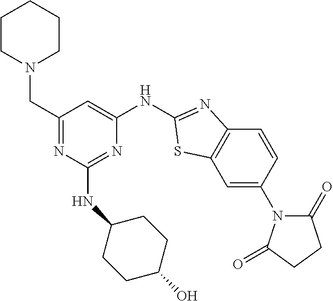

A mixture of trans-4-{[4-[(6-bromo-1,3-benzothiazol-2-yl)amino]-6-(1-piperidinylmethyl)-2-pyrimidinyl]amino}cyclohexanol (200 mg, 0.386 mmol), succinimide (115 mg, 1.159 mmol), caesium carbonate (252 mg, 0.773 mmol) and copper(I) iodide (147 mg, 0.773 mmol) in dry N,N-dimethylformamide (5 mL) was thoroughly degassed by the repeated alternate application of vacuum and nitrogen pressure, then treated with N,N′-dimethylethylenediamine (0.330 mL, 3.092 mmol) and the mixture was heated at 110° C. for 16 hours. The reaction mixture was filtered under reduced pressure and the filtrate was evaporated to dryness. The product was purified by mass-directed automated preparative HPLC (formic acid modifier) to afford the title compound (5 mg, 0.0093 mmol, 2.4% yield). LCMS (Method A): Rt 0.58 minutes; m/z 536 (MH+).

The compound shown in the table was prepared in an analogous manner to that for 1-(2-{[2-[(trans-4-hydroxycyclohexyl)amino]-6-(1-piperidinylmethyl)-4-pyrimidinyl]amino}-1,3-benzothiazol-6-yl)-2,5-pyrrolidinedione by reacting succinimide with trans-4-{[4-[(6-bromo-1,3-benzothiazol-2-yl)amino]-6-(4-morpholinylmethyl)-2-pyrimidinyl]amino}cyclohexanol:

| | | | | Purification |
|---|---|---|---|---|
| Example | Structure | Name | Analytical Data | Method |
| 200 | 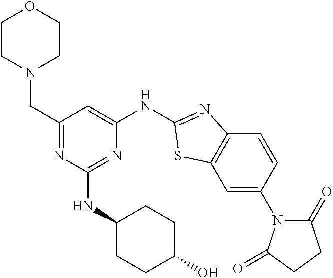 | 1 -(2-{[2-[(trans-4- hydroxycyclohexyl)amino]- 6-(4- morpholinylmethyl)-4- pyrimidinyl]amino}- 1,3-benzothiazol-6-yl)- 2,5-pyrrolidinedione | LCMS (Method A): Rt 0.67 minutes; m/z 538 (MH+). | MDAP, formic acid modifier |

### Example 201

### 1-(2-{[2-[(trans-4-hydroxycyclohexyl)amino]-6-(1-piperidinylmethyl)-4-pyrimidinyl]amino}-1,3-benzothiazol-6-yl)-2-pyrrolidinone

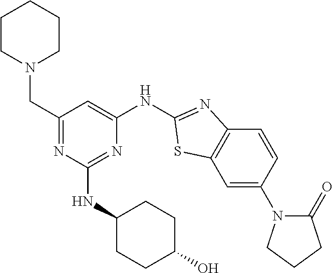

Under an atmosphere of nitrogen, a mixture of trans-4-{[4-[(6-bromo-1,3-benzothiazol-2-yl)amino]-6-(1-piperidinylmethyl)-2-pyrimidinyl]amino}cyclohexanol (200 mg, 0.386 mmol), 2-pyrrolidinone (0.089 mL, 1.159 mmol), caesium carbonate (252 mg, 0.773 mmol) and copper(I) iodide (147 mg, 0.773 mmol) in dry N,N-dimethylformamide (5 mL) was thoroughly degassed by the repeated alternate application of vacuum and nitrogen pressure, then treated with N,N′-dimethylethylenediamine (0.165 mL, 1.546 mmol) and the mixture was heated at 110° C. for 16 hours. The reaction mixture was filtered under reduced pressure and the filtrate was evaporated to dryness. The product was purified by mass-directed automated preparative HPLC (formic acid modifier) to afford the title compound (28.5 mg, 0.055 mmol, 14% yield). LCMS (Method A): Rt 0.65 minutes; m/z 522 (MH+).

### Example 202

### 3-(2-{[2-[(trans-4-hydroxycyclohexyl)amino]-6-(1-piperidinylmethyl)-4-pyrimidinyl]amino}-1,3-benzothiazol-6-yl)-1,3-oxazolidin-2-one

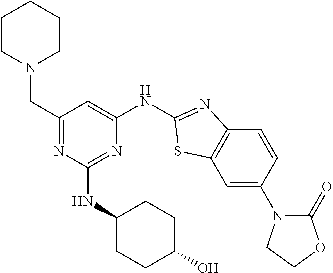

Under an atmosphere of nitrogen, a mixture of trans-4-{[4-[(6-bromo-1,3-benzothiazol-2-yl)amino]-6-(1-piperidinylmethyl)-2-pyrimidinyl]amino}cyclohexanol (250 mg, 0.483 mmol), 2-oxazolidinone (126 mg, 1.449 mmol), caesium carbonate (315 mg, 0.966 mmol) and copper(I) iodide (184 mg, 0.966 mmol) in dry N,N-dimethylformamide (5 mL) was thoroughly degassed by the repeated alternate application of vacuum and nitrogen pressure, then treated with N,N′-dimethylethylenediamine (0.412 mL, 3.864 mmol) and the mixture was heated at 110° C. for 2 days. The reaction mixture was filtered under reduced pressure and the filtrate was evaporated to dryness. The product was purified by mass-directed automated preparative HPLC (ammonium bicarbonate modifier) to afford the title compound (13.5 mg, 0.026 mmol, 5% yield). LCMS (Method A): Rt 0.62 minutes; m/z 524 (MH+).

### Example 203

### 3-(2-{[2-[(trans-4-hydroxycyclohexyl)amino]-6-(4-morpholinylmethyl)-4-pyrimidinyl]amino}-1,3-benzothiazol-6-yl)-1,3-oxazolidin-2-one

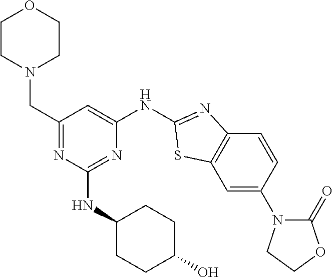

Under an atmosphere of nitrogen, a mixture of trans-4-{[4-[(6-bromo-1,3-benzothiazol-2-yl)amino]-6-(4-morpholinylmethyl)-2-pyrimidinyl]amino}cyclohexanol (370 mg, 0.712 mmol), 2-oxazolidinone (186 mg, 2.137 mmol), caesium carbonate (464 mg, 1.425 mmol) and copper(I) iodide (271 mg, 1.425 mmol) and N,N′-dimethylethylenediamine (0.304 mL, 2.85 mmol) in dry N,N-dimethylformamide (5 mL) was sealed and heated in a Biotage “Initiator” microwave at 100° C. for 30 minutes. Water (30 mL) and ethyl acetate (30 mL) were added to the mixture and the organic phase was collected and washed repeatedly with water until no blue colour was evident in the aqueous phase. The organic phase was dried over magnesium sulfate and filtered. The filtrate was evaporated to dryness and the product was purified by mass-directed automated preparative HPLC (ammonium bicarbonate modifier) to afford the title compound (13 mg, 0.025 mmol, 3% yield). LCMS (Method A): Rt 0.58 minutes; m/z 526 (MH+).

The compounds shown in the table were prepared in an analogous manner to that for 3-(2-{[2-[(trans-4-hydroxycyclohexyl)amino]-6-(4-morpholinylmethyl)-4-pyrimidinyl]amino}-1,3-benzothiazol-6-yl-1,3-oxazolidin-2-one by reacting 2-oxazolidinone with the appropriate aryl bromide:

| | | | | Purification |
|---|---|---|---|---|
| Example | Structure | Name | Analytical Data | Method |
| 204 | 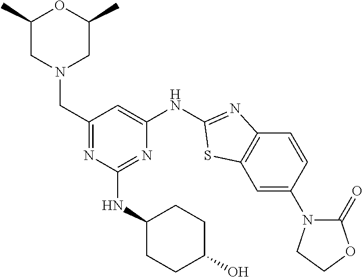 | 3-[2-({6-{[(2R,6S)-2,6- dimethyl-4- morpholinyl]methyl}-2- [(trans-4- hydroxycyclohexyl)amino]-4- pyrimidinyl}amino)-1,3- benzothiazol-6-yl]-1,3- oxazolidin-2-one | LCMS (Method A): Rt 0.71 minutes; m/z 554 (MH+). | MDAP, ammonium bicarbonate modifier |
| 205 | 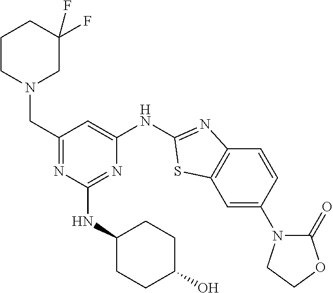 | 3-[2-({6-[(3,3-difluoro-1- piperidinyl)methyl]-2- [(trans-4- hydroxycyclohexyl)amino]-4- pyrimidinyl}amino)-1,3- benzothiazol-6-yl]-1,3- oxazolidin-2-one | LCMS (Method A): Rt 0.72 minutes; m/z 560 (MH+). | MDAP, ammonium bicarbonate modifier |

### Example 206

### trans-4-[(4-(1-piperidinylmethyl)-6-{[6-(1H-pyrazol-4-yl)-1,3-benzothiazol-2-yl]amino}-2-pyrimidinyl)amino]cyclohexanol

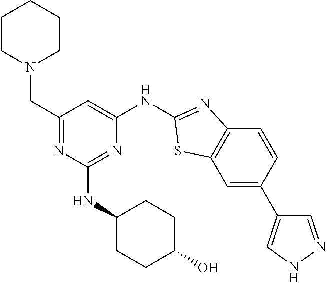

A mixture of trans-4-{[4-[(6-bromo-1,3-benzothiazol-2-yl)amino]-6-(1-piperidinylmethyl)-2-pyrimidinyl]amino}cyclohexanol (250 mg, 0.483 mmol), 1,1-dimethylethyl 4-(4,4,5,5-tetramethyl-1,3,2-dioxaborolan-2-yl)-1H-pyrazole-1-carboxylate (142 mg, 0.483 mmol), caesium carbonate (472 mg, 1.449 mmol), tetrakis(triphenylphosphine)palladium(0) (167 mg, 0.145 mmol) in 1,4-dioxane (6 mL) and water (2 mL) was sealed and heated in a Biotage “Initiator” microwave at 150° C. for 45 minutes. The cooled reaction mixture was treated with water (20 mL) and ethyl acetate (20 mL) and filtered. The filtrate was separated and the aqueous phase was extracted with ethyl acetate (20 mL). The organic phases were combined, dried over magnesium sulfate, filtered and evaporated to dryness. The product was purified by mass-directed automated preparative HPLC (formic acid modifier) to afford the title compound (14.9 mg, 0.030 mmol, 6% yield). LCMS (Method A): Rt 0.60 minutes; m/z 505 (MH+).

The compounds shown in the table were prepared in an analogous manner to that for trans-4-[(4-(1-piperidinylmethyl)-6-{[6-(1H-pyrazol-4-yl)-1,3-benzothiazol-2-yl]amino}-2-pyrimidinyl)amino]cyclohexanol by reacting 1,1-dimethylethyl 4-(4,4,5,5-tetramethyl-1,3,2-dioxaborolan-2-yl)-1H-pyrazole-1-carboxylate with the appropriate bromide:

| | | | | Purification |
|---|---|---|---|---|
| Example | Structure | Name | Analytical Data | Method |
| 207 | 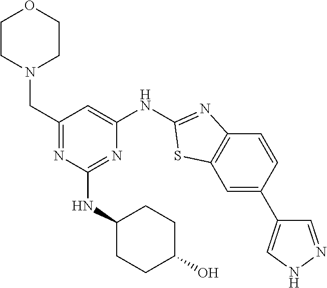 | trans-4-[(4-(4- morpholinylmethyl)- 6-{[6-(1H- pyrazol-4-yl)-1,3- benzothiazol-2- yl]amino}-2- pyrimidinyl)amino]cyclohexanol | LCMS (Method A): Rt 0.57 minutes; m/z 507 (MH+). | Formic acid modified Mass Directed Auto- Preparative purification. |
| 208 | 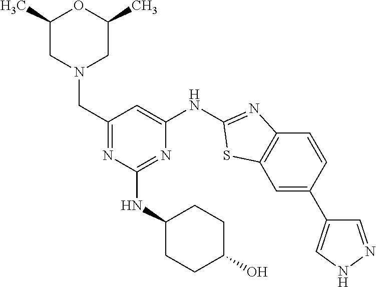 | trans-4-[(4- {[(2R,6S)-2,6- dimethyl-4- morpholinyl]methyl}- 6-{[6-(1H- pyrazol-4-yl)-1,3- benzothiazol-2- yl]amino}-2- pyrimidinyl)amino]cyclohexanol | LCMS (Method A): Rt 0.65 minutes; m/z 535 (MH+). | Formic acid modified Mass Directed Auto- Preparative purification. |
| 209 | 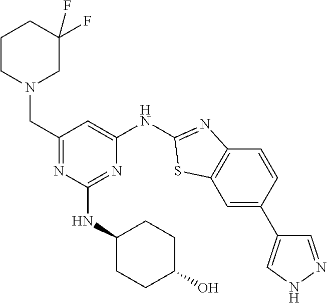 | trans-4-[(4-[(3,3- difluoro-1- piperidinyl)methyl]- 6-{[6-(1H- pyrazol-4-yl)-1,3- benzothiazol-2- yl]amino}-2- pyrimidinyl)amino]cyclohexanol | LCMS (Method A): Rt 0.71 minutes; m/z 541 (MH+). | Ammonium Bicarbonate modified Mass Directed Auto- Preparative purification |

### Example 210

### 3-(4-{[4-(1-piperidinylmethyl)-6-([1,3]thiazolo[5,4-b]pyridin-2-ylamino)-2-pyrimidinyl]amino}phenyl)propanoic acid

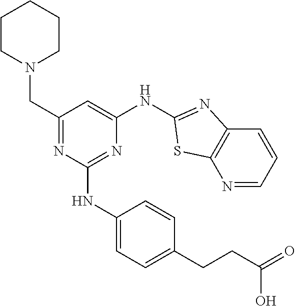

A mixture of N-[2-[(phenylmethyl)sulfonyl]-6-(1-piperidinylmethyl)-4-pyrimidinyl][1,3]thiazolo[5,4-b]pyridin-2-amine (50 mg, 0.104 mmol) and 3-(4-aminophenyl)propanoic acid (20.62 mg, 0.125 mmol) in acetonitrile (1 mL) was treated with hydrochloric acid (aqueous, 2M, 2 drops), sealed and heated in a Biotage “Initiator” microwave at 150° C. for 2 hours. The mixture was evaporated to dryness and the product was purified by mass-directed automated preparative HPLC (ammonium bicarbonate modifier) to afford the title compound (7.8 mg, 0.016 mmol, 15% yield). LCMS (Method B): Rt 0.73 minutes; m/z 490 (MH+)

### Example 211

N²-[trans-4-(4-morpholinyl)cyclohexyl]-6-(1-piperidinylmethyl)-N⁴-[1,3]thiazolo[5,4-b]pyridin-2-yl-2,4-pyrimidinediamine

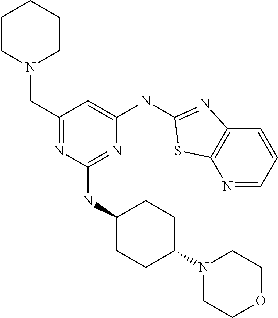

A mixture of N-[2-[(phenylmethyl)sulfonyl]-6-(1-piperidinylmethyl)-4-pyrimidinyl][1,3]thiazolo[5,4-b]pyridin-2-amine (50 mg, 0.104 mmol) and trans-4-(4-morpholinyl)cyclohexanamine (80 mg, 0.434 mmol) in isopropanol (2 mL) was sealed and heated in a Biotage “Initiator” microwave at 160° C. for 45 minutes. The reaction mixture was evaporated to dryness and the product was subjected to purification by mass-directed automated preparative HPLC (ammonium bicarbonate modifier) to afford the title compound (14.7 mg, 0.029 mmol, 28% yield). LCMS (Method B): Rt 0.49 minutes; m/z 509 (MH+)

### Example 212

### 1-{[2-[(trans-4-hydroxycyclohexyl)amino]-6-([1,3]thiazolo[5,4-b]pyridin-2-ylamino)-4-pyrimidinyl]methyl}-2-piperazinone

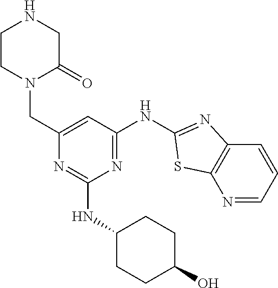

A solution of 1,1-dimethylethyl[2-({[2-[(trans-4-hydroxycyclohexyl)amino]-6-([1,3]thiazolo[5,4-b]pyridin-2-ylamino)-4-pyrimidinyl]methyl}amino)ethyl]carbamate (80 mg, 0.155 mmol) in tetrahydrofuran (5 mL) was treated with diisopropylethylamine (0.054 mL, 0.311 mmol) and then with chloroacetyl chloride (0.016 mL, 0.202 mmol). The mixture was stirred at ambient temperature for 30 minutes and then treated with trifluoroacetic acid (5 mL). After 1 hour the mixture was evaporated to dryness and then treated with chloroform (10 mL) and saturated sodium bicarbonate (aqueous, 10 mL) and stirred for 1 hour. The organic fraction was collected, evaporated to dryness the product was purified by mass-directed automated preparative HPLC (ammonium bicarbonate modifier) to afford the title compound (24 mg, 0.053 mmol, 34% yield). LCMS (Method B): Rt 0.47 minutes; m/z 455 (MH+)

### Example 213

### 1-{[2-[(trans-4-hydroxycyclohexyl)amino]-6-([1,3]thiazolo[5,4-b]pyridin-2-ylamino)-4-pyrimidinyl]methyl}-L-proline

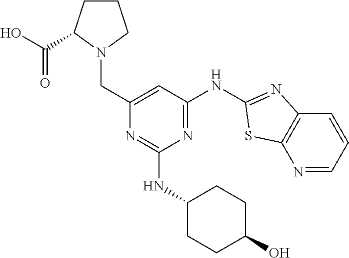

1,1-dimethylethyl 1-{[2-[(trans-4-hydroxycyclohexyl)amino]-6-([1,3]thiazolo[5,4-b]pyridin-2-ylamino)-4-pyrimidinyl]methyl}-L-prolinate (15 mg, 0.029 mmol) was treated with trifluoroacetic acid (2 mL) and the resulting solution was stirred for 3 hours. The mixture was evaporated to dryness and the product was purified by mass-directed automated preparative HPLC (formic acid modifier) to afford the title compound (11 mg, 0.023 mmol, 79% yield). LCMS (Method B): Rt 0.55 minutes; m/z 470 (MH+)

### Example 214

### trans-4-{[4-{[(1,1-dimethylbutyl)amino]methyl}-6-([1,3]thiazolo[5,4-b]pyridin-2-ylamino)-2-pyrimidinyl]amino}cyclohexanol

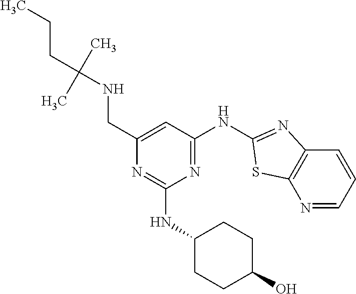

A suspension of 2-[(trans-4-hydroxycyclohexyl)amino]-6-([1,3]thiazolo[5,4-b]pyridin-2-ylamino)-4-pyrimidinecarbaldehyde (40 mg, 0.108 mmol) in tetrahydrofuran (0.4 mL) was treated with (1,1-dimethylbutyl)amine (0.027 mL, 0.216 mmol) and the mixture was allowed to stir for 15 minutes. The mixture was then treated with dichloromethane (1.6 mL) and then with sodium triacetoxyborohydride (34.3 mg, 0.162 mmol). The reaction was stirred for 3 hours and then treated with saturated aqueous sodium bicarbonate (1 mL) and stirred for 30 minutes. Chloroform (1 mL) and methanol (0.2 mL) were added and the mixture was separated; the organic phase evaporated to dryness and the product was then purified by mass-directed automated preparative HPLC (ammonium bicarbonate modifier) to afford the title compound (20 mg, 0.044 mmol, 41% yield). LCMS (Method B): Rt 0.68 minutes; m/z 456 (MH+)

The compounds shown in the table was prepared in an analogous manner to that for trans-4-{[4-{[(1,1-dimethylbutyl)amino]methyl}-6-([1,3]thiazolo[5,4-b]pyridin-2-ylamino)-2-pyrimidinyl]amino}cyclohexanol by reacting 2-[(trans-4-hydroxycyclohexyl)amino]-6-([1,3]thiazolo[5,4-b]pyridin-2-ylamino)-4-pyrimidinecarbaldehyde with the appropriate amine:

| | | | | Purification |
|---|---|---|---|---|
| Example | Structure | Name | Analytical Data | Method |
| 215 | 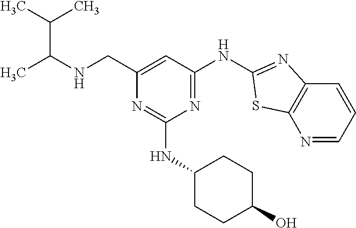 | trans-4-{[4-{[(1,2- dimethylpropyl)amino]methyl}- 6-([1,3]thiazolo[5,4- b]pyridin-2-ylamino)-2- pyrimidinyl]amino}cyclohexanol | LCMS (Method B): Rt 0.66 minutes; m/z 442 (MH+) | MDAP, ammonium bicarbonate modifier |
| 216 | 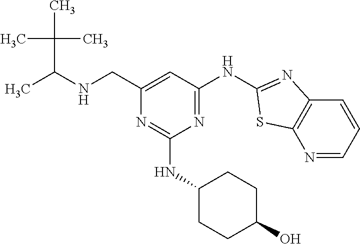 | trans-4-[(4- ([1,3]thiazolo[5,4- b]pyridin-2-ylamino)-6- {[(1,2,2- trimethylpropyl)amino]methyl}-2- pyrimidinyl)amino]cyclohexanol | LCMS (Method B): Rt 0.69 minutes; m/z 456 (MH+) | MDAP, ammonium bicarbonate modifier |
| 217 | 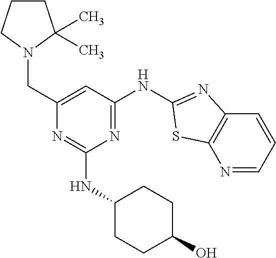 | trans-4-{[4-[(2,2-dimethyl- 1-pyrrolidinyl)methyl]-6- ([1,3]thiazolo[5,4- b]pyridin-2-ylamino)-2- pyrimidinyl]amino}cyclohexanol | LCMS (Method B): Rt 0.60 minutes; m/z 454 (MH+) | MDAP, ammonium bicarbonate modifier |
| 218 | 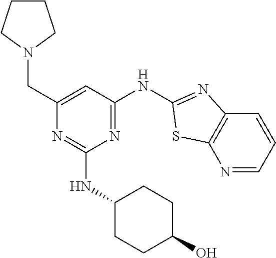 | trans-4-{[4-(1- pyrrolidinylmethyl)-6- ([1,3]thiazolo[5,4- b]pyridin-2-ylamino)-2- pyrimidinyl]amino}cyclohexanol | LCMS (Method B): Rt 0.57 minutes; m/z 426 (MH+) | MDAP, ammonium bicarbonate modifier |
| 219 | 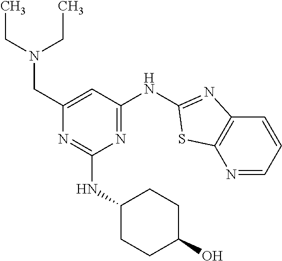 | trans-4-{[4- [(diethylamino)methyl]-6- ([1,3]thiazolo[5,4- b]pyridin-2-ylamino)-2- pyrimidinyl]amino}cyclohexanol | LCMS (Method B): Rt 0.59 minutes; m/z 428 (MH+) | MDAP, ammonium bicarbonate modifier |
| 220 | 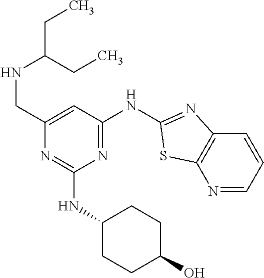 | trans-4-{[4-{[(1- ethylpropyl)amino]methyl}- 6-([1,3]thiazolo[5,4- b]pyridin-2-ylamino)-2- pyrimidinyl]amino}cyclohexanol | LCMS (Method B): Rt 0.66 minutes; m/z 442 (MH+) | MDAP, ammonium bicarbonate modifier |
| 221 | 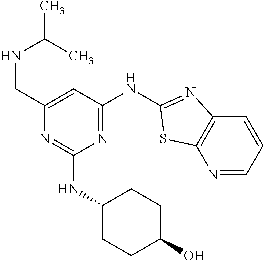 | trans-4-{[4-{[(1- methylethyl)amino]methyl}- 6-([1,3]thiazolo[5,4- b]pyridin-2-ylamino)-2- pyrimidinyl]amino}cyclohexanol | LCMS (Method B): Rt 0.59 minutes; m/z 414 (MH+) | MDAP, ammonium bicarbonate modifier |
| 222 | 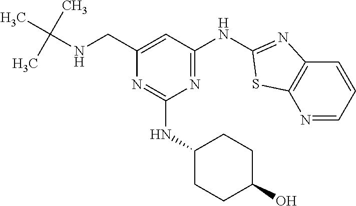 | trans-4-{[4-{[(1,1- dimethyiethyl)amino]methyl}- 6-([1,3]thiazolo[5,4- b]pyridin-2-ylamino)-2- pyrimidinyl]amino}cyclohexanol | LCMS (Method B): Rt 0.60 minutes; m/z 428 (MH+) | MDAP, ammonium bicarbonate modifier |
| 223 | 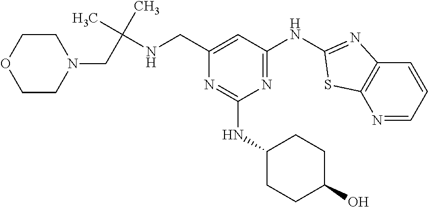 | trans-4-{[4-({[1,1- dimethyl-2-(4- morpholinyl)ethyl]amino} methyl)-6- ([1,3]thiazolo[5,4- b]pyridin-2-ylamino)-2- pyrimidinyl]amino}cyclohexanol | LCMS (Method B): Rt 0.62 minutes; m/z 513 (MH+) | MDAP, ammonium bicarbonate modifier |
| 224 | 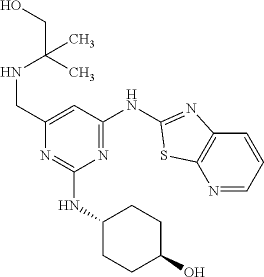 | trans-4-{[4-{[(2-hydroxy- 1,1- dimethylethyl)amino]methyl}- 6-([1,3]thiazolo[5,4- b]pyridin-2-ylamino)-2- pyrimidinyl]amino}cyclohexanol | LCMS (Method B): Rt 0.56 minutes; m/z 444 (MH+) | MDAP, ammonium bicarbonate modifier |
| 225 | 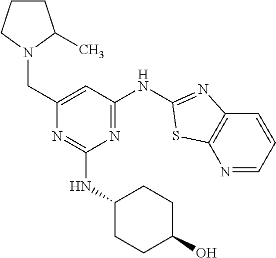 | trans-4-{[4-[(2-methyl-1- pyrrolidinyl)methyl]-6- ([1,3]thiazolo[5,4- b]pyridin-2-ylamino)-2- pyrimidinyl]amino}cyclohexanol | LCMS (Method B): Rt 0.58 minutes; m/z 440 (MH+) | MDAP, ammonium bicarbonate modifier |
| 226 | 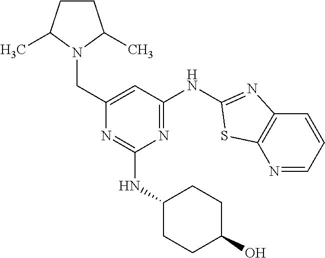 | trans-4-{[4-[(2,5-dimethyl- 1-pyrrolidinyl)methyl]-6- ([1,3]thiazolo[5,4- b]pyridin-2-ylamino)-2- pyrimidinyl]amino}cyclohexanol | LCMS (Method B): Rt 0.61 minutes; m/z 454 (MH+) | MDAP, ammonium bicarbonate modifier |
| 227 | 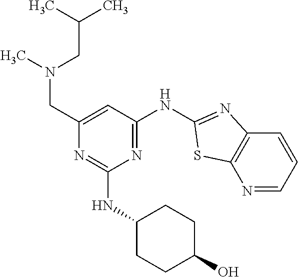 | trans-4-{[4-{[methyl(2- methylpropyl)amino]methyl}- 6-([1,3]thiazolo[5,4- b]pyridin-2-ylamino)-2- pyrimidinyl]amino}cyclohexanol | LCMS (Method B): Rt 0.64 minutes; m/z 442 (MH+) | MDAP, ammonium bicarbonate modifier |
| 228 | 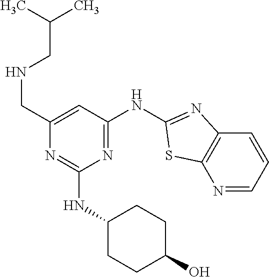 | trans-4-{[4-{[(2- methylpropyl)amino]methyl}- 6-([1,3]thiazolo[5,4- b]pyridin-2-ylamino)-2- pyrimidinyl]amino}cyclchexanol | LCMS (Method B): Rt 0.62 minutes; m/z 428 (MH+) | MDAP, ammonium bicarbonate modifier |
| 229 | 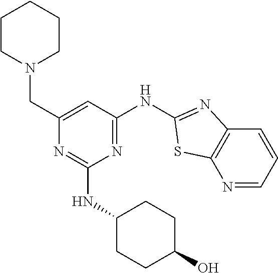 | trans-4-{[4-(1- piperidinylmethyl)-6- ([1,3]thiazolo[5,4- b]pyridin-2-ylamino)-2- pyrimidinyl]amino}cyclchexanol | LCMS (Method B): Rt 0.61 minutes; m/z 440 (MH+) | MDAP, ammonium bicarbonate modifier |
| 230 | 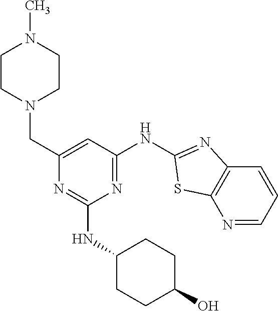 | trans-4-{[4-[(4-methyl-1- piperazinyl)methyl]-6- ([1,3]thiazolo[5,4- b]pyridin-2-ylamino)-2- pyrimidinyl]amino}cyclohexanol | LCMS (Method B): Rt 0.44 minutes; m/z 455 (MH+) | MDAP, ammonium bicarbonate modifier |
| 231 | 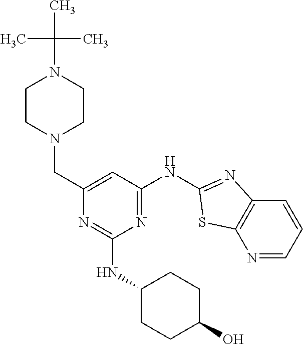 | trans-4-{[4-{[4-(1,1- dimethylethyl)-1- piperazinyl]methyl}-6- ([1,3]thiazolo[5,4- b]pyridin-2-ylamino)-2- pyrimidinyl]amino}cyclohexanol | LCMS (Method B): Rt 0.52 minutes; m/z 497 (MH+) | MDAP, ammonium bicarbonate modifier |
| 232 | 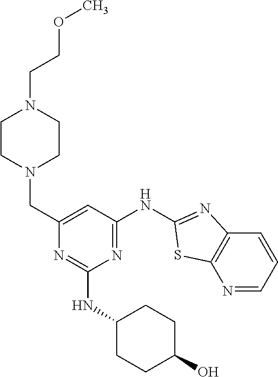 | trans-4-{[4-({4-[2- (methyloxy)ethyl]-1- piperazinyl}methyl)-6- ([1,3]thiazolo[5,4- b]pyridin-2-ylamino)-2- pyrimidinyl]amino}cyclohexanol | LCMS (Method B): Rt 0.47 minutes; m/z 497 (MH+) | MDAP, ammonium bicarbonate modifier |
| 233 | 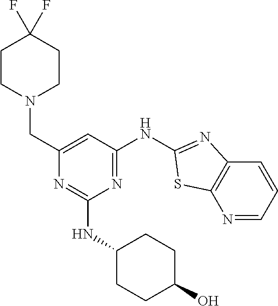 | trans-4-{[4-[(4,4-difluoro- 1-piperidinyl)methyl]-6- ([1,3]thiazolo[5,4- b]pyridin-2-ylamino)-2- pyrimidinyl]amino}cyclohexanol | LCMS (Method B): Rt 0.63 minutes; m/z 476 (MH+) | MDAP, ammonium bicarbonate modifier |
| 234 | 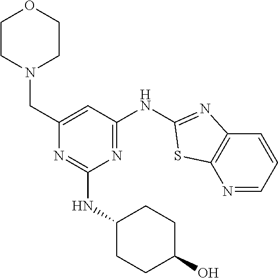 | trans-4-{[4-(4- morpholinylmethyl)-6- ([1,3]thiazolo[5,4- b]pyridin-2-ylamino)-2- pyrimidinyl]amino}cyclohexanol | LCMS (Method B): Rt 0.55 minutes; m/z 442 (MH+) | MDAP, ammonium bicarbonate modifier |
| 235 | 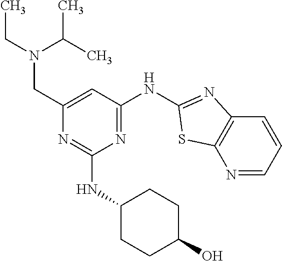 | trans-4-{[4-{[ethyl(1- methylethyl)amino]methyl}- 6-([1,3]thiazolo[5,4- b]pyridin-2-ylamino)-2- pyrimidinyl]amino}cyclohexanol | LCMS (Method B): Rt 0.61 minutes; m/z 442 (MH+) | MDAP, ammonium bicarbonate modifier |
| 236 | 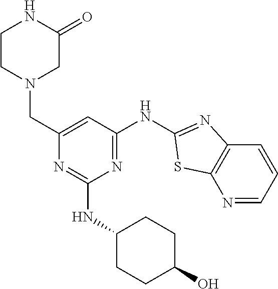 | 4-{[2-[(trans-4- hydroxycyclohexyl)amino]- 6-([1,3]thiazolo[5,4- b]pyridin-2-ylamino)-4- pyrimidinyl]methyl}-2- piperazinone | LCMS (Method B): Rt 0.51 minutes; m/z 455 (MH+) | MDAP, ammonium bicarbonate modifier |
| 237 | 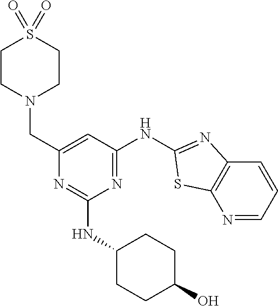 | trans-4-{[4-[(1,1-dioxido- 4-thiomorpholinyl)methyl]- 6-([1,3]thiazolo[5,4- b]pyridin-2-ylamino)-2- pyrimidinyl]amino}cyclohexanol | LCMS (Method B): Rt 0.55 minutes; m/z 490 (MH+) | MDAP, ammonium bicarbonate modifier |
| 238 | 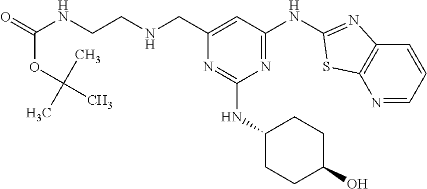 | 1,1-dimethylethyl [2-({[2- [(trans-4- hydroxycyclohexyl)amino]- 6-([1,3]thiazolo[5,4- b]pyridin-2-ylamino)-4- pyrimidinyl]methyl}amino) ethyl]carbamate | LCMS (Method B): Rt 0.66 minutes; m/z 515 (MH+) | MDAP, ammonium bicarbonate modifier |
| 239 | 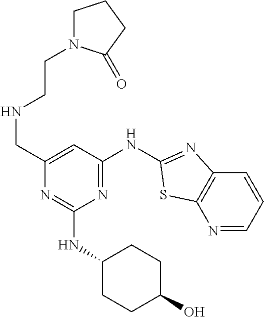 | 1-[2-({[2-[(trans-4- hydroxycyclohexyl)amino]- 6-([1,3]thiazolo[5,4- b]pyridin-2-ylamino)-4- pyrimidinyl]methyl}amino) ethyl]-2-pyrrolidinone | LCMS (Method B): Rt 0.56 minutes; m/z 483 (MH+) | MDAP, ammonium bicarbonate modifier |
| 240 | 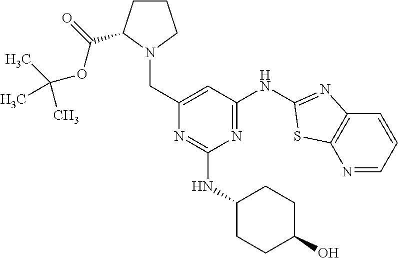 | 1,1-dimethylethyl 1-{[2- [(trans-4- hydroxycyclohexyl)amino]- 6-([1,3]thiazolo[5,4- b]pyridin-2-ylamino)-4- pyrimidinyl]methyl}-L- prolinate | LCMS (Method B): Rt 0.71 minutes; m/z 526 (MH+) | MDAP, ammonium bicarbonate modifier |
| 241 | 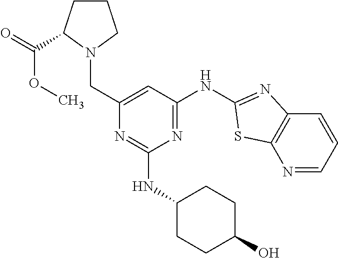 | methyl 1-{[2-[(trans-4- hydroxycyclohexyl)amino]- 6-([1,3]thiazolo[5,4- b]pyridin-2-ylamino)-4- pyrimidinyl]methyl}-L- prolinate | LCMS (Method B): Rt 0.60 minutes; m/z 484 (MH+) | MDAP, ammonium bicarbonate modifier |
| 242 | 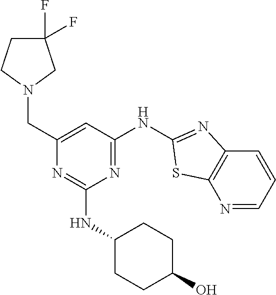 | trans-4-{[4-[(3,3-difluoro- 1-pyrrolidinyl)methyl]-6- ([1,3]thiazolo[5,4- b]pyridin-2-ylamino)-2- pyrimidinyl]amino}cyclohexanol | LCMS (Method B): Rt 0.64 minutes; m/z 462 (MH+) | MDAP, ammonium bicarbonate modifier |
| 243 | 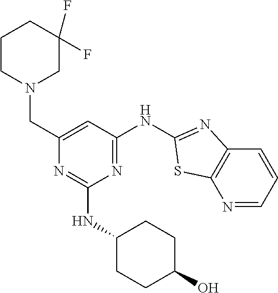 | trans-4-{[4-[(3,3-difluoro- 1-piperidinyl)methyl]-6- ([1,3]thiazolo[5,4- b]pyridin-2-ylamino)-2- pyrimidinyl]amino}cyclohexanol | LCMS (Method B): Rt 0.67 minutes; m/z 476 (MH+) | MDAP, ammonium bicarbonate modifier |
| 244 | 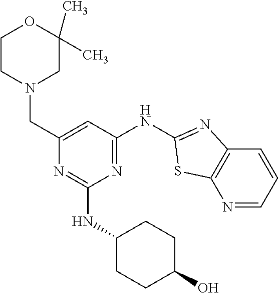 | trans-4-{[4-[(2,2-dimethyl- 4-morpholinyl)methyl]-6- ([1,3]thiazolo[5,4- b]pyridin-2-ylamino)-2- pyrimidinyl]amino}cyclohexanol | LCMS (Method B): Rt 0.60 minutes; m/z 470 (MH+) | MDAP, ammonium bicarbonate modifier |
| 245 | 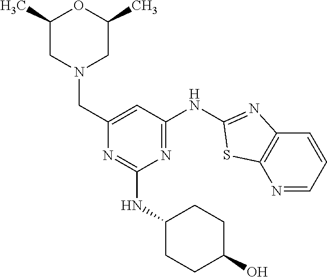 | trans-4-{[4-{[(2R,6S)-2,6- dimethyl-4- morpholinyl]methyl}-6- ([1,3]thiazolo[5,4- b]pyridin-2-ylamino)-2- pyrimidinyl]amino}cyclohexanol | LCMS (Method B): Rt 0.60 minutes; m/z 470 (MH+) | MDAP, ammonium bicarbonate modifier |
| 246 | 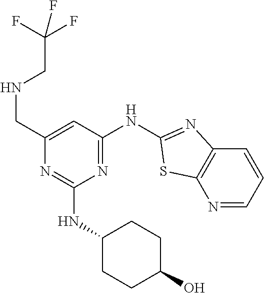 | trans-4-[(4- ([1,3]thiazolo[5,4- b]pyridin-2-ylamino)-6- {[(2,2,2- trifluoroethyl)amino]methyl}-2- pyrimidinyl)amino]cyclohexanol | LCMS (Method B): Rt 0.63 minutes; m/z 454 (MH+) | MDAP, ammonium bicarbonate modifier |
| 247 | 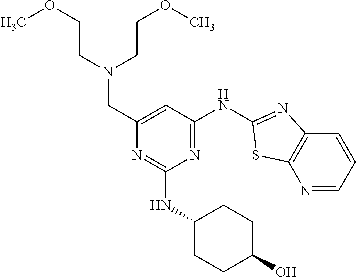 | trans-4-{[4-({bis[2- (methyloxy)ethyl]amino} methyl)-6-([1,3]thiazolo[5,4- b]pyridin-2-ylamino)-2- pyrimidinyl]amino}cyclohexanol | LCMS (Method B): Rt 0.60 minutes; m/z 488 (MH+) | MDAP, ammonium bicarbonate modifier |
| 248 | 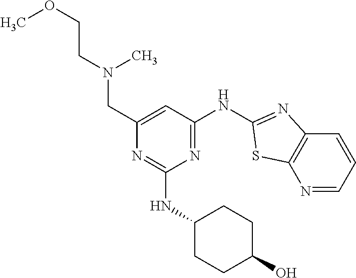 | trans-4-{[4-({methyl[2- (methyloxy)ethyl]amino} methyl)-6-([1,3]thiazolo[5,4- b]pyridin-2-ylamino)-2- pyrimidinyl]amino}cyclohexanol | LCMS (Method B): Rt 0.56 minutes; m/z 444 (MH+) | MDAP, ammonium bicarbonate modifier |
| 249 | 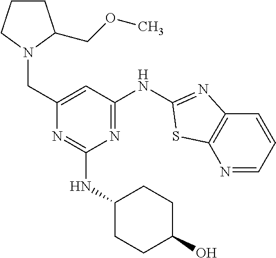 | trans-4-{[4-({2- [(methyloxy)methyl]-1- pyrrolidinyl}methyl)-6- ([1,3]thiazolo[5,4- b]pyridin-2-ylamino)-2- pyrimidinyl]amino}cyclohexanol | LCMS (Method B): Rt 0.59 minutes; m/z 470 (MH+) | MDAP, ammonium bicarbonate modifier |
| 250 | 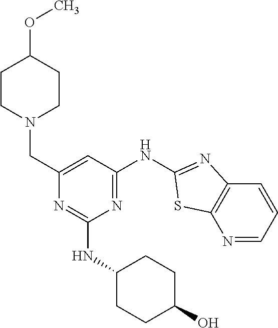 | trans-4-{[4-{[4- (methyloxy)-1- piperidinyl]methyl}-6- ([1,3]thiazolo[5,4- b]pyridin-2-ylamino)-2- pyrimidinyl]amino}cyclohexanol | LCMS (Method B): Rt 0.58 minutes; m/z 470 (MH+) | MDAP, ammonium bicarbonate modifier |
| 251 | 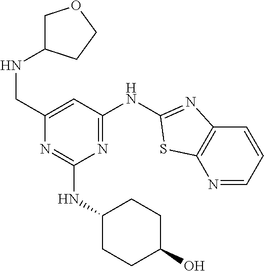 | trans-4-{[4-[(tetrahydro-3- furanylamino)methyl]-6- ([1,3]thiazolo[5,4- b]pyridin-2-ylamino)-2- pyrimidinyl]amino}cyclohexanol | LCMS (Method B): Rt 0.53 minutes; m/z 442 (MH+) | MDAP, ammonium bicarbonate modifier |
| 252 | 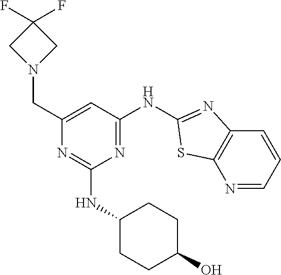 | trans-4-{[4-[(3,3-difluoro- 1-azetidinyl)methyl]-6- ([1,3]thiazolo[5,4- b]pyridin-2-ylamino)-2- pyrimidinyl]amino}cyclohexanol | LCMS (Method B): Rt 0.60 minutes; m/z 448 (MH+) | MDAP, ammonium bicarbonate modifier |
| 253 | 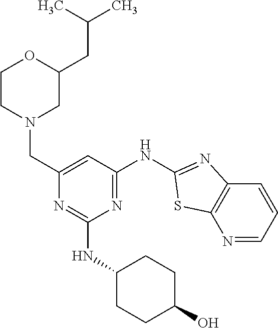 | trans-4-{[4-{[2-(2- methylpropyl)-4- morpholinyl]methyl}-6- ([1,3]thiazolo[5,4- b]pyridin-2-ylamino)-2- pyrimidinyl]amino}cyclohexanol | LCMS (Method B): Rt 0.71 minutes; m/z 498 (MH+) | MDAP, ammonium bicarbonate modifier |
| 254 | 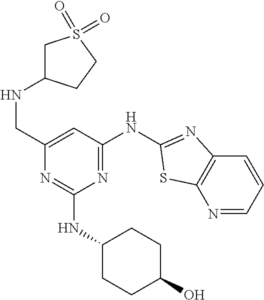 | trans-4-{[4-{[(1,1- dioxidotetrahydro-3- thienyl)amino]methyl}-6- ([1,3]thiazolo[5,4- b]pyridin-2-ylamino)-2- pyrimidinyl]amino}cyclohexanol | LCMS (Method B): Rt 0.54 minutes; m/z 490 (MH+) | MDAP, ammonium bicarbonate modifier |
| 255 | 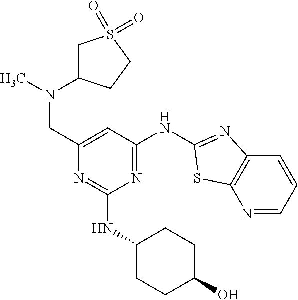 | trans-4-{[4-{[(1,1- dioxidotetrahydro-3- thienyl)(methyl)amino]methyl}- 6-([1,3]thiazolo[5,4- b]pyridin-2-ylamino)-2- pyrimidinyl]amino}cyclohexanol | LCMS (Method B): Rt 0.57 minutes; m/z 504 (MH+) | MDAP, ammonium bicarbonate modifier |
| 256 | 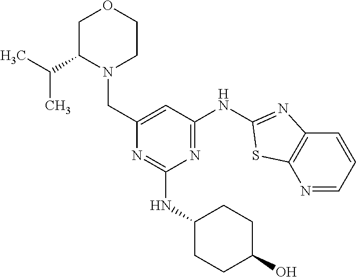 | trans-4-{[4-{[(3R)-3-(1- methylethyl)-4- morpholinyl]methyl}-6- ([1,3]thiazolo[5,4- b]pyridin-2-ylamino)-2- pyrimidinyl]amino}cyclohexanol | LCMS (Method B): Rt 0.63 minutes; m/z 484 (MH+) | MDAP, ammonium bicarbonate modifier |
| 257 | 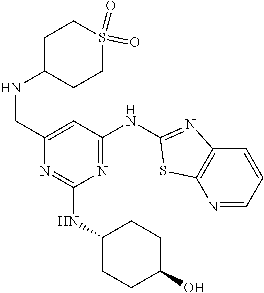 | trans-4-{[4-{[(1,1- dioxidotetrahydro-2H- thiopyran-4- yI)amino]methyl}-6- ([1,3]thiazolo[5,4- b]pyridin-2-ylamino)-2- pyrimidinyl]amino}cyclohexanol | LCMS (Method B): Rt 0.54 minutes; m/z 504 (MH+) | MDAP, ammonium bicarbonate modifier |
| 258 | 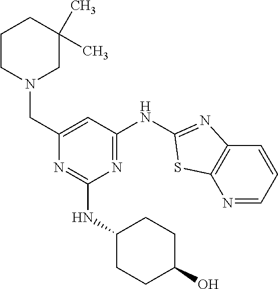 | trans-4-{[4-[(3,3-dimethyl- 1-piperidinyl)methyl]-6- ([1,3]thiazolo[5,4- b]pyridin-2-ylamino)-2- pyrimidinyl]amino}cyclohexanol | LCMS (Method B): Rt 0.66 minutes; m/z 468 (MH+) | MDAP, ammonium bicarbonate modifier |

### Example 259

### trans-4-{[4-(hydroxymethyl)-6-([1,3]thiazolo[5,4-b]pyridin-2-ylamino)-2-pyrimidinyl]amino}cyclohexanol

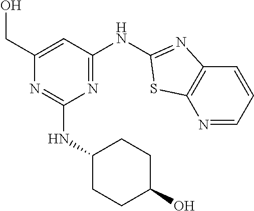

A suspension of 2-[(trans-4-hydroxycyclohexyl)amino]-6-([1,3]thiazolo[5,4-b]pyridin-2-ylamino)-4-pyrimidinecarbaldehyde (40 mg, 0.108 mmol) in tetrahydrofuran (0.4 mL) was treated with bis(2,2,2-trifluoroethyl)amine (0.027 mL, 0.216 mmol) and the mixture was allowed to stir for 15 minutes. The mixture was then treated with dichloromethane (1.6 mL) and then with sodium triacetoxyborohydride (34.3 mg, 0.162 mmol). The reaction was stirred for 3 hours and then treated with saturated aqueous sodium bicarbonate (1 mL) and stirred for 30 minutes. Chloroform (1 mL) and methanol (0.2 mL) were added and the mixture was separated; the organic phase evaporated to dryness and the product was then purified by mass-directed automated preparative HPLC (ammonium bicarbonate modifier) to afford the title compound (8 mg, 0.022 mmol, 20%) LCMS (Method A): Rt 0.51 minutes; m/z 373 (MH+).

### Example 260

### 4-{[2-[(trans-4-hydroxycyclohexyl)amino]-6-([1,3]thiazolo[5,4-b]pyridin-2-ylamino)-4-pyrimidinyl]methyl}-6-methyl-3(2H)-pyridazinone

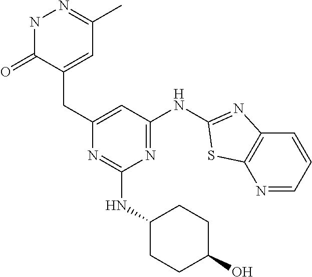

A mixture of 2-[(trans-4-hydroxycyclohexyl)amino]-6-([1,3]thiazolo[5,4-b]pyridin-2-ylamino)-4-pyrimidinecarbaldehyde (150 mg, 0.405 mmol) and powdered potassium hydroxide (68.2 mg, 1.215 mmol) in ethanol (10 mL) was treated with 6-methyl-4,5-dihydro-3(2H)-pyridazinone (49.9 mg, 0.445 mmol) and the mixture was stirred and heated at 60° C. for 8 hours. The mixture was cooled and acidified to pH2 with 2M aqueous hydrochloric acid. The mixture was evaporated to dryness and the product was subjected to purification by mass-directed automated preparative HPLC (ammonium bicarbonate modifier) to afford the title compound (47 mg, 0.101 mmol, 25% yield). LCMS (Method A): Rt 0.55 minutes; m/z 465 (MH+).

### Biological Data

### Itk Homogeneous Time Resolved Fluorescence (HTRF)

The activity of recombinant human Itk (full length) is assessed using an HTRF assay with truncated human SAM-68 (R331-Y443) as the substrate.

Recombinant human Itk (full length) is expressed in insect cells (in pFastBac-1 vector Invitrogen) fused to a Flag tag at its N terminus. The sequence of the Itk part is identical to Genbank entry L10717. The FLAG-Itk fusion protein is extracted from insect cells and purified by immunoaffinity chromatography on anti-FLAG (M2) agarose affinity resin. Further purification is by size exclusion chromatography. Purified protein is stored at −80° C. in Tris-HCl (50 mM), NaCl (200 mM), sorbitol (500 mM), DTT (2 mM), pH 8.0.

Truncated human SAM-68 (R331-Y443) is expressed in E. coli (using a pGex-4T vector Pharmacia) as a GST-thrombin cleavage site-Avi-tag-Sam68:331-443 fusion. The Sam68 part of the fusion (R331-Y443) is identical to the sequence of Genbank database entry NM_{—}006559. GST-SAM68 is purified by affinity chromatography on glutathione-sepharose. Specific biotinylation of the Avitag sequence of GST-SAM68 is performed at room temperature in the presence ofmg:ATP, (5 mM), D-biotin, (1 mM), DTT, (1 mM) and biotin ligase, (1 uM), and is complete in 2 hours. The biotinylated protein is further purified by size exclusion chromatography and stored at −80° C. in Tris-HCl (50 mM), NaCl (250 mM), glycerol (10%), DTT (2 mM), pH 8.0.

Itk (typically 5-50 μM) is pre-activated by incubation with 100 μM ATP and 10 mMmgCl₂ for 30 minutes at room temperature before dilution in assay buffer (50 mM HEPES, 1 mM dithiothreitol, 0.0025% Tween-20, pH7.4) to give a concentration which ensures linearity proportional to time and enzyme concentration (typically a 5 nM final concentration in the assay).

Compounds at various concentrations (typical range 25 pM-25 μM) or DMSO vehicle (at less than 5% final assay concentration) are incubated with 3 μl substrate (final assay concentrations 50 nM biotinylated GST SAM68, 10 mMmgCl₂, 20 μM ATP in 50 mM HEPES, 1 mM DTT, 0.0025% Tween 20, pH7.4). The activated Itk enzyme (3 μl volume) is added to initiate the phosphorylation reaction. Following an incubation at 20° C., (for a time determined to ensure the assay remains in linear initial rate phase, typically 30 minutes), the reaction is halted by adding stop/read reagent (3 μl). The stop/read reagent comprises streptavidin APC (50 nM final assay concentration; Perkin Elmer), europium-anti-phosphotyrosine antibody (0.5 nM final assay concentration; Wallac) diluted in 40 mM HEPES, 150 mM NaCl, 0.03% w/v BSA, 60 mM EDTA. The assay plates are left to equilibrate for at least 45 minutes at 20° C., before reading on a suitable HTRF reader.

The compounds of Examples 1 to 260 were tested in the above or a similar assay and were found to have a mean pKi of 5 or greater.

What is claimed is:

1. A compound of formula (I): 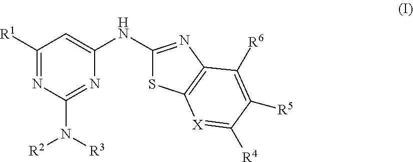
wherein
R¹ is hydrogen, —CR⁷R⁸R⁹, —CH₂OR²⁴, —CH₂NR²⁵R²⁶ or —CH₂-6-membered heteroaryl wherein the 6-membered heteroaryl contains one or two nitrogen atoms and is optionally substituted by one or two substituents independently selected from C₁₋₆alkyl and —OH;
R² is hydrogen or methyl;
R³ is C₁₋₆alkyl substituted by —OH or —NH₂,
C₃₋₆cycloalkyl substituted by C₁₋₆alkyl, —OH, —NR²⁷R²⁸, —CO₂H or —CONH₂,
—(CH₂)ₘ6-membered heterocyclyl wherein the 6-membered heterocyclyl contains one or two heteroatoms independently selected from nitrogen and oxygen and is optionally substituted by —SO₂CH₃ or C₁₋₆alkyl optionally substituted by —CO₂H,
naphthyl substituted by —CO₂H, or
—(CH₂)ₙphenyl wherein the phenyl is substituted by one or two substituents independently selected from −OR¹⁰, —SR¹¹, halo, —CO₂H, —SO₂NHR¹², C₁₋₆alkyl optionally substituted by —OH, —CO₂H or —CONR¹³R¹⁴, C₂₋₆alkenyl optionally substituted by —CO₂H and C₃₋₆cycloalkyl optionally substituted by —CO₂H;
R⁴ is hydrogen, —OR¹⁵, halo, —CF₃, —CN, —NO₂, —NR¹⁶R¹⁷, —CO₂R¹⁸, —SO₂CH₃, —NHSO₂CH₃, C₁₋₆alkyl optionally substituted by —OH, —CN, —CO₂R¹⁹ or —CONH₂, pyridinyl optionally substituted by —OR²⁹, —CH₂NR³⁰R³¹ or —CN, or 5-membered heteroaryl wherein the 5-membered heteroaryl contains one or two heteroatoms independently selected from oxygen and nitrogen and is optionally substituted by C₁₋₆alkyl;
R⁵ and R⁶ are each independently hydrogen or fluoro;
R⁷ and R⁸ are both hydrogen, or R⁷ and R⁸ are both fluoro;
R⁹ is hydrogen, C₁₋₆alkyl, or phenyl optionally substituted by fluoro;
R¹⁰ is hydrogen or C₁₋₆alkyl optionally substituted by —CO₂R²⁰;
R¹¹ is C₁₋₆alkyl optionally substituted by —CO₂H;
R¹² is hydrogen or —COC₁₋₆alkyl;
R¹³ and R¹⁴ are each independently hydrogen or C₁₋₆alkyl optionally substituted by —OH, or
R¹³ and R¹⁴, together with the nitrogen atom to which they are attached, are linked to form a 6-membered heterocyclyl optionally containing an oxygen atom;
R¹⁵, R²¹ and R²² are each independently C₁₋₆alkyl;
R¹⁶ and R¹⁷ are each independently hydrogen, —COR²¹, —CO₂R²², or C₁₋₆alkyl optionally substituted by one or two —OH groups, or R¹⁶ and R¹⁷, together with the nitrogen atom to which they are attached, are linked to form a 4-, 5- or 6-membered heterocyclyl wherein the 4-membered heterocyclyl is optionally substituted by oxo and the 5- or 6-membered heterocyclyl optionally contains an oxygen atom, a sulphur atom or a further nitrogen atom and is optionally substituted by one or two substituents independently selected from oxo, —OH, —NH₂ and C₁₋₆alkyl optionally substituted by —OH or —NH₂;
R¹⁸, R¹⁹, R²⁰, R²⁴, R³², R³³, R³⁶, R³⁷, R³⁸, R³⁹, R⁴⁰, R⁴¹, R⁴² and R⁴³ are each independently hydrogen or C₁₋₆alkyl;
R²³ is hydrogen or halo;
R²⁵ is hydrogen or C₁₋₆alkyl optionally substituted by —OR³² and R²⁶ is C₁₋₈alkyl optionally substituted by —OR³³, —NR³⁴R³⁵ or —CF₃, or 5- or 6-membered heterocyclyl wherein the 5- or 6-membered heterocyclyl contains a heteroatom selected from oxygen, sulphur and nitrogen and is optionally substituted by one or two oxo substituents, or R²⁵ and R²⁶, together with the nitrogen atom to which they are attached, are linked to form a 4-, 5- or 6-membered heterocyclyl wherein the 4-membered heterocyclyl is optionally substituted by one or two substituents independently selected from halo and the 5- or 6-membered heterocyclyl optionally contains an oxygen atom, a sulphur atom or a further nitrogen atom and is optionally substituted by one or two substituents independently selected from oxo, C₁₋₆alkyl optionally substituted by —OR³⁶, halo, —OR³⁷ and —CO₂R³⁸;
R²⁷ and R²⁸ are each hydrogen, or R²⁷ and R²⁸, together with the nitrogen atom to which they are attached, are linked to form a 6-membered heterocyclyl wherein the 6-membered heterocyclyl optionally contains an oxygen atom;
R²⁹ is tetrahydropyran, or C₁₋₆alkyl optionally substituted by —OR³⁹ or —NR⁴⁰R⁴¹;
R³⁰ is hydrogen and R³¹ is C₁₋₆alkyl optionally substituted by —OR⁴², or R³⁰ and R³¹, together with the nitrogen atom to which they are attached, are linked to form a 6-membered heterocyclyl wherein the 6-membered heterocyclyl optionally contains an oxygen atom or a further nitrogen atom and is optionally substituted by one or two substituents independently selected from C₁₋₆alkyl;
R³⁴ is hydrogen or C₁₋₆alkyl and R³⁵ is —CO₂R⁴³, or R³⁴ and R³⁵, together with the nitrogen atom to which they are attached, are linked to form a 5- or 6-membered heterocyclyl wherein the 5- or 6-membered heterocyclyl optionally contains an oxygen atom or a sulphur atom and is optionally substituted by one or two oxo substituents;
X is —N— or —CR²³—;
and
m and n are each independently 0, 1, 2 or 3;
or a salt thereof.

2. A compound according to claim 1, or a salt thereof, wherein R¹ is —CR⁷R⁸R⁹ or —CH₂NR²⁵R²⁶.

3. A compound according to claim 1, or a salt thereof, wherein R² is hydrogen.

4. A compound according to claim 1 or a salt thereof, wherein R³ is C₁₋₆alkyl substituted by —OH, C₃₋₆cycloalkyl substituted by —OH or —CO₂H, or —(CH₂)ₙphenyl wherein the phenyl is substituted by one or two substituents independently selected from −OR¹⁰, halo, —SO₂NHR¹² and C₁₋₆alkyl optionally substituted by —CO₂H.

5. A compound according to claim 1 or a salt thereof, wherein R⁴ is —NR¹⁶R¹⁷, -pyridinyl optionally substituted by —OR²⁹, —CH₂NR³⁰R³¹ or —CN, or 5-membered heteroaryl wherein the 5-membered heteroaryl contains one or two heteroatoms independently selected from oxygen and nitrogen and is optionally substituted by C₁₋₆alkyl.

6. A compound according to claim 1 or a salt thereof, wherein R⁵ is hydrogen.

7. A compound according to claim 1 or a salt thereof, wherein R⁶ is hydrogen.

8. A compound substantially as described in any one of Examples 1 to 260, or a salt thereof.

9. A compound which is:
trans-4-{[4-{[5-(methyloxy)[1,3]thiazolo[5,4-b]pyridin-2-yl]amino}-6-(phenylmethyl)-2-pyrimidinyl]amino}cyclohexanol;
trans-4-{[4-(phenylmethyl)-6-([1,3]thiazolo[5,4-b]pyridin-2-ylamino)-2-pyrimidinyl]amino}cyclohexanol;
trans-4-{[4-[(6-nitro-1,3-benzothiazol-2-yl)amino]-6-(phenylmethyl)-2-pyrimidinyl]amino}cyclohexanol;
(2-{[2-[(trans-4-hydroxycyclohexyl)amino]-6-(phenylmethyl)-4-pyrimidinyl]amino}-1,3-benzothiazol-6-yl)acetonitrile;
5-{[4-{[5-(methyloxy)[1,3]thiazolo[5,4-b]pyridin-2-yl]amino}-6-(phenylmethyl)-2-pyrimidinyl]amino}-1-pentanol;
trans-4-{[4-[(5-ethyl[1,3]thiazolo[5,4-b]pyridin-2-yl)amino]-6-(phenylmethyl)-2-pyrimidinyl]amino}cyclohexanol;
2-{[2-[(trans-4-hydroxycyclohexyl)amino]-6-(phenylmethyl)-4-pyrimidinyl]amino}-1,3-benzothiazole-6-carbonitrile;
3-(4-{[4-[(5-ethyl[1,3]thiazolo[5,4-b]pyridin-2-yl)amino]-6-(phenylmethyl)-2-pyrimidinyl]amino}phenyl)propanoic acid;
3-(2-{[2-[(trans-4-hydroxycyclohexyl)amino]-6-(phenylmethyl)-4-pyrimidinyl]amino}-1,3-benzothiazol-6-yl)propanamide;
trans-4-{[4-{[6-(2-hydroxyethyl)-1,3-benzothiazol-2-yl]amino}-6-(phenylmethyl)-2-pyrimidinyl]amino}cyclohexanol;
trans-4-{[4-{[6-(hydroxymethyl)-1,3-benzothiazol-2-yl]amino}-6-(phenylmethyl)-2-pyrimidinyl]amino}cyclohexanol;
1-(2-{[2-[(trans-4-hydroxycyclohexyl)amino]-6-(phenylmethyl)-4-pyrimidinyl]amino}-1,3-benzothiazol-6-yl)-2-azetidinone;
1-(2-{[2-[(trans-4-hydroxycyclohexyl)amino]-6-(phenylmethyl)-4-pyrimidinyl]amino}-1,3-benzothiazol-6-yl)-2-pyrrolidinone;
1-(2-{[2-[(trans-4-hydroxycyclohexyl)amino]-6-(phenylmethyl)-4-pyrimidinyl]amino}-1,3-benzothiazol-6-yl)-2,5-pyrrolidinedione;
3-(2-{[2-[(trans-4-hydroxycyclohexyl)amino]-6-(phenylmethyl)-4-pyrimidinyl]amino}-1,3-benzothiazol-6-yl)-2,4-imidazolidinedione;
3-(2-{[2-[(trans-4-hydroxycyclohexyl)amino]-6-(phenylmethyl)-4-pyrimidinyl]amino}-1,3-benzothiazol-6-yl)-1,3-oxazolidin-2-one;
3-[(2-{[2-[(trans-4-hydroxycyclohexyl)amino]-6-(phenylmethyl)-4-pyrimidinyl]amino}[1,3]thiazolo[5,4-b]pyridin-5-yl)(methyl)amino]-1,2-propanediol; or
a salt thereof.

10. A compound according to claim 1 in the form of a pharmaceutically acceptable salt thereof.

11. A pharmaceutical composition comprising a compound as defined in claim 1, or a pharmaceutically acceptable salt thereof, and one or more pharmaceutically acceptable excipients.

12-14. (canceled)

15. A method of treating a disorder mediated by inappropriate Itk activity comprising administering a safe and effective amount of a compound as defined in claim 1, or a pharmaceutically acceptable salt thereof, to a patient in need thereof.

16. A method according to claim 15 wherein the disorder mediated by inappropriate Itk activity is a respiratory disease; an allergic disease; an autoimmune disease; transplant rejection; graft versus host disease; an inflammatory disorder; HIV; aplastic anemia; or pain.

17. A method according to claim 15 wherein the disorder mediated by inappropriate Itk activity is asthma, chronic obstructive pulmonary disease (COPD), bronchitis, allergic rhinitis, atopic dermatitis, rheumatoid arthritis, multiple sclerosis, psoriasis, type I diabetes, T cell mediated hypersensitivity, Guillain-Barre Syndrome, Hashimoto's thyroiditis, transplant rejection, graft versus host disease, conjunctivitis, contact dermatitis, inflammatory bowel disease, chronic inflammation, HIV, aplastic anemia, or inflammatory pain.

18. A method according to claim 15 wherein the disorder mediated by inappropriate Itk activity is asthma.
